(19) United States
(12) Patent Application Publication
(10) Pub. No.: US 2022/0033851 A1
(43) Pub. Date: Feb. 3, 2022

(54) mRNA, episomal and genomic integrated lentiviral and gammaretroviral vector expression of dimeric immunoglobulin A and polymeric immunoglobulin A to Enable Mucosal and Hematological Based Immunity/Protection via Gene Therapy for Allergens, viruses, HIV, bacteria, pneumonia, infections, pathology associated proteins, systemic pathologies, cancer, toxins and unnatural viruses. CAR engineered and non-CAR engineered immune cell expression of dimeric immunoglobulin A and polymeric immunoglobulin A.

(71) Applicant: Roger B. Swartz, Clifton Park, NY (US)

(72) Inventor: Roger B. Swartz, Clifton Park, NY (US)

(21) Appl. No.: 17/368,957

(22) Filed: Jul. 7, 2021

Related U.S. Application Data
(60) Provisional application No. 63/060,300, filed on Aug. 3, 2020.
(60) Provisional application No. 63/076,527, filed on Sep. 10, 2020.
(60) Provisional application No. 63/214,612, filed on Jun. 24, 2021.

(51) Int. Cl. C12N 15/86; C12N 7/00; C07K 14/705; C07K 14/725; C07K 16/12; C07K 16/30; C07K 16/10

(52) U.S. Cl. CPC C12N 15/86; C12N 7/00; C07K 14/70521; C07K 14/70517; C07K 14/70578; A61K 2039/54; C07K 16/1275; C07K 16/30; C07K 16/1045; C12N 2740/15043; C12N 2750/14143; C07K 14/7051

(57) ABSTRACT

The present invention contemplates mRNA, episomal and retroviral genomic gene therapy based short-term, intermediate or long-term vaccine, immunization, immune protection or cancer—that can also be administered as a retroviral genomic gene therapy both in vivo and ex vivo—method to provide epithelial and hematological protection to humans to protect against cancer especially carcinomas, pandemic and non-pandemic viruses, bacterial infections, allergens or the cause of allergic reactions, systemic pathological conditions, cancer and anti-biowarfare agents (e.g. natural and unnatural viruses and toxins) where mucosal immunity and for some diseases hematological immunity is achieved through mRNA, episomal or genomic integrated lentiviral and gammaretroviral vector expression of dimeric immunoglobulin A1 (dIgA1), dimeric immunoglobulin A2 (dIgA2) and engineered variants. Additionally, in some embodiments a method to agglutinate cancers including carcinomas and hematological cancers to prevent metastasis with polymeric immunoglobulin A and dimeric immunoglobulin A and engineered variants. The present invention provides methods, immunoglobulin compositions and vector constructs to express potent immunoglobulins that are derived from human blood of a human currently infected with, affected by, exposed to or recovered from any of a wide range of allergens or the cause of allergic reactions, pathogens (including, viruses, virus mutants, bacterial infections and fungi) and systemic pathological ailments (including cancer and other disorders), developed from phage display technology or mice or other non-human vertebrates with engineered immune systems or humanized immune systems, transgenic mice or chimeric antibodies a fusion of non-human vertebrates (e.g. mouse or rabbit), mouse antibody V-regions, human antibodies. The immunoglobulin compositions include the heavy chain variable, diversity and joining (VDJ or Variable Heavy Region genes) segment immunoglobulin DNA and/or polypeptide sequence from humans identified to have therapeutically relevant affinity immunoglobulins against the antigen, protein or proteins of interest and either to use the exact immunoglobulin heavy chain and light chain polypeptide sequences identified from the B-cell that produced them or to modify or engineer some of the immunoglobulin heavy chain and light chain constant domains to modulate effector functions. Although, ideally there are no changes made to the immunoglobulins light and heavy chains as identified from the B-cell that produced them. Modifications may occur at the Hinge region, Constant Heavy 2 (C_{H}2) domain and Constant Heavy 3 (C_{H}3) domain for the immunoglobulin heavy chain polypeptide with possible modification or change of Constant Heavy 1 (C_{H}1), possible modification or change constant light (C_{L}) chain domain. The resulting antibodies can either be used as a monoclonal or antibody cocktail of (Immunoglobulin Class G subclass1) IgG1, IgG2, IgG3 and other subclasses, IgA1 monomer and IgA2 monomer and dimeric IgA1 (dIgA1) and dimeric IgA2. Immunoglobulins are coded for as necessary to represent the binding affinity (e.g. such as based on complementarity determining Regions (CDRs) or V-regions) in the monoclonal or antibody cocktail). Alternatively, combinatorial libraries of single chain variable fragments (scF_{V}) will generated from human B-cells or other animal B-cells that may or may not have been exposed to the allergen, pathogen, cancer, or pathological ailment, or suspected or identified biowarfare agent or protein where phage display technology and mutagenesis can be used to identify potent VH and V_{L} immunoglobulin fragments that can be incorporated into full-length immunoglobulin heavy and light chains and even reduced length immunoglobulin heavy chains incorporated into vectors for mRNA expression, episomal expression or retroviral gene delivery (retroviral insertion into genomic DNA) based gene-therapy. Further, mice or other animals can also achieve humanized immune system by implanting human hematopoietic progenitor cells into the animal or transplanting human thymus, liver and bone marrow into mice. Additionally, transgenic mice where human immunoglobulin (Ig) genes are inserted into the genome to replacing the endogenous Ig genes making the mice or other non-human vertebrate such as rabbits or hamsters capable of producing fully human antibodies from exposure to antigen may be used to identify potent immunoglobulins. Non-human vertebrates (e.g., mouse or rabbit) may be used to identify potent immunoglobulin binding regions or potent immunoglobulin complementarity determining regions (CDRs) for fusion with human antibodies giving rise to chimeric antibodies. The identified immunoglobulins from these methods will optionally be further optimized through mutagenesis techniques and will be expressed in the recipient via mRNA, via an episome or via retroviral insertion into their genomic DNA of the cells of interest to be expressed via intramuscular administration, intravenous administration, endoscopy based administration to the lamina propria of the stomach and/or small intestine or even the lung, via ingestion or administration proximal to lymph nodes or as an ex vivo administration into any of B-cells, T-cells, Natural Killer (NK) Cells and other immune cell types. Preferred cells to target to receive the vector include muscle cells, liver cells especially hepatocytes and B-cells including memory B-cells, Germinal Center B-cells, memory plasma B-cells (also referred to as a long-lived plasma cell), naïve B-cells, NK cells, T-cells, including chimeric antigen receptor T-cells (CAR T-cells) as well as any CAR engineered immune cell. Additionally, the vector may encode for both the CAR and the polymeric and dimeric immunoglobulin in a single vector construct. In cases where the CAR engineered immune cell is selected to receive the polymeric immunogloublin A and dIgA encoding vector the retrovirus may optionally be pseudotyped with a protein that is anti to the CAR single chain variable fragment (scFv) such that conditional transduction occurs only on CAR engineered cells. The vector will be ideally delivered as a naked vector, in a vesicle based delivery system such as a lipid nano-particle, in a recombinant Adeno Associated Virus (rAAV) with preference for AAV serotype 8 (AAV8) containing a single-stranded Deoxyribonucleic acid (ssDNA), an adenovirus delivery system, a lentivirus delivery system, gammaretroviral delivery system, lentiviral mRNA delivery via mutated reverse transcriptase protein, gammaretroviral mRNA delivery via mutated reverse transcriptase protein, lentiviral retroviral vector, gammaretroviral vector or episomal delivery via mutated integrase protein, or a vesicle-based delivery system using mRNA, single-stranded DNA or double-stranded DNA. When designing an mRNA, AAV viral vector, adenovirus vector, integration deficient lentivirus retroviral vector or gammaretroviral vector, integration deficient lentivirus retroviral vector or gammaretroviral vector, encoding for dIgA1, dIgA2 or polymeric immunoglobulin A a single vector will code for the entire immunoglobulin and J Chain (Joining Chain) expression for dIgA1 or dIgA2, where expression may occur with a single start codon and stop codon for each transgene and in some embodiments a second start codon for J Chain expression. The use of a single start and stop codon is enabled by placing in the 5′ to 3′ direction a furin cleavage site concomitantly followed by a 2A self-processing peptide or furin cleavage site between each gene of any number of consecutive transgenes as a single open reading frame. The specific DNA of the human donor can be identified as follows: Cluster of Differentiation 27+ (CD27+) IgG+ and CD27+ IgA+ memory B-cells, other memory B-cells, or plasmablast B-cells, germinal center B-cells, and even potentially memory plasma B-cells (also referred to as a long-lived plasma cell) will be isolated from blood using established methods. Each resulting isotype of memory B-cell or together will be subjected to a competitive binding assay using magnetic pull down and Fluorescence Activated Cell Sorting (FACS) methods to identify the memory B-cells with therapeutically relevant binding affinity to the virus, bacteria, antigen, allergens, self-antigen, pathogenic protein, or other foreign and non-foreign bodies and proteins of interest.

### REFERENCE TO AN ELECTRONIC SEQUENCE LISTING

In accordance with 37 CFR 1.52(e)(5) on Sep. 27, 2021 the ASCII text filed named “Roger_B_Swartz_Sequence_listing_17_368_957_ST25.txt” was uploaded electronically to the EFS-web. The file was created on Sep. 25, 2021 and the size of the file is 93,401 bytes (94 KB on Disk). The contents of file named “Roger_B_Swartz_Sequence_listing_17_368_957_ST25.txt” containing the sequence listing is herein incorporated by reference in its entirety.”

### BACKGROUND OF THE INVENTION

### 1. Field of the Invention

The present invention is in the field of healthcare biotechnology and pertains particularly to a therapeutic gene therapy engendering mRNA, episomal or genomic expression via lentiviral vector or gammaretroviral vector integration into genomic DNA of dimeric immunoglobulin A1 (dIgA1) and polymeric immunoglobulin A1, dimeric immunoglobulin A2 (dIgA2) and polymeric immunoglobulin A2, and engineered variants against Cancer, Viruses, Allergens, Fungi, HIV, H. pylori other bacterial infections, systemic pathologies, IgE, cytokines, cancerous tumors or as anti-biowarfare protection. This invention includes a method to encode for dIgA1 (inclusive of polymeric immunoglobulin A1), dIgA2 (inclusive of polymeric immunoglobulin A2) and engineered variants (such as dimeric immunoglobulin A with modified hinge lengths or other modified constant regions with restrictions placed on CH3 so that the cysteine bond and other structural interactions with J Chain are not disrupted) on a vector as a means to deliver a gene therapy to humans and non-human vertebrates. Additionally, polymeric and dimeric immunoglobulin A (A1 or A2) encoded for on a single vector or multiple vectors, co-expressing a Chimeric Antigen Receptor (CAR) is part of the invention. Dimeric immunoglobulin A and other polymeric higher valency forms can be encoded for on a single DNA or RNA vector with the use of a furin cleavage site (SEQ ID NOs: 10, 12, 13 and 14) and 2A self-processing peptide (SEQ ID NOs: 15, 16, 17 and 19) between consecutive transgenes. The transgenes that make up dIgA (inclusive of polymeric immunoglobulin A) include (1) the immunoglobulin heavy chain, (2) the immunoglobulin light chain and (3) J Chain. These three proteins may be encoded for a on a single genetic element in one, two or even three open reading frames. Where all the transgenes in any one open reading frame will have in the 5″ to 3′ direction a furin cleavage site followed by a 2A self-processing peptide placed between any two adjacent and consecutive transgenes. The use of a furin cleavage site directly followed by a 2A self-processing peptide allows for efficient cleavage points between the consecutive transgenes completely removing any trace of the 2A self-processing peptide in the final immunoglobulin. In one embodiment MZB1 may also be encoded for by the vector. In another embodiment mRNA encodes for dIgA1, dIgA2 or engineered variants over one or more mRNA vectors. In an alternative configuration, a 2A self-processing peptide will be used between consecutive transgenes in a single open reading frame in the absence of the furin cleavage site leaving the 2A residue on the C-terminal end of the protein encoded for by the gene that is directly 5′ of the 2A gene. In another configuration mRNA encodes for polymeric immunoglobulin A and dIgA or dIgA over one or more mRNA vectors. In another configuration dimeric single chain variable Fragment-Fragment crystallizable immunoglobulin A Fusion (dscFV-FcIgA) is considered (see FIG. 44) which is a fusion of a single chain variable fragment (scFv) and Fragment crystallizable (Fc) region of the immunoglobulin that includes the hinge, the C_{H}2 and C_{H}3 domains where the single chain variable fragment-fragment crystallizable immunoglobulin A (scFv-FcIgA) is dimerized while forming higher valency polymeric-scFv-FcIgA via the immunoglobulin J Chain.

In the invention one may treat polymeric immunoglobulin A—a mixture of dimeric immunoglobulin A, trimeric immunoglobulin A, tetrameric immunoglobulin A and pentameric immunoglobulin—similar to dIgA where the class and subclass is A1 or A2. Although, this patent's concern with polymeric Immunoglobulin A is more focused but not exclusively focused on its co-expression via CAR engineered immune cells and its expression in non-CAR engineered NK cells both that are contemplated to prevent cancer metastasis in carcinomas and hematological malignancies, as well as the expression of polymeric IgA from any cell for the prevention of metastasis in hematological malignancies and in some embodiments the prevention of metastasis in carcinomas. Although, this does not preclude the use of polymeric immunoglobulin A for non-cancer applications. And further it is understood that any mention of producing dIgA1 may include producing at least a minor stoichiometric fraction of polymeric immunoglobulin A1 and any mention of producing dIgA2 may include producing some or a significant if not a major stoichiometric fraction of polymeric immunoglobulin A2. In the invention dIgA1, dIgA2 and engineered variants specific against Viruses, Allergens, Fungi, HIV, H. pylori other bacterial infections, systemic pathologies, IgE, cytokines, cancerous tumors, the prevention of cancer metastasis or as anti-biowarfare protection may have such binding affinity of dIgA and engineered variants based on one or more of individuals affected by such ailments, developed by Phage Display Technology with mutagenesis techniques, discovered and optimized from mice or other animals with humanized immune systems, derived from transgenic mice or derived non-human vertebrate (e.g. mouse, hamster or rabbit) intended for fusion with human antibodies giving rise to chimeric antibodies and in some embodiments the mouse developed antibody.

For immunoglobulin encoded vectors the entire immunoglobulin genetic sequence would be derived or identified from one of several sources including plasmablasts, CD27+ memory B-cells—expressing immunoglobulins with binding affinities specific for an antigen, virus antigen, allergen, foreign protein, pathogenic protein or cancer related protein—from humans that have been exposed to such antigens, allergens or live with such ailments, mice or other animals with a humanized immune systems, non-human vertebrates intended for chimeric antibodies, transgenic mice or combinatorial libraries that may use phage display technology and mutagenesis. Those B-cells would be CD27+ or other CD+ IgG+ and IgA+ memory B-cells, germinal center B-cells, or plasmablasts B-cells that bear cell surface immunoglobulins easily allowing for their isolation based on binding affinity for an antigen, allergen, foreign protein, native protein, or pathogen related protein of interest through competitive binding cell sorting methods such as flow cytometry or fluorescence activated cell sorting (FACS) or competitive binding magnetic pull down methods that incorporate magnetic beads biotinylated to an antigen, allergen or foreign protein of interest and a competing protein for the antigen. Plasmids may be created that encode for those immunoglobulins to be further evaluated in addition to subjecting them to targeted mutagenesis. Alternatively, combinatorial libraries may be generated, and phage display technology can be used to identify potent single chain variable fragments (scF_{V}) that can be used to identify potent V_{H} and V_{L} pairs of high avidity and binding affinity that can be incorporated into a plasmid expressing a full-length immunoglobulin heavy and light chains for evaluation. Other models such as non-human vertebrates (e.g. mouse or rabbit) intended for chimeric antibodies, transgenic mice or mice or other animals with humanized immune systems may be used to identify potent immunoglobulins or potent binding regions. Additionally, the V-region DNA of the heavy (IgH) and light chain (IgL) of the immunoglobulins will be incorporated into a vector construct that could use either the constant region DNA identified in humanized or transgenic mice, filamentous phage or B-cell expressing the identified immunoglobulin of interest or another source of genetic information that may be engineered may replace all or part of the constant region DNA that may also include isotype switching of C_{H}1 or C_{H}2 but not C_{H}3 constant region genetic information, mixes of two constant regions from two isotypes. The mRNA, episome, or retroviral incorporation of the DNA via lentivirus or gammaretrovirus delivery system may be delivered to B-cells, muscle cells, liver cells (such as hepatocytes), T-cells including chimeric antigen receptor T-cells (CAR T-cells), CAR immune cells, NK cells, any human cell, endoscopy-based delivery to the lamina propria of the lungs, stomach and/or small intestine destined for memory B-cells in the lamina propria and the supporting lymph nodes as well as other quiescent cells potentially. B-cells targeted to receive the gene therapy may include memory B-cells, Germinal Center B-cells, memory plasma B-cells (also referred to as a long-lived plasma cell), a plasma blast, and naïve B-cells. T cells including chimeric antigen receptor T-cells (CAR T-cells), NK cells including CAR NK cells and non-CAR NK cells as well as other CAR engineered immune cells may also receive the DNA vector both as an episome or as retroviral insertion into genomic DNA (See FIGS. 37, 41, 43 and 46). Anti CAR (that is specific to the scFv on the CAR) pseudotyped retroviruses containing vectors encoding for dIgA can selectively transduce CAR engineered immune cells among non-CAR engineered immune cells. In some embodiments retroviruses will encode for both the CAR and dIgA in a single vector. In other embodiments NK cells, dendritic cells, any T-cell and B-cells are engineered with both a CAR and dIgA either sequentially of with a single vector encoding for both the CAR and dIgA. In one embodiment a dimeric single chain variable Fragment-Fragment crystallizable immunoglobulin A Fusion (dscFV-FcIgA) is considered (see FIG. 44) as well as polymeric-scFv-FcIgA which is a fusion of a single Chain Variable Fragment (scFv) and Fragment crystallizable (Fc) region of the immunoglobulin that includes the hinge, the C_{H}2 and C_{H}3 domains where the single chain variable fragment-fragment crystallizable immunoglobulin A (scFv-FcIgA) is dimerized via the immunoglobulin J Chain. When dIgA1 (inclusive of polymeric immunoglobulin A1) or dIgA2 (inclusive of polymeric immunoglobulin A2) and a Chimeric Antigen Receptor (CAR) or dscFV-FcIgA and the CAR are encoded for in a single vector the use of one or more open reading frames may be used where when two reading frames are used an internal ribosome entry site (IRES) may be used in to separate any one or more proteins or protein fusions encoded for in the vector. When episomes are used in the gene therapy, the episome may be delivered to the cells of interest via an adeno-associated virus, adenovirus, lentivirus-based deliver system, gammaretrovirus-based delivery system, or via a vesicle-based delivery system such as a lipid nano particle as one of many examples. Additionally, in other embodiments the reinvigoration of an FDA approved antibody or the repurposing of antibodies by placing their V-regions in a vector encoding for dIgA and engineered variants as well as polymeric immunoglobulin A is part of the present invention.

### 1. Discussion of the State of the Art

The primary mode of infection, carcinogen exposure and allergen entry for the vast majority of carcinogens, allergens, pathogens such as viruses, bacteria affecting the lungs, including gut bacteria, viruses and other disease-causing foreign bodies is through our mucosal barriers and exocrine channels. Similarly, the primary way that allergens such as fungi, dust mite feces, peanuts, tree nuts and pollen cause allergic reactions is also through our mucosal barriers especially the respiratory tract and in some cases the digestive tracts. Cancer has a specific term for cancers affecting the mucosal or epithelial and exocrine barriers referred to as carcinomas. Carcinomas make up 80%-90% of all cancer diagnosis. Almost, the entirety of our body that is not covered in skin is covered in a mucus barrier and is referred to as the mucosa. Thus, it is safe to say that if mankind had effective means of mucosal protection against infectious agents, allergens or bio-warfare agents that their transmission and thus their allergenic and pathogenic consequences will be nearly eliminated. Our mucosal barriers include our respiratory tract, that is the entirety of the pathways where air flows to the alveoli in the lungs, our digestive tract where food and liquids flow and are absorbed and our reproductive tract, our urinary tract, our skin and breasts. In order for an individual's immune system to effectively prevent infection from foreign bodies and also avert an allergic reaction to foreign bodies mucosal immunity via immunoglobulins is required. Secretory immunoglobulin A (SIgA1 and SIgA2) is by far the most prevalent of antibodies in the human body and represents a major mode of defense in mucosal immunity. SIgA1 is made from dIgA1 and other polymeric forms of immunoglobulin A1 that may be present such as trimeric immunoglobulin A1, tetrameric immunoglobulin A1 and pentameric immunoglobulin A1.

Chimeric Antigen Receptor (CAR) engineered immune cells including T-cells, NK cells and other CAR engineered immune cells represent a relatively new field in the area of cancer therapy. Additionally, activated NK cells that are not engineered with a CAR are also effective in the area of cancer therapy. A 2^{nd} generation CAR is a receptor that is an engineered single polypeptide combination—in the N-terminal to C-terminal direction—of a single chain variable fragment (scFV) using the immunoglobulin Variable Heavy (V_{H}) and Variable Light (V_{L}) regions and linking them together with a flexible linker where the scFv is able to communicate binding activity through a hinge derived from (Cluster of differentiation 8 alpha) CD8α or (Cluster of differentiation 28) CD28 followed by a transmembrane domain derived from CD8α or CD28, a co-stimulatory domain such as from (Cluster of Differentiation 137) 4-1BB or CD28 and a (Cluster of Differentiation 3 zeta) CD3ζ signaling domain. The two signaling domains are required in order for the CAR engineered cell to achieve the right level of cytotoxicity upon detecting the cancer. The CAR technology has shown significant efficacy in the ex vivo gene therapy of immune cells for the purpose of homing in on cancer and being activated and expanded in numbers and cytotoxic activity upon detection of cancer providing site specific delivery of chemokines and cytokines such as but not limited to perforin and granzyme A and B, interleukin-(IL-2), tumor necrosis factor alpha (TNFα) and interferon gamma (IFNγ).

Although, FDA approved CAR engineered cells are focused on hematological malignancies rather than solid tumors. Significant efforts are undertaken to fully exploit the anti-tumor effects of CAR T-cells and CAR NK cells but failure to achieve primary clinical end points for the treatment of solid tumors are common. Further CAR engineered cells are somewhat limited to hematological based cancer rather than cancer occurring in proximity or within to the mucosa or epithelium. Although, a significant benefit of the CAR technology is the ability of CAR engineered cells to localize their cytotoxic effects thereby minimizing damage to healthy tissue. The major challenges associated with tumors is that they metastasize, and metastasis is the leading cause of cancer death. Further, carcinomas represent 80-90% of all cancer cases. Thus, despite great promise in the CAR technology most of cancer remains unaddressed by the CAR technology. In 2016 a report disclosed a CAR T-cell that was engineered to co express a CAR and also an anti PD-L1 single chain Fragment Variable-Fragment Crystallizable immunoglobulin class G (scFV-FcIgG) that blocked the PD-L1 ligand on the cancer cells from engaging the CAR T-cell thereby preventing exhaustion. The authors reported that the CAR T-cell showed progress in mitigating solid tumors in mice. (See, e.g. Suarez, E. R., Chang, d., Sun, J., Sui, J., Freeman, G. J., Signoretti, S., Zhu, Q., & Marasco, W. A., 2016, Oncotarget, 7:34341-34355. https://doi.org/10.18632/oncotarget.9114. Also, see U.S. Pat. Publication No. US 2017/0362297 A1). Although, neither the report nor the patent application speaks of a mechanism to prevent metastasis that is the primary cause of cancer death. Nature evolved dimeric immunoglobulin A (dIgA) and polymeric immunoglobulin A to agglutinate and immobilize pathogens at the epithelium including at the mucosa and exocrine channels and dIgA, polymeric immunoglobulin A and SIgA specific to an upregulated protein expressed on tumors can agglutinate and immobilize carcinomas including solid tumors to stop metastasis. Further, dIgA is actively transported across the epithelium and can reach cancer of both faces of a carcinoma whereas other IgG immunoglobulins must rely on passive transport across the epithelium limiting their numbers on the face of the tumor facing into the lumen or exocrine channel (the apical face of the epithelium). This patent contemplates the engineering of a T-cell, NK cell, or other immune cell to express both the CAR and dIgA in a single retroviral vector construct (See, FIG. 41). Additionally, this patent contemplates the conditional engineering of CAR immune cells with an anti-CAR scFv pseudotyped retrovirus containing a vector encoding for dIgA (See FIG. 37). Thereby making the presence of the CAR on the engineered cell required for lentiviral or gammaretroviral transduction of the dIgA encoded vector to be integrated with the host genome or function as an episome. Conditionally engineering CAR immune cells with an anti-CAR scFv (that is specific to the scFv on the CAR) pseudotyped retrovirus containing a vector encoding for dIgA ensures that other present immune cells that are not engineered with a CAR do not get traversed by the retrovirus that would otherwise have resulted in the upregulation of dIgA upon the unintended activation of those immune cells without a CAR. Further, this patent contemplates incorporating into vector constructs encoding for dIgA the use of endogenous and abbreviated endogenous promoters for cytokines whose expression is upregulated upon activation of the CAR and non-CAR expressing cell so that dIgA secretion of the non-CAR immune cell or CAR engineered immune cell is more closely coupled with NK cell or CAR activation.

As a means to address cancer and potentially a gene therapy that encodes for immunoglobulins as a single gene element, researchers have developed single chain fragment variable-fragment crystallizable fusions of immunoglobulin class G (scFV-FcIgG). Although, such fusions while potentially effective against cancer and promoting antibody mediated cellular cytotoxicity these scFV-FcIgG fusion antibodies cannot traverse the epithelium via active transport by pIgR. Thus, in many instances they will not be able to achieve concentrations at high levels on both faces of a carcinoma. Thus, the face of the carcinoma facing into the lumen or exocrine duct can metastasize and spread the cancer. Additionally, these scFV-FcIgG fusions nor IgG antibodies do not have properties associated with agglutination. Thus, they are unlikely to inhibit metastasis and there no evidence nor reports that they exhibit such anti-metastasis properties that polymeric immunoglobulin A and dimeric immunoglobulin A exhibit.

Potent IgA1 s and IgA2s as well as IgA variants such as IgAs with varying hinge lengths or modifications to their constant regions can be identified from CD27+ or other CD+ IgA memory B-cells, the V regions of CD27+ or other CD+ IgG memory B cells, other CD memory B-cells, plasmablasts B-cells, germinal center B-cells from mice or other non-human vertebrates with engineered immune systems or humanized immune systems, from mice or non-human vertebrates B-cells or may have their binding regions discovered via phage display technology such as in the single chain variable fragment or may be discovered from mice or other animals with humanized immune systems.

Expression of dIgA1 and dIgA2 and derivatives of dIgA1 and dIgA2 from mRNA, an episome or genomic DNA via retroviral incorporation of retroviral vector such as one delivered by a lentivirus or gammaretrovirus is claimed in this instant patent. The conversion of dIgA (dIgA1 and dIgA2) and polymeric immunoglobulin A (A1 and A2) to secretory immunoglobulin A (SIgA1 and SIgA2 respectively) is a natural process that is part of active transport to the mucosa in humans and is described in FIG. 2. Thus, mucosal immunity depends on two events the production of dIgA (inclusive of polymeric immunoglobulin A) its conversation to SIgA. Further, it is mucosal immunity provided by SIgA is required to greatly reduce the probability of infection and allergic reactions from foreign bodies. It is mucosal immunity provided by SIgA that is required to prevent foreign bodies from crossing the epithelial barriers that line nearly all of the mucosa. It is mucosal immunity provided by SIgA that can effectively prevent humans that get exposed to an infectious agent from passing it on to others. It is mucosal immunity provided by SIgA can eliminate the possibility of antibody-dependent enhancement of infection (ADE) and cytokine storm. In other words, to stop the spread and harmful effects of pathogens, cancers, bacteria, infectious agents and to protect against on all members of society including those at-risk mucosal immunity by SIgA is required. Similarly, to stop the harmful effects of allergens especially as related to Type 1 Hypersensitivity on those capable of allergic reactions mucosal immunity by SIgA is required. When at the basolateral face of epithelial cells with pIgR receptors dIgA or polymeric immunoglobulin A is actively transported to the mucosa via complexing with polymeric immunoglobulin secreting receptor (pIgR) where upon reaching the apical face of the epithelial cell pIgR is cleaved and releases secretory component onto dIgA or polymeric immunoglobulin A, where a cysteine bond has been formed (see FIG. 4) between secretory component and dIgA or polymeric immunoglobulin A to become secretory immunoglobulin A (SIgA) (see FIG. 2). (For a review on mucosal immunity see e.g., Pilette, C., et. al., 2001, European Respiratory Journal, 18:571-588, Terauchi, Y., et. al., 2018, Human vaccines & immunotherapeutics, 14:1351-1361.)

Antibodies have enjoyed widespread success in their efficacy against a host of ailments. Antibodies further enjoy exquisite specificity to their targets as a result of the virtually limitless possible combinations of amino acids that make up their binding regions especially the CDRs and their large binding region surface area that allow binding specificity to be dependent on a large portion of the target surface topology including Van der Waals interactions, surface charge distribution, dipole-dipole interactions, salt bridges, and surface hydrogen bonding. FDA approved monoclonal antibodies that are fully representative of human immunoglobulins are almost universally based on Immunoglobulin class G (IgG) constant regions (when they have constant regions) as attributed to their long half-life of about 21 days that can be increased through modifications of their fragment crystallizable (Fc) regions. Antibody cocktails that are mixtures of 2 or more antibodies have received emergency authorization by the FDA as well for their application against the 2019 novel Coronavirus (COVID-19) and mutant forms. One challenge of direct administration of the antibody is the required repeated administration of such antibodies to sustain any benefit derived from them. In addition they can be very costly to produce making their cost sometimes the factor that prevents their administration in lieu of a lower cost and treatment that may be less effective or have more side effects.

Providing people with the genetic instructions or a gene therapy to make the antibodies with their own molecular machinery overcomes the problem of repeated administration in the case of retroviral insertion of the gene encoding for the antibody or makes administration much more long-lived in the case of episomal or even retroviral integration-based administration of the genes encoding for the antibody. Additionally, encoding for antibodies through gene therapy overcomes the high cost associated with manufacturing them. Although, as attributed to their longer half-lives IgG based antibodies have been widely prevalent in absence of a gene therapy encoding for them. IgA and dIgA have half-lives of 6 days making them require frequent administrations in their protein form and has been the reason that life sciences companies have not conducted clinical trials with IgA immunoglobulins. And while IgG can reach the mucosa of the lungs they may only do so via passive transport that results in very low levels of IgG in the mucosa while also being more rapidly degraded at a rate faster than that of SIgA in the mucus environment. Further, IgG does not reach the mucosal linings and channel linings of exocrine glands at high levels as it relies on passive diffusion to those regions. Gene therapy encoded episomal or genomic expression via integration competent lentivirus delivery systems or gammaretroviral delivery systems of dIgA solves the challenged associated with their short half-lives and solves the challenge of achieving therapeutically relevant levels of antibodies in the mucosa. In some cases the time period needed for effective immune protection is rather short and also the speed with which antibodies are needed may be rather fast as in the case of pneumonia (see FIG. 20) where mRNA-based administration of the dIgA and polymeric immunoglobulin A encoding genetic medicine will be effective. Immunoglobulins may be engineered to extend their half-lives or reduce antibody dependent enhancement of infection (ADE). Typically when IgG binds to its target in the mucosa of the lungs it may result in antibody dependent enhancement of infection (ADE) when either the IgG antibodies lack high binding affinities or when antibodies decline in numbers. Immunoglobulins may be engineered to reduce receptor binding to the fragment crystallizable region (Fc) reducing Antibody Dependent Enhancement (ADE) of infection that can result in a cytokine storm type reaction where the resulting immune reactions could cause damage to the host tissue. (See e.g., Iwasaki, A. & Yang, Y., 2020, Nat. Rev. Immunol. 20:339-341). Further, IgG and all subclasses are not privileged to enter the mucosa of the stomach and intestines and enter the mucosa of the lungs and other tissues at much lower levels than dIgA1 as a result of their reliance on passive diffusion to those areas where dIgA on the other hand is actively transported to the mucosa via polymeric immunoglobulin secreting receptor (pIgR).

Unquestionably, due to its active transport to the mucosa by pIgR, dIgA and polymeric immunoglobulin A that each become SIgA are privileged immunoglobulin to provide mucosal protection over other immunoglobulins such as immunoglobulin class G (IgG) and its 4 subclasses (IgG1, IgG2, IgG3 and IgG4) as they offer numerous advantages over IgG in the mucosa and complementary properties in cancer. Those properties include two (or more in the case of higher valency polymeric immunoglobulin A) distinct binding faces (What is meant by distinct binding faces of dIgA is either the two separate faces on a single dIgA that are more distal from each other as opposed to the two faces of dIgA that are more proximal to each other), a rigid backbone that allows dIgA and SIgA to agglutinate foreign bodies together neutralizing them and preventing their escape. Other advantages and properties of dIgA and polymeric immunoglobulin A include frequently more potent binding affinities over IgGs with as much as 1 to 2 orders of magnitude greater binding affinity than IgGs with the same V regions, active and unidirectional transport to the mucosa via binding to polymeric immunoglobulin secreting receptor (pIgR) at the basolateral face of the epithelium to ensure high levels in the mucosa, a naturally high level of production of dIgA and in some cases polymeric immunoglobulin A, resistance to degradation in the mucosa as part of their conversion to SIgA at the conclusion of active transport to the mucosa, dIgA is specifically and naturally tailored for the mucosa, naturally high levels of production at about 5 grams a day, in its SIgA form it has very low cytotoxicity and anti-inflammatory properties in the mucosa, while having both proinflammatory and anti-inflammatory properties in its dIgA or polymeric form protecting against cases of a damaged epithelium in the lamina propria and bloodstream, and SIgA does not cause antibody dependent enhancement of infection that could otherwise result in a cytokine storm in the lungs and thus makes targeted mucosal protection, neutralization and degradation of foreign bodies safe and free of SIgA induced pathology in the mucosa. (See, Iwasaki, A., Yang, Y., 2020, Nat. Rev. Immunol. 20:339-341).

T-cells including chimeric antigen receptor T-cells (CAR T-cells) have previously been programmed to express scFV-FvIgG as an approach to mitigate cancer, especially tumors. This has been accomplished by incorporating the genetic information necessary for the CAR T-cell apparatus in addition to encoding for a single chain Fragment Variable-Fragment Crystallizable fusions of immunoglobulin class G (scFV-FcIgG) all on a single vector or more than one vector. This T-cell is typically transduced with an integration competent or integration deficient lentivirus (See e.g., Suarez, E. R., Marasco, W. A., et al., 2016, Oncotarget, 7:34341-55). Although, Moloney murine leukemia virus (M-MLV) gammaretroviral vector has also been used (See, e.g. Powell, A. B., Ren, Y., et al., 2020, Methods & clinical development, 19: 78-88). Upon T-cell activation via the detection of the antigen through the CAR T-cell detection apparatus the T-cell becomes activated and releases the vector encoded scFV-FvIgG. Such an approach to engineer T-cells to express monoclonal immunoglobulins such as scFV-FcIgG upon activation has seen success in recruiting Natural Killer (NK) cells to the tumor site. Although, the most-deadly aspect of cancer that is metastasis cannot be addressed with monomeric antibodies because there is no specific property about them that would achieve such an outcome.

### SUMMARY OF THE INVENTION

This invention primarily relates to a method to encode for dimeric immunoglobulin A (dIgA1 or dIgA2), polymeric immunoglobulin A and engineered variants on a single or multiple DNA or RNA vectors. Examples of dIgA and polymeric immunoglobulin A engineered variants include modifications such as modified hinge lengths, Fab substitutions, domain substitutions and other modified Fc regions so long as the C_{H}3 cysteine bond with J Chain as well as other structural interactions such as formation of the Beta sheet complex (Beta sandwich) with J Chain between the C_{H}3 tails and J Chain are not disrupted to an extent to inhibit the formation of dimeric immunoglobulin A) on a vector as a means to deliver a gene therapy to humans. dIgA may be encoded for on a single DNA, or RNA with the use of a furin cleavage site and 2A self-processing peptide between consecutive transgenes. The three transgenes that make up dIgA (inclusive of polymeric immunoglobulin A) include (1) the immunoglobulin heavy chain (A1, A2 or engineered variant), (2) the immunoglobulin light chain and (3) the immunoglobulin J Chain. These three proteins may be encoded for a on a single genetic element in one or two open reading frames. Where all the transgenes in any one open reading frame will have in the 5′ to 3′ direction a furin cleavage site followed by a 2A self-processing peptide placed between any two consecutive transgenes. The use of a furin cleavage site and 2A self-processing peptide allows for efficient cleavage points between the consecutive transgenes completely removing any trace of the 2A self-processing peptide in the final immunoglobulin. In another embodiment a 2A self-processing peptide is used between consecutive transgenes in a single open reading frame in the absence of a furin cleavage site (see FIGS. 9B and 22B). In another configuration mRNA encodes for dIgA (dIgA1, dIgA2 or engineered variants) over one or more mRNA vectors (see FIG. 22). When a single mRNA encodes for dIgA it will include a furin cleavage site and 2A self-processing peptide or only a 2A self-processing peptide between any two consecutive transgenes in a single open reading frame. Such vectors will be delivered to cells in vivo or ex vivo.

T-cells including chimeric antigen receptor T-cells (CAR T-cells), CAR NK cells as well as other CAR engineered cells can be programmed with the dIgA and polymeric immunoglobulin A encoding vector constructs to upregulate express dimeric immunoglobulin A and polymeric immunoglobulin A upon activation of the CAR T-cell detection apparatus with this dIgA and polymeric immunogloulbin A vector construct. Such a CAR T-cell can more effectively mitigate tumors and minimize metastasis (See FIG. 41) over CAR T-cell technologies discussed in the literature: Both those that do express monomeric immunoglobulins and those that do not. This has the benefit of not only producing dimeric immunoglobulin A and polymeric immunoglobulin A at the site of the tumor thereby minimizing exposure to healthy tissue but they can also have increased expression upon the CAR T-cell's detection of the tumor and activation of the stimulatory and co-stimulatory domains of the CAR. Because of the multivalent binding faces of dimeric immunoglobulin A and polymeric immunoglobulin A these immunogloublins can prevent tumor metastasis because they can bind two different tumor cell receptors on each of dimeric immunoglobulin's or polymeric immunoglobulin's distinct binding faces effectively agglutinating tumor cells together. Additionally, dimeric immunoglobulin A and polymeric immunoglobulin A can contribute to the down modulation of specific tumor cell receptors in addition to inhibiting tumor growth and achieving effective tumor cell lysis by the recruitment of neutrophils and monocytes. (See, e.g. Lohse, S., Derer, S., Beyer, T., Klausz, K., Peipp, M., Leusen, J. H., van de Winkel, J. G., Valerius, T., 2011, J Immunol., 186:3770-8).

This patent contemplates thus used of retroviral vectors encoding for a both CAR and also dIgA (inclusive of polymeric immunoglobulin A) by both lentiviruses delivered lentiviral vectors and gammaretrovirus delivered gammaretroviral vectors to transduce immune cells for the purposes of mitigating carcinomas and solid tumors. Additionally, this patent contemplates the used of pseudotyped anti scFv CAR retroviruses to selectively deliver integration competent retroviral vectors to CAR engineered cells that are among other cells that are not engineered with a CAR as an ex vivo administration. Further, this patent contemplates generating dIgA (inclusive of polymeric immunoglobulin A) expressing CAR engineered NK memory cells. Similarly, this patent contemplates the generation of dIgA (inclusive of polymeric immunoglobulin A) expressing NK cells (See FIGS. 43 and 46). Such cell lines may be used as an off the shelf product. As a means of more closely coupling T-cell, NK cells or another immune cells secretion of cytokines and expansion associated with their activation this patent contemplates incorporating into vector constructs encoding for dIgA and polymeric immunoglobulin A the use of endogenous and abbreviated endogenous promoters for cytokines (perforin and granzyme B) that are placed in granules of NK cells or with cytokines or chemokines that are upregulated upon activation of the immune cell (IL-2, IFNγ and TNFα) or activation of the CAR so that dIgA and polymeric immunoglobulin A secretion in the non-CAR expressing immune cell and the CAR engineered immune cell is more closely coupled with immune cell and CAR engineered immune cell activation respectively.

Potent dimeric immunoglobulins of class A1, A2 or variants specific to (1) a virus (s) (2) a systemic ailment such as allergies (3) fungi (4) bacterial infection (4) cancer (5) biowarfare agent (6) microbial infection, (7) any ailment (8) an allergen or cause of allergies including IgE and cytokines, (9) a target protein or variant may be discovered or have their V regions or CDR regions derived or discovered from one or more sources exposed to the protein or protein source of interest (such as any of 1 though 9 and variants) where such sources of antibodies may include B-cells from humans, mice or other animals with humanized immune systems, transgenic mice, non-human vertebrates (e.g. mouse or rabbit) intended for chimeric antibodies or combinatorial libraries using human or mice B-cells to identify immunoglobulin DNA or using phage display technology to identify potent scF_{V} (single chain variable fragments) or even potent antibody binding fragments (Fab) against the protein or pathogen of interest (e.g. any of 1 through 9 or the pathology associated source) or allergen(s) of interest. In the invention one may take the gene sequence encoding for the safest and most potent immunoglobulins or immunoglobulin V-regions, CDR regions, or even binding regions that can be identified or evolved through mutagenesis, use the gene sequence or a redundant sequence that encodes for the same amino acids of those potent immunoglobulins, CDRs or immunoglobulin V-regions that were discovered in mice or other animals with humanized immune systems, non-human vertebrates that may optionally be intended for chimeric antibodies, transgenic mice, or derived from phage display technology and deliver to humans via mRNA, as an episome—via AAV, integration deficient lentivirus or integration deficient gammaretrovirus; or via integration competent lentivirus or gammaretrovirus for retroviral insertion into host genomic DNA the genetic information to produce the antibodies themselves in a dIgA1 or dIgA2 construct encoding for either monoclonal antibodies or an antibody cocktail of 2 or more immunoglobulins all based on those regions responsible for binding the target (e.g. Complementary determining regions, (CDRs)), V-regions, or the entire polypeptide sequence of the potent immunoglobulins they are based on. In humans, mice, animals with humanized immune systems, animals with engineered immune systems such antibodies may optionally be discovered from CD27+ IgG or IgA memory B-cells or CD+ memory B-cells, plasmablasts B-cells, germinal center B-cells or B-cells from mice or other animals with humanized immune systems exposed to some or all of the pathogen, pathogenic protein, pathogen associated protein or allergen of interest and affinity matured against it, non-human vertebrates intended for chimeric antibodies, transgenic mice or other non-human vertebrate with an engineered immune system or derived from phage display technology such as when using the scFv derived from human B-cells or mouse B-cells. In one embodiment infusion of mRNA, episomes or retroviral incorporation of DNA via integration competent lentiviral vectors or gammaretroviral vectors will engender humans with the ability to express dIgA1 and dIgA2—potent for the infectious agent, antigen or allergen of interest—that is modified into secretory immunoglobulin A (SIgA) as part of binding to polymeric immunoglobulin secreting receptor (pIgR) at the basolateral face of epithelial cells (See FIG. 2) and entering the mucus of organs such as the upper respiratory tract including the lungs, reproductive tract, digestive tract, our urinary tract, our skin exocrine glands of the breasts, all exocrine glands throughout the body. The mucosa of reproductive tract has both SIgA and dIgA as only portions of the epithelial cells of the reproductive tract have pIgR on the basolateral face. In the invention in cases where the respiratory tract of digestive tract is the mode of allergen or pathogen entry especially the stomach and upper duodenum e.g. to protect against a Helicobacter pylori infection the gene therapy or mRNA encoding protein-based vaccine may be administered through absorption or via endoscopic injection into the lamina propria of the stomach and upper duodenum. In other examples the invention will deliver the gene therapy through endoscopic microinjection into the lamina propria of the lungs, trachea, or esophagus may be used to administer the gene therapy. Targeting the lamina propria emphasizes the use of integration competent pseudotyped lentiviral vectors which is preferred to transduce memory B-cells but is not limited to this delivery vehicle. Further both ex vivo and in vivo administration is considered.

When a gene therapy cocktail is used, the infusion of mRNA, episomes or retroviral incorporation of DNA with a gammaretrovirus or a lentivirus may also engender humans with the ability to potentially produce any one Immunoglobulin Class G (IgG) subclass 1 (IgG1), subclass (IgG2) and subclass 3 (IgG3), IgA1, IgA2 in addition to dIgA1 and dIgA2. This infusion of mRNA, episomes or retroviral insertion of DNA coding for dIgA1 or dIgA2 engenders humans with two lines of defense against the any of (1) a virus (s) (2) a systemic ailment such as allergies (3) fungi (4) bacterial infection (4) cancerous tumor (5) biowarfare agent (6) microbial infection, (7) any ailment (8) an allergen or cause of allergies including IgE and cytokines, (9) a target protein or variant. The value of dIgA which provides SIgA based mucosal immunity is that it is possible to stop foreign bodies and foreign proteins before they breach the epithelial barrier while also avoiding the setting off of destructive inflammatory responses such as allergic reactions or antibody dependent enhancement of infection (ADE). If pathogens are neutralized in the mucus by SIgA1 or SIgA2 or engineered variants it will significantly reduce if not completely eliminate the probability of pathogen or allergen related events that are destructive to human tissue that may be dangerous or life altering to human life. Although, if the pathogen, antigen or allergen did manage to enter the bloodstream by breaching the epithelium a second line of defense dIgA directly beneath the epithelium and directly beneath the basement membrane would neutralize it there with an additional pro-inflammatory response.

### BRIEF DESCRIPTION OF THE DRAWINGS

FIG. 1A. depicts a basic schematic of dimeric immunoglobulin class A (dIgA). Additionally depicted is a disulfide where the hinge meets the C_{H}2 domain. That is the disulfide bond is said to be between the final amino acid of the C_{H}2 domains of one heavy chain and its nearest neighboring heavy chain. Further, each constant domain, the hinge or and the V-regions is labeled for one of the four tetramers where each tetramer is a heterodimer. The V-region of the heavy chain is referred to as V_{H}. Like all immunoglobulins dimeric immunoglobulin A1 (dIgA1) and dimeric immunoglobulin A2 (dIgA2) are made up of a heavy chain and light chain. The heavy chain of both dIgA1 and dIgA2 is a single polypeptide. That for dIgA1 and dIgA2 at the N-terminal end begins with the Variable Heavy (V_{H}) region that is generally about 119 to 120 amino acids in length, followed by the C_{H}1 domain, followed by the hinge region, followed by the C_{H}2 domain and followed by the C_{H}3 domain. The light is a single polypeptide that is a single polypeptide that at the N-terminal end begins with the Variable Light (V_{L}) that is about 106 to 109 amino acids in length region followed by the Constant Light (C_{L}) region that is either Kappa (For an example of a Human Kappa constant region gene of allotype Km(1) see SEQ ID NO: 26) or lambda (For an example of a Human lambda constant region gene see SEQ ID NO: 25). Dimeric immunoglobulin A also utilizes J Chain (For the Human J Chain found in the final dimeric immunoglobulin product that does not have the signal peptide see SEQ ID NO: 7) to form a tetramer of heterodimers that is effectively dimeric as related to IgG immunoglobulins that is a dimer of heterodimers and is thought of as monomeric. FIG. 1B depicts the cross section of crystal structure of a dimeric immunoglobulin A constant heavy fragment engaging in a complex with J Chain from Mus musculus where J Chain forms B-sheets with all 4 Immunoglobulin A Heavy Chains Beta Strands engaging in beta sheet formation with J Chain. Human dimeric immunoglobulin A is believed to form the same type of complex.

FIG. 2 depicts the mechanism by which any one of dimeric immunoglobulin A1 and polymeric immunoglobulin A1, dimeric immunoglobulin A2 and polymeric immunoglobulin A2 each one of which includes J Chain crosses the epithelium, where dimeric immunoglobulin A is depicted in FIG. 2, from the basolateral face—or face that is exposed to the tissue—of the epithelium by first binding to Polymeric Immunoglobulin Secreting Receptor (pIgR) that includes secretory component where it undergoes endocytosis into the epithelium and undergoes transcytoses across the epithelium to the apical face—the face that is exposed to the lumen of the organ of interest or interior of the channel in the exocrine gland that also inclusive of the mouth and nasal cavity and mucus in the lumen of the lungs, lumen of the digestive tract, lumen of the urinary tract, skin, exocrine glands of the breasts, and portions of the reproductive tract—where upon exocytosis to the apical face of the epithelial cells pIgR is cleaved releasing secretory component which has formed a disulfide bond with dIgA that is then referred to as secretory immunoglobulin class A subclass 1 (SIgA1) or subclass 2 (SIgA2) which refers to the fact that dIgA now has secretory component bound to it via a cysteine bond. This figure is not drawn to scale nor drawn to convey any structural or molecular information.

FIG. 3 depicts a basic schematic of immunoglobulin class G. For one light chain the Variable Light chain constant region is labeled V_{L} and the constant light chain is labeled CL. The immunoglobulin heavy chain consists of a constant region that is made up of C_{H}3 domain, C_{H}2 domain, the Hinge and C_{H}1 domain. The Fab or antibody-binding fragment is circumscribed and does not include the hinge. Additionally, the Fragment crystallizable (Fc) constant regions is circumscribed to include the hinge.

FIG. 4 left depicts a schematic of Human Secretory Immunoglobulin A1 (SIgA1) that in FIG. 4 is derived from dIgA1. In SIgA1 there are disulfide bonds between J Chain's cysteine 15 and 69 and a IgA1 heavy chain penultimate cysteine peptide from each immunoglobulin A1 participating in the dimer. Also, shown is the disulfide bond between secretory component cysteine 467 and immunoglobulin class A1 heavy chain cysteine 311 located in the C_{H}2 domain. The 5 domains D1, D2, D3, D4 and D5 of secretory component are also depicted in this figure. The crystal structure of SIgA from the organism Mus musculus is also shown with truncated immunoglobulin A heavy chains limited to the Fc regions and is labeled by domain.

FIG. 5 Omitted

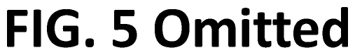

FIG. 6A depicts an Adeno-Associated Virus (AAV) single-stranded DNA vector from left to right or 5′ to 3′ containing a 5′ inverted terminal repeat (5′ ITR), a Promoter which could include an enhancer, the 5′ UTR, a DNA sequence encoding the immunoglobulin heavy chain IgHA with no stop codon, a DNA encoding for a furin cleavage site, DNA encoding for a 2A self-processing peptide, a DNA sequence encoding the immunoglobulin light chain IgLκ or IgLλ with stop codon, an internal ribosome entry site (IRES), DNA encoding for the immunoglobulin J Chain polypeptide necessary for the formation of dimeric Immunoglobulin A (dIgA) and polymeric forms, the 3′ UTR, a polyadenylation element and the 3′ ITR. In this drawing and most other drawings a box labeled “Kozak then start codon” means that the Kozak consensus sequence from positions −6 to −1 is present (generally “gccacc”) followed by the start codon “atg”. “Kozak then start codon” optionally means “gccaccatg” or it means that the Kozak sequence encoded in the natural or artificial 5′ UTR at its 3′ end and is immediately followed by the “atg” start codon. FIG. 6B depicts an mRNA that may be used to encode for dIgA1. FIG. 6B is an mRNA form that encodes for the same cassette (protein encoding region) as FIG. 6A where the 5′ 7-methylguanylate (m⁷G) cap with a 5′-5′ triphosphate linkage to the 5′ end of the mRNA where the ribose-2′-O position of the first and second nucleotides are methylated where the 5′ cap depicted in FIG. 6B and also in FIG. 20 is termed m⁷G(5′)pppN^{m}- and the poly(A) signal is generally between 120 and 150 adenosine polynucleotides.

FIG. 7A is similar to FIG. 6A and only differs by the relative location of the DNA encoding for the immunoglobulin heavy chain (IgHA1) IgH and the immunoglobulin light chain IgL(κ or λ). Additionally, FIG. 7A does not depict the 5′ untranslated region (5′ UTR) as separate elements. This speaks to the multiple ways to represent the vector constructs that have the same meaning. That is it is understood that 5′ UTRs are generally used in vectors although those sequences may be less than 10 bases in length, but that information is often not included in the vector constructs. FIG. 7B is identical in meaning to FIG. 7A although in FIG. 7B the signal peptides for each of the immunoglobulin chains (Heavy, Light and J) are shown as separate elements. Although, in FIG. 7A and all other figures in this patent it is understood that the signal peptides are incorporated into the vector constructs for DNA and RNA based vectors as well as the proviruses.

FIG. 8 is similar to FIG. 9A but a Woodchuck hepatitis virus post transcriptional regulatory element (WPRE) is not used.

FIG. 9A is similar to FIG. 6A but a furin cleavage site followed by a 2A self-processing peptide are used in place of the IRES. FIG. 9B is similar to FIG. 9A but furin cleavage sites are not used and only a 2A self-processing peptide separates any two consecutive genes.

FIG. 10 is similar to FIG. 11 but a Woodchuck hepatitis virus posttranscriptional regulatory element (WPRE) is not used.

FIG. 11 depicts cancer that could be any form of cancer and is not further specified as to the type of cancer nor the location in the body. The function of polymeric immunoglobulin A and dimeric immunoglobulin A (depicted) in FIG. 11 can be understood to be similar to the function of polymeric immunoglobulin A and dimeric immunoglobulin A (depicted) as described in FIG. 23 in that a major function of polymeric immunoglobulin A and dimeric immunoglobulin A is to bind to a cell surface protein generally upregulated or mutated or both on the cancer cell to prevent metastasis and to prevent cancer cells from escaping.

FIG. 12 is similar to FIG. 9A but the relative positions of the immunoglobulin heavy chain (IgHA1) and J Chain are swapped. Additionally, the immunoglobulin light chain is depicted as the kappa light chain (IgL(K)).

FIG. 13—depicts an Adeno-Associated Virus (AAV) single-stranded DNA vector from left to right or 5′ to 3′ containing a 5′ inverted terminal repeat (5′ ITR), a Promoter which could include an enhancer, the 5′ UTR, a DNA sequence encoding the immunoglobulin heavy chain IgA1 with no stop codon, DNA encoding a furin cleavage site, DNA encoding a 2A self-processing peptide, a DNA sequence encoding the immunoglobulin light chain IgLκ or IgLλ, with no stop codon, DNA encoding a furin cleavage site, DNA encoding a 2A self-processing peptide, DNA encoding for the immunoglobulin J Chain protein with no stop codon, DNA encoding a furin cleavage site, DNA encoding a 2A self-processing peptide, DNA encoding for a MZB1 protein with stop codon, the 3′ UTR, a polyadenylation element and a 3′ ITR

FIG. 14 is similar to FIG. 12 but the positions of the immunoglobulin light and heavy chains are swapped.

FIG. 15—depicts an Adeno-Associated Virus (AAV) single-stranded DNA vector from left to right or in the 5′ to 3′ direction containing a 5′ inverted terminal repeat (5′ ITR), a Promoter which could include an enhancer, the 5′ UTR, DNA encoding for a MZB1 protein with no stop codon, DNA encoding a furin cleavage site, DNA encoding a 2A self-processing peptide, DNA encoding for the immunoglobulin J Chain protein with no stop codon, DNA encoding a furin cleavage site, DNA encoding a 2A self-processing peptide, a DNA sequence encoding the immunoglobulin heavy chain IgA1 with no stop codon, DNA encoding a furin cleavage site, DNA encoding a 2A self-processing peptide, a DNA sequence encoding the immunoglobulin light chain IgLκ or IgLλ, with stop codon, a polyadenylation element and a 3′ ITR.

FIG. 16—is similar to FIG. 15 but the positions of IgHA and IgLκ or IgLλ, are swapped where IgHA has a stop codon and IgLκ or IgLλ, does not have a stop codon.

FIG. 17 depicts a Lentiviral vector from a first or second generation lentiviral delivery system shown at three levels. First in plasmid form with portions cut out as depicted by the double curved lines. Followed by the RNA form after transcription that is packaged into the lentivirus capsid and further packaged into the lentivirus envelope. Finally depicted last is the DNA form after reverse transcription of the RNA vector that is packaged in a 2^{nd} generation lentivirus delivery system. This vector may optionally be used as a retroviral or viral vector as determined by the functioning of the protein Integrase. FIG. 17 in the 5′ to 3′ direction depicts a 5′ LTR left orientation includes the U3 enhancer and promoter, the R repeat signal and the U5 polyadenylation signal. In this example the vector in the 5′ to 3′ direction depicts the vector to express dimeric immunoglobulin A (dIgA) including the, 5′ LTR, the Promoter that will optionally include an enhancer, including in the vector but the “psi” packaging signal that is located just 3′ of the 5′ LTR, followed by the rev response element (RRE), followed by the central polypurine tract-central termination sequence (cPPT/CTS), the promoter, the 5′ UTR, the immunoglobulin heavy chain for isotype A subclass 1 (IgA1), a furin cleavage site followed by a 2A self-processing peptide depicted as (Fur-2A) a stop codon not shown followed by the immunoglobulin light chain (IgL) that will be optionally kappa or lambda followed by a stop codon, followed by an IRES, followed by J Chain, followed by an IRES, and Marginal Zone B1 Cell Specific Protein (MZB1), the 3′ UTR, a Woodchuck hepatitis virus posttranscriptional regulatory element (WPRE), and the 3′ LTR containing the U3, R and U5 polyadenylation element that is used as the polyadenylation signal for the cassette encoding for dimeric immunoglobulin A. The 3′ LTR contains elements equivalent to the U3, R and U5 in the 5′ LTR.

FIG. 18 is generally similar to FIG. 17 except it depicts a Lentiviral vector from a 3rd Generation self-inactivating lentiviral delivery system. In FIG. 18 the U3 promoter/enhancer on the 5′ end of FIG. 17 is replaced with a CMV promoter to drive transcription as a result of the mutations or deletions of the U3 promoter/enhances (referred to as AU3). The AU3 on the 3′ end is copied to the 5′ end following reverse transcription.

FIG. 19A depicts the vector after reverse transcription referred to as the provirus and is generally similar to FIG. 18 except IgHA2 is depicted in place of IgHA1.

FIG. 20 depicts 3 separate mRNA vectors that could be delivered in a single vesicle such as a lipid nanoparticle to the cell of interest to express dIgA1 or dIgA2. As an example such a lipid nanoparticle could be used to deliver the mRNA that codes for a dIgA1, dIgA2 or an engineered variant. This encapsulated set of mRNA vectors could be used to provide a temporary and immediate remedy such as when immune protection is needed immediately or on a short time scale. Similarly FIG. 20 could depict mRNA encapsulated in an LNP where the mRNA encodes for dIgA that could be specific to adhesin SpsA of Streptococcus pneumoniae the most common bacteria to cause pneumonia. Such targeting for Streptococcus pneumoniae may take place with highly potent binding affinity for one of the highly conserved cell surface Streptococcus pneumonia epitopes on adhesin SpsA.

FIG. 21A is similar to the provirus depicted in FIG. 19 that is the vector following reverse transcription except MZB1 is not encoded for by the vector. FIG. 21B is similar to FIG. 21A but the dIgA encoding cassette is encoded for in the opposite direction relative how the vector is transcribed by from the parent plasmid when expressing the lentiviral vector for packaging into the lentivirus.

FIG. 22A is generally similar to FIG. 19 except IgHA1 is depicted in place of IgHA2. FIG. 22B is similar to FIG. 22A but a furin cleavage site is not used in the single open reading frame only a 2A self-processing peptide separates the immunoglobulin heavy and light chains.

FIG. 23 polymeric immunoglobulin A and dIgA (depicted) and SIgA binding a protein on upregulated on non-small cell lung cancer cells in such a way that the dIgA or SIgA binds neighboring cancer cells with its two distinct faces thereby preventing metastasis. Additionally, because of their two or more distinct binding faces of SIgA, dIgA and polymeric immunoglobulin A can prevent metathesis by binding escaping cancer cells on the distinct immunoglobulin face opposite the distinct immunoglobulin face bound to the malignancy. The polymeric immunoglobulin A and dIgA forms are capable being detected by macrophages and neutrophils via the FcαRI to disrupt cancer cell activity and eliminate cancer cells through both opsonization and phagocytosis. At the apical face of the epithelium in the mucosa SIgA is bound to the lumen facing face of the same malignant tumor. This figure is not limited in application to non-small cell lung cancer and could be represent other carcinomas and other hematological cancers.

FIG. 24 depicts a memory B-cell that has received a Lentiviral vector with the vector depicted as a provirus that is also the vector depicted in FIG. 21. What is shown is that the memory B-cell will initially express as a B-cell receptor (BCR) the naturally encoded immunoglobulin heavy chain and naturally expressing the immunoglobulin light chain. Upon incorporation of the pseudotyped lentivirus integrating the integration competent lentiviral vector through retroviral incorporation into the genomic DNA or alternatively with a integration deficient lentivirus delivery the cell will express the BCR as the natural IgH and vector encoded IgL as a result of the strong promoter encoded for in the vector that will result in a significantly higher level of expression of the vector encoded IgHA1, IgL and J Chain vs. the naturally encoded IgHG1 and IgL. Upon activation of the BCR by the protein target e.g. by vector encoded IgL detection of the targeted protein the memory B-cell will see one of two differentiation paths. In one path it will differentiated into a memory plasma B-cell (also referred to as a longlived plasma cell) that the vector programs to secrete dIgA and polymeric immunoglobulin A or engineered variants whose expression can persist for decades with the support of the right microenvironment secreting immunoglobulins at a rate of 500-1,000 per second. Alternatively, the memory B-cell can differentiate into a Germinal center B-cell.

FIG. 25 omitted

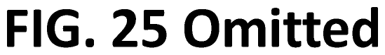

FIG. 26 depicts a vaccination strategy for HIV1, HIV2, Ebola, a rapidly spreading airborn virus, a virus, or any pathology promoting microbe or protein that has a highly conserved protein sequence on the surface that would allow for antibody targeting. This strategy will result in long term expression of dIgA and polymeric immunoglobulin A (that can would include mixtures of dIgA, trimeric immunoglobulin A, tetrameric immunoglobulin A and pentameric immunoglobulin A) or engineered variants for decades where T follicular helper (T_{FH}) cell development will require exposure to antigen. The T_{FH} cell can in turn activate GC B-cells carrying the integrated and episomally delivered lentiviral vector or even gammaretroviral vector.

FIG. 27 depicts the three lines of defense that is provided with SIgA and dIgA and in some embodiments polymeric immunoglobulin A (that can would include mixtures of trimeric immunoglobulin A, tetrameric immunoglobulin A and pentameric immunoglobulin A) or engineered variants to prevent type I hypersensitivity. SIgA specific to the allergen of interest can bind and agglutinate the allergen in the mucosa of the respiratory tract or in the lungs minimizing the number of allergens that passively cross the epithelium. Additionally, SIgA can bind allergens before they have a chance to bind dendritic cell extensions in the mucosa where in cases that dendritic cell (DC) extensions bound allergens and subsequently interact with the T-cell receptor or Tₕ2 Helper cells via DC MHCII presenting allergen oligopeptide fragments that causes the release of IL-4 and IL-13 causing the activation of IgE secreting B-cells increasing the IgE concentration and increasing the incidence of mast cells with two neighboring FcαRIs bound to 2 neighboring IgEs. If allergens traverse the epithelium dIgA or polymeric immunoglobulin A would bind to allergens and could promote a proinflammatory response or would be transported to the mucosa while bound to the allergen of sufficiently small size with polymeric immunoglobulin secreting receptor (pIgR).

FIG. 28 depicts a lentiviral vector as a provirus. The vector encodes for both dIgA and a Chimeric Antigen Receptor (CAR) whose design is not specified. The vector in the 5′ to 3′ direction includes the 5′ LTR followed by the psi packaging signal, the rev response element (RRE) and the central polypurine tract (cPPT), the promoter, the 5′ UTR, dIgA cassette is consistent with that described in FIG. 14 and is followed by an internal ribosome entry site the CAR cassette, the 3′ UTR, the WPRE, followed by the 3′ LTR.

FIG. 29 depicts a lentiviral vector as a provirus. The vector encodes for a Chimeric Antigen Receptor (CAR) whose design is not specified. Additionally, the vector encodes for either dIgA or polymeric immunoglobulin A (that can would include mixtures of dIgA, trimeric immunoglobulin A, tetrameric immunoglobulin A and pentameric immunoglobulin A). The vector in the 5′ to 3′ direction includes the 5′ LTR followed by the psi packaging signal, the rev response element (RRE) and the central polypurine tract (cPPT), the promoter, the 5′ UTR, DNA encoding for the the immunoglobulin heavy Alpha chain—that can be subclass 1, 2 or an engineered variant, a furin cleavage site, a 2A self-processing peptide, the immunoglobulin light chain that can be a Kappa or Lambda light chain, followed by an internal ribosome entry site, J Chain, a furin cleavage site, a 2A self-processing peptide, a sequence that encodes for a CAR that is not further specified, the 3′ UTR, the WPRE, 3′ LTR containing the U3, R and U5 polyadenylation element that is used as the polyadenylation signal for the gene elements encoding for the CAR and dIgA.

FIG. 30A depicts a gammaretroviral vector that can be from the Murine Lukemia Virus (MLK) that is depicted as a provirus. The vector cencodes for both dIgA and a Chimeric Antigen Receptor (CAR) whose design is not specified. The vector in the 5′ to 3′ direction includes the 5′ LTR followed by the primer binding site (PBS), the psi packaging signal, the promoter, the 5′ UTR, DNA encoding for the immunoglobulin heavy chain of class A, followed by DNA encoding for a furin cleavage site, followed by DNA encoding for a 2A self-processing peptide, followed by DNA encoding for the immunoglobulin light chain that can be a Kappa or Lambda light chain, followed by DNA encoding for a furin cleavage site, followed by DNA encoding for a 2A self-processing peptide, followed by J Chain, followed by a stop condon, followed by an internal ribosome entry site, followed by DNA encoding for the CAR with signal peptide, followed by a stop codon, followed by the 3′ UTR, the WPRE, the polypurine tract (PPT), followed by the 3′ LTR that contains the U5 polyadenylation signal for the gene elements encoding for dIgA and the CAR that will yield a single mRNA. FIG. 30B is similar to FIG. 30A but J Chain is located following the IRES where J Chain and the CAR are expressed in a single open reading frame as a result of placing DNA encoding for a furin cleavage site, followed by DNA encoding for a 2A self-processing peptide between DNA encoding for J Chain and the CAR.

FIG. 31 depicts a gammaretroviral vector depicted as a provirus similar to FIGS. 30A and 30B that can be from the Murine Lukemia Virus (MLK). The vector encodes for both dIgA and a Chimeric Antigen Receptor (CAR) whose design is not specified. Where the dIgA and CAR encoding regions are similar to FIG. 28.

FIG. 32 depicts a lentiviral vector as a provirus similar to that described in FIG. 28 although a the dIgA and CAR related sequences are separated by a furin cleavage site followed by 2A self-processing peptide where no IRES is used. Additionally, the CAR details are specified to include in the 5′ to 3′ direction of the CAR, the Signal Peptide from the CD8α protein, the scFv which is comprised in the 5′ to 3′ direction of the Variable light chain or heavy chain region, a linker then the variable light chain or heavy chain region. Following the scFv in the 5′ to 3′ direction is the hinge derived from CD28 or CD8α, the transmembrane domain derived from CD28 or CD8α (it should be noted that use of the CD8α hinge does not preclude the use of the CD28 transmembrane domain), followed by the co-stimulatory signaling domain of CD28 or 4-1BB, followed by the CD3ζ signaling domain.

FIG. 33 is omitted

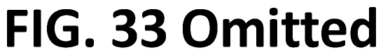

FIG. 34 is similar to FIG. 32 but the Eukaryotic Translation Elongation Factor 1 alpha (EF-1α) Promoter is specified.

FIG. 35 is similar to FIG. 34 but FIG. 35 uses an internal ribosome entry site (IRES) between the elements coding for dIgA and the CAR. Additionally, the dIgA and the CAR encoding cassettes are encoded for in the opposite direction relative how the vector is transcribed by from the parent plasmid when expressing the lentivirus plasmid for packaging into the lentivirus.

FIG. 36 depicts an activated T-cell or activated NK cell that is transduced by an anti-CD8+ pseudotyped lentivirus or gammaretrovirus that may be either integration competent or integration deficient that encodes for both dIgA as well as the CAR. The CAR could be one that is used in an FDA approved CAR cell technology. The T-cell may optionally be genetically engineered, so it does not express its endogenous T-cell receptor eliminating graft vs. host disease (GVHD) making the engineered cell more suitable to be used as an off the shelf technology. Where the vector is similar to that vector described in FIG. 32 for lentivirus or alternatively FIG. 31 for gammaretroviruses. Upon viral integration in the cell genome the cell will express both the CAR as a cell surface receptor and secrete dIgA and polymeric Immunoglobulin A in small amounts. Where upon activation of the cell by the CAR scFv e.g. at the site of a carcinoma will secrete increasing amounts of dIgA and polymeric immunoglobulin A at the tumor location in addition to secreting IL-2, perforin, granzyme, TNFα and IFNγ.

FIG. 37 depicts a CAR engineered cell transduced by an anti-scFV pseudotyped retrovirus (lentivirus or gammaretrovirus) that is optionally pseudotyped with a protein that that the CAR scFv is specific to. The promoter used in the vector may optionally be any one of a CMV Immediate Early enhancer and CMV promoter or EF-1α. But could also be a full promoter or truncated promoter or an enhancer and full promoter or truncated promoter for any one of but not limited to IL-2, granzyme B, perforin, TNFα and IFNγ where such promoters or enhancer and promoter combinations would increase the sensitivity of dIgA and polymeric immunoglobulin A expression to the activation of the CAR. Where upon activation of the cell by the CAR detection apparatus e.g. at the site of a carcinoma will produce and secrete increasing amounts of dIgA and also chemokines and cytokines.

FIG. 38 depicts a Chimeric Antigen Receptor (CAR) made up of a single polypeptide that is located at the surface of the CAR engineered cell. At the extracellular face the CAR begins with the single chain variable fragment (scFv) which includes in the N-terminal to C-terminal direction the Variable Light (V_{L}) chain region the linker the Variable Heavy (V_{H}) chain region the hinge that is derived from CD8α or CD28. Continuing from the scFv in the N-terminal to C-terminal direction of the polypeptide is the hinge that is derived from CD8α or CD28 that is followed by a CD28 domain necessary for co-stimulatory signaling and finally the CD3ζ signaling domain.

FIG. 39 is similar to FIG. 38 but the CD28 domain necessary for co-stimulatory signaling is replaced with a 4-1BB domain necessary for co-stimulatory signaling. Additionally, the relative positions of the V_{H} region and the V_{L} region are switched.

FIG. 40 is similar to FIG. 39 but the CD28 domain necessary for co-stimulatory signaling is replaced with an unspecified domain necessary for co-stimulatory signaling. Additionally, the scFv shows flexibility with the use of V_{H}/L and V_{H}/L in thAT V_{H} or V_{L} may be used at either position that is from the N-terminal end to the C-terminal end of the protein with pairs of V_{L} and V_{L}, V_{H} and V_{H}, V_{L} and V_{H} or V_{H} and V_{L}.

FIG. 41 depicts a CAR engineered T-cell (or other CAR engineered cell such as a CAR engineered NK cell) cell that is also engineered to express dIgA (depicted) and polymeric immunoglobulin A that is at the site of a cancer tumor where non-small cell lung cancer is depicted and the T-cell, NK cell or immune cell has been activated by the CAR. Where upon activation the CAR engineered cell releases dIgA and polymeric immunoglobulin A specific to a protein upregulated on the cancer tumor. The dIgA and polymeric immunoglobulin A will prevent or reduce metastasis as shown by binding neighboring cancer cells on their multivalent faces in addition to capturing escaping cells that are metastasizing. Where also none, one or more of IL-2, granzyme A and B, perforin, TNFα and IFNγ will be released by the CAR engineered T-cell, NK cell or other CAR engineered cell. Additionally, if the T-cell is in the interstitial space facing the cancer tumor the dIgA and polymeric immunoglobulin A will also be transported across the epithelium and converted into SIgA where it will engage in similar binding to the tumor that will not be readily accessible by other immunoglobulins.

FIG. 42 is similar to FIG. 23 although FIG. 23 represents non-small cell lung cancer and FIG. can represent any carcinoma such as but not limited small cell lung cancer. FIG. 42 like FIG. 23 depicts dIgA and polymeric immunoglobulin A binding to a receptor that is upregulated on the cancer cells has an opportunistic mutation that allows for selective binding over healthy cells or both where a single dIgA or polymeric immunoglobulin A binds to neighboring cancer cells with each of its distinct binding faces thereby preventing metastasis and thus preventing the primary means by which cancer causes death.

FIG. 43 is similar to FIG. 41 but the carcinoma is not necessarily lung cancer and the dIgA and polymeric immunoglobulin A expressing cell may be an NK cell that does not express a Chimeric Antigen Receptor (CAR).

FIG. 44 depicts a lentiviral vector encoding for dimeric single chain variable Fragment-fragment crystallizable fusions of immunoglobulin A (dscFV-FcIgA). This protein encoding region of the vector in the 5′ to 3′ direction includes the signal peptide, the variable domain of the light chain, a linker, the variable domain of the heavy chain where no signal peptide is used, a linker, the fragment crystallizable region from constant heavy A followed by an internal ribosome entry site (IRES) followed by J Chain.

FIG. 45 depicts a CAR engineered immune that has detected cancer and secretes polymeric immunoglobulin A and dimeric immunoglobulin A or even dimeric and polymeric single chain variable Fragment-fragment crystallizable immunoglobulin A and is similar to FIG. 41. Although, FIG. 45 could also be understood—by replacing the depicted cell that expresses a CAR with a cell that does not express a CAR.

FIG. 46 is similar to FIG. 37 but the NK cell receiving the vector is depicted to not be a CAR engineered cell but could optionally be a CAR engineered cell.

### DETAILED DESCRIPTION OF THE INVENTION

This present invention arises out of a need to prevent cancer metastasis, treat carcinomas and to neutralize a variety of systemic pathologies and pathology promoting substances and organisms before they have a chance to cross mucosal tissue or spread far from the mucosal barrier. This invention is a gene therapy focused on expressing immunoglobulins that are naturally produced in large concentrations at mucosal and exocrine tissue that is dIgA1 (inclusive of polymeric immunoglobulin A1), dIgA2 (inclusive of polymeric immunoglobulin A2) and engineered variants that respectively becomes SIgA1, SIgA2 or an SIgA engineered variant upon being actively transported from the basolateral face of the epithelium to the mucosa or exocrine channel. Further, contemplated in the invention are dimeric single chain fragment variable-fragment crystallizable fusions of immunoglobulin A (dscFV-FcIgA). The present invention is designed to achieve safety and effectiveness by embracing the immunoglobulins that achieve the therapeutic effect or immunological protection against cancer, the foreign body, bacteria, virus, antigen, pathogenic body, pathogenic protein, biowarfare agent or allergen of interest that are developed in humans, developed in mice or other animals with humanized immune systems, developed in transgenic animals, developed in mice and intended for chimeric immunoglobulins, developed in mice not intended for chimeric antibodies, developed from phage display technology where single chain variable fragment (scF_{V}) regions from combinatorial libraries and identified in phage display technology to be therapeutically effective against the pathogen, protein of interest or other antigen, or where immunoglobulins are developed artificially or in humans exposed to the foreign body, pathogenic body, pathogen associated protein, antigen or allergen of interest all which may be broadly classified for the purposes of this patent application as the target of interest. The invention describes the method to identify the DNA sequence and/or polypeptide sequence of high affinity immunoglobulins expressed in individuals that were exposed to the target of interest. The invention further describes the method to design vectors expressing those immunoglobulins and also dimeric immunoglobulins class A (dIgA1 and dIgA2) necessary for mucosal immunity that encode at a minimum part of the binding regions, V-regions as identified from phage display technology or non-human vertebrates (e.g. mouse or rabbit) intended for chimeric antibodies using some or all of the CDR regions, but more generally at least the Fab regions if not the entire polypeptide sequence of the potent immunoglobulins identified from mice or other animal with humanized immune systems, transgenic mice or the B-cells, plasmablasts, germinal center B-cells, CD27+ IgG memory B-cells, CD27+ IgA memory B-cells from persons that were exposed to or were exposed to and recovered from the foreign body, bacteria, virus, micro-organism, antigen, pathogenic body, pathogenic protein, biowarfare agent or allergen of interest. Delivery systems are also described, which include AAV, adenovirus, lentivirus, gammaretrovirus, lipid nanoparticles and vesicle-based delivery systems. While dIgA is privileged to reach the mucosa it can also function effectively in the tissue with increased binding affinities over IgG counterparts. Thus, dIgA1 and dIgA2 may be considered in multiple different regions of the body to provide a therapeutic benefit.

Blood will be collected from any of an individual, a transgenic animal, a mouse with humanized immune system, or a mouse that was infected with, exposed to and ideally has undergone affinity maturation, or lives with any of (1) a virus (s) (2) a systemic ailment such as allergies (3) fungi (4) bacterial infection (4) cancerous tumor (5) biowarfare agent (6) microbial infection, (7) any ailment (8) an allergen or cause of allergies, (9) a target protein or variant. Memory B-cells will be collected from the buffy coat layer (layer that contains white blood cells) of fractionated blood. From humans the Immunoglobulin class A (IgA) CD27+ and class G (IgG) CD27+ memory B-cells, germinal center B-cells or other B-cells with cell surface immunoglobulins will be subsequently isolated. Memory B-cells, germinal center B-cells and plasmablasts are the only B-cells that have both undergone some affinity maturation and bear cell surface immunoglobulins in any appreciable quantity that is required for investigation. Thus, the process of separating cells—that have undergone affinity maturation—based on the affinity of their immunoglobulins for a target of interest can most reliably take place with memory B-cells, germinal center B-cells or even plasmablasts but is also possible with other B-cells bearing a cell-surface immunoglobulin. There is always the possibility that an IgM B-cell that bears the IgM cell surface immunoglobulin will have high binding affinity and that will be considered as well. Additionally, there is always the possibility that one may isolate a plasma B-cell or memory plasma B-cells that produces immunoglobulins with potent binding affinity for the target of interest. A technique known as B-cell Elispot may be used to identify plasma B-cells or memory plasma B-cells secreting antibodies with potent binding affinity for the target of interest. (See e.g., Crotty, S., Aubert, R. D., Glidewell, J., & Ahmed, R., 2004, J Immunol Methods, 286:111-122.) Generally, B-cell Ellispot may involve using memory B-cells and stimulating them to differentiate into plasma secreting B-cells to detect for secreted antibodies potent for the target of interest. The plasma secreting B-cells are placed in proximity to a plate coated with the antigen of interest (whether self-antigen, foreign antigen or allergen is irrespective of the procedure) where after some time period the cells are washed away and biotinylated detection antibodies are added to the plate to detect for the presence of the secreted antibody bound to the antigen on the plate. Thus, this instant patent contemplates the use of memory B-cells, germinal center B-cells, plasmablast B-cells, memory plasma B-cells and plasma B-cells to identify immunoglobulins, immunoglobulin V-regions with potent binding affinity for the target of interest.

Alternatively, phage display technology that collectively expresses a combinatorial library of scF_{V} with the use of mutagenesis as a means of in vitro affinity maturation of repeated cycles of scF_{V} evolution and bio panning techniques to achieve increasing binding affinities for the target of interest may be used to identify the cDNA encoding for the potent binding affinity. The source of the immunoglobulin cDNA encoding for the scFᵥ may or may not be from persons that were exposed to the target of interest. Another possibility is the use of mice or other animals with humanized immune system or transgenic mice as a means to affinity mature human immunoglobulins in these animals by exposure to the target of interest or part of the V regions may be identified with the use of non-human vertebrates (e.g. mouse or rabbit) intended for chimeric antibodies.

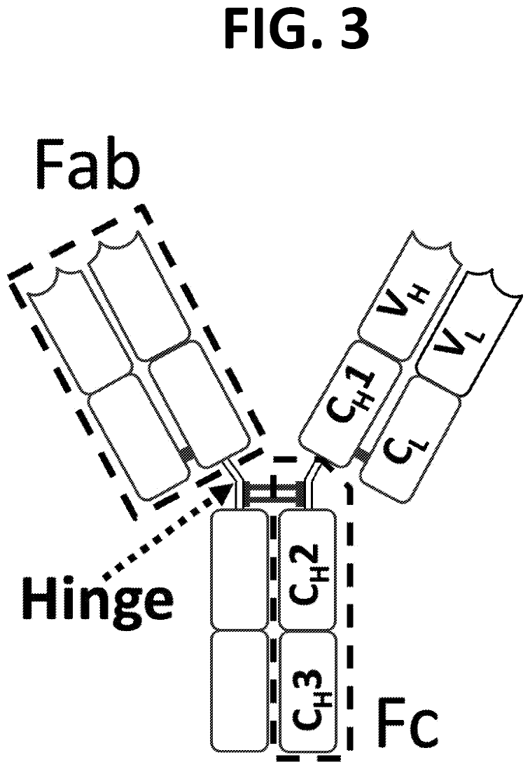

In the invention the genetic information that encodes for that potent binding affinity or the polypeptide sequence that results in that immunity will be determined, evaluated for safety, coupled with constant regions potentially engineered and then incorporated into an mRNA vector, episomal expression vector for expression in muscle cells, B-cells including memory B-cells, Germinal Center B-cells, memory plasma B-cells, a plasma blast, and naïve B-cells), liver cells (such as hepatocytes), splenocytes, T-cells including chimeric antigen receptor T-cells (CAR T-cells), and other potential cells or retroviral incorporation into genomic DNA via a gammaretrovirus or lentivirus which is preferred. In the invention those episomes would be delivered as via an adeno-associated-virus (AAV) vehicle as single-stranded DNA that would be converted into double-stranded DNA by the host genetic machinery, adenovirus vehicle, mRNA, lentivirus or gammaretrovirus as RNA that would be reverse transcribed into double-stranded DNA in the host by reverse transcriptase contained in the lentivirus capsid or gammaretrovirus capsid. In the invention if human donors or mice or other animals with humanized immune systems, transgenic mice or non-human vertebrates (e.g. mouse or rabbit) intended for chimeric antibodies are used as the source of potent immunoglobulins or potent binding affinity episomes that encodes for at a minimum immunoglobulin's Complementarity determining regions (CDR) or V regions if not the Fab (See FIG. 3) or even F(ab′)2 that match with at least 85%, 88%, 91%, 94%, 97%, or greater than 99% overlapping amino acid identity to those CDR regions, V Regions if not the entire polypeptide sequence of potent immunoglobulins specific to the target of interest expressed in the mice or other animal with humanized immune system, transgenic mice, or humans that were exposed to the target of interest. Additionally non-human vertebrates (e.g. mouse or rabbit) intended for chimeric antibodies may have some or all of the CDR regions incorporated into a human immunoglobulin construct. In the invention if phage display technology is used the recombination of the identified V_{H} and V_{L} regions will be evaluated against heavy chain constant regions of different isotypes and subclasses and the two different light chain constant regions IgL(κ or λ) respectively, where the immunoglobulin constant regions may be engineered to modulate effector functions. If an antibody cocktail of immunoglobulins is used it could include both immunoglobulin class G (IgG), immunoglobulin class A (IgA) and dIgA, which is converted to secretory IgA (SIgA) as part of being transported across the epithelium into the lumen of the upper respiratory tract, lungs, gastrointestinal tract, urinary tract, skin, endocrine glands and reproductive tract is the primary immunoglobulin responsible for mucosal immunity will also be encoded for in the antibody cocktail. The mucosa of reproductive tract has both SIgA and dIgA because only portions of the epithelial cells of the reproductive tract have polymeric immunoglobulin secreting receptor (pIgR) on the basolateral face. Thus, it is through the specific method of gene therapy (e.g. mRNA, DNA, retroviral insertion) that collectively code for a single dIgA antibody, engineered variant or an antibody cocktail mixture that mucosal protection from the target of interest will be achieved. The immunoglobulins of interest will produce full or even engineered immunoglobulins to modulate effector functions and could be modified to increase half-life. (See e.g., Iwasaki, A. & Yang, Y., 2020, Nat. Rev. Immunol. 20:339-341.)

The various compositions and methods of the invention are described below. Although particular compositions and methods are exemplified herein, it is understood that any of a number of alternative compositions and methods are applicable and suitable for use in practicing the invention. It is understood that vector constructs may include additional DNA, such as a 5′ Untranslated Region (5′ UTR) and 3′ UTR between or preceding cis-acting signals and transgenes as well as spacer DNA between or before cis-acting signals and viral signals such as LTRs, ITRs, psi, RRE, cPPT/CTS, etc. It is also understood that evaluation of the immunoglobulin expression constructs for the vectors specified in this invention can be implemented using methods standard in the art. New vector constructs are also proposed to enable the expression of dimeric immunoglobulin A (dIgA) and polymeric immunoglobulin A, which becomes secretory immunoglobulin A (SIgA) as part of crossing an epithelial cell from the basal to apical face. Additionally, it is understood that there may be a number of methods and assay modifications that can be used to isolated memory B-cells based on relative affinity for antigens of interest by a flow cytometry or Fluorescence activated cell-sorting (FACS) technique. Additionally, the value is further justified by the incorporation of genetic information to expression all or part of those high affinity immunoglobulins CDR, Fab, V-region, scFᵥ or partial binding element from non-human vertebrates (e.g. mouse or rabbit) intended for chimeric antibodies, in the context of complete immunoglobulin a mRNA, non-viral, viral or retroviral vector construct.

The methods of this invention will utilize unless specified otherwise modern techniques of oncology, immuno-oncology, cell molecular biology, chemical biology, microbiology, biochemistry and immunology. Many of such techniques are explained substantially in the publication literature and are well understood to those skilled in the art.

### 1. Definitions

Unless stated otherwise all terms used herein have the same meaning that they would to one skilled in the science, art and practice that is utilized in the present invention which include terms from methods in oncology, immuno-oncology, molecular biology, immunology, microbiology, chemical biology, recombinant DNA technology, biochemistry, synthetic biology and virology. These terms are well within the knowledge of those with in depth knowledge or skill of the art.

The term “conserved polypeptide sequence” as used herein refers to polypeptide sequences that have remained evolutionally conserved possibly due to functional constraints. This bears relevance in the innate immune system, which relies on pattern recognition of conserved polypeptide sequences to recognize antigens of pathogens.

The term “affinity maturation” as used herein refers to the maturation of an immature B-cell through both isotype switching and somatic hyper mutation and a memory B-cell through somatic hyper mutation that occurs through CD4+ helper T-cell activation of B-cells that have engulfed and degraded an antigen.

The term “potent” as used herein refers to binding affinity of immunoglobulins. Potent is meant to refer to a binding affinity of the immunoglobulin for the foreign antigen produced by the pathogen where the binding affinity is sufficiently high to warrant further investigation for the purposes of expressing the immunoglobulin or a derivative of that immunoglobulin as intended for episomal expression of immunoglobulins. For cancer potent binding affinity can be understood to be a binding affinity that is therapeutically relevant and may not be considered potent based on comparisons to binding affinities of necessary to prevent viruses from infecting humans. It is further understood that potent binding affinity may be interchanged with therapeutically relevant binding affinity.

The term “coding DNA” or “cDNA” as used herein refers to DNA that encodes for the polypeptide of interest. cDNA is effectively the part of mRNA that encodes for amino acids that the ribosome machinery translates the nucleic acid code into a chain of amino acids but in mRNA the thymine in cDNA is replaced with uracil in mRNA. However, for the purposes of this instant patent the cDNA is more concerned with the polypeptide sequence that it codes for. In that sense the degeneracy of the genetic codes considers any codon that codes for the amino acid to be an acceptable replacement for a cDNA codon. This is because the concern is the polypeptide sequence where the cDNA that codes for it does not matter due to the degeneracy of the genetic code. However, it is recognized that the efficiency with which an amino acid can be polymerized onto the protein being synthesized by the ribosome is dependent on the codon specifically even though three different codons may code for the same amino acid they may be polymerized onto the developing protein at different rates in a codon dependent manner.

The term “dissociation constant” or (K_{d}) as used herein refers to that immunoglobulin relative proportion or ratio of unbound immunoglobulin to the antigen of interest vs. immunoglobulin bound to the antigen of interest at any instantaneous moment or as a portion of time or ratio that an immunoglobulin is unbound to its antigen vs. bound. K_{d} is often described as (K_{off}/Kₒₙ).

The term “delivery system” as used refers to any system that contains and delivers molecules to the host cell.

The term “donor” as used refers to a human source or other organism source of biological material and thus anything derived from the donor. Specifically, the terms donor is used to refer to immunoglobulin polypeptide sequences or the coding DNA (cDNA) that encodes for them that is derived from the blood of a person that is infected with or has been exposed to or exposed to and recovered from the virus, pathogen, bacteria, antigen, pathogenic protein, systemic pathogenic ailment, biowarfare agent or allergen of interest. However, the cDNA is not required because of the degeneracy of the genetic code and thus, the polypeptide sequence matters most. What is meant by a donor-based immunoglobulin polypeptide sequence or domain is that the sequence is derived from the cell of the donor or human that provided the cell producing the immunoglobulin of interest, the V-region of interest or the CDR of interest and refers to an immunoglobulin or immunoglobulin element with therapeutic binding affinity for its target as per the context.

The term “episome” as used herein refers to DNA that resides in the nucleus of the host cell as an extra-chromosomal DNA and does not integrate into the genomic DNA of the host. The episome is said to be a separate artificial chromosome. Although, an episome can be made to be non-replicating or could be designed to be replicating. When an episome is non-replicating when the cell divides into 2 daughter cells only one of the two daughter cells will contain the episome. The concentration of episomes in the host animal for example will reduce with time that is a function of the lifespan of the different cells where the episomes resides.

The term “therapeutic binding affinity” as used herein refers to the antibody binding to any protein of interest including variants of that protein when specified, as well as other body of interest such as a molecule or macromolecule where the desired therapeutic effect is achieved with that binding affinity in the immunoglobulin of interest. That is the binding affinity that most effectively prevents pathology associated with the antibody's target such as a virus, pathogen, bacteria, microorganism, systemic pathological ailment, cancer, antigen, biowarfare agent or allergen.

The term “J Chain” also referred to as “Joining Chain” as used herein refers to a 159 amino acid polypeptide that includes a signal peptide that is cleaved to a 137 amino acid polypeptide that forms a cysteine bond to (A) 2 immunoglobulin class A subclass 1 through a cysteine bond between the penultimate cysteine on each immunoglobulin heavy chain to each of the C15 and C69 cysteines of J Chain. (B) 2 immunoglobulin class A subclass 2 through a cysteine bond between the penultimate cysteine on each immunoglobulin heavy chain to each of the C15 and C69 cysteines of J Chain.

The term “immunogenicity” as used herein refers to the ability of a foreign substance, such as an antigen, molecule, macromolecule or allergen to provoke an immune response in the body of a human and that may result in side effects and health complications.

The term “immunocompetent” as used herein refers to individuals that have the ability to produce and further develop an immune response following exposure to an antigen.

The term “pseudotyped” as used herein is the process of producing viruses that is viral or retroviral vectors in combination with foreign viral envelope proteins. A virus that is said to be pseudotyped is also referred to as a pseudovirus.

The term “daughter” cell as used herein refers to any of the cells formed when the cell undergoes cell division by mitosis.

The term “effector functions” refers to the signaling activity of antibodies such as the immunoglobulins. The immunoglobulin heavy chain constant regions specifically the Fc and the pFc′ constant domains are inclusive of effector signaling regions of the immunoglobulin. Different immunoglobulin isotypes and subclasses have different effector functions via different receptors for their Fc regions. Effector functions of immunoglobulins communicate with other cells or soluble proteins in the immune system through receptor mediated processes and signal to them to carry out a function or relay a signal to another type of cell. Effector functions include but are not limited to neutralization, opsonization, sensitization for killing by natural killer cells, activation of the complement system, activation of macrophages or phagocytes and activation of mast cells and basophils, antibody-dependent cellular cytotoxicity and antibody-dependent cellular phagocytosis.

The term “small pre-B-cell” refers to a state in B-cell development while the destined B-cell is in the bone marrow. V to J gene rearrangements of the immunoglobulin light chain on chromosome 2 (Kappa locus) or chromosome 22 (Lambda locus) takes place.

The terms “genomic DNA” refers to chromosomal DNA specific to the organism and thus is expected to be found in most cells of the organism. Genomic DNA represents the bulk of the genetic material of the organism. Other genetic material may be mitochondrial DNA and gamete DNA.

The term “non-viral vector” may refer either to a virus or viral particle containing DNA capable of functioning as an episome in the nucleus but is not contained within a capsid. Non-viral vectors and transfer plasmids contain structural and/or functional genetic elements that are primarily derived from a virus but not part of a viral delivery system.

The term “self-antigen” may refer to a protein, polypeptide of macromolecule that is produced by the organism. Self-antigens are to be distinguished from “antigens” that are proteins, polypeptides or macromolecules produced by an external organism or “viral antigens” that are produced by a virus.

The term “systemic pathologies” may refer to a health disorder whose basis of pathology is due to internal factors related to and encoded for in protein levels, mutations, DNA, RNA or cancer. This is to be distinguished from an “external pathogen” that is a pathogenic protein, virus, fungi or other microorganism that is capable to causing pathology.

The term “pathology” may refer to the specific damage sustained by the host organism as a result of any cause. Pathology is broadly defined and may include tissue damage, tissue compromise, cell loss, cancer, allergic reactions, cell malfunction, organ malfunction, organ malformation, tumors, imbalances in cell levels or imbalances in metabolic levels, as well as the psychological consequences of any imbalance.

The term “retroviral vector” refers to a viral vector or plasmid containing structural and functional genetic elements, or portions thereof, that are primarily derived from a retrovirus that is intended for retroviral incorporation into DNA.

The term “lentiviral vector” refers to a vector or plasmid containing structural and functional genetic elements, or portions thereof, including long terminal repeats (LTRs), psi encapsidation signal, central polypurine tract/central termination sequence (cPPT/CTS), rev response elements (RRE) that are primarily derived from a lentivirus.

The terms “integration deficient lentiviral vector,” may be used to refer to lentiviral viral vectors that are capable of functioning in the host cell as an episome and cannot integrate into the host genomic DNA or is highly unlikely to integrate into host genomic DNA.

The term “somatic hypermutation” may be used to refer to a process that can occur in B-cells. Where B-cell V-regions undergo mutation to produce variant immunoglobulins some of which may have higher affinity for the antigen that was the cause of the somatic hypermutation process. Somatic hypermutation occurs during many stages of B-cell development and during affinity maturation when germinal center B-cells enter the GC dark zone and undergo proliferation. When memory germinal center B-cells become activated by CD4+ helper T-cells they can assume one of three states of differentiation becoming a memory B-cell, memory plasma secreting B-cell or may re-enter the dark zone and undergo further proliferation where each daughter cell will have undergone different mutations in the V-regions as part of the process of affinity maturation. (See, e.g. Janeway, C., Travers, P., Walport, M., and Shlomchik, M., Janeway's Immunobiology, Ninth Edition. 2016. Garland Science: New York, N.Y. ISBN: (Paperback) 978-0815345053)

The term “therapeutic binding affinity” refers to a binding affinity of an immunoglobulin, immunoglobulin V-region or immunoglobulin CDR region where there is evidence of a potential of the immunoglobulin, V-region or CDR to potentially provide a health benefit that may include mitigation of a pathology including a systemic pathology or an external pathology, avoidance of virus contraction or protection from any foreign body that could promote pathology in humans.

### 2. Isolation, Identification and Characterization of a Human and Non-Human Vertebrate Source of Immunoglobulins or Human Immunoglobulins with High Affinity or Therapeutic Binding Affinity for the Antigen or Pathogen Associated Protein, Biowarfare Agent, Bacteria, Fungi or Allergen, Immune System Protein, or Self-Antigen of Interest

The peak affinity of one's immunoglobulins for key antigens or protein targets such as highly conserved cell surface antigens in viruses, bacteria, fungi, microorganisms and other pathogens after affinity maturation has taken place is an important factor in one's ability to fight pathogens that evade the innate immune system. The innate immune system relies in part on evolutionarily conserved polypeptide sequences to detect pathogens. When the peptide sequences of proteins produced and especially presented on the surface of viruses lack a minimal degree of sequence similarity necessary to detect such evolutionarily conserved peptide sequences the participation of the adaptive immune system is required to directly detect such specific pathogens. With an increased number of iterations of exposure to pathogenic proteins and antigens the adaptive immune system can increase its affinity for antigens through a process of affinity maturation. When a B-cell becomes immature it typically starts out as an immunoglobulin class M (IgM) or immunoglobulin class D (IgD) B-cell or expressing both that also bears a cell surface immunoglobulin class M receptor or IgD receptor or both IgD and IgM B-cell receptors (BCRs). Part of the B-cell development process consists of going from the early pro-B-cell to small pre-B-cell stage where immunoglobulin gene element rearrangements occur and are evaluated to ensure productive matches and also the undeveloped B-cell detects in the bone marrow for self-antigens. In the Bone marrow when the affinity for a self-antigen is above a specific threshold the undeveloped B-cell can be rescued by gene rearrangements of gene elements of the V-region and is known as “receptor editing”. If repeated receptor-editing fails to produce an immunoglobulin chain that are not strongly reactive to self-antigens the undeveloped B-cell will undergo apoptosis. Immature B-cells have heightened sensitivity to self-antigens and can continue to undergo receptor editing or antigen-induced apoptosis. (See e.g., Melamed, D., 1998, Cell. 92:173-182) However, about 20% of immature B-cells have some reactivity to self-antigens (See e.g., Wardemann, H., et. al., 2003, Science 301:1374-1377). As without this we would not be able to sufficiently generate an adaptive immune response to a broad range of antigens. Immature B-cells undergo further differentiation into mature naïve B-cells that continue to bear cell surface immunoglobulins of IgM or IgD.

In phase 1 of the primary immune response mature naïve B-cells leave circulation and enter secondary lymph nodes and can engage in antigen encounter with follicular dendritic cells. B-cells then process antigen and present it on their Major Histocompatibility Complex class II MHC-II to the T-cell receptor (TCR) of T-helper cells. Upon activation by T-helper cells at the T-cell/B-cell border the B-cell undergoes proliferation in the follicle with three possible fates: A Germinal Center independent memory B-cell bearing an IgM receptor, short-lived plasma secreting IgM B-cell with a half-life of a few days or a Germinal Center B-cell that will enter the dark zone and undergo proliferation and somatic hypermutation. A secondary encounter with antigen from a follicular dendritic cell followed by processing presentation on the MHC class II to the TCR of T-follicular-helper cells results in activation of the germinal center B-cell that can undergo isotype switching generally to IgG(1, 2, 3 or 4) or IgA(1 or 2) and can be dependent on the lymph node. For example, lymph nodes that support the mucosa of any organ tend to cause IgM B-cells to class switch to IgA. They can become a memory B-cell bearing the cell surface immunoglobulin, a memory plasma B-cell that can persist for decades depending on the support from the local environment, which is often the bone marrow, a plasma blast that is in the process of converting to a memory plasma secreting B-cells or can reenter the dark zone for further proliferation and somatic hypermutation repeating the cycle. IgG and IgA Memory B-cells upon activation can differentiate into a long-lived memory plasma B-cell or can reenter the dark zone for further proliferation and somatic hypermutation. T follicular helper cell (T_{FH} cell) Germinal Center B-cell interactions are important for the generation of germinal center B-cells. (See, e.g. Akkaya, M., Kwak, K. & Pierce, S. K., 2020, Nat Rev Immunol., 20:229-238). The long-lived plasma B-cells may migrate to the bone marrow where they can be sustained for decades as a result of the microenvironment in the bone marrow and even potentially the stomach and small intestine. Although, the memory plasma B-cells in the small intestine and stomach may be derived directly from the local lymphnodes supporting the organ or may migrate to the interstitium of the stomach and small intestine while not being supplied from local lymphnodes. (See, e.g. Akkaya, M., Pierce, S. K., 2020, Nat Rev Immunol., 20:229-238; Also see, Landsverk, O. J., Jahnsen, F. L, et al., 2017, J. Exp. Med. 214:309-317; Khodadadi, L., Hiepe, F., 2019, Frontiers in immunology, 10:721)

The repeated cycle of re-entry into the dark zone allows the affinity maturation process where cell surface immunoglobulin bearing B-cells undergo a repeated cycle of proliferation and somatic hypermutation potentially increasing the affinity for the antigen of interest. Both these processes can potentially result in B-cells with reactivity to self-antigens. Naïve B-cells give rise to both lymphoblasts that secrete antibodies and memory B-cells upon activation from a pathogenic antigen and the activation of CD4+ helper T-cells. Through, repeated cycles of proliferation and somatic hypermutation of germinal center memory B-cells the resulting memory B-cells and the resulting daughter long-lived plasma B-cells can develop a greater affinity for the antigen of interest. (See, e.g. Akkaya, M., Pierce, S. K., 2020, Nat Rev Immunol., 20:229-238; Also See e.g., Ziegner, et. al., 1994, Eur. J. Immunol. 24:2393-2400; Shlomchik, M. J., et. al., 1990, Prog. in Immunol. Proc. 7:415-423; Berek, C., & Milstein, C., 1987, Immunol Rev. 96:23-41; Ochiai, K., Sciammas, R., 2013, Immunity, 38:918-929). It is through this process that affinity-matured immunoglobulins can be produced by B-cells and memory B-cells can for the most part generally be considered to express immunoglobulins that have high affinity for an antigen of interest. Additionally, during the course of any somatic mutation process that results in memory B-cells the possibility exists that memory B-cells can have a higher affinity for self-antigens. Although, these memory B-cells are unlikely to be activated by CD4+ helper T-cells. There is a panel of assays and additional assays that are used to determine if immunoglobulins have reactivity to self-antigens. Although, it is not comprehensive because of the vast number of proteins produced in the human body. Thus, perhaps the best assay would be based on human bone marrow. (See e.g., Tiller, T., et. al. 2007, Immunity, 26:205-213; Also see e.g., Fsuiji, M., et. al. 2006, The Journal of Experimental Medicine 203:393-400, 2006; Wardemann, H., et. al., 2003, Science 30:1374-1377).

Vectors may be created that encode for those immunoglobulins to be evaluated. Other biological sources such as a transgenic animal, a mouse with humanized immune system, or a mouse that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment (8) an allergen or cause of allergies, (9) a target protein or variant (10) an immune system protein such as IgE or a cytokine may be used to identify potent immunoglobulins for the target of interest. Additionally, in some embodiments the CDR regions and in other embodiments the V-region polypeptide encoding nucleic acids of the heavy (IgH) and light chain (IgL) of the immunoglobulins may be incorporated into a vector construct that could use either the constant region DNA identified in the B-cell expressing the identified immunoglobulin of interest or another source of genetic information may replace all or part of the constant region DNA that may also include isotype switching of one or more of C_{H}1, hinge, and C_{H}2 constant regions but C_{H}3 constant regions are limited to the Cα₁ or Cα₂ gene locus, mixes of two constant regions from two isotypes.

Additionally, the immunoglobulin hinge length may be modified. Central towards this end is the production of dIgA1 and dIgA2 to enable mucosal immunity against the (1) virus (s) (2) systemic ailment (3) fungi (4) bacterial infection (4) cancerous tumor (5) biowarfare agent (6) microbial infection, (7) any ailment (8) an allergen or cause of allergies such as IgE or cytokines, (9) target protein or variant of interest.

A human (ideally between 21 and 55 years old), a transgenic animal, a mouse or other animal with humanized immune system, or a mouse that was infected with, exposed to, has immune specificity to or lives with any of (1) a virus (s) (2) a systemic ailment such as allergies (3) fungi (4) bacterial infection (4) cancerous tumor (5) biowarfare agent (6) microbial infection, (7) any ailment (8) an allergen (9) a target protein or variant (10) in the case of non-human vertebrates a human immune system protein such as Immunoglobulin class E (IgE) or a cytokine has their blood drawn. The blood sample will be allowed to separate into its three layers or can be subjected to blood fractionation method such as density gradient centrifugation. The buffy coat layer, which contains the peripheral blood mononuclear cells (PBMCs) that includes memory B-cells is collected.

In addition to a number of published approaches to separate Human CD27+ memory B-cells from a blood sample one may isolate Human CD27+ IgG+ memory B-cells would be with EasySep™ Human IgG+ Memory B-Cell Isolation Kit¹. This kit can be purchased from STEMCELL Technologies in Cambridge, Mass. This kit also includes an option to purchase an additional separation assay to separate out CD27+ IgA+ memory B-cells. In a similar sense all of the memory B-cells could also be isolated and analyzed together. Although, it is preferred to analyze the CD27+ IgG+ and CD27+ IgA memory B-cells with option to analyze separately or together. The separation of different memory B-cell isotypes is achieved through the use of immunoglobulins specific for the heavy chain Fragment crystallizable (Fc) constant regions of a specific memory B-cell isotype. Using the STEMCELL Technologies method primary human CD27+ memory B-cells can be isolated by immunomagnetic bead isolation followed by a depletion cocktail to deplete Human IgM/IgD/IgA B-cell Depletion Cocktail leaving the CD27+ IgG memory B-cells as a pure sample. Also available is a solution to deplete Human IgA B-cells. Thus, both IgG and IgA memory B-cells if not all memory B-cells will be collected. (Catalog #17868, STEMCELL; Website: https://www.stemcell.com/easysep-human-igg-memory-b-cell-isolation-kit.html#section-data-and-publications) Patient PBMCs may be stored frozen and thawed before use. (For an alternative method to isolate CD27+ B-cells see e.g., Tiller, T., et. al. 2007, Immunity, 26:205-213) Other methods to separate CD+ memory B-cells may be used where such B-cells can be subsequently separated with FACS or magnetic isolation experiments that take advantage of differences in the immunoglobulin constant regions on the cell surface immunoglobulins as a means to separate out IgG from IgA B-cells or plasmablasts or other B-cell that expresses an IgG or IgA B-cell receptor on its surface. It is difficult to perfectly separate the IgG memory B-cells by their subclasses e.g. IgG1, IgG2, IgG3 etc. IgG1+ memory B-cells generally make up 60%-70% of IgG memory B-cells in healthy donor serum. IgG2+ memory B-cells make up about 25% of IgG memory B-cells and IgG3+ memory B-cells make up about 9% of IgG memory B-cells. This difficulty in separation suggests that all IgG subclasses have heavy chain constant regions that are topographically similar. (See e.g., Tiller, T., et. al. 2007, Immunity, 26:205-213) However, this is not important because downstream steps will address these differences accordingly. ¹ Website: https.//wwwstemcell.com/easysep-human-igg-memory-b-cell-isolation-kit.html#section-data-and-publications

Additionally, tt is understood that there are established methods to isolate memory B-cells from transgenic mice or rabbit, mouse with humanized immune system and from mice that are well known to those familiar with the art and are consistent with the approach described to isolate the human memory B-cells. IgG memory B-Cells or IgA memory B-cells will then be separated by relative binding affinity through a flow cytometry technique such as fluorescence activated cell sorting (FACS). The flow cytometry analysis of the relative binding affinities of memory B-cells will be dependent on labeling proteins such as antigens with a fluorescent tag to allow detection of antigen bound B-cells by the FACS that will employ the use of a competing binding protein such as the protein that the target protein binds. (E.g. if the target protein binds the CCR5 receptor, then it could be used as a competing protein in the assay e.g. in a soluble form). There are a number of well-established methods to identify, isolate and characterize B-cells and their immunogloublins with cell surface immunoglobulins that have binding affinities that warrant further investigation. (See e.g., Weiss, G. E., et. al., 2012, Journal of immunological methods, 375:68-74; Hunter, S. A., & Cochran, J. R., 2016, Methods Enzymol. 580:21-44; Li, P., Selvaraj, P., & Zhu, C., 1999, Biophysical journal, 77:3394-3406; Amanna, I. J., & Slifka, M. K., 2006, Journal of immunological methods, 317:175-185)

Immunoglobulin DNA sequences of the CD27+ IgG+ and CD27+ IgA+ memory B-cells, Germinal Center B-cells, CD+ memory B-cells or plasmablasts can be obtained through established methods. (Niu, X., et al. 2019, Emerging microbes & infections, 8:749-759; Cao, Y., 2020, Cell, 182:73-84) These are well-established methods known to those of skill in the art and also available from a variety of commercial sources with standardized protocols. Further, such a protocol to obtain the DNA encoding for potent immunoglobulins was previously established to allow for efficient isolation and identification of neutralizing monoclonal antibodies (mAbs) in different infectious diseases. (See e.g., Setliff, I., et. al., 2019, Cell, 179:1636-1646; Wu, X., et. al., 2010, Science, 3295:856-861)

Alternatively, memory B-cells specific to the antigen can be subjected to an antigen isolation assay where antigen biotinylated to magnetic beads is competed for between memory B-cell surface immunoglobulins such as IgG+ or IgA+ of different memory B-cells. Magnetic pull downs will only capture those memory B-cells with the greatest relative binding affinity for the antigen of interest. B-cells can also compete against a protein of interest that is present in the solution with known binding affinity to the biotinylated antigen. (See e.g., Cao, Y., 2020, Cell, 182:73-84; Niu, X., et al. 2019, Emerging microbes & infections, 8:749-759) These established methods and associated kits known to those of skill in the art are also available from a variety of commercial sources with standardized protocols. However, for any particular antigen, assays may have to be developed using established methods known to those of skill in the art. If magnetic beads are used what would result are a cohort of memory B-cells that could then be separated with a subsequent competitive binding flow cytometry assay such as FACS as described herein. Alternatively, the memory B-cells isolated from the magnetic pulldown separation. Cell surface immunogloublins can be assessed for binding affinity by producing the free individual monoclonal immunolgoublins the B-cell encodes for and recombinant isotypes of them and assessing their actual binding affinity or dissociation constant with the use of surface plasmon resonance. It is understood that converting an IgG immunoglobulin into an IgA1 recombinant isotype can result in a 1 to 2 order of magnitude increase in binding affinity. Those memory B-cells with among the more desirable affinities that is dependent on disease model may be identified from one or more of (A) the magnetic pulldown isolations assay followed by FACS separation (B) magnetic pulldown isolations (C) FACS assays. (Niu, X., et al. 2019, Emerging microbes & infections, 8:749-759)

Additionally a neutralization titer (NT₅₀) inhibitory dose may be determined to assess the neutralization potential of immunoglobulins of known concentration in solution for an antigen of interest. The NT₅₀ is defined as neutralization titers [50% inhibitory dose (ID50) or the 50% inhibitory concentration (IC50)] that is defined as the reciprocal of the serologic reagent dilution (or concentration for purified reagents) that caused a 50% reduction in relative luminescence (RLU) compared to virus control wells after subtraction of background RLUs. (See e.g., Robbiani, D. F., et al., 2020, Nature, 584:437-442; Antonio, E., et. al., 2020 bioRxiv, 2020 May 21; Sarzotti-Kelsoe, M., et. al., 2014, Journal of immunological methods, 409:147-160).

The DNA sequences that codes for the immunoglobulins expressed by the memory B-cells may be determined by one of several established methods. Such techniques are well established and understood by those that practice the art. Antigen-binding memory B-cells or plasmablasts may be identified after a magnetic pulldown isolation experiment and/or flow cytometry. Identification of the immunoglobulin heavy and light chain polypeptide sequences may be used to incorporate such genetic information into a plasmid transfected into e.g. a HEK-cell or human hepatocytes for expression and evaluation of the secreted antibody. (see e.g., Setliff, I., et. al., 2019, Cell, 179:1636-1646; Cao, Y., 2020, Cell, 182:73-84;)

Discussed throughout this instant patent application the possibility exists that memory B-cells can have a high affinity for self-antigens. There is a panel of assays and additional assays that are used to determine if immunoglobulins have reactivity to self-antigens. There are well-established methods to carry out these panel assays with monoclonal antibodies known to those of skill in the art. (See e.g., Tiller, T., et. al. 2007, Immunity, 26:205-213; Also see e.g., Fsuiji, M., et. al. 2006, The Journal of Experimental Medicine 203:393-400, 2006; Wardemann, H., et. al., 2003, Science 30:1374-1377). Another described global self-antigen assays can also be used to evaluate immunoglobulin for self-reactivity. (See e.g., Vale, et. al., 2016, Front. Immunol. 7; Nobrega, A., et. al., 1993, Eur J Immunol., 23:2851-2859; Haury, M., et. al., 1997, Scand. J. Immunol. 39:79-87) (See e.g., Weiss, G. E., et. al., 2012, Journal of immunological methods, 375:68-74)

### 3. Modification or Incorporation of Constant Regions of Natural or Engineered Sources of Human Derived or Humanized Mice, Transgenic Mice, Non-Human Vertebrates (e.g. Mouse or Rabbit) Intended for Chimeric Antibodies, Derived Human Immunoglobulins or V_{H} and V_{L} Regions Identified from Phage Display Technology with High Affinity for the Targets of Interest

Those immunoglobulins discovered from human B-cells, mice, transgenic mice or mice with humanized immune systems that are deemed to have “therapeutic binding affinity” generally high affinity for antigen of interest while also being at sufficiently low reactivity for self-antigens will have their DNA incorporated into an expression vector for further evaluation. Additionally, high affinity V_{H} and V_{L} regions identified from phage display technology will be incorporated into full length immunoglobulin heavy and light chains for further evaluation.

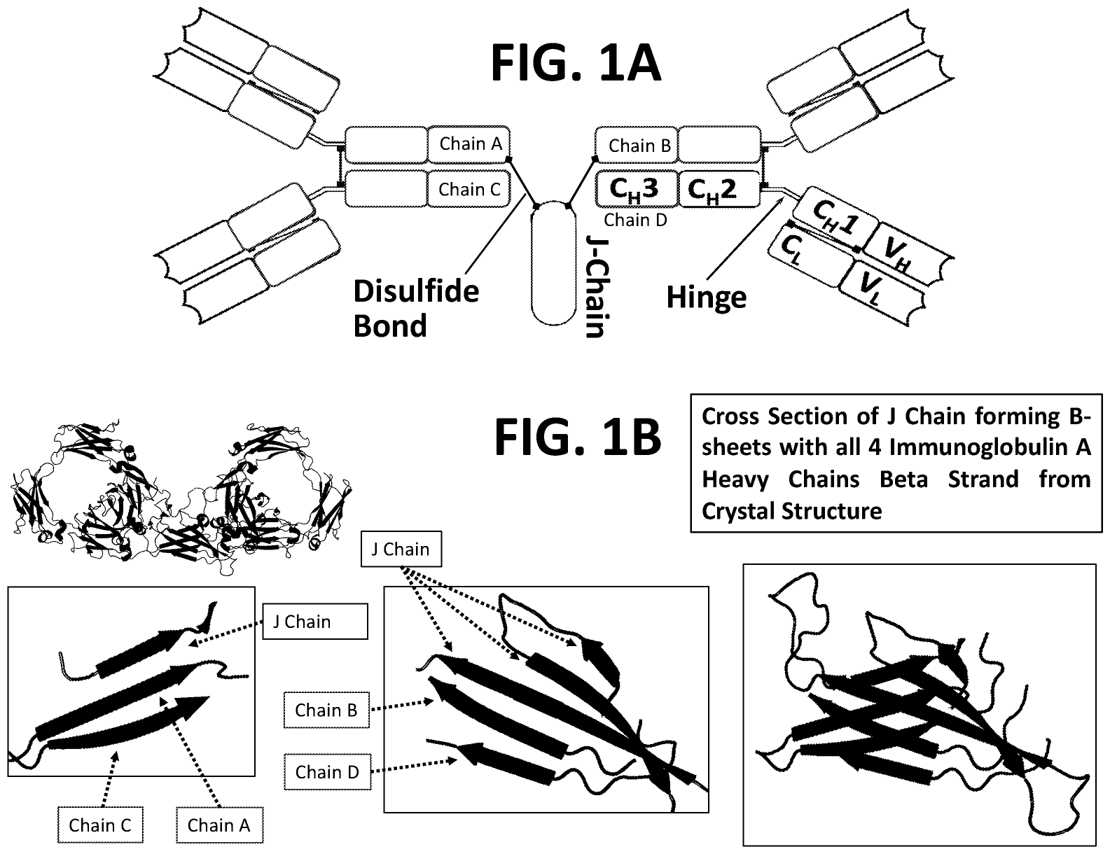

That evaluation of immunoglobulins identified from any of the sources may further include modifying the constant regions of the immunoglobulins using a variety of different sources of human genetic information or through engineering the constant regions to modulate effector functions as dependent on the application. All such immunoglobulins may be evaluated in the battery of tests that include self-reactivity assays and binding assays as described in this document. There are specific limitations on how constant regions may be modified so as to ensure the formation of polymeric immunoglobulin A. As an example the use of the C_{H}3 constant domain of IgHA1 or IgHA2 is used in the present invention to ensure the formation dimeric immunoglobulin A and polymeric immunoglobulin A as a result of productive interactions between the tail of the C_{H}3 domain and J Chain in forming a central beta sandwich complex (FIG. 1B). Additionally, in some embodiments the C_{H}3 constant domain of IgHA1 or IgHA2 may be engineered to enhance its participation in complexing with J Chain to favor formation of high valency (e.g. pentameric immunoglobulin A and tetrameric immunoglobulin A) polymeric immunoglobulin A.

Plasmids will be created that encode for immunoglobulins to be evaluated. The V-region and some of the constant region DNA of the heavy (IgH) and light chain (IgL) of the immunoglobulins will be incorporated into a vector construct that could use either the constant region DNA of another isotype and/or subclass or another source of human genetic information may replace all or part of the constant region DNA for the immunoglobulin heavy and/or light chain that may also include (A) class or subclass switching of constant region genetic information (e.g. replacing all of the IgG2 constant domain with that of Ig2), (B) mixes of two constant regions from two isotypes or subclasses (e.g. replacing part of the IgG2 constant region with an IgA1 or IgA2 constant region. Although, generally at a minimum to ensure the formation of dimeric immunoglobulin A engineered variants the C_{H}3 (Constant Heavy 3) domain is derived from IgA1 or IgA2 because it is responsible for complexing with J Chain.

In the production of dimeric immunoglobulin A for both dIgA1 and dIgA2 from any cell invariably there are mixtures of higher ordered polymeric immunoglobulins of class A including trimeric immunoglobulin A, tetrameric immunoglobulin A and pentameric immunoglobulin A. Collectively, dimeric immunoglobulin A, trimeric immunoglobulin A, tetrameric immunoglobulin A and pentameric immunoglobulin A are referred to as polymeric immunoglobulin A and does not make any reference to the distribution of the different polymeric forms but rather acknowledges that more than one form in present and where each form can contribute to neutralization. Although in producing dimeric immunoglobulin A2 with the immunoglobulin heavy chain A2 of Allotype A2m(2) a more significant portion of polymeric forms of higher valency than dIgA2 are observed including trimeric immunoglobulin A2 allotype A2m(2), tetrameric immunoglobulin A2 allotype A2m(2) and pentameric immunoglobulin A2 allotype A2m(2). Whereas in the production of dIgA1 there is generally a minor fraction of higher valency polymeric forms although higher valency forms are present.

A major advantage of the secretory forms of trimeric immunoglobulin A, tetrameric immunoglobulin A and pentameric immunoglobulin A over the secretory form of dimeric immunoglobulin A is their enhanced neutralizing activity. By mass the mean neutralizing concentration of the secretory form of trimeric, tetrameric or pentameric form of immunoglobulin A can be greater than 4 to 5 times more neutralizing by mass which equates to about 6 to 10 times more neutralizing by concentration since the protein mass of higher ordered forms of secretory immunoglobulins nearly doubles over the dimeric form in going from the dimer to the pentamer. This enhancement in the neutralizing activity of higher ordered forms of polymeric immunoglobulin A is attributed to the increased valency or increasing number of antigen binding sites with higher ordered forms of secretory immunoglobulin A. (See, e.g. Suzuki, T., et al., 2015, Proceedings of the National Academy of Sciences of the United States of America, 112:7809-7814.)

This instant patent utilizes J Chain to form higher valency structures of immunoglobulin A including dimeric immunoglobulin A, trimeric immunoglobulin A, tetrameric immunoglobulin A and pentameric immunoglobulin A collectively referred to as polymeric immunoglobulin A. dIgA1 (inclusive of polymeric immunoglobulin A1) means that when dIgA1 is produced as the primary product or if dIgA1 is present other polymeric forms of IgA1 including trimeric immunoglobulin A1, tetrameric immunoglobulin A1 and pentameric immunoglobulin A1 may also be produced or present in unspecified stoichiometries. Central towards the formation of polymeric immunoglobulin A is a central Beta sheet complex (Beta sandwich) that formed from beta strands of J Chain and the C-terminal end or tail of the immunoglobulin A heavy chains (see FIG. 1B). (See, e.g. Kumar, S., et al. bioRxiv 2020.02) As an additional example, this interaction with J Chain is reported with the tail at the C-terminal end of immunoglobulin M where J Chain forms part of the Beta sheet complex (Beta sandwich) contributing 4 beta strands and each of the 10 Beta strands from the tails of each immunoglobulin A also form part of the extensive beta sheet complex that is a beta sandwich. (See, e.g. Li Y, Wang G, Li N, Wang Y, Zhu Q, Chu H, Wu W, Tan Y, Yu F, Su X D, Gao N, Xiao J., 2020, Science. 367:1014-1017.)

In consideration of human derived immunoglobulins or immunoglobulins derived from mice with humanized or engineered immune systems likely, the immunoglobulin light chain (IgL) constant region would be coded for in the vector as identified in addition to being prepared in other isotypes. Additionally, the V_{H}-region of the immunoglobulin heavy chain may be paired with the constant domains of one or more of C_{H}1, hinge and C_{H}2 but not C_{H}3) of another isotype while leaving the immunoglobulin light chain unchanged. Increased binding affinity can occur simply as a result of an isotype switch from IgM or IgD to IgG, IgA or IgE without changing the amino acid sequence of the V-region nor modifying the immunoglobulin light chain. Further, the constant regions of each class and subclass have different effector functions. Additionally, enhanced or reduced signal transfer from Fab to the Fragment crystallizable (Fc) region to the can occur simply as a result of isotype switching. (See e.g., Pritsch, O., et al., 1996, J Clin Invest. 98:2235-2243; Janda, A., et. al., 2016, Front Microbiol., 7:22). Not only will immunoglobulins V-regions be investigated for different isotype functions, but they will be investigated for their Fab and F(ab)₂ fragments.

### 4. Incorporation of the Immunoglobulin Genetic Information of IgG1, IgG2, IgG3, IgG4, IgA1, IgA2, dIgA1 or dIgA2 into Non-Viral, Viral or Retroviral Expression Vectors Intended for Different Delivery Vehicles Such as Adeno-Associated Virus, Adenovirus, Lentivirus, Gammaretrovirus or Vesicle Based Delivery Systems to Enable a Polyclonal Expression of Immunoglobulins

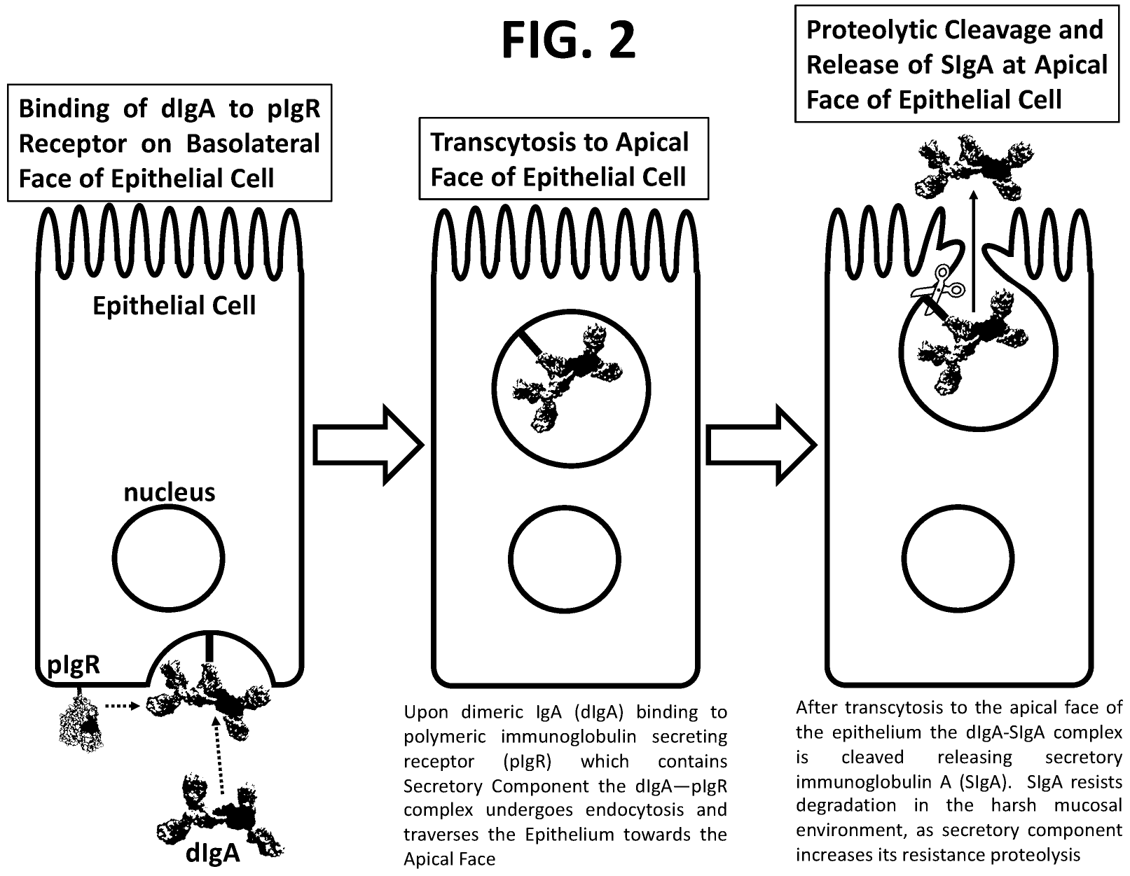

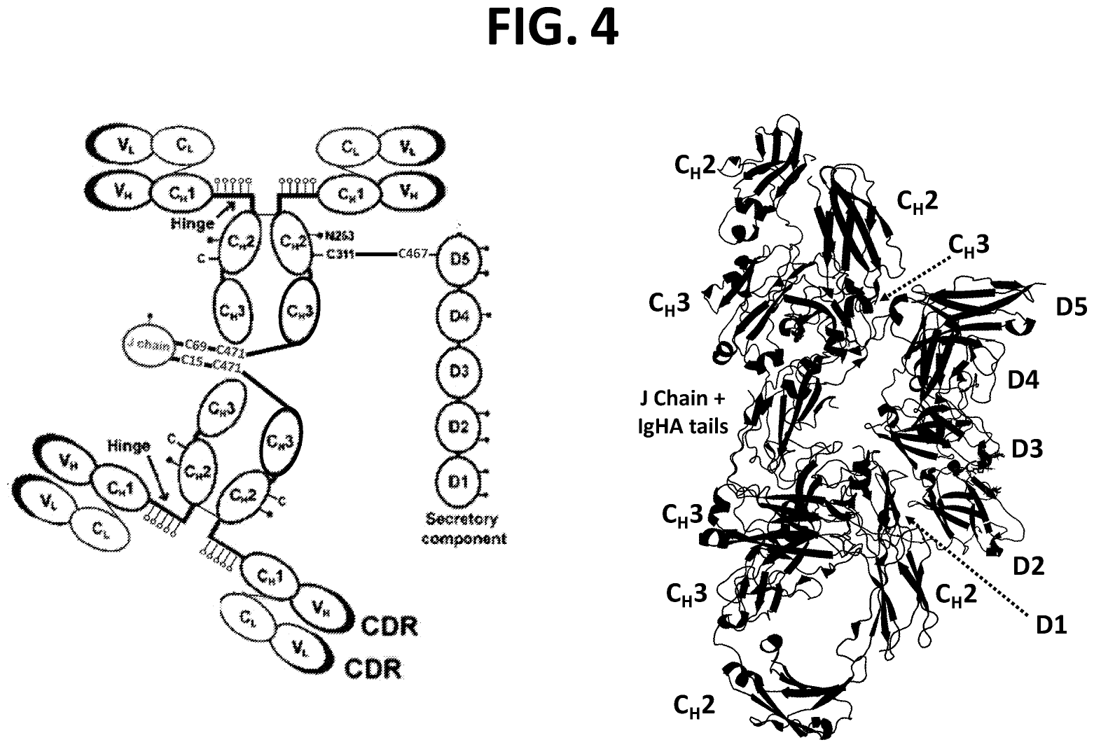

In the invention the immunoglobulins identified to both have “therapeutic binding affinity” are intended to be expressed from episomal DNA in the host cells. AAV, lentivirus, gammaretrovirus, vesicle based delivery systems will be used to transport the vector constructs to their target cells with destination of the nucleus of the target cell. In the invention A mix of episomes each of which codes for a unique immunoglobulin and collectively which code for a polyclonal mix of immunoglobulins. As an example, there may be a total of 3 unique immunoglobulins that are each separately coded for by separate single stranded DNA in an AAV, double stranded DNA in an adenovirus, mRNA or DNA in a vesicle-based delivery system, or mRNA in a lentivirus or gammaretrovirus. Those immunoglobulins will include one or more of the following immunoglobulin classes: dIgA1 and dIgA2, IgG1, IgG2, IgG3, IgA1, IgA2, (See FIG. 3). In one embodiment a single immunoglobulin may be used as well as part of a monoclonal antibody expression from an episome. Mucosal immunity against the pathogen or pathology of interest is best achieved with SIgA1 or even SIgA2 that is a product of dIgA1 or dIgA2 respectively following transcytosis from the basal to apical face of an epithelial cell where dIgA1 forms a disulfide bond (See FIG. 4) with secretory component that is found on polymeric immunoglobulin secreting receptor (pIgR) as part of crossing the epithelial cell (See FIG. 2). It is proposed that dIgA1 would provide more effective mucosal immunity over dIgA2 in cases where the hinge is not heavily subjected to cleavage by proteolysis such as in the digestive tract; although, SIgA1 can function effectively in the digestive tract but is more prone to cleavage than SIgA2. IgA1 has a 19 amino acid hinge length, which affords great flexibility between the Fab and Fc region and also allows for a more rapid rate of agglutination between multiple virions, bacteria, allergens, or any binding target with multiple binding faces and the secretory immunoglobulin A1 (SIgA1). In the absence of secretory component the hinge of IgA1 and dIgA1 is readily cleaved by proteases in the mucus where there are multiple cleavage sites on the hinge where the 6 amino acid hinge of IgA2 and dIgA2 do not have any cleavage sites in the hinge and thus would not be degraded as quickly in the mucus. Although, SIgA1 is not cleaved quickly at the hinge by proteases in the harsh environment of the mucus as because secretory component effectively reduces the rate with which proteases access cleavage sites on the immunoglobulin chains and also the dIgA1 part of secretory immunoglobulin A blocks proteases from accessing cleavage sites on secretory component. IgA2 has a 6 amino acid hinge length, which protects it from proteases but does not afford it a high degree of flexibility. (See e.g., Bonner, A. et. al., 2009, Mucosal Immunol 2:74-84; Kumar, S., et al. bioRxiv 2020.02) This short hinge length of dIgA2 makes it more difficult to achieve agglutination with 4 targets that a single dIgA1 is capable of where dIgA2 is more likely to achieve agglutination with two binding targets of the dIgA2 immunoglobulin. Another challenge accorded with the short hinge of dIgA2 is the lack of flexibility between the Fab and the Fc region making this immunoglobulin more constrained in sampling the immediate vicinity of nearby binding targets. Although, at the same time in producing dIgA2 allotype A2m(2) invariably produces a large fraction of polymeric immunoglobulin A2 that may be more neutralizing that dIgA1 in at least some applications. Additionally, in the gut mucosa because the environment is even more harsh than other mucosal environments SIgA1 could be more subjected to cleavage than SIgA2. In further embodiments of the invention a hinge longer than dIgA2 but shorter than dIgA1 is considered. In additional embodiments a hinge longer than that of dIgA1 is considered. In further embodiments replacing one or more of the amino acids that make up the hinge of dIgA1 or dIgA2 with another amino acid is considered.

Immunoglobulin constant regions can have isotypic variants known as allotypes which are generally substitutions of one or more amino acids between allotypes. For example the constant region of the IgA2 immunoglobulin heavy chain has two allotypes referred to as A2m(1) and A2m(2). Allotype A2m(2) differs from allotype A2m(1) in that there are 23 amino acid substitutions in addition. IgA1 on the other hand does not have allotypes and also unlike both allotypes of IgA2, IgA1 does not have a disulfide bond between the immunoglobulin light and heavy chains. The immunoglobulin Kappa light chain also has three allotypes Km(1), Km(2) and Km(3) that differ by only one or two amino acids between each allotype. Although interestingly Km(3) is the most commonly used allotype in monoclonal antibodies as some reports cite enhanced specificity from Km(3) over Km(2) and Km(1) where Km(3) has alanine at position 153 and valine at position 191. The Lambda light chain also does not have allotypes discovered in nature. There are several reports that speak of potential immunogenicity from using natural allotypes in therapeutics and even in convalescent plasma and blood transfusions. Although, there is definitive reports of anti-allotype responses patients do routinely express anti-therapeutic antibodies. Although, these anti-therapeutic antibodies could be the result of the V-regions and even more specifically the CDR regions rather than the constant regions of the immunoglobulin. (See, e.g. Toraño, A., & Putnam, F. W., 1978, Proceedings of the National Academy of Sciences of the United States of America, 75:966-969; Li, Z., 2013, Exp Hematol Oncol 2:6)

Immunoglobulins are dimers of heterodimeric proteins that consist of light and heavy chain proteins of moderate size linked together through disulfide bonds and thus, they can be difficult to express from adeno-associated virus (AAV) viral vector constructs due to the AAV capsid limited packaging capacity. Reports of the expression of immunoglobulin vector constructs have focused on the expression of immunoglobulins that are dimers of the heterodimers. That is the immunoglobulin consists of two identical heavy chains and two identical light chains linked together through disulfide bonds. One reason immunoglobulins can be difficult to express in vector constructs due to the limited capacity of the vectors. For example, the maximum capacity of an Adeno-Associated Virus (AAV) capsid is typically about 4.9 kilobases of single-stranded DNA. Typically, to express two gene elements from a single vector construct there must be enough room for both DNA sequences, a promoter that includes a ribosome binding site, an intermediate promoter between the two gene elements that includes a ribosome binding site, a polyadenylation element (e.g. Simian Virus 40 polyadenylation element (SV40 polyA) (For an example see SEQ ID NO: 4) or Bovine Growth Hormone polyadenylation element (BGA polyA)) and potentially a post transcriptional regulatory element (e.g. Woodchuck Hepatitis Virus (WHP) Posttranscriptional Regulatory Element (WPRE). In this context collectively, the sum of these elements approaches or exceeds the 4.9 kilobase capacity of the AAV vector or in the case of an IRES the expression of the section transgene is much lower. Although, in addition even if there is enough room to house the two genes with separate promoters and regulatory elements the expression level of one of the two genes if not both genes is often substantially reduced resulting in a substantial excess of one of the two immunoglobulin chains. There have been a few reports and patents that address the challenge associated with obtaining sufficiently high expression of immunoglobulins that are dimers of heterodimers. There are two major strategies that may be used. The first is to express the immunoglobulin light and heavy chains as a single open reading frame. When two genes of the immunoglobulin light and heavy chains are expressed as a single open reading frame (that is the stop codon of the upstream gene is not encoded) a furin cleavage site and a 2A self-processing peptide are used to ensure efficient yields and viable immunoglobulin production. (See e.g., U.S. Pat. Nos. 7,498,024 B2 and 7,709,224 B2 expressly incorporated by reference herein in their entirety). Alternatively, a 2A self-processing peptide placed between two consecutive transgenes encoded for in a single open reading frame may be used in the absence of the furin cleavage site.

In one embodiment A furin cleavage site is used in conjunction with a 2A self-processing peptide to eliminate the large 2A polypeptide byproduct residue that would otherwise be left on the C-terminal end of the immunoglobulin chain encoded directly upstream of the 2A self-processing peptide. Furin cleavage sites of four amino acids in length have a consensus sequence of (N-terminus-RXKR-cleavage point-(C-terminus)—where X can be any amino acid—and furin cleavage sites range from 4 to 6 peptides in length and are cleaved by furin or other proteases. If the furin cleavage site gene is located directly downstream to a transgene as part of a single open reading frame in a nucleic acid vector the expression of the transgene will occur with a 4-6 amino acid furin cleavage site residue on the C-terminal end of the resulting protein. Additionally, a 2A self-processing peptide has a consensus sequence following cleavage of (N-terminus)-D(X)E(X)NPG-cleavage point/ribosomal skip-P-(C-terminus). Thus, if a 2A self-processing peptide is followed by a transgene as part of a single open reading frame the expression of the transgene will occur with a 1 amino acid (Proline) 2A self-processing peptide residue on the N-terminal end of the resulting protein. Although, if there is a leader sequence or signal peptide that is cleaved from the protein as part of processing such as transporting the protein outside the nucleus then there would be no 2A cleavage or ribosomal skip residue left on the N-terminal end of the functioning protein. The ribosomal skip residue byproduct of 2A self-processing peptides are efficiently removed from the C-terminus of the adjoining upstream immunoglobulin chain by placing a furin cleavage site directly upstream of the 2A residue resulting in an immunoglobulin chain with a furin cleavage site on the C-terminus end following by the 2A self-processing peptide directly C-terminus to the furin cleavage site. (See e.g., Fang, J., et al., 2005, Nat. Biotechnol., 23: 584-590; Krysan, D. J., et. al., 1999, J Biol Chem. 274:23229-23234; U.S. Pat. No. 7,498,024).

An alternative and earlier reported strategy utilized a dual promoter rAAV vector to express immunoglobulins. CMV and SV40 small T-antigen intron was used as the promoter for the first transcriptional unit that was followed by the leader sequence (or signal peptide) and heavy chain sequence. The leader sequence is a 19 amino acid cleavable sequence that is expressed N-terminal on the heavy (Leader Sequence Mouse: MGWSCIFLFLLSVTVGVFS) (Most common Leader Sequence Human: MDWTWRILFLVAAATGAHS) and light chain (Among most common Leader Sequence found on Kappa light chains MDMRVPAQLLGLLLLWLPG) chain immunoglobulins and is necessary for efficient translocation into the endoplasmic reticulum. For a list of Leader sequences found in human contemplated in the invention see Table 1 (SEQ ID NOs: 27-41). A Bovine Growth Hormone (BGH) polyadenylation (polyA) (For an example see SEQ ID NO: 5) site was used as the post-transcriptional regulatory element for the first transcriptional subunit. The second transcriptional unit utilized a human elongation factor 1α (EF1-α) promoter that was modified to improve stability of DNA and RNA that also contained an intron 1117 (SEQ ID NO: 9) 5′ Untranslated Region (5′ UTR). An SV40 polyadenylation site was used as the post-transcriptional regulatory element for the second transcriptional subunit. (See e.g., Lewis, A. D., et. al., 2002, J. Virol. 76:8769-8775; Huang, M. T., et. al., 1990, Molecular and cellular biology, 10:1805-1810; Wu, C., et. al., 2004, Molecular and cellular biology, 24:2789-2796). Although, in this report the level of expression of the immunoglobulin was not considered to be a therapeutically relevant concentration.

Previously, these two considered vector constructs were used to express immunoglobulins that are dimers of heterodimers in mammalian models. (See e.g., Fang, J., et al., 2005, Nat. Biotechnol., 23: 584-590; Lewis, A. D., et. al., 2002, J Virol. 76:8769-8775) Although, these reports were not concerned with the expression of dimeric immunoglobulin A nor polymeric immunoglobulin A, which only reported exist for isotype A in human (isotype M exists in pentameric form or monomeric form). dIgA and Polymeric immunoglobulin A requires J Chain for efficient formation. No such reports or patents concerned with the expression of dIgA from a single AAV vector nor multiple AAV vectors have been identified, nor have such reports been identified for the expression of dIgA from a lentiviral vector, nor a gammaretroviral vector, nor any vector including an mRNA vector.

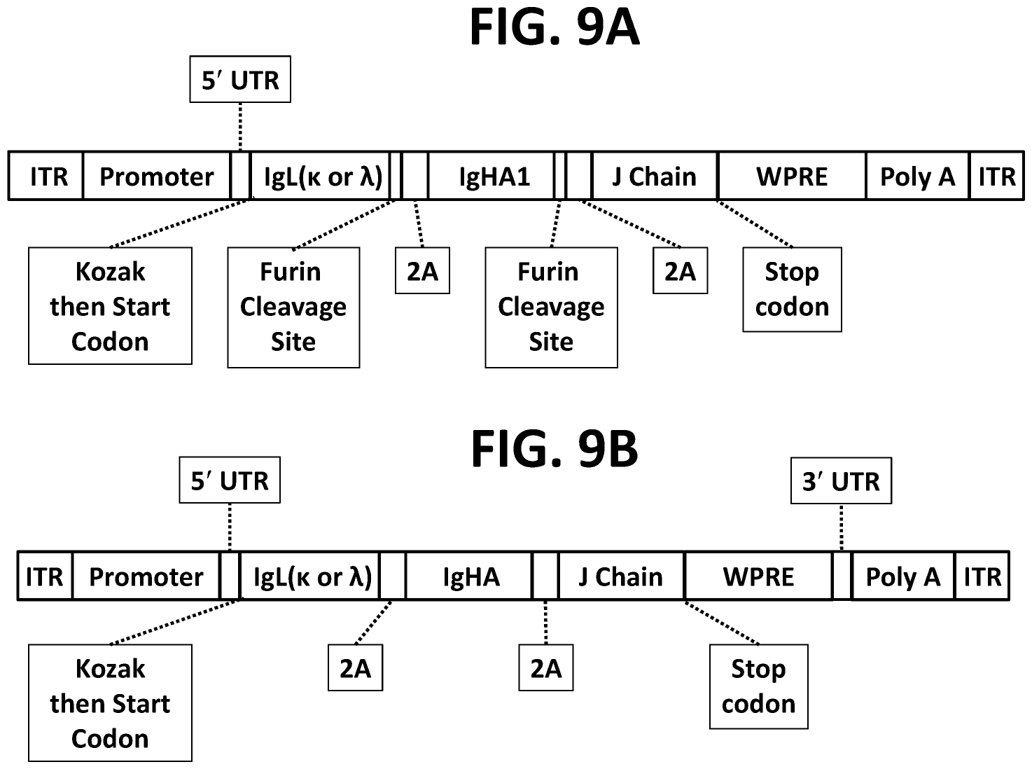

J Chain a 159 amino acid (SEQ ID NO: 11) protein with a 22 amino acid peptide signal leader sequence MKNHLLFWGVLAVFIKAVHVKA (SEQ ID NO: 27) that is cleaved to a 137 amino acid protein (SEQ ID NO: 7) is shown to be required for efficient production of dIgA. (See e.g., Lycke, N., et. al., 1999, J. Immunol. 163:913-919; Sorensen, V., et. al., 1999, J. Immunol., 162:3448-3455; Schroeder, H., et. al., 2008, Fundamental Immunology (Book) 6th:125-151; Castro, C. et. al., 2014, Journal of Immunology, 193:3248-3255; Koshland, M. E., 1985, Annu Rev Immunol. 3:425-453) Without J Chain there is a substantial reduction in the amount of dIgA produced by an organism such as mice. J Chain forms 2 disulfide bonds (See FIG. 9) with two IgA1 s at the penultimate cysteine (C471) on the heavy chain tail of each IgA1 and C15 and C69 of J Chain. Additionally, J Chain forms a central beta sheet complex (beta sandwich) that is formed from four beta strands of J Chain and the C-terminal end or tail of the immunoglobulin A heavy chains (see FIG. 1B). J Chain has been called the glue for the efficient formation of dIgA. (Bonner, A. et. al., 2009, Mucosal Immunol 2:74-84) J Chain has also been shown to be necessary for binding to secretory component on pIgR. (See e.g., Johansen, F. E., et. al. 2000, Scandinavian Journal of Immunology, 52:240-248) The partially buried ligand binding motifs of secretory component are thought to interact with J Chain of Dimeric IgA as part of inducing a conformational change to free domain 5 of secretory component on pIgR and form a disulfide bond between C467 of secretory component and C_{H}2 cysteine C311 of the immunoglobulin heavy chain of dIgA1. (See e.g., Wang, Y., Wang, G., Li, Y. et al., 2020, Cell Res 30:602-609).

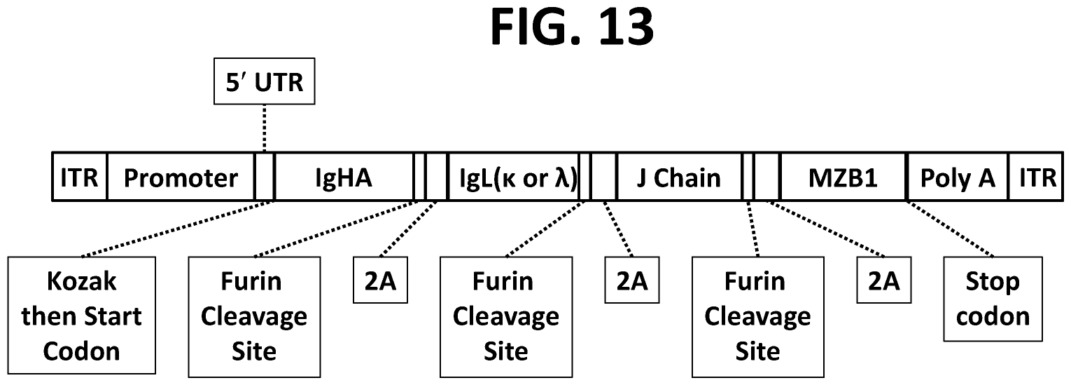

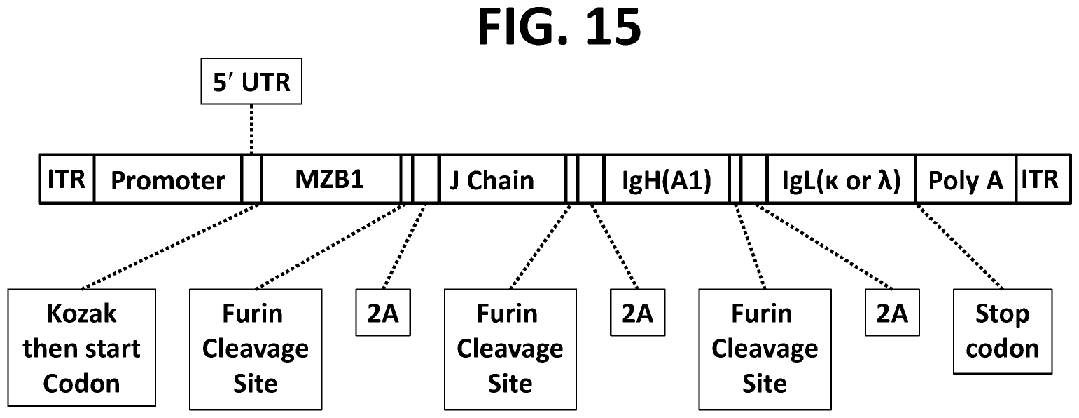

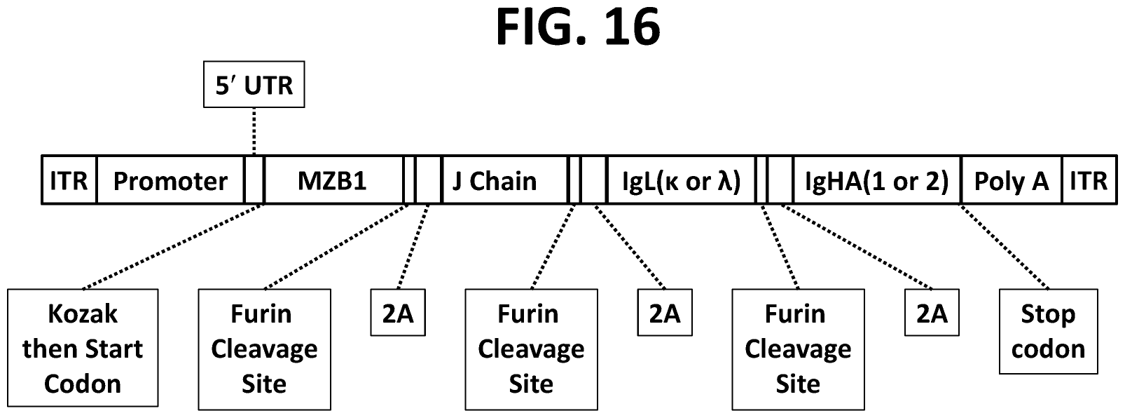

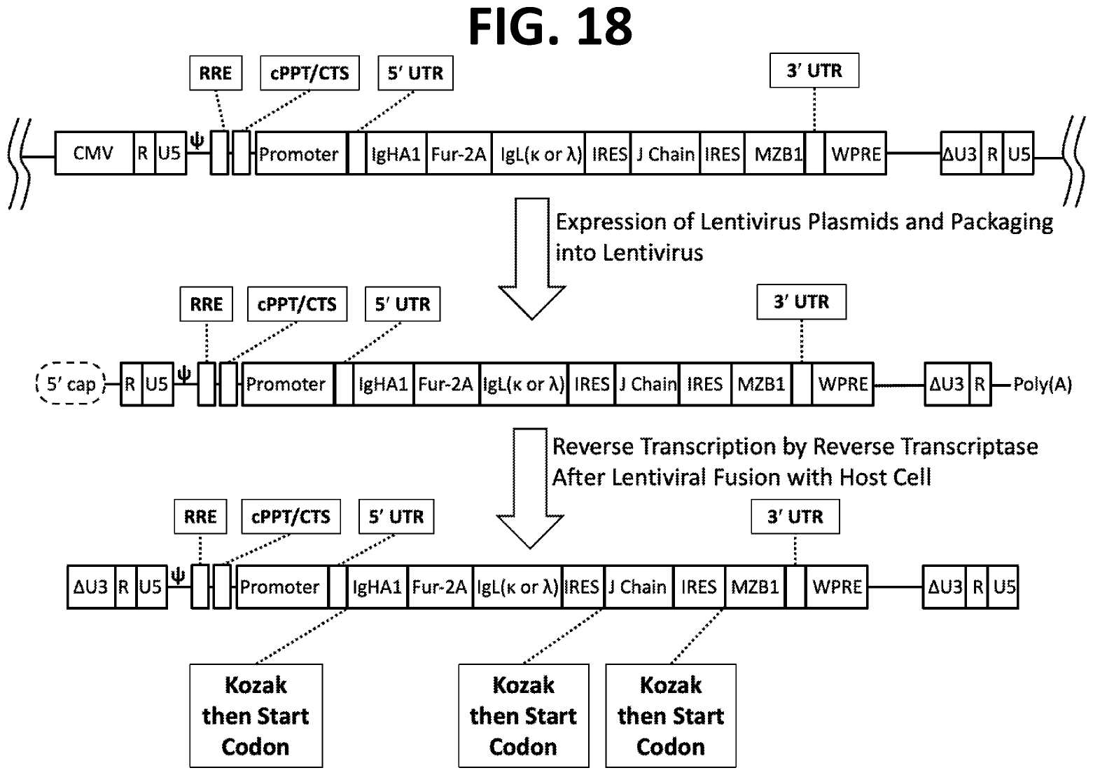

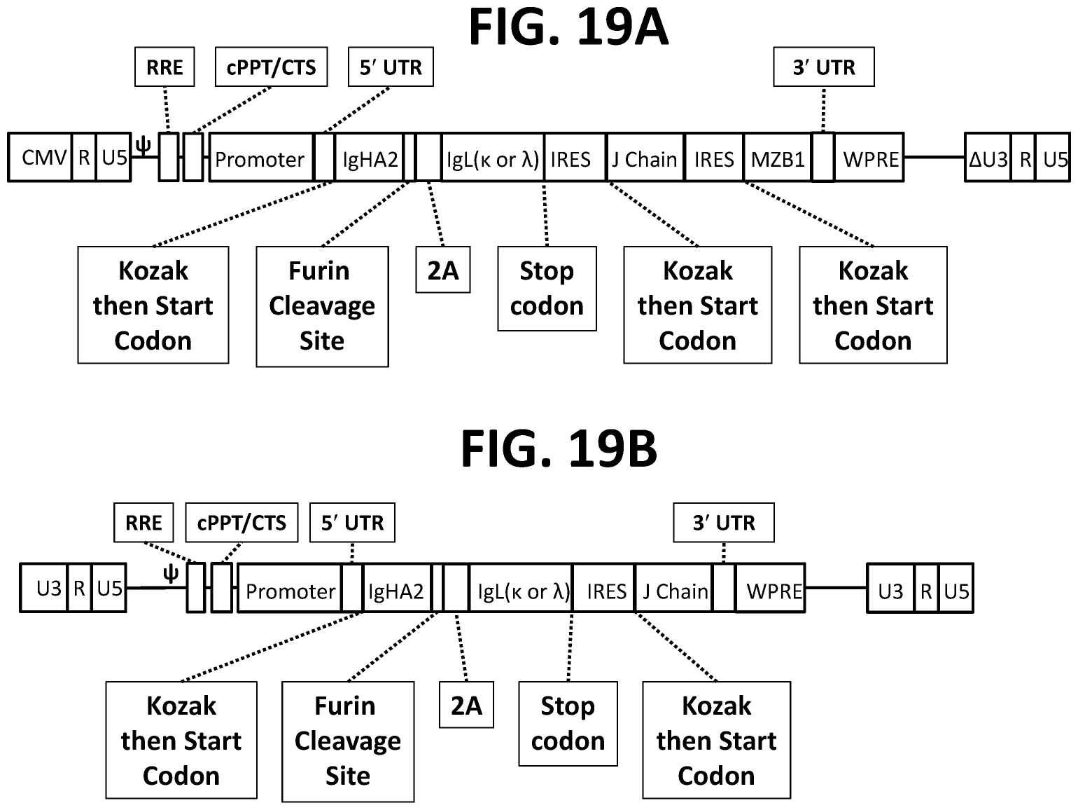

In some embodiments Marginal Zone B1 Cell Specific Protein (MZB1) (SEQ ID NO: 8) is co expressed in the vector for more efficient formation of some allotypes of dIgA2 and polymeric immunoglobulin A2. MZB1 is proposed to interact with IgA through the α-heavy chain (αHC) tailpiece dependent on the penultimate cysteine residue (where two αHCs form a disulfide bond with J Chain cysteine C15 and C69) and prevents intracellular degradation of α-heavy chain—Light chain (αHC-LC) IgA Dimer complexes. MZB1 promotes J Chain binding to IgA. Additionally, this instant patent optionally includes the expression of dIgA with MZB1 (See FIGS. 13, 15, 16, 17, 18, 19, and 20 as examples) and without encoding for MZB1 in the non-viral vector, viral vector or retroviral vector. In some vector constructs dIgA expression may occur through the incorporation of IgH, IgL and J Chain in the vector in the absence of encoding for MZB1.

### Immunoglobulin Regions

Human immunoglobulins are complex proteins with different structural/functional regions. FIG. 1 and FIG. 3 depict the different structural/functional regions of immunoglobulins. The fragment of the immunoglobulin responsible for binding to the antigen of interest is referred to as the antigen-binding fragment (Fab) region. The Fab region is considered to be made up of the entire immunoglobulin light chain (IgL) that includes its V-region (V_{L}) domain and constant region domain (C_{L}) and the immunoglobulin heavy chain's V-region (V_{H}) domain and C_{H}1 domain. The delineation of the Fab is depicted in FIG. 3 and can be applied to any immunoglobulin. The remaining part of the immunoglobulin is referred to as the Fragment crystallizable region and its delineation is depicted in FIG. 3 to include the C_{H}2 domain and the C_{H}3 domain in all IgA and IgG immunoglobulins of all subclasses. Researchers may also classify the Fab to include the hinge domain of the heavy chain and refer to it as F(ab′)2 fragment. The classification of F(ab′)₂ is related to dividing the immunoglobulin into two parts based on pepsin cleavage where the remaining part of the immunoglobulin is referred to as pFc′. The delineation by pepsin cleavage is not consistent across all immunoglobulins.

It is reasonably likely that one may be able to use the Fab or F(ab′)2 fragments of an immunoglobulin with therapeutically relevant affinity for a target of interest and show in many cases that making some modification to the Fc region and pFc′ and show there is little if any change in affinity of the Fab and F(ab′)2 for their target protein. One may incorporate IgA1 and IgA2 constant regions and immunoglobulin light chain constant regions IgL(κ or λ) into expression vectors that express V_{H} and V_{L} respectively as identified from scF_{V} identified in phage display libraries and also co-express J Chain (with optional co-expression of MZB1) to enable expression of dIgA1 and dIgA2. SIgA-virion polymerization/agglutination or SIgA-bacteria polymerization/agglutination is an effective potential means of virus or bacteria immobilization and neutralization in the mucosa. Further, dIgA-virion polymerization/agglutination or dIgA-bacteria polymerization/agglutination is an effective potential means of virus or bacteria immobilization and neutralization. These agglutination and polymerization properties are contemplated as a property of the invention. (see e.g., Terauchi, Y., et. al., 2018, Human vaccines & immunotherapeutics, 14:1351-1361). Additionally, in the invention the gene therapy-based expression of dIgA1 (inclusive of polymeric immunoglobulin A1), dIgA2 (inclusive of and polymeric immunoglobulin A2) and engineered variants and the corresponding SIgA are all contemplated to prevent cancer metastasis.

During affinity maturation B-cells can alter their immunoglobulins by both somatic hyper mutation of the DNA coding for the V_{H} and V_{L} domains. Additionally, during affinity maturation B-cells can alter their immunoglobulins through isotype switching that is through switching the entire constant region polypeptide. This isotype switching can result in changing the Cμ constant region with any downstream constant region. All immunoglobulin class switch recombination events occur from IgM or IgD to IgG, IgA or IgE. For example, IgG to IgA class switching is not reported to be observed. (See e.g., Stavnezer, J., et al., 2014, Journal of immunology, 193:5370-5378.) However, class switching can see an observed increased in affinity. Pairing the V-region of one Ig isotype with that of another can result in increased binding affinity simply as a result of an isotype switch from IgM or IgD to IgG or IgA without changing the amino acid sequence of the V-region. (See e.g., Janda, A., et. al., 2016, Front Microbiol. 7:22).

### Engineering Fc Region of Immunoglobulin to Prevent Antibody Mediated Viral Enhancement or to Improve Other Effector Functions

Engineering the Fc region of immunoglobulins can serve a large variety of functions including altering their half-life, to enhance complement dependent effector function, to enhance or reduce Fc receptor effector functions and to enhance binding affinity of immunoglobulins. Enhancing complement dependent effector functions as enhancing the complement effector functions of an immunoglobulin leads to more rapid response by supporting immune cells and immune signaling proteins allowing the pathogen to be eliminated more rapidly. In cases where antibody cocktails are used and including immunoglobulins other than dIgA there is an inherent probability of cytokine storm with some classes of IgG immunoglobulins binding to some antigens or even potentially allergens as a recent example if the epithelial barrier is not breached. However, should some pathogens breach the epithelial barrier the probably of cytokine storm is significantly higher. In some cases immunoglobulins may target pathogen cell surface proteins and prevent interactions between cell surface proteins and host receptors.

It may be beneficial to reduce or eliminate effector function to prevent cytokine secretions and the secretion of other pro-inflammatory agents that occurs with antibody mediation viral enhancement or antibody dependent enhancement (ADE) where it is possible for pathogen specific antibodies to promote pathology in the lung tissue, urinary tract, reproductive tract and even potentially the stomach without infecting the epithelial lining. Generally, this only occurs when antibody levels specific to the target are low or antibody-binding constants are not high enough and are thus non-neutralizing. This instant patent aims to identify those antibodies developed by humans that are of a high binding affinity. However, in the case where the most potent natural antibodies against the target protein falls below a specific threshold value or below the optimal “therapeutic binding affinity” one may consider engineering Fc fragments and C_{H}1 and CL domains to reduce ADE. ADE occurs mostly due to the interactions between the antibody and Fc gamma receptors (FcγRs) which IgA does not have, rather IgA is detected by Fc alpha receptors (FcαRI) where ADE does not happen as readily as with IgG. ADE may be mediated by the binding of the Fc regions of antigen-bound or allergen-bound antibodies to immune cells. One may also change or engineer the immunoglobulin constant light (CL) domains by adding a furin cleavage site or 2A self-processing peptide to their C-terminal end. These changes might interfere with Fcγ receptor binding to the immunoglobulin C_{H}2 constant region. (See e.g., Lu, J., Sun, P. D., et al. 2015, Proceedings of the National Academy of Sciences of the United States of America, vol. 112, pp. 833-838.) When an immunoglobulin binds to an antigen there is a conformational change in the immunoglobulin that causes the C_{H}1 and CL domains of the Fab to undergo a conformational change and engage the Fc region. (See e.g., Pritsch, O., et al., 1996, J Clin Invest. 98:2235-2243; Janda, A., et. al., 2016, Front Microbiol., 7:22; Iwasaki, A. & Yang, Y., 2020, Nat. Rev. Immunol. 20:339-341). ADE occurs due to the interactions between the antibody and Fc receptors including Fc gamma receptors (FcγRs). ADE may also be mediated by the binding of the Fc regions of protein-bound antibodies to immune cells. ADE may occur in the lumen of the lungs without the pathogen breaching the epithelial lining. Internalization of the antibody bound to its target in the lungs has the potential to promote inflammation and tissue damage through increased levels of pro-inflammatory chemokines CCL2 and CCL3 and reduced levels of decreased levels of the anti-inflammatory cytokines IL-10 and TGFβ. CC chemokines such as CCL2 and CCL3 promote the migration of monocytes to the lung. CCL2 and CCL3 attract monocytes through the receptor CCR2B, inducing their migration from the bloodstream to become tissue macrophages. SIgA1 and SIgA2 are not efficient activators of the complement cascade that is involved in cytokine storm nor are they efficient activators of proinflammatory responses but rather are anti-inflammatory in nature. This is a result of partial blockage of the FcαRI by secretory component preventing efficient access by resident macrophages, monocytes, neutrophils and eosinophils of the myeloid lineage that are often resident innate immune cells in the mucosa and exocrine lumen that decreases the chance of activating the complement cascade and initiating damaging inflammation. For example, opsonic activity of SIgA is poor compared with dIgA or IgA this is due to partial blockage of the FcαRI binding site by secretory component. This is consistent with the more anti-inflammatory role of SIgA. In contrast dIgA and IgA have a dual role in immunity providing both anti-inflammatory and proinflammatory roles. (See e.g., F. et al., 2008, J. Immunol., 181:6337-6348; Iwasaki, A. & Yang, Y., 2020, Nat. Rev. Immunol. 20:339-341; Yip, M. S. et al.; 2016, Hong Kong Med. J. 22:25-31; Wang, S. F. et al., 2014, Biochem. Biophys. Res. Commun. 451:208-214; Mak, T., 2014, Primer to the Immune Response 2nd Ed:269-292; Bakema, J., van Egmond, M., 2011, Mucosal Immunol, 4:612-624)

IgA, dIgA and SIgA all variants of IgA are unique in that they each are capable of a different role in inflammation including antibody mediated cellular cytotoxicity, proinflammatory, anti-inflammatory responses. One IgA can simultaneously bind to two FcαRI molecules on the surface of the myeloid derived innate immune cell. Crosslinking of FcαRI by an IgA immune complex induces FcRγ-chain independent redistribution of FcαRI to plasma membrane rafts (glycosphingolipids and cholesterol. These domains, termed membrane “rafts,” have estimated average sizes ranging from 70 nm). Monomeric IgA in unbound form inhibits the activation of heterologous receptors (e.g. other FcRs, cytokine receptors, chemotactic receptors and TLRs) For example, experiments with eosinophils, monocytes and transfectants demonstrated that FcαRI shows low capacity to interact with IgA immune complexes in a resting state, but ligand binding capacity increases profoundly after stimulation with cytokines such as granulocyte-macrophage-colony stimulating factor (GM-CSF) and IL-4 or IL-5. The ambivalent functions of IgA in human cells are regulated by the CD-89 associated FcRγ chain. dIgA while involved in proinflammatory responses on the other hand is not capable of crosslinking two receptors on an innate immune cell with FcαRI thus is not able to promote the same level of inflammatory response as a crosslinked IgA1 in an immune complex. dIgA like IgA1 has a fully exposed region to bind to FcαRI receptors found on myeloid lineage monocytes, macrophages, neutrophils and eosinophils. When in an immune complex dIgA triggers proinflammatory responses in these cells with the FcαRI receptors it can trigger opsonization and phagocytosis in innate immune cells such as neutrophils with an FcαRI receptor. Tissue distribution of FcαRI is mostly defined by the presence of neutrophils and some macrophages SIgA has partial blockage of FcαRI by secretory component this dampens the proinflammatory response and favors an anti-inflammatory response. Additionally, only a few FcαRI positive cells are observed in mucosal areas in homeostatic conditions. For instance, intestinal macrophages lack FcαRI expression which is consistent with an anti-inflammatory role of SIgA to protect mucosal immunity. Langerhans cells (tissue resident macrophages of the skin) do not express FcαRI. The role of SIgA is to preserve the epithelium in the mucosa and exocrine channels. SIgA binding by FcαRI is increased when complement receptor 3 (CR3) functions as a coreceptor. (See, e.g. Bakema, J., van Egmond, M., 2011, Mucosal Immunol, 4:612-624; Ben Mkaddem, S., Benhamou, M., & Monteiro, R. C., 2019, Frontiers in immunology, 10:811.)

### Minimizing Immunogenicity of the Vector Design

The introduction of foreign oligopeptides greater than 8 amino acids is capable of eliciting an immune response in individuals. Most nucleated non-lymphocyte cells express the major histocompatibility complex class I (MHCI) which primarily bind oligopeptides 8-14 amino acids in length generated from degradation of cytosolic proteins. B-cells and T-cells on the other hand present major histocompatibility complex class II (MHCII) that present 13-25 amino acid oligopeptides where there is an increasing affinity between the MHCII and a 16-20 amino acid oligo peptide where there is an order of magnitude reduction in binding affinity for the oligopeptide to the MHCII for each amino acid reduction in oligopeptide length below a 16 amino acid oligopeptide. (See, e.g. O'Brien, C., Flower, D. R., & Feighery, C., 2008, Immunome research, 4:6.) T-cell receptors (TCR) on CD8+ Cytotoxic T-cells detect the oligopeptides presented in MHCI to determine if they are self-antigens or not. Although TCR on CD4+ helper T cells detect the oligopeptides presented in MHCII to determine if they are foreign in nature and if they are determined foreign the B-cell is activated. Thus, B-cells and CD8+ T-cells are quite well suited to express the integration competent lentiviral gene therapy to express dIgA and also an integration competent gammaretroviral vector where a lentiviral vector is preferred. Hepatocytes present MHCI but virally infected hepatocytes are largely resistant to perforin/granzyme-mediated killing by CD8+ cytotoxic T-cells. In mouse and chimpanzee models of Hepatitis B (HBV) infection IFNγ produced by virus specific CD8+ T-cells correlates with a profound reduction in serum and liver HBV DNA by disrupting viral capsid integrity and helps to reduce hepatocyte damage by inducing cyto-protective proteins that confer resistance to granzyme B-mediated killing. (See, e.g. Gehring, A. J., Sun, D., M., Bertoletti, A., et al., 2007, J Virol., 81:2940-9.) Thus, hepatocyte expression of the gene therapy will be short-lived if CD8+ T-cells recognizes foreign antigen peptide sequences. The potential sequences in the antibody gene therapy that could be recognized by T-cells include 2A self-processing peptide fragments. E2A peptide fragments will not elicit a CD8+ cytotoxic T-cell response.

Skeletal muscle cells do not constitutively express or display MHC class I molecules, although they can possibly be induced to do so by proinflammatory cytokines such as IFNγ and tumor necrosis factor α (TNFα). Although, muscle cells themselves cannot be made to produce IFNγ nor TNFα under stimulating conditions consistent with gene therapy production of proteins and DNA based immunizations. Also, it was shown that overexpression of MHCI in muscle cells that is observed in some muscular and neuromuscular disorders may be the event that leads to autoimmune disorders of the skeletal muscle such as idiopathic inflammatory myopathies. Muscle has proved resistant to the development of inflammation caused by immunization with heterologous muscle, and inflammation does not persist long after acute viral injury. Although, research has shown that sufficient proinflammatory stimuli in muscles cells of mice induces the expression of other cytokines such as IL-6, transforming growth factor-beta (TGF-β), and granulocyte-macrophage colony-stimulating factor (GM-CSF) by muscle cells themselves, as well as the up-regulation of MHCI and MHCII. Although, none of IL-1α, IL-4, IL-10, IL-12 or IFNγ synthesis by human skeletal myoblasts was detected in reports. Further, Cytokine proteins for IFNγ, TNFα, MIP-1α were not detected to be expressed by muscle cells. Consequently, muscle is considered an attractive target for gene therapy and for the administration of DNA based immunizations speaking to their robustness for their purpose for most individuals in the population. Thus, administering the mRNA, AAV and non-integrating DNA based gene therapies into skeletal muscle cells for the expression of dIgA is an attractive target for individuals that do not have neuromuscular disorders, muscular disorders nor constitutively high levels of inflammation in their skeletal muscle or from immune cells acting on the skeletal muscle. (See, e.g. Nagaraju, K., Plotz, P., 2000, Proceedings of the National Academy of Sciences of the United States of America, 97:9209-9214.)

There is the potential that differences in the leader sequences could elicit an immune response I expressed in MHCI presenting cells. Although, for J Chain the leader sequence (SEQ ID NO: 27) in consistent across the human population. Additionally for the immunoglobulin heavy chain a common leader sequence found in the human population is reported in ENSEMBLE to be MDWTWRILFLVAAATGAHS (SEQ ID NO: 28). For the immunoglobulin light chain the Kappa light chain has high sequence similarity across most if not all of the human population where the small differences in the sequences are unlikely to elicit an immune response. Kappa light chain leader sequences are less likely to elicit an immune response that lambda light chain leader sequences. Kappa light chain is expressed in 60% of B-cells making it the predominate light chain expressed.

TABLE 1
Table of Human Signaling Peptides
(Most Common Bolded)

| SEQ | Signaling | | |
|---|---|---|---|
| ID NO: | Peptide | Length | Protein |
| 27 | MKNHLLFWGVL | 22 | J Chain |
| | AVFIKAVHVKA | | |
| 28 | MDWTWRILFLV | 19 | Immunoglobulin Heavy |
| | AAATGAHS | | Variable |
| 29 | MDTLCSTLLLL | 19 | Immunoglobulin Heavy |
| | TIPSWVLS | | Variable |
| 30 | MDMRVPAQLLG | 19 | Immunoglobulin Kappa |
| | LLLLWLPG | | Variable |
| 31 | MDMMVPAQLLG | 19 | Immunoglobulin Kappa |
| | LLLLWFPG | | Variable |
| 32 | MRVPAQLLGLL | 20 | Immunoglobulin Kappa |
| | LLWLPGARC | | Variable |
| 33 | MRLPAQLLGLL | 19 | Immunoglobulin Kappa |
| | MLWVPGKD | | Variable |
| 34 | MRLLAQLLGLL | 19 | Immunoglobulin Kappa |
| | MLWVPGSS | | Variable |
| 35 | MAWTPLWLTLL | 19 | Immunoglobulin Lambda |
| | TLCIGSVV | | Variable |
| 36 | MAWTPLFLFLL | 19 | Immunoglobulin Lambda |
| | TCCPGSNS | | Variable |
| 37 | MAWALLLLTLL | 19 | Immunoglobulin Lambda |
| | TRDTGSWA | | Variable |
| 38 | MPWALLLLTLL | 19 | Immunoglobulin Lambda |
| | THSAVSVV | | Variable |
| 39 | MAWSSLLLTLL | 19 | Immunoglobulin Lamba |
| | AHCTGSWA | | Variable |
| 40 | MAWSPLFLTLI | 19 | Immunoglobulin Lamba |
| | THCAGSWA | | Variable |
| 41 | MIYEVSHRPSG | 19 | Immunoglobulin Lambda |
| | VSTRFSAS | | Variable |

Selecting for random recombination of V_{H} and V_{L} regions or Fab regions of immunoglobulin coding DNA (cDNA) through the generation of combinatorial libraries and the use of phage display technology.

In cases where isolated B-cells does not express immunoglobulins with “therapeutic binding affinity” that is may be required for broad neutralization, mitigation of the effect of allergens or as a means to treat cancer it may be necessary to randomly recombine V_{H} and V_{L} regions expressed by different B-cells and also to potentially use mutagenesis to further increase binding affinity. This instant patent contemplates the design of vectors of dIgA1 and dIgA2 from libraries that selected for high affinity pairs of randomly and even purposefully recombined V_{H} and V_{L} immunoglobulin regions from different B-cells and even developed in other animals such as transgenic mice or mice or other animals with humanized immune systems. One of the basic principles is to use single chain variable fragments (scF_{V}) in phage display technology to select for highly potent immunoglobulins. One may assemble large libraries of plasmids that code for the random recombination of V_{H} and V_{L} immunoglobulin regions from different B-cells to select for high avidity and high affinity immunoglobulins where V_{H} and V_{L} regions are linked together with a short flexible peptide linker between the V_{H} and V_{L} fragments may occur by either N-terminus-V_{H}-linker-V_{L}-C-terminus or N-terminus-V_{L}-linker-V_{H}-C-terminus where both orientations have been applied but where N-terminus-V_{H}-linker-V_{L}-C-terminus is more common. This linked protein may be linked by a short peptide chain to the N-terminal end of a protein that is displayed on the surface of a filamentous phage for example.

Combinatorial libraries may be assembled in Escherichia coli as one typical example where E. coli contains a plasmid containing the gene encoding for the V_{H} gene and antibiotic resistance from one type of antibiotic may be transfected with a phagemid containing the V_{L} gene where both plasmids may be cleaved at a restriction site resulting in cleaved DNA where sticky ends undergo homologous recombination between the two cleaved plasmids to generate one larger circular plasmid that can result in productive expression of a phage that may be positively selected with the co-expression of a gene coding for antibiotic resistance from a different antibiotic that requires the other part of an antibiotic resistant gene found on the phagemid. One restriction site would be located in the region of the gene encoding for the linker that would be expressed between the V_{H} and V_{L} genes or V_{L} and V_{H} genes depending on the intended relative locations of V_{H} and V_{L} to that of the phage cell surface fusion protein. The resulting phagemid expressed a randomly recombined V_{H} and V_{L} gene that is then expressed as a fusion protein to a phage cell surface protein with the configuration N-terminal-V_{H}-linker-V_{L}-linker-phage cell surface protein-C terminal.

Phage expressing single chain variable-fragments (scF_{V}) with high binding affinity to the antigen may be selected for by coating the antigen to a plate which can be accomplished with biotinylation and then submerging the plate in a solution containing the E. coli and phage where following washing the plate with eluting solutions the most strongly binding phage that are not washed away by elution may be identified and have their DNA encoding for the scF_{V} amplified via PCR and sequenced. Such phage may also be used to re-infect E. coli for undergo amplification which being subjected to random mutagenesis of the V_{H} or V_{L} coding regions to create more diversity in the scF_{V} and potentially most strongly binding phage. Random mutagenesis typically occurs at fixed regions along the antibody sequence normally targeted at the complementary-determining regions (CDRs). Site-specific mutagenesis is also employed generally after multiple cycles of random mutagenesis and phage selection based on binding affinity to the antigen biotinylated to the plate. Site-specific mutagenesis selects a defined locus of the V_{H} and/or V_{L} genes or even very specific DNA sequences. Enzyme bases mutagenesis is one form of site-specific mutagenesis where a restriction endonuclease cleaves the DNA at a very specific region where oligonucleotide-mediated mutagenesis is widely employed to assist with site-specific mutation by providing internal mismatches that direct point mutations or multiple mutations to the target DNA sequence. Also, commonly used in phage display technology generally after multiple cycles of random mutagenesis is saturation mutagenesis, which employs the evaluation or substitution of a given residue against the 19 other amino acids at that specific residue.

### HIV Immunization Approach

This patent contemplates an immunization strategy to HIV and potentially a treatment for those infected with HIV. HIV continues to be a top priority to date and no vaccine strategy has induced antibodies with sufficient neutralizing coverage of the quasi-species. Once the host is infected with HIV control of the viral reservoir is significantly dependent on the Fc effector functions of broadly neutralizing antibodies as any loss in Fc effector activity results in the rapid loss of viral control despite potent neutralizing activity. Although, this is not necessarily true prior to infection of the host. As there are other important factors such as agglutination of HIV-virions in the mucosa pre infection that may be an equally important part of immune protection against HIV. To the surprise of many researchers, tissue resident natural killer (NK) cells express trivial levels of Fc receptors implying that they are unlikely to contribute to immunoglobulin mediated protection at the site of infection. It was previously reported that FcγRII and FcγRIII expressing macrophages and neutrophils were present in tissues collected from both HIV-seronegative and -seropositive subjects. Additionally, tissue-resident neutrophils while less abundant, mediated more effective phagocytic clearance of immune complexes. It has been suggested that antibody-driven functional activities mediated by cells other than NK cells are more likely to afford protection from infection as well as have therapeutic activity within mucosal and lymphoid tissues. (See e.g. Sips, M., et. al., 2016 Mucosal Immunol., 9:1584-1595.)

Human immunodeficiency virus type-1 (HIV-1) enters the host in virtually all infections through the mucosa of the genital tract or gastrointestinal tract. Once HIV traverses the epithelium in the reproductive tract HIV-1 then may encounter potential target cells such as CD4+ bearing lymphocytes including macrophages, monocytes and CD4+ helper T-cells in the lamina propria. However, it is also possible for HIV-1 to infect CD4+ bearing macrophages, monocytes and helper T-cells in the mucosa of the reproductive tract. In Memory CD4+ T cells and macrophages and monocytes of the myeloid lineage are believed to be an important reservoir for HIV-1. (See e.g. Kruize, Z., 2019, Frontiers in Microbiology, 10:1-17) Although, relatively little is known about HIV-1 infecting macrophages and monocytes in the mucosa as a result of the difficulty associated with isolating macrophages from mucosal tissue. Reproductive tract mucosal macrophages but not intestinal mucosal macrophages support the replication of the R5 HIV-1 strain. Although, intestinal macrophages have been reported to express no detectable, or very low levels of, innate response receptors and HIV-1 receptor/coreceptors and were determined to not support HIV-1 replication. However, these results are not definitive with regard to HIV-1 to infect intestinal tissue macrophages. (See E.g. Shen, R., et. al., 2009. Journal of virology, 83:3258-3267). Although, intestinal epithelial cells have been shown to selectively transport R5 virions to the lamina propria, which may lead to the preferential spread of R5 infection to activated CD4+ T cells. (See, e.g. Meng, G., X. Wei, X. Wu, M. T. Sellers, J. M. Decker, Z. Moldoveanu, J. M. Orenstein, M. F. Graham, J. C. Kappes, J. Mestecky, G. M. Shaw, and P. D. Smith, 2002, Nat. Med. 8:150-156. Also, see Schweighardt, B., Roy, A. M., Meiklejohn, D. A., Grace, E. J., 2nd, Moretto, W. J., Heymann, J. J., & Nixon, D. F., 2004, Journal of virology, 78: 9164-9173.)

There are three major strains of HIV: R5, X4 and X4R5 which are distinguished by their chemokine coreceptor usage. Non-syncytium-inducing (NSI), macrophage-tropic viruses utilize the CCR5 co-receptor and are called R5 viruses. Syncytium-inducing (SI) isolates use CXCR4 and are known as X4 viruses. Syncytium-inducing (SI) or X4 type 1 human immunodeficiency viruses (HIV-1) causes T-cells to fuse together into multinucleate cells from virus derived proteins presented on the T-cell surface. R5 strains are generally the dominant viral population during early stages of HIV-1 of infection. X4 strains on the other hand are commonly detected in the later stages of infection and are associated with rapid CD4+ T-cell loss. R5 strains are transmitted with an increased frequency in comparison with X4 strains. Individuals with a specific 32 base pair deletion of the CCR5 gene are resistant to HIV-1 infection. Although, these individuals are not liberated of susceptibility to infection via the X4 strain. The low infection rate by X4 strains on R5 resistant individuals in consistent with that observation that X4 infection universally occurs at a lower frequency. Additionally, R5 bears other advantages including dendritic cells preferentially transport R5 to the lymphnodes. X4 infection is also more susceptible to CD8+ T-cell antiviral immune response as X4 replication is no longer detected after such an immune response. With equal concentration of the X4 and R5 strains more cells are infected with the X4 strain. Although, R5 infected cells produce more HIV virions per cell infected resulting in a replication advantage. This replication advantage in R5 is also observed over the dual tropic X4R5 strain which also suggests that the X4 strain brings about conditions that impair replication. (See, e.g. Schweighardt, B., Roy, A. M., Meiklejohn, D. A., Grace, E. J., 2nd, Moretto, W. J., Heymann, J. J., & Nixon, D. F., 2004, Journal of virology, 78: 9164-9173.)

HIV isolates are generally classified on the basis of neutralization tier phenotype. These tiers form the basis for evaluation of neutralizing antibodies where antibody neutralization percentages are often reported as tier specific. The HIV GP120 viral envelope glycoprotein is a trimer that can change conformations between three states: a closed conformation, open confirmation or intermediate conformation. The conformation that is dominate on a basis of time spent in that conformation is the basis of tier classification. Tier 2 is predominately a closed conformation, tier 1A is predominately an open conformation and tier 1B is predominately an intermediate conformation. The accessibility of an antibody's epitope can be blocked by sterics of the tier e.g. of the antibody's epitope is found on the portion that becomes inaccessible in one conformation over another or if the epitope becomes partially buried in one conformation over another. The inaccessibility of an antibody's epitope due to the GP120 tier phenotype has the consequence of effectively reducing the antibody's apparent binding affinity with its epitope. Thus, one antibody that is effective at neutralizing one tier may not be effective at neutralizing another tier. Although, changing an antibody's isotype while maintaining its V region—known as a recombinant isotype—can result in an improve accessibility to its epitope. This may be due to changes in hinge length favoring more flexibility in the Fab region relative to the Fc region and factors that influence the antibodies binding affinity as a result of the isotype switch. That is an antibody that may be non-neutralizing to one tier of HIV may become neutralizing as a result of an enhanced binding affinity that makes a transient and unfavored conformation of a tier on a significant enough time scale to enable neutralization. In the case of dIgA1 (inclusive of polymeric immunoglobulin A1), dIgA2 (inclusive of and polymeric immunoglobulin A2) and engineered variants one antibody paratope may favor agglutination over another paratope as related to the epitope on the pathogen's protein it is specific to and that will in some instances drive a significant enhancement in neutralization when converting a monomeric antibody into a dimeric immunoglobulin A and even a polymeric immunoglobulin A recombinant isotype. (See e.g., Montefiori, D. C., Roederer, M., Morris, L., & Seaman, M. S., 2018, Current opinion in HIV and AIDS, 13:128-136.) Similarly, HIV-1 may be categorized by clades and evaluated by antibodies by Glade. Clades are similar in genetic distance each other and are associated geographically or epidemiologically.

One mechanism by which HIV-1 infects CD4+ bearing cells by binding with its HIV-1 envelope glycoproteins which is a trimer of glycoproteins120 (gp120) and gp41. There is a conserved CD4-binding site on the HIV-1 envelope glycoprotein. Additionally, there is a conserved crest on the V3 loop that is necessary for chemokine receptor type 5 (CCR5) or chemokine receptor type 4 (CXCR-4) co-receptor binding that is a key event for HIV envelope fusion with the CD4 bearing T-cell, macrophage or monocyte. Thus, each of the X4, R5 and X4R5 strains of HIV-1 use the conserved crest of the V3 loop of gp120 to fuse with target cells. Additionally, HIV-1 endocytosis by a macropinocytosis-like mechanism can lead to productive infection in macrophages. In order for macropinocytosis to occur in HIV-1 endocytosis the CCR5 receptor is thought to be present in the engulfed endosome. This pathway specifically requires CCR5 engagement at the cell surface, which in turn suggests that the virus and its coreceptor are present in the endosomal environment simultaneously since the principle of fusion with the endosome membrane is no different than the principle of viral fusion of HIV with an endosome. While HIV undergoes efficient viral degradation following endocytosis, analyses haves supported that HIV-1 transport through the endolysosomal pathway occurs through delayed viral degradation following endosomal internalization, possibly allowing the virus to complete its fusion. (See e.g. Gobeil, et. al., 2013, J Virol. 87:735-45, 2013; Carter, G. C., Bernstone, L., Baskaran, D., James, W., 2011, Virology, 409:234-250) This pathway of viral fusion during or after endocytosis is a critical pathway for HIV-1 that must be blocked by antibodies in the mucosal environment for any immunization to be effective. Although, it has generally been thought that endocytosis represents a dead end for HIV infection with the caveat of this viral fusion pathway that is thought to require CCR5 or CXCR4 binding to the HIV envelope glycoprotein which occurs at the conserved crest of the V3 loop. In one embodiment a dIgA1 antibody is considered for neutralization via binding to the conserved crest of the V3 loop is considered. In another embodiment dIgA2 is considered.

Although, there are additional sites on the HIV envelope glycoprotein that could be targeted as part of a cocktail strategy to maximize efficacy. Important clues to such a strategy would target conserved regions of the HIV envelop glycoprotein two such conserve regions are the Variable Region 1-Variable Region 2 (V1N2) regions and the CD4 binding site. A recent HIV-1 clinical trial in Thailand known as RV144 resulted in 31.2% efficacy. (See Tomaras, G. D., et. al., 2013, Proceedings of the National Academy of Sciences of the United States of America, 110:9019-9024.) In this clinical trial the authors reported that “Env-specific plasma IgA/IgG ratios are higher in infected than in uninfected vaccine recipients in RV144.” The report also discussed “Though plasma Env variable region 1 and 2 (V1/V2) IgG correlated with decreased infection risk, high levels of anti-HIV-1 Env plasma IgA correlated with increased infection risk.” The authors attributed this to the difference in effector functions between IgG and IgA. IgG1 for example has FcγRIIIa binding sites for macrophages and neutrophils that IgA does not have where IgA may be less effective at eliciting a pro-inflammatory response from macrophages and neutrophils in the mucosa and also less effective at recruiting natural killer (NK) cells in the bloodstream. The authors reference the following report that stated in the report “the binding of IgG antibodies to variable regions 1 and 2 (V1V2) of HIV-1 envelope proteins (Env) correlated inversely with the rate of HIV-1 infection (estimated odds ratio, 0.57 per 1-SD increase; P=0.02; q=0.08), and the binding of plasma IgA antibodies to Env correlated directly with the rate of infection” (Haynes B F, et al., 2012, N Engl J Med vol. 366:1275-1286). It may be beneficial to incorporate an IgG antibody into an HIV Immunization gene therapy cocktail specific to the V1/V2 regions while having no competing IgA or dIgA specific to the IgG binding site. This is only possible with direct administration of antibodies or a gene therapy-based approach. As using a traditional vaccination approach of developing immunity in one immune system from exposure of virus antigens cannot guarantee the ratios of IgG to IgA nor can it guarantee the specificity of the antibodies and their ability to be broadly neutralizing.

The third important binding site is the elusive CD4+ binding site that is typically responsible for HIV's initial binding to the CD4 presenting immune cell. Where a third potential gene therapy antibody cocktail immunoglobulin could be a IgG1, dIgA1 or polymeric immunoglobulin A specific to the HIV CD4 binding site. Such an antibody would block what may be the initiation event of HIV binding to immune cells that precedes the binding to the CCR5 or CXCR-4 binding sites. Although, it is recognized that such binding with the CD4 receptor may not be necessary during HIV-1 endocytosis by a macropinocytosis-like mechanism suggesting that targeting the crest of the V3 loop with dIgA1 or even dIgA2 (inclusive of polymeric immunoglobulin A2) is necessary to achieve an effective HIV-1 immunization.

In one embodiment an integration competent lentiviral vector or an integration-deficient lentiviral vector or gammaretroviral vector encoding for a dIgA1 (inclusive of polymeric immunoglobulin A1) antibody specific to a conserved target and receptor binding site on the HIV envelope glycoprotein is delivered via a anti CD20+ pseudotyped lentiviral delivery system or gammaretroviral delivery system to B-cells that bears a CD20+ receptor that include naïve B-cells, memory B-cells and germinal center memory B-cells. The vector will be integrated into genomic DNA or be delivered as episomal DNA and will code for a strong promoter such as a Feek promoter (SEQ ID NO: 105) that results in a higher level of expression of the vector encoded dIgA1 that the naturally encoded immunoglobulin encoded for by the B-cell resulting in the vector encoded dIgA1 being the major product. After some period following lentivirus administration an mRNA vaccine encoding for the HIV envelop glycoprotein is administered and even a second mRNA booster will be administered some period following. This, will have the effect of activating a large number of B-cells to become memory plasma B-cells potentially persisting for decades to express the dIgA1 specific to the conserved sequence of the envelope glycoprotein. In additional the administration of the mRNA B-cell activation will also result in the development of T follicular helper cells to active Germinal Center B-cells that contain the lentiviral vector. Both in vivo and ex vivo administration of the gene therapy to B-cells and other immune cells is considered.

H. pylori and Other Bacteria

This patent contemplates a strategy to neutralize a variety of bacteria. SIgA is known to neutralize bacterial by binding and agglutinating them preventing the formation of colonies and also binding to their flagella preventing their motility. SIgA also neutralizes bacterial products such as enzymes and toxins. This patent contemplates a strategy to encode for dIgA1 (inclusive of polymeric immunoglobulin A1), dIgA2 (inclusive of polymeric immunoglobulin A2) or engineered variants specific to the gram-negative, spiral shaped and microaerophilic bacteria Helicobacter pylori (H. pylori). H. pylori is a leading cause of gastric cancer death worldwide possibly causing over 700,000 deaths yearly and about 15,000 deaths in the U.S. yearly. Additionally, H. pylori is the only bacteria classified by the WHO as a class I carcinogen meaning it is the only bacteria well established to cause cancer. Most bacterial show tropism to specific tissues or cell types and often can use many different adherence mechanisms for attachment. Some speculate, that H. pylori may use as many as 5 different adhesins to attach to gastric epithelial cells. H. pylori has extensive gene variation within its genome that is thought to occur through slipped-strand mispairing within repeats as mechanisms for antigenic variation and adaptive evolution. H. pylori features a genome with high plasticity and high genetic heterogeneity especially in some parts of outer membrane proteins making them difficult targets. Although, there is some sequence identity is observed across some of the H. pylori outer membrane proteins that are thought to be necessary for adherence to the gastric epithelium. A common attachment site on epithelial cells is the Lewis histo-blood group antigens. H. pylori has a large number of sequence-related genes that are expressed on its outer surface. While the exact mechanism related to how H. pylori causes cancer is under investigation there are generally a few requirements for H. pylori to be cancer causing. H. pylori must be able to bind to the gastric epithelium and colonize in addition to burrowing through the epithelium causing stomach or small intestine ulcers in order to cause cancer. Additionally, 50% of H pylori induce vacuolation of epithelial cells that may lead to cancer through the action of vacuolating cytotoxin A (VacA) that is thought to induce apoptosis of epithelial cells that results in vacuoles that have traits of both endosomes and lysosomes. (See e.g., Atherton, J. C., Blaser, M. J., 1995, Cover, T. L., J Biol Chem, Jvol. 270:17771-17777; Kuck, D., Rudi, J., et al. 2001, Infection and immunity, 69:5080-5087) Although, while VacA induces apoptosis in epithelial cells it also disrupts endolysosomal vesicular trafficking and impairs the autophagy pathway. This makes it more difficult for the epithelial cell that has underwent apoptosis to be cleared away and replaced by new epithelial cells. Additionally, the virulence factor cytotoxin associated gene A (CagA) is a known oncoprotein that contributes to the development of gastric cancer. CagA has been shown to be dependent on VacA. It has been shown that in the absence of VacA two different cellular mechanisms proteosome degradation and autophagy degrade CagA. (See e.g., Abdullah, M., Bronte-Tinkew, D. et al., 2019, Sci Rep, vol. 9, pp. 38; Palframan, S. L., Gabriel, K., 2012, Frontiers in Cellular and Infection Microbiology, 2:1-9) Overall, results suggests that H. pylori may cause stomach cancer through more than one mechanism. Thus, targeting the cancer-causing mechanism may not be an effective stand-alone means to mitigate the effects of H. pylori. What is equally important is that H. pylori does not act virulent unless it colonizes. That is the Beta Barrel opening resulting from VacA and resulting cleavage of the virulent pepetide does not occur unless H. pylori is adhered to the epithelial cell and colonized and H. pylori does not secrete CagA and other toxins through a membrane channel unless it is colonized. These facts make VacA and CagA difficult targets because they are less likely to present on the outer membrane unless H. pylori is virulent. In order to colonize H. pylori must effectively adhere to epithelial cells. Attachment is mediated through cell surface adhesins. This results in a restricted range of hosts and tissues utilized for colonization. If bacteria are unable to adhere to epithelial cells they tend to be rapidly removed by the shedding of the surface cells and mucus layer. (See, e.g. Borén T, Falk P, Roth K A, Larson G, Normark S., 1993, Science., 262:1892-5) Thus, the most effective preventative measure against H. pylori infection would ideally involve targeting at least one protein that is important in binding to epithelial cells that has a well-conserved sequence on the surface that can be targeted.

The most ideal protein to target would ideally be common among H. pylori strains with a conserved sequence on the outer surface. HopQ I has been reported to be represented in 72.5% of H. pylori strains. HopQ is a porin that facilitates transfer of the CagA. HopQ can exploit the carcinoembryonic antigen-related cell adhesion molecule family (CEACAMs). HopQ binds to the IgV-like domain at the N-terminal of CEACAMs to facilitate the transfer of crucial pathogenic factor CagA to host cells. The HopQ-CEACAM interaction has been measured to be of high affinity (K_{D} from 23 to 268 nM), independent of CEACAM glycosylation. Data has supported that the HopQ-CEACAM interaction contributes to gastric colonization or Hp-induced pathologies. (Königer, V., Sundberg, E. J., et. al., 2018, The EMBO journal, 37:e98664; Xu, C., Soyfoo, D. M., Wu, Y., & Xu, S., 2020, European journal of clinical microbiology & infectious diseases: official publication of the European Society of Clinical Microbiology, 39:1821-1830)

Use of dIgA1 (inclusive of polymeric immunoglobulin A1), dIgA2 (inclusive of polymeric immunoglobulin A2) or engineered variants which becomes SIgA1, SIgA2 or SIgA respectively for this purpose satisfies two requirements first key binding sites can be blocked on H. pylori reducing its ability to bind to epithelial cells. Second, SIgA1 and SIgA2 can effectively agglutinate H. pylori together immobilizing their locomotion with its flagella and facilitating its passage through the digestive system followed by its excretion. For example, it is that cell specific attachment of H. pylori to human gastric surface mucous cells is inhibited by human colostrum secretory immunoglobulin A (SIgA) which is a glycoprotein carrying a highly variable set of N- and O-linked oligosaccharides. The inhibitory activity of SIgA is reduced when it is deglycosylated by digestion with alpha-L-fucosidase. (See, e.g. Boren T, Falk P, Normark S., et al., 1993, Science., 262:1892-5).

### Biodistribution of Antibodies in Interstitial Tissues Underlying the Mucosal Epithelium

This instant patent contemplates the major advantage of dIgA1 and dIgA2 over other immunoglobulins and achieving therapeutically relevant levels of gene therapy-based expression of dIgA1 and dIgA2 in the regions where they are needed in humans to exert their therapeutic benefit. To achieve therapeutically relevant levels of SIgA in the mucosa the underlying interstitium of the lamina propria must reach therapeutically relevant concentrations of dIgA1, dIgA2 or dIgA engineered variants. Lymph nodes typically align the areas directly outside the smooth muscle tissue (muscularis) of the lamina propria. These lymph nodes supply the interstitium of the lamina propria with plasma B-cells, memory plasma B-cells (that may ultimately migrate to the bone marrow) and some memory B-cells that are specialized in that most of them produce dIgA1 (inclusive of polymeric immunoglobulin A1) or dIgA2 (inclusive of polymeric immunoglobulin A2) as opposed to monomeric immunoglobulins more typically found for B-cell in the circulating blood. Thus, typically, dIgA and polymeric immunoglobulin A antibodies are supplied right at the site where they undergo transcytosis upon binding with pIgR and the interstitium. Thus, if dIgA and polymeric immunoglobulin A is supplied from muscle cells, liver cells, memory plasma secreting B-cells localized in the bone marrow or other region in the blood stream they must be supplied through the blood stream to the lamina propria and their concentration will likely be lower than it is in the blood even after an equilibration period. Alternatively, they may be ultimately supplied by memory plasma B-cells derived from memory B-cells in the circulating blood or Germinal center memory B-cells that received integration competent or integration-deficient lentiviral vectors. Antibody Levels in organs and interstitial tissues such as those that underlie the mucosal epithelium in the digestive tract, reproductive tract, respiratory tract, kidneys and the skin have been evaluated for Immunoglobulin class G monoclonal antibodies. In the stomach and small intestine the IgG monoclonal antibody concentration in the interstitium of the lamina propria is 2-4% of that of the concentration of the circulating blood after subtracting out the residual plasma in the interstitial vasculature. In the lungs the IgG monoclonal antibody concentration is about 7% of that of the circulating blood after subtracting out the residual plasma in the interstitial vasculature. (See E.g. Eigenmann, M. J., Karlsen, T. V., Krippendorff, B. F., et. al., 2017, J Physiol.; 595:7311-7330) In the skin the IgG monoclonal antibody concentration is about 10% of that of the circulating blood. The unidirectional and active transport of dIgA and polymeric immunoglobulin A from the lamina propria to the mucosa will lead to an overall higher concentration in the lung, digestive and parts of the reproductive tracts in the likely in area of a 50% or greater increase over that of IgG up to some threshold concentration that will unquestionably be therapeutically significant since one can achieve an therapeutic effect in the same interstitial areas with IgG antibodies administered to the blood and the neutralizing concentrations of dIgA and polymeric immunoglobulin A are lower than that of IgG as a result of their increased valency. The, lung lamina propria and mucosa dIgA levels will reach a dIgA concentration in the area of 11% of that of the circulating blood level concentration of dIgA. In the stomach and small intestine circulating antibodies can pass freely through the capillary endothelium in the interstitium that has fenestrations of 60-80 nm allowing for fast equilibration and replacement of any dIgA that is transported to the mucosa. Both dIgA and polyermic immunoglobulins A is 22-26 nm in length. The digestive tract lamina propria and mucosa are likely to see even higher concentration increases in dIgA over that reported for IgG due to the rapid equilibration that occurs between the fenestrated capillary endothelium and the lamina propria thus dIgA concentrations of 4-7% of the circulating blood level are possible. Although, the capillary endothelium of the lung interstitium are continuous and lack fenestrations. Because dIgA and polymeric immunoglobulin A is actively and unidirectionally undergoes transcytoses across the epithelium with pIgR the dIgA mucosa distribution to blood distribution can be enhanced favorably despite low interstitial concentrations relative to dIgA blood levels. In one embodiment dIgA and polymeric immunoglobulin A is supplied from hepatocytes, in another embodiment dIgA and polymeric immunoglobulin A is supplied by muscle cells. In another embodiment dIgA is supplied by splenocytes that receive and express the gene therapy vectors. In another embodiment dIgA and polymeric immunoglobulin A is supplied by memory plasma B-cells residing in the bone marrow. In another embodiment dIgA and polymeric immunoglobulin A are supplied by resident memory plasma B-cells e.g. residing in the lamina propria of the digestive tract or small intestine as an example. In another embodiment dIgA and polymeric immunoglobulin A are supplied by resident memory T-cells or resident NK cells. (See E.g. Borrok, M. J., DiGiandomenico, A., Beyaz, N., Marchetti, G. M., Barnes, A. S., Lekstrom, K. J., Phipps, S. S., McCarthy, M. P., Wu, H., Dall'Acqua, W. F., Tsui, P., & Gupta, R., 2018, JCI insight, 3:97844:1-9).

### The Interstitium of the Lamina Propria

This patent contemplates direct administration of the gene therapy via endoscopic injection or absorption into the lamina propria that may also be administered with microneedles. The presence of SIgA in mucosal surfaces is locally produced as dIgA and polymeric immunoglobulin A by antibody secreting B-cells in the lamina propria directly beneath the epithelial cells. The main source of these dIgA and polymeric immunoglobulin A plasma secreting B-cells is the local lymph nodes for the organ. As an example, the stomach has 4 supporting groups of lymph nodes support 4 distinct regions of the stomach. The small intestine also has a supporting lymph nodes in addition to Payer's patches that are sometimes located in the upper duodenum but more often found in the mid to lower duodenum and in other regions of the small intestine. Nutrient and fluid absorption in the Gastrointestinal tract requires lymphatic networks to both regulate interstitial fluid balance and transport lipids. Lymphatic flow in the stomach begins within the initial lymphatic sinuses near the pyloric glands. These fuse to link lymphatic networks between the muscularis mucosa and the submucosa. (See, e.g., Spencer, J., Sollid, L., 2016, The human intestinal B-cell response. Mucosal Immunol 9, 1113-1124; Lycke, N., Bemark, M., 2017, Mucosal Immunol, 10:1361-1374; Eichmann, A., 2019, Cellular and Molecular Gastroenterology and Hepatology, 7:503-513)

The embodiment of the invention related to gene therapy delivery to memory B-cells or germinal center B-cells in lymph nodes supporting an organ with a mucosa is achieved through the initial lymphatics in the lamina propria which contain button like openings in parts of the respiratory tract and digestive tract that allow for flow from the fluid filled interstitum into the lymph vessels into the afferent lymphatic vessels that carry unfiltered lymph fluid passing through local lymph nodes to be filtered. Such initial lymphatics are found in the lungs and GI tract. At the initial lymphatics there are distinct leaf shaped endothelial cells that are the general locations of fluid entry into the lymphatic system. The borders of these epithelial cells have been thought to be the primary valves that permit unidirectional flow of fluid into lymphatics. In the trachea and thought to be the case for the stomach and small intestine the borders of these epithelial cells are thought to contain discontinuous tight junctions made by VE-cadherin that is consistent with their function as connection points along the sides of flaps that are effectively the borders for the button like openings for fluid passage without junctional disassembly. Fluid entry into the button like openings is driven by muscle contraction. The buttons can be thought of as linear segments that are parallel to each other with openings of about 3.2 μm (micrometers) or 3,200 nm (nanometers) that were spaced about 2.9 μm apart in the trachea. (See, e.g., Baluk, P., et. al., The Journal of experimental medicine, vol. 204, pp. 2349-2362, 2007; Eichmann, A., 2019, Cellular and Molecular Gastroenterology and Hepatology, 7:503-513)

When a lymphangion, the functional unit of a lymph vessel, has contractile force exerted on it e.g. as due to muscle contraction of smooth muscle linking lymph vessels lymph can be propelled forward in a unidirectional manner. The semilunar valves are directed towards the flow of the lymph and open when the pressure in the first lymphangion is greater than the pressure in the next lymphangion. Pressure in the first lymphangion may increase because of smooth muscle contraction (in lymph vessel) or because of pressure on the walls from outside. Once the lymph flows into the next lymphangion, it cannot return to the previous lymphangion, as the semilunar valves close tightly. (See, e.g. Venugopal, A. M., Stewart, R. H., Laine, G. A., Dongaonkar, R. M., Quick, C. M., 2007, Am J Physiol Heart Circ., 293: H1183-9)

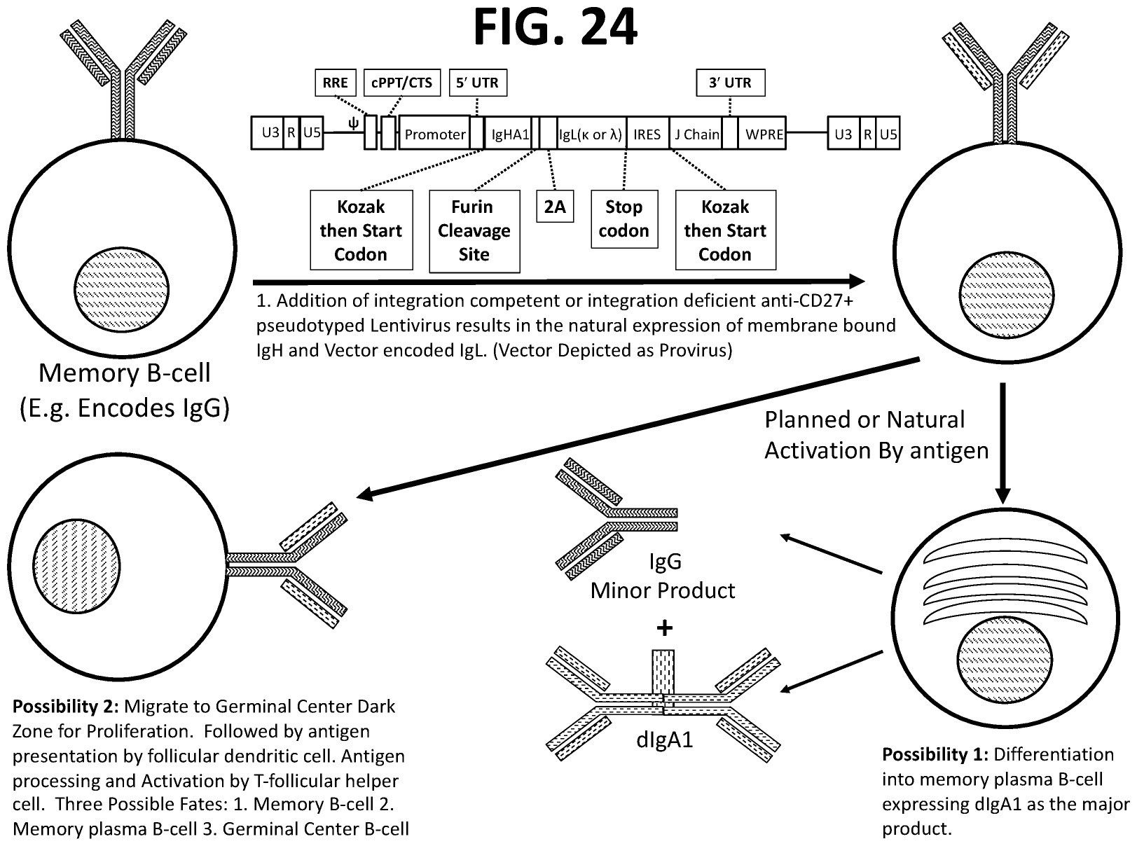

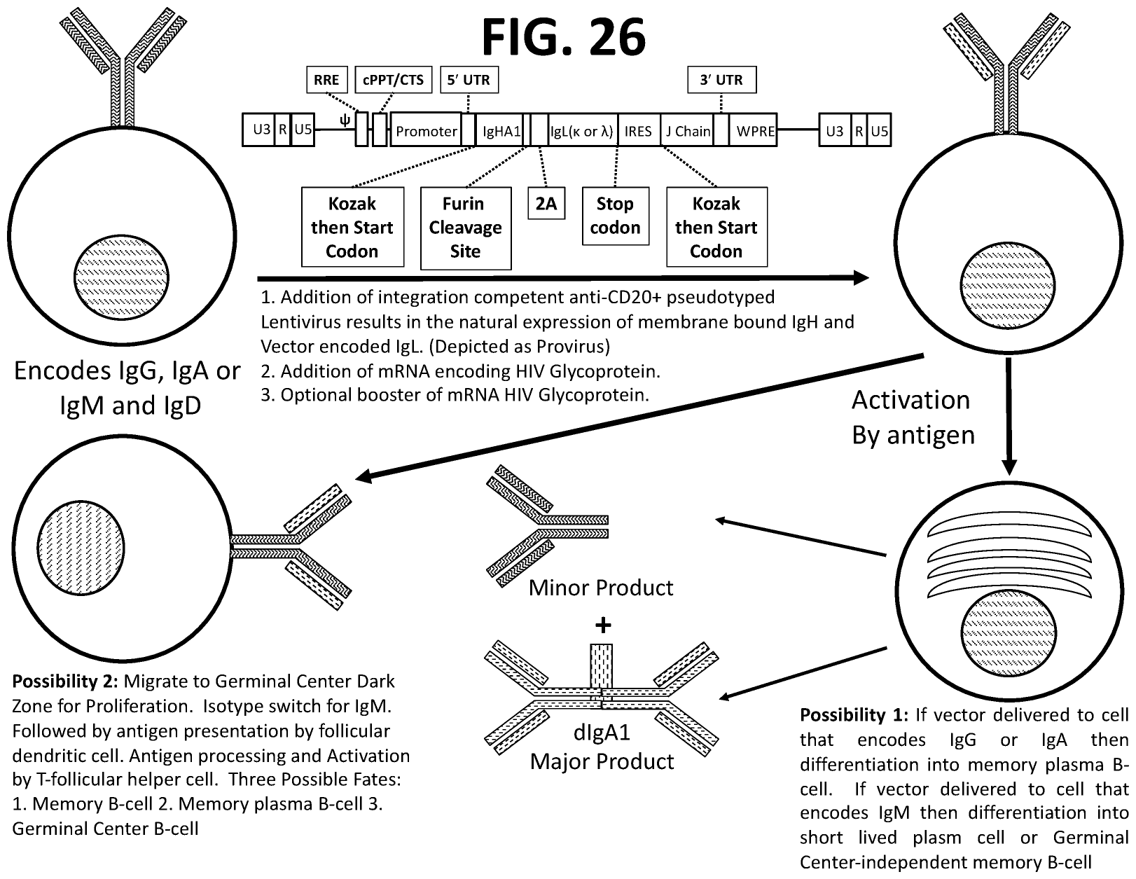

In the stomach and small intestine, lymphatic capillaries, or lacteals, are located exclusively in intestinal villi. Spontaneous lacteal contraction, in concert with adjacent smooth muscles, is essential for drainage into lymph nodes. Lacteal contraction is regulated by the autonomic nervous system, and to be increased by acetylcholine and decreased by norepinephrine. The lacteals lead to lymph nodes supporting the organ. (See e.g., Cifarelli, V., Eichmann, A., 2019, Cellular and Molecular Gastroenterology and Hepatology, 7:503-513) In one embodiment those lymph nodes that house germinal center memory B-cells will receive the gene therapy delivery vehicle such as a Lentivirus that is known to be absorbed by memory B-cells via a pseudotyped lentivirus that can target the CD receptors such as but not limited to CD19, CD20 or CD27 on the memory B-cell that can be a germinal center memory B-cell. (See FIG. 24) (See, e.g. Cascalho, M., et al., 2018, Sci Rep, 8:11143; Also see, Fusil, F., Cosset, F. L. et. al., 2015, Molecular therapy: the journal of the American Society of Gene Therapy, 23:1734-1747) The use of a Feek promoter in the vector construct enables the vector encoded immunogloublin specific to the target of interest to be expressed at a much higher level than the B-cells naturally encoded immunoglobulin. Upon activation the memory B-cell can differentiate into a memory plasma B-cell (see FIGS. 24 and 26) that will migrate to the tissues supported by the lymph node they are derived from e.g. the lungs or stomach and upper duodenum via local blood flow where they would secrete the dIgA and polymeric immunoglobulin A encoded for by the vector delivered gene therapy delivery vehicle as the major immunoglobulin product through the use of a strong promoter such as a Feek promoter (SEQ ID NO: 105). In another embodiment CD45+ Plasma B-cells in the Lamina Propria will also be targeted by the integration competent or integration deficient lentivirus. (See e.g., Spencer, J., Sollid, L., 2016, The human intestinal B-cell response. Mucosal Immunol 9, 1113-1124). It was previously demonstrated that a pseudotyped lentiviral vector could target B-cells retrovirally incorporate the retroviral vector and conditionally express both the membrane anchored and secreted forms of antibodies encoded for by the vector as the major immunoglobulin product with the use of a strong promoter such as the Feek Promoter (SEQ ID NO: 105) and through alternative splicing that was dependent on the B-cell maturation state. (See, e.g., Fusil, F., Cosset, F. L. et. al., 2015, Molecular therapy: The Journal of the American Society of Gene Therapy, 23:1734-1747). (Also see the international patent filed by the authors WO 2017/005923 and U.S. patent filed by the authors US 2018/0371064 A1 expressly incorporated by reference herein in its entirety) The authors of this report focused on the expression of IgG1 and considered it as a strategy for both HIV and cancer. In this strategy the authors used the naturally encoded splice sites found in human to enable the alternate splicing. Looking at FIG. 1 of WO 2017/005923 and in the specifications and claims of U.S. Pat. Publication No. 2018/0371064 A1 and International No. WO 2017/005923 the authors utilized the M1 and M2 exons in the integrated lentiviral vector to encode for membrane bound IgG for the naïve B-cell mature B-cell and memory B-cell cell differentiation states. Where, as an example upon the membrane bound IgG1 binding to its target with sufficient affinity the memory B-cell underwent activation and differentiated into a long-lived plasma memory B-cell where through alternate splicing the M1 and M2 exons in the pre-mRNA were not spliced into the mature mRNA and the secreted form of IgG1 was generated. One may observe the IgG1 human transmembrane domains and cytoplasmic domains and the splice sites on Ensemble at https://useast.ensembl.org/Homo_sapiens/Transcript/Exons?db=core;g=ENSG00000211896; r=14:105736343-105743071;t=ENST00000390548 where Transcript IgHG1-202 ENST00000390548.6 is considered. The IgA1 and IgA2 human transmembrane and cytoplasmic domains and the splice sites are reported on Ensemble at https://useast.ensembl.org/Homo_sapiens/Transcript/Exons?db=core;g=ENSG00000211895; r=14:105703995-105708665J=ENST00000641837 where Transcript IgHA1-202 ENST00000641837.1 is considered and https://useast.ensembl.org/Homo_sapiens/Transcript/Exons?db=core;g=ENSG00000211890; r=14:105586889-105588395J=ENST00000497872 where Transcript IgHA2-202 ENST00000497872.4 is considered. In IgA1 and IgA2 and in turn dIgA1 (inclusive of polymeric immunoglobulin A1) and dIgA2 (inclusive of polymeric immunoglobulin A1) respectively there is a single protein encoding exon ENSE00003812052 for IgA1 and ENSE00002100284 for IgA2 encoding for the transmembrane and cytoplasmic domain. The resulting polypeptide sequence encoding for the transmembrane domain is also different.-For example the polypeptide sequence that is added by encoding for the membrane bound IgG1 with the M1 and M2 exons is “ELQLEESCAEAQDGELDGLWTTITIFITLFLLSVCYSATVTFFKVKWIFSSVVDLKQT IIPDYRNMIGQGA” where this polypeptide sequence replaces the final two amino acids “GK” of the secreted form of IgG1. Although, the polypeptide sequence that is added by encoding for the membrane bound IgA1 with the M exon is “DWQMPPPYVVLDLPQETLEEETPGANLWPTTITFLTLFLLSLFYSTALTVTSVRGPS GNREGPQY” where there is little amino acid similarity with the transmembrane and cytoplasmic domains of IgG1 and where this polypeptide sequence replaces the final twenty amino acids “GKPTHVNVSVVMAEVDGTCY” of the heavy chain C-terminal tail of the secreted IgA1 form is excluded. The excluding of the oligopeptide tail of IgA1 can be explained by its necessity in participating with J Chain (see FIG. 1) in the formation of a Beta sheet complex (Beta sandwich) in secreted dIgA2 and polymeric immunoglobulin A2. Additionally, the polypeptide sequence that is added by encoding for the membrane bound IgA2 with the M exon is “GSCCVADWQMPPPYVVLDLPQETLEEETPGANLWPTTITFLTLFLLSLFYSTALTVT SVRGPSGKREGPQY” where there is little amino acid similarity with the transmembrane and cytoplasmic domains of IgG1 and where this polypeptide sequence replaces the final twenty amino acids “GKPTHINVSVVMAEADGTCY” of the heavy chain C-terminal tail of the secreted IgA2 form is excluded. The excluding of the oligopeptide tail of IgA2 can be explained by its necessity in participating with J Chain (see FIG. 1) in the formation of a Beta sheet complex (Beta sandwich) in secreted dIgA2 and polymeric immunoglobulin A2. 98.71% of splice sites contain canonical dinucleotides GT and AG for donor and acceptor sites. In pre-mRNA splicing the canonical donor splice has nucleotides GT exactly after the point where the spliceosome cuts the 5′-end of the intron sequence in the pre-mRNA, and the canonical acceptor site has nucleotides AG exactly at the point where the spliceosome cuts the 3′-end of the intron sequence in the pre-mRNA. The consensus sequence for an intron is: A-G-[cut]-G-U-R-A-G-U (donor site) . . . intron sequence . . . Y-U-R-A-C (branch sequence 20-50 nucleotides upstream of acceptor site) . . . 15-Y-rich-N-C-A-G-[cut]-G (acceptor site). This is a general guide that does not convey all the information found in a motif logo.

This patent further contemplates the use of proteins or mRNA encoding for proteins delivered to the lamina propria of the at risk organ identifiable as or derived from the (1) virus (s) (2) systemic ailment (3) fungi (4) bacterial infection (4) cancerous tumor (5) biowarfare agent (6) microbial infection, (7) any ailment (8) target protein or variant of interest. This patent targets the lamina propria and underlying interstitium as a means to reach the lymph nodes supporting the organ of interest. In one embodiment the gastrointestinal tract such as stomach and small intestine is targeted. In another embodiment the lungs are targeted. The lymph nodes would be reached by the vaccine traveling though the underlying lymph vessels in the underlying organ.

### 3′ Untranslated Regions (3′ UTR) and 5′ Untranslated Regions (5′ UTR)

Important elements of RNA are the 5′ UTR that is upstream of the beneficial gene as mRNA (SEQ ID NOs: 74-86) and as DNA (SEQ ID NOs: 74-86) and the 3′ UTR that is downstream of the beneficial gene as mRNA (SEQ ID NOs: 55-70) and as DNA (SEQ ID NOs: 87-102). UTRs serve a variety of roles including enhancing the amount of protein that is produced from a single RNA, efficient transport outside the nucleus, stability of the mRNA increasing the mRNA half-life and the ability to control the rate of gene translation.

The 5′ UTR is also known as the leader sequence (not to be confused with the immunoglobulin leader sequence or signal peptide) and is the region of mRNA directly upstream of the initiation codon. 5′ UTRs can form complex secondary structures to regulate translation on two levels by preventing translation or as a tertiary structure necessary for binding and recognition by translation factors. Regulatory elements in the 5′ UTR are also linked to mRNA export outside the nucleus. 5′ UTRs that allow for efficient translation are short, have a low GC content, do not contain upstream AUG codons and are relatively unstructured. A major aim of gene therapy is to minimize the dosage necessary to achieve a therapeutically relevant amount of the protein that is being encoded for which this instant patent contemplates to ensure high expression of vector encoded immunoglobulin in this instant patent. In general it has been shown that shorter 5′ UTR sequences are responsible for higher levels of protein expression when the same 3′ UTR is used to express the same protein. (See e.g., Trepotec, Z., et. al., 2019, Tissue Eng Part A., 1-2:69-79; Leppek, K., Das, R. & Barna, M., 2018, Nat Rev Mol Cell Biol, 19:158-174; Calvo, S. E., Pagliarini, D. J., et. al., 2009, PNAS, 18:7507-7512; Babendure, J. R., et. al., 2006, RNA, 12:851-861; Barrett, L. W., et. al., 2012, Cellular and molecular life sciences: CMLS, 69:3613-3634)

Within eukaryotic genes the 5′ UTR is located downstream of the TATA box. The TATA box is the transcription initiation site. Although, there is spacer DNA between the 5′ UTR and the TATA box that is typically about 20 bases. This spacer DNA is necessary for the transcriptional machinery to assemble and begin transcription about 20 bases downstream from the TATA box. For example, TATA box binding protein binding the TATA box with other cofactors followed by RNA polymerase which pushes the transcription start site downstream from the TATA box.

5′ UTRs for proteins intended for high expression typically are very short in the area of 20 to 30 bases including the (−1 through −6 position on the Kozak consensus sequence). For example, the protein J Chain that is expressed in a dIgA and polymeric immunoglobulin A plasma B-cell and in a dIgA and polymeric immunoglobulin A memory plasma B-cell at a rate high enough to support 400-500 dIgA1 (inclusive of polymeric immunoglobulin A1) immunoglobulins per second (requiring the translation of about 200-250 immunoglobulin J Chains per second) has a 5′ UTR of 18 bases including the Kozak consensus sequence. A list of human 5′ UTRs contemplated in the invention include those human 5′ UTRs used for immunoglobulins and also those human 5′ UTRs used for selected highly expressed genes in cells that are ideal to express the vector encoding for dimeric immunoglobulin can be found in DNA form as Table 2 (SEQ ID NOs: 42-54) and mRNA form as Table 2b (SEQ ID NOs: 74-86) Although, this list is not exhaustive, and the invention is not limited to these sequences.

### 3′ UTR

The 3′ UTR is located downstream of the protein coding sequence is involved in numerous regulatory processes including transcript cleavage, stability, translation and mRNA localization. Sequence constrains are more relaxed in the 3′ UTR allowing for a higher degree of regulation. Although, there are regions of high conservation within the mammalian genome. (Barrett, L. W., et. al., 2012, Cellular and molecular life sciences: CMLS, 69:3613-3634; Siepel, A., et. al., 2005, Genome research, 15:1034-1050). The 3′ UTR often contains conserved motifs for regulatory proteins regulate translation and even localization by engaging with the conserved motifs.

Recently, it was shown that in human cell lines that 3′ UTRs allow for differentially regulated localization of some proteins to membranes. As an example, the long 3′ UTR of the mRNA of CD47 enables efficient transport to the cell surface where it is localized on the surface on the cell. Alternatively the short 3′ UTR of the mRNA of CD47 localizes to the endoplasmic reticulum. The authors of this study concluded that the long 3′ UTRs contain additional regulatory elements that can regulate localization and protein abundance. Although, the localization step occurs at the protein level where the mRNA with both the long 3′ UTRs and short 3′ UTRs of CD47 have a similar distribution at the perinuclear endoplasmic reticulum. Thus, the localization of the protein is independent of the localization of mRNA. (See, e.g. Berkovits, B., Mayr, C., 2015, Nature 522:363-367). Immunoglobulins that are expressed on the cell surface likewise have a 3′ UTR that is much longer than the 3′ UTR of the same class and subclass of immunoglobulins that are expressed for secretion. Although, both these immunoglobulins must ultimately reach the surface of the cell. Although, through different pathways. Additionally, 3′ UTRs may encode for alternate polyadenylation. Human genes for example, contain more than one polyadenylation site that is likely related to UTR-alternate polyadenylation (UTR-APA) that would result in an mRNA isoform. This APA is different than the polyadenylation that results from coding region-polyadenylation (CR-APA) where the coding region. In UTR-APA the alternate poly(A) signal are often proximal to the stop codon. UTR-APA often do not possess non-canonical poly(A) signals such as ATTAAA, AGTAAA or TATAAA and thus are considered to be weaker signals. Although, non-canonical poly(A) signals in some instances can be favored over canonical poly(A) signals. (See, e.g., Yeh, H. S., & Yong, J., 2016, Molecules and cells, 39: 281-285.)

A list of human 3′ UTRs contemplated in the invention include those human 3′ UTRs used for immunoglobulins and also those human 3′ UTRs used for selected highly expressed genes in cells that are ideal to receive the vector encoding for dimeric immunoglobulin A can be found in DNA form in Table 3 (SEQ ID NOs: 55-70) and mRNA form in Table 3b (SEQ ID NOs: 87-102) but the 3′ UTRs that may be considered further the 3′ UTRs of these tables may be modified, truncated or have intermediate regions removed or replaced to prevent microRNA binding or to prevent premature polyadenylation. Although, this list is not exhaustive, and the invention is not limited to these sequences.

B-Cell Differentiation into Plasma Secreting Cells

The differentiation of B cells into Ig-secreting plasma cells (both memory plasma B-cells and short-lived plasma B-cells) requires the expansion of secretory organelles to cope with the increased cargo load. When any B-cells differentiates into a short-lived plasma B-cell or a memory plasma B-cell the cell increases in size significantly while the endoplasmic reticulum (ER) continues to remain close to the nucleus of the cell. Later during interphase the ER fills the entire cell for efficient secretion of immunoglobulins via the extension of ER tubules under the plasma membrane. At the same time the Golgi remains near the nucleus while expanding to 6.5 fold in linear volume mostly linearly while remaining close to the nucleus. Immunoglobulin secretion rate increases dramatically by day 4 following B-cell activation as by day 3 ER proliferation is quite significant and can more readily support high expression rates of immunoglobulins. From this it is evident that without an unusually relatively large dedication to the ER and Golgi plasma secreting B-cells would secrete immunoglobulins at a much lower rate as was empirically observed. (See e.g. Kirk, S. J., Cliff, J. M., Thomas, J. A., Ward, T. H., 2010, J Leukoc Biol. 87:245-255).

This patent contemplates the delivery of gene therapy vectors encoding for immunoglobulins to cells with a large dedication to production of proteins and secretion of proteins as evident by extensive resources dedicated to the ER and Golgi in addition to a large ER and Golgi. For example hepatocytes a type of liver cell, secrete a large amount of albumin and fibrinogen, alpha-1-globulin, alpha-2-globulin and beta globulin. (See e.g. Feldmann, G., Penaud-Laurencin, J., Crassous, J., Benhamou, J. P., 1972, Gastroenterology, 63:1036-1049; Woo, D. H., et. al., 2012, Gastroenterology. 142:602-11; Sharma, N. S., Nagrath, D., & Yarmush, M. L., 2011, PloS one, 6:e20137) Muscle cells must produce large amounts of actin and myosin necessary to support the muscular-skeletal structure. T-cells and NK cells must produce large amounts of chemokines and cytokines for secretion including IL-2, perform, granzymes A and B, INFγ and TNFα upon activation. For this reason muscle cells, hepatocytes, T-cells and NK cells can produce dIgA at therapeutically relevant levels specific to the application of interest. (See e.g. Lei, Y., Huang, T., Su, M. et al., 2014, Lab Invest 94:1283-1295; also see Perez, N., et. al., 2005, Genetic vaccines and therapy, 2:1-5). For the same reason, memory B-cells and even germinal center B-cells (that are a type of memory B-cell since they possess immunological memory) are also well suited for genomic integration of lentiviral DNA or episomal addition of lentiviral DNA coding for immunoglobulins as upon their differentiation into memory plasma B-cells will have ample resources dedicated to producing and secreting antibodies. (See, e.g. Cascalho, M., et al., 2018, Sci Rep, 8:11143) This patent contemplates targeting memory B-cells for integration-competent lentiviral vectors and integration deficient lentiviral vectors encoding for dIgA1 (inclusive of polymeric immunoglobulin A1), dIgA2 (inclusive of polymeric immunoglobulin A2) and engineered variants. In one embodiment (See FIGS. 24 and 26) the memory B-cell will present on its surface both the endogenous heavy and light chain immunoglobulins as a B-cell receptor as well as the endogenous immunoglobulin heavy chain with the genome integrated lentiviral vector or episomal encoded integration-deficient lentiviral vector encoded immunoglobulin light chain as a B-cell receptor. Having the gene therapy encoded immunoglobulin light chain presented on the memory B-cell will increase the probability that it will be activated from the presence of the protein the vector encoded immunoglobulin is specific to that may be artificially induced or occur as part of natural exposure to the pathogen or pathology promoting source. The accessibility of any antigen or protein the B-cell surface immunoglobulin has sufficient affinity for will result in differentiation to a memory plasma B-cell. As the immunoglobulin light chain is ideally suited for antigen recognition as it tends to play a dominant role in target binding. Thus, the light chain binding the target it is specific to will be sufficient in most cases to activate the memory B-cell to differentiate into a memory plasma B-cell. (See, e.g. Sun, M., Li, L., Sheng Gao, Q., Pad S., 1994, The Journal of Biological Chemistry, 269:734-738; Also see, Hadzidimitriou, A., Darzentas, N., Belessi, C., et. al., 2009, Blood, 113:403-411).

In some instances plasma B-cells and memory plasma B-cells may code for truncated immunoglobulin heavy or light chains through somatic hypermutations nonsense mutations and would then still produce the vector encoded dIgA and in other instances the cell will undergo apoptosis. Although, it is important to point out that even with the presence of the naturally encoded immunoglobulins by the cell by using a strong promoter such as a FEEK promoter (SEQ ID NO: 105) in the vector encoded immunoglobulin the major product of a memory plasma B-cell with the vector encoded immunoglobulin will be the vector encoded dIgA1 immunoglobulin. (For the Feek Promoter Sequence see International Patent Number WO 2017/005923 Fusil et al. and the U.S. patent filed by the authors US 2018/0371064 A1 expressly incorporated by reference herein in its entirety; Also see Fusil, F., Cosset, F. L. et. al., 2015, Molecular therapy: the journal of the American Society of Gene Therapy, 23:1734-1747) In contrast to the immunoglobulin Heavy chain that has a large 3′ UTR when it is expressed as a cell surface receptor because the immunoglobulin light chain has the same 3′ UTR whether it is expressed as part of a B-cell receptor or secreted it will serve as an adequate substitute for the immunoglobulin light chain that is endogenously encoded for by the cell.

mRNA 5′ Cap, Production and Design Framework

mRNA intended for gene therapy may be synthesized in vitro as part of template directed synthesis. Common methods include the T3, T7 and SP6 systems. The T7 system derived from the T7 phage of E. Coli is the most common method. The sequence of interest is placed downstream of the T7 promoter which covers the sequence from −17 to +6 (the first 6 RNA nucleotides to be synthesized) Thus, the first 6 RNA nucleotides do not have full flexibility of choice in the T7 system. One such class III T7 promoter is 5′-TAATACGACTCACTATAGGGAGA-3′. Transcription termination occurs at terminator sites called Rho-independent terminators. Here the 3′ end of mRNA forms a hairpin loop structure about 7-20 base pairs in length directly following the U heavy stretch. This hairpin loop formation results in pausing of RNA polymerase and disrupts the transcription complex. Alternatively, the termination of transcription may occur by the RNA polymerase running off at the end of the template where the formation of a hairpin structure is not necessary.

The 5′ cap and the polyadenylation (polyA) tail—a long stretch of untemplated adenosine—are two important elements of mRNA for use human gene therapy. In eukaryotes the 5′ 7-methylguanylate (m⁷G) cap with a 5′-5′ triphosphate linkage to the penultimate ribosemethylated nucleotide termed m⁷G(5′)ppp(5′)N^{m}—is an evolutionary conserved modification of eukaryotic mRNA. The m⁷G cap serves many essential roles including cap-dependent initiation of protein synthesis, the 5′ cap servers as a unique identifier to recruit protein factors for pre-RNA splicing, polyadenylation and nuclear export. Additionally, the 5′ cap protects against 5′ to 3′ exonuclease cleavage and is important for the recruitment of initiation factors that initiate protein synthesis in addition to the 5′ to 3′ looping of mRNA during translation. As part of the innate immune response the 5′ cap serves a function for self-discrimination where the 2′O methylation of the first RNA nucleotide is an important part of self-recognition in the innate response against foreign RNA. (See e.g., Ramanathan, A., & Chan, S. H., 2016, Nucleic acids research, 44:7511-7526; Also see, e.g. Martin, S. A., Paoletti, E., Moss, B., 1975, J Biol Chem. 250:9322-9329). There are a variety of other synthetic and modified 5′ caps that may be used in place of m⁷G(5′)ppp(5′)N^{m}-.

In producing mRNA using a T7, T3 or Sp6 phage RNA polymerase various version of 5′ caps can be added during or after transcription using a vaccinia virus capping enzyme or by incorporating a synthetic cap. Vaccinia capping enzyme is able to cap RNA using 3′-modified GTP. Vaccinia capping enzyme is used to cap 5′ triphosphate ends as well as 5′ diphosphate ends of RNA using 3′-desthiobiotin GTP. The sequences m⁷G(5′)pppN^{m}- are located at the 5′ termini of vaccinia mRNAs. The polyadenylation (poly(A)) tail plays an important regulatory role in mRNA translation and stability and an optimal length of polyA must be added to mRNA that can be done directly from the encoding polyA signal in the DNA template or by using a polyA polymerase following in vitro transcription. The poly(A) tails with increasing length enhances polysome formation and favorably impacts protein expression levels. The optimal length of the poly(A) tail is between 120 and 150 nucleotides. Although, a poly(A) tail even between 100 and 170 nucleotides can produce good protein yields. As, an example a poly(A) tail 100 adenosine nucleotides in length can yield a 35-fold greater amount of protein than a poly(A) tail 64 adenosine nucleotides in length in dendritic cells. (See, e.g. Mockey, M., Gonsalves, C., Dupuy, F. P., Lemoine, F. M., Pichon, C., and Midoux, P., 2006, Biochem. Biophys. Res. Comm. 340: 1062-1068.)

In eukaryotes the long stretch of untemplated adenosines, that is the poly(A) tail, is added to the 3′ end of mRNAs involving a 2-step process of endonucleic cleavage of the pre-mRNA and the addition of a poly(A) tail to the cleavage site. In the poly(A) signal essential sequence elements include a hexanucleotide poly(A) signal with a canonical sequence of AATAAA that is generally flanked by auxiliary upstream elements (USE) with many U repeats or UGUA repeat elements and downstream elements (DSE) with many U repeats or GU repeat elements. The strength of the PAS is influenced by these auxiliary USE and DSE elements. The endonuclease Cleavage and Polyadenylation Factor 3 (CPSF3) that cleaves the pre-mRNA at the cleavage site that is typically after a CA dinucleotide 15-30 nucleotides downstream of the poly(A) signal canonical sequence of AATAAA and 0-20 neucleotides upstream of the DSE found in the polyA signal where poly(A) polymerase is recruited to the cleavage site to catalyze the addition of the poly(A) tail. Generally, eukaryotic mRNAs undergo polyadenylation during mRNA processing. 3′ UTRs may encode for alternate polyadenylation. Human genes for example, contain more than one polyadenylation site that is likely related to UTR-alternate polyadenylation (UTR-APA) that would result in an mRNA isoform. This APA is different than the polyadenylation that results from coding region-polyadenylation (CR-APA) where the coding region. In UTR-APA the alternate poly(A) signal are often proximal to the stop codon. UTR-APA often possess non-canonical poly(A) signals such as ATTAAA, AGTAAA or TATAAA and thus are considered to be weaker signals. Although, non-canonical poly(A) signals in some instances can be favored over canonical poly(A) signals. (See, e.g., Yeh, H. S., & Yong, J., 2016, Molecules and cells, 39: 281-285.)

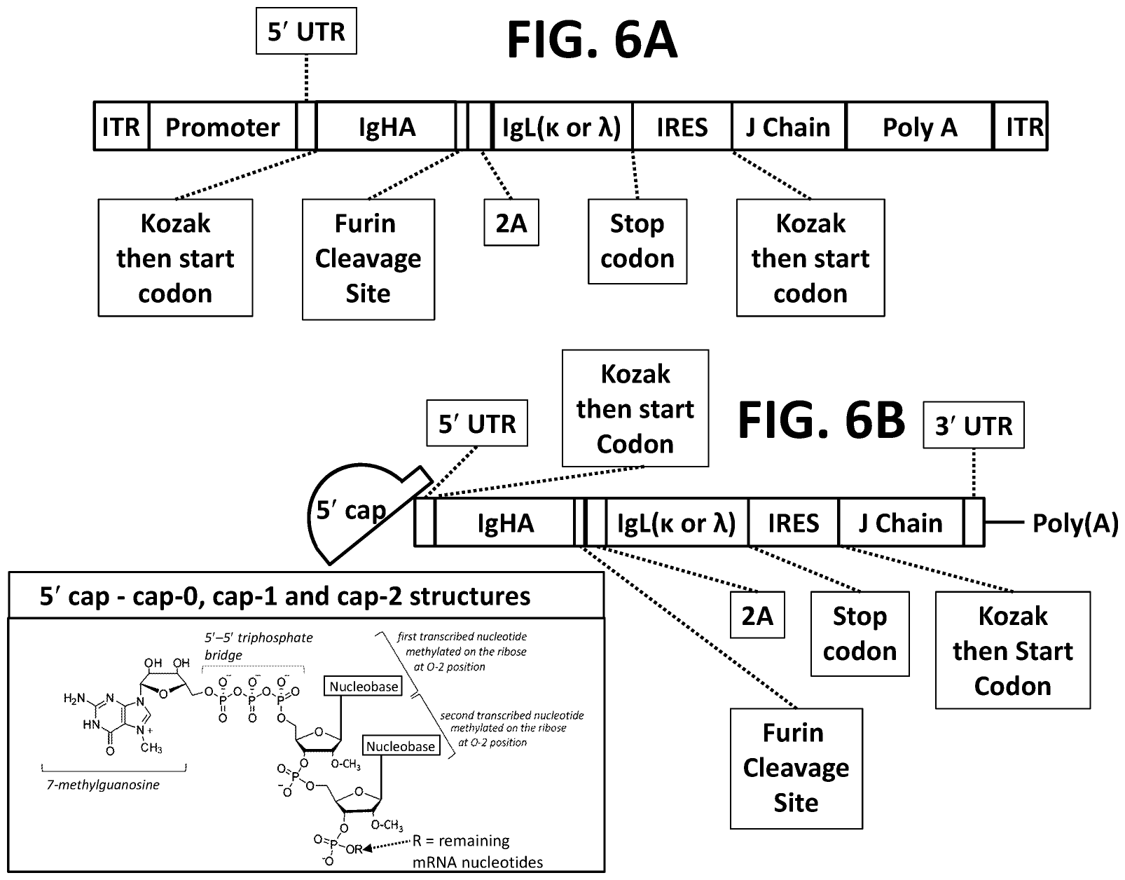

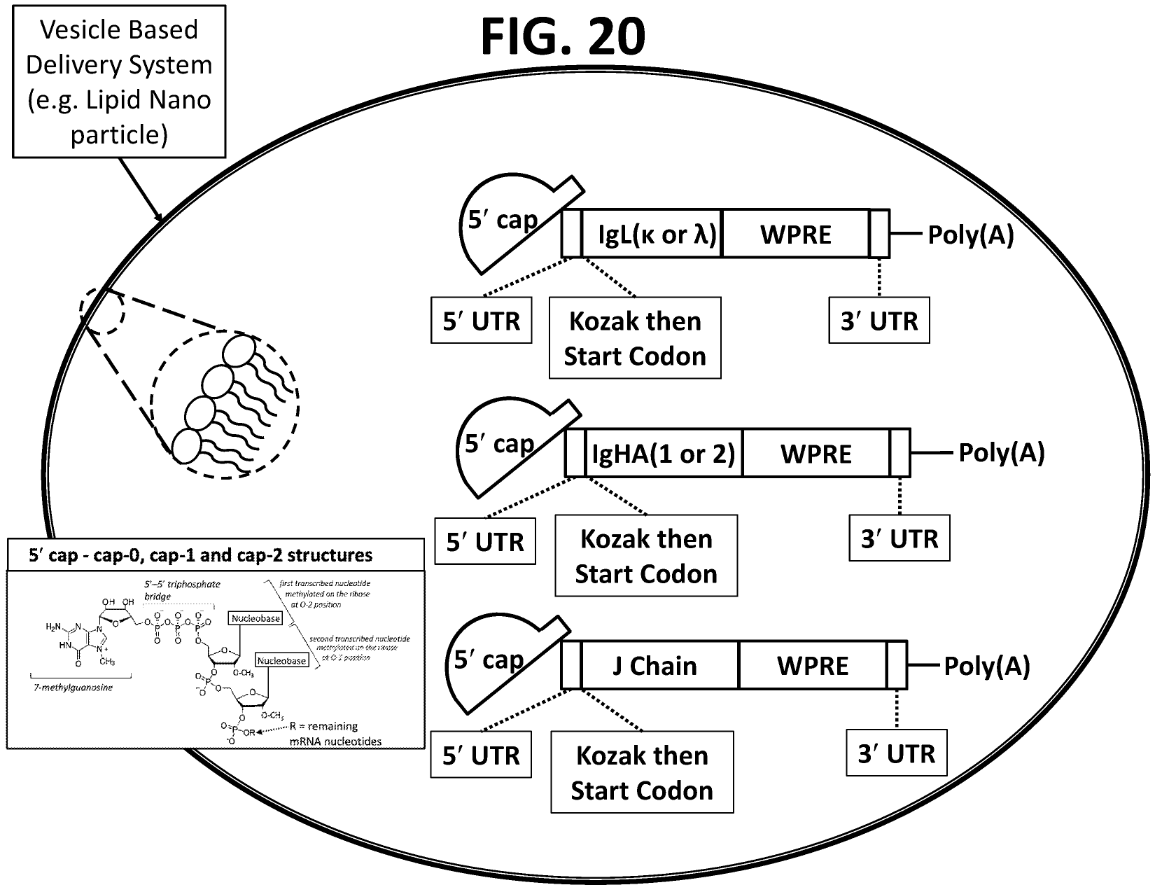

In some embodiments the vector framework for mRNA may encode for dIgA (see FIG. 6B) in a single vector or across 2 or even three vectors (see FIG. 20) that will optionally be encapsulated in a vesicle-based delivery system with emphasis on a lipid nanoparticle-based delivery system. When expressing dIgA in a single mRNA vector the vector framework inclusive of the 5′ UTR, transgenes and intermediate elements up to the stop codon is consistent with that used in DNA based vectors (see FIG. 6B). But in using an in vitro production system, post-translational modifications are often made such as adding the 5′ cap with the vaccinia virus capping enzyme. The mRNA begins at the 5′ cap that is added enzymatically with vaccine capping enzyme and is linked to the transcription start site that is highlighted as the 5′ untranslated region (5′ UTR). The 5′ UTR (See SEQ ID NOs: 74-86) can include the Kozak consensus sequence or the Kozak consensus sequence can be the 6-base sequence (SEQ ID NO: 73) concomitant and directly 3′ of the 5′ UTR. Following the Kozak consensus sequence the RNA sequence that encodes for the protein may code for one or more proteins where each protein will be separated from the other by one of (A) an internal ribosome entry site (IRES) (SEQ ID NO: 71), (B) a 2A self-processing peptide or (C) a concomitant furin cleavage site followed by as 2A self-processing peptide. 3′ of the stop codon of the final encoded protein in the mRNA vector would be the optional 3′ UTR if a 3′ UTR is used (See SEQ ID NOs: 87-102 for examples of 3′ UTRs that are optionally considered in this patent). Following the 3′ UTR is the optional use of a WPRE (SEQ ID NO: 72). The final element of the mRNA is the polyadenylation tail that results from polyadenylation of the polyadenylation signal in the DNA that is an important part of transcription termination. The poly(A) tail will be optimized to be between 120 and 150 adenosine nucleotides in length that may optionally be completed in vitro with a poly(A) polymerase.

### Allergens Including Type I Hypersensitivity

This patent contemplates the use of dIgA1, dIgA2, and engineered variants of dIgA as a gene therapy to mitigate allergies. In one embodiment allergies of the respiratory tract are targeted via gene therapy with dIgA1. In another embodiment allergies of the gastrointestinal tract are targeted with dIgA1 or dIgA2 (inclusive of polymeric immunoglobulin A2) or engineered variants. Type I hypersensitivity (or immediate hypersensitivity) is an allergic reaction provoked by re-exposure to a specific type of antigen referred to as an allergen. When an antigen is not associated with a pathogen or infectious agent causes hypersensitivity to those exposed to such antigens the hypersensitivity is referred to as an allergic reaction and the antigen is referred to as an allergen. Allergic reactions can be caused by allergens binding 2 adjacent Immunoglobulin class E (IgE) antibodies bound to adjacent IgE receptors (FcεRI) on mast cells.

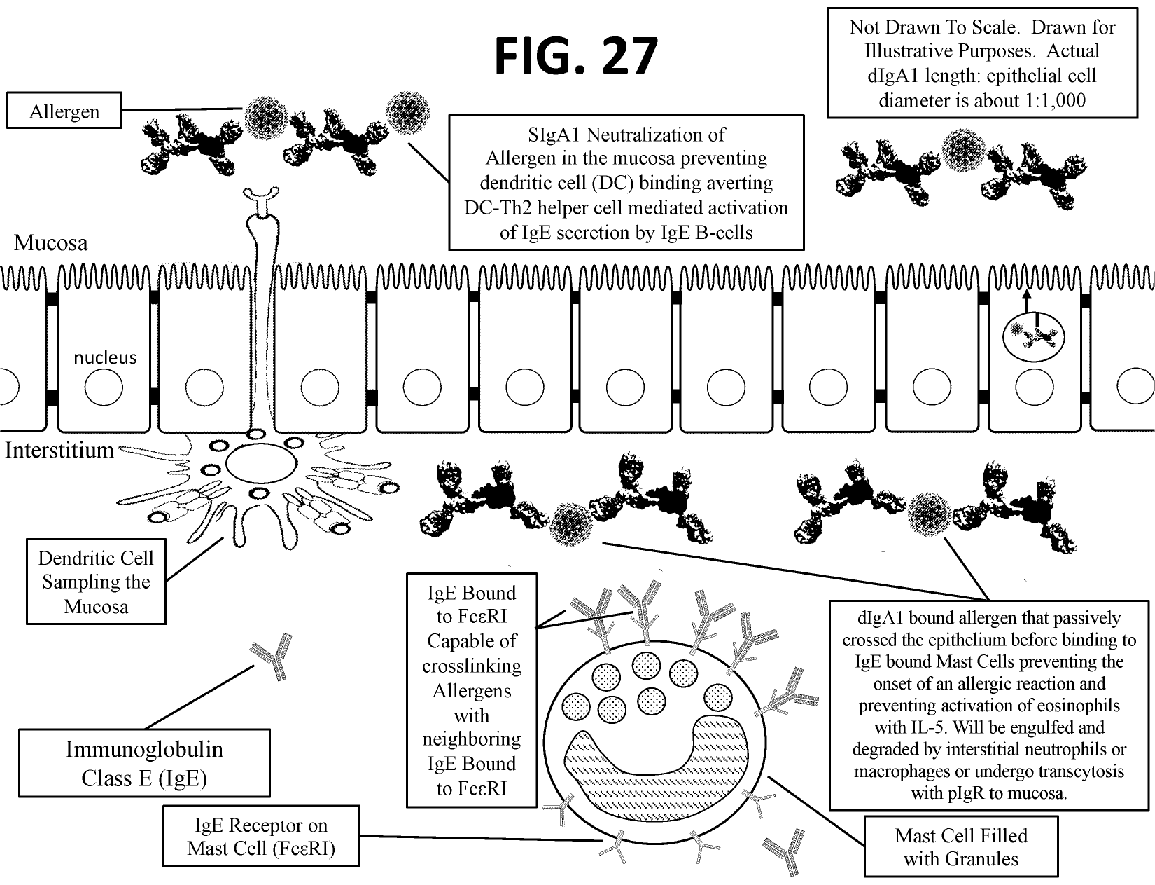

In many allergic reactions humans become sensitized to the innocuous antigen and produce IgE antibodies against it. Future exposure to the antigen causes the activation of IgE binding cells mainly mast cells and basophils. Allergies may occur when the allergens cross the mucus membrane of the nasal cavity, eyes, lungs or skin as examples. And genetic factors are thought to play a role in allergies. For those with allergies to an allergen IgE specific to that antigen tends to be highly elevated in the interstitium lining airways and thus, the most effective means of combat to prevent the allergen from binding IgE would be dIgA that is present both in the interstitium and at the mucus barrier in its SIgA form (see FIG. 27). There are other allergies such as forms of asthma that are due to cytokines including interleukins (IL) and do not have a causal link to IgE.

Allergen induced allergies occur through contact with the mucosa of the respiratory tract, digestive tract, through skin absorption or through blood circulation in some cases such as a bee sting. In the respiratory tract common allergies include pollen, birch, dust mite feces, and cat dandruff causing asthma. About half of the U.S. population is sensitive to at least one innocuous antigen or allergen. The most common allergies in developed countries are from airborne allergens resulting in symptoms mainly affecting the nasal passage and lower airways including the lungs which is classified as asthma. In some cases people are sensitive to one specific allergen and in other cases have nonspecific sensitivity to a range of allergens. When people are sensitive to specific allergens or allergen classes strategies that employ neutralization and degradation of the antigens respectively with SIgA and dIgA may effectively be used. Allergens that are ingested such as proteins found in specific foods that result in allergies are sometimes but not always limited to the gastrointestinal tract.

Mast cells have high affinity IgE receptors (FcεRT) that tightly bind to IgE and when an allergen that the individual is sensitized to crosses the epithelial cell mast cells can detect them generally via two adjacent IgE bound to the FcεRT. Thus, allergens generally have to have more than one identical or nearly identical binding faces (as far as the immunoglobulin is able to detect) to result in an allergic reaction. When an antigen is bound to IgEs bound to FcεR on mast cells it results in the mast cell to release granules containing cytokines, histamine, tryptase, Platelet-activating factor that causes allergic symptoms and in individuals with asthma cause smooth muscle contraction and mucosal edema and can cause anaphylaxis. Eosinophils are also activated by Mast cells with IgE bound to antigens with IL-5 and can result in eczema and deadly anaphylaxis. There are two phases of sensitization to allergens: The induction phase and the effector phase. The induction phase involves many different cells and proteins including epithelial cells, chemokine ligand 27 (CCL27), immature dendritic cells, antigen presenting dendritic cells, T_{H}2 T-helper cells, cytokines, such as interleukin (IL)-4, IL-5 and IL-13, class switching of B cells to IgE secreting B-cells, IgE secretion and binding to the high-affinity IgE receptor (FcεRI) on the membrane of mast cells and basophils, forming sensitized mast cells and basophils. When immature dendric cells respond via pattern recognition receptors to danger signals they mature into competent antigen-presenting myeloid-type dendritic cells. Generally, allergen detection occurs when these mature dendritic cells subsequently bind and processes the allergen presenting a polypeptide fragment of the allergen via the major histocompatibility complex class II (MHCII) receptor where the dendritic cell subsequently migrates to the local lymph node where they interact with naïve T-cells (T_{N}) through their T-cell receptor (TCR) via MHCII and co-stimulatory molecules that results in T_{H}2 T-helper cells maturation and migration to the local tissue such as the lungs interstitium subsequent interactions as a result of the same or sufficiently similar allergen between the MHCII of dendritic cells and the TCR of T_{H}2 T-helper cells results in the secretion of IL-4 and IL-13. IL-4 and IL-13 stimulate immature IgM or IgD B-cells bound to the allergen to class-switch into IgE B-cells and secrete IgE that can bind IgE FcεRI receptors on mast cells. (See, e.g. He, S. H., Zhang, H., Yang, P. C., 2013, Acta pharmacologica Sinica, 34:1270-1283)

The effector phase begins when the same or a sufficiently similar allergen cross-link two adjacent IgEs on sensitized mast cells or basophils. Activated mast cells or basophils subsequently degranulate releasing granules housing proinflammatory mediators and cytokines including histamine, tryptase LTC4, PGD2 thereby causing the clinical manifestations of allergy. Soluble allergens, sIgEs and mast cells or basophils are three key factors in the patho-physiological process of allergic inflammation, representing causative factors, messengers and primary effector cells, respectively. In contrast to primary effector cells, eosinophils and neutrophils are secondary effector cells, which can be accumulated and activated through the mediators released from mast cells or basophils. (See, e.g. He, S. H., Yang, P. C., et al., 2013, Acta pharmacologica Sinica, 34:1270-1283.) A similar sensitization response to that of respiratory allergies occurs when allergens bind to IgE bound mast cells in the gastrointestinal tract where there is an outflow of fluid across the gut epithelium induces vomiting and diarrhea. (See, e.g. Renz, H., Allen, K., Sicherer, S. et al., 2018, Nat Rev Dis Primers vol. 4, pp. 17098)

The immune response that leads to an excess of IgE production in response to an allergen is caused by two processes. One process causes naïve T cells to differentiate into T_{H}2 T-helper cells that produce cytokines that are specialized for promoting responses against parasites or extracellular bacteria. The second comprises T_{H}2 cells to stimulate B-cells to class switch to produce IgE. This results in higher levels of IgE in the interstitium of the lungs for example and the mast cells in the lung are subsequently heavily bound to IgE through the FcεRI receptor making the individual highly sensitive to that antigen the IgE is specific to making an individual asthmatic or results in GI disorders when the stomach is affected by the allergen. This instant patent contemplates blocking allergens from binding IgE by targeting the allergens. In one embodiment dIgA1 would provide mucosal protection in its SIgA1 form by binding to allergens in the respiratory mucosa and also in its dIgA1 form in the interstitium above and below the basement membrane providing three lines of protection against allergens binding IgE bound mast cells and one or two lines of protection—depending if the dendritic cell is sampling the mucosal environment—against allergens binding IgE bound dendritic cells (see FIG. 27). (See, e.g. Holgate, S., Wenzel, S., Postma, D. et al., 2015, Nat Rev Dis Primers vol. 1, pp. 15025)

In some embodiments dIgA1 encoded gene therapy specific to the allergen of interest is the therapeutic approach that prevents allergic reactions. Because dIgA1 and dIgA2 are actively transported to the mucosa of the lungs and gastrointestinal tract where it becomes SIgA it can bind to allergens before they have a chance to bind to dendritic cell extensions sampling the mucosa or cross the mucosa and reach IgE bound mast cells that are largely responsible for the allergic response in asthma (See FIG. 27). Additionally, any allergens that cross the mucosal barrier will also be targeted by dIgA1 in the interstitium. Thus, dIgA provides two or three levels of protection against allergens. The discovered potent dIgA1 immunoglobulins or immunoglobulin binding regions incorporated into a dIgA vector construct will code for dIgA1 or dIgA2 and be able to bind to the allergen on multiple faces neutralizing the antigen and causing agglutination where macrophages, monocytes and neutrophils can degrade the antigen bound antibodies without causing allergies nor ADE or any adverse reactions.

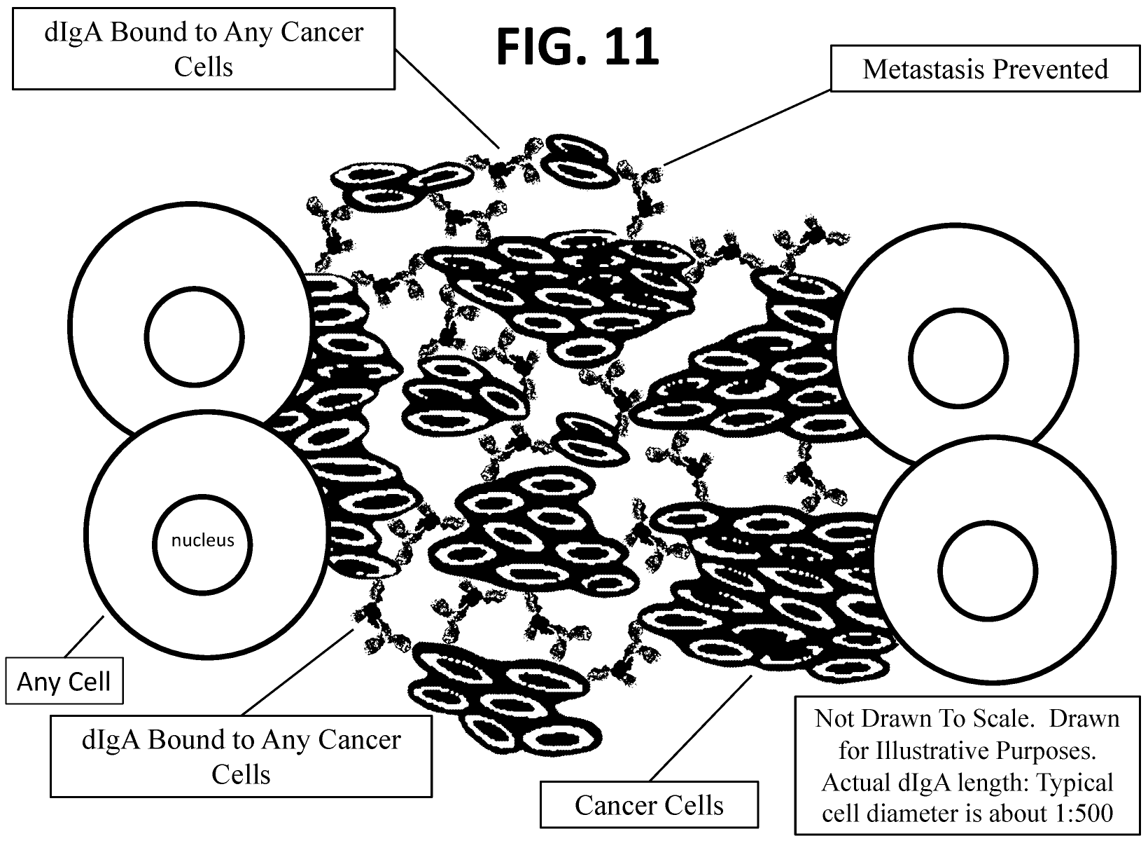

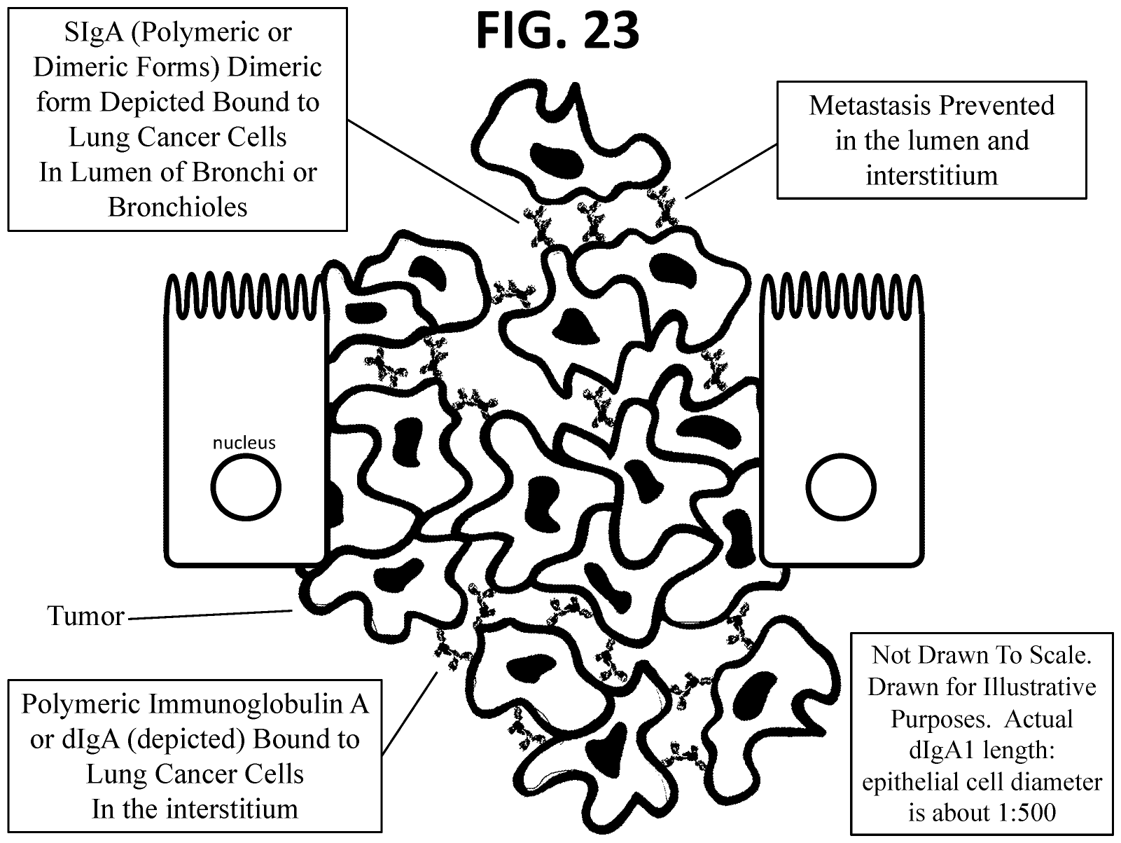

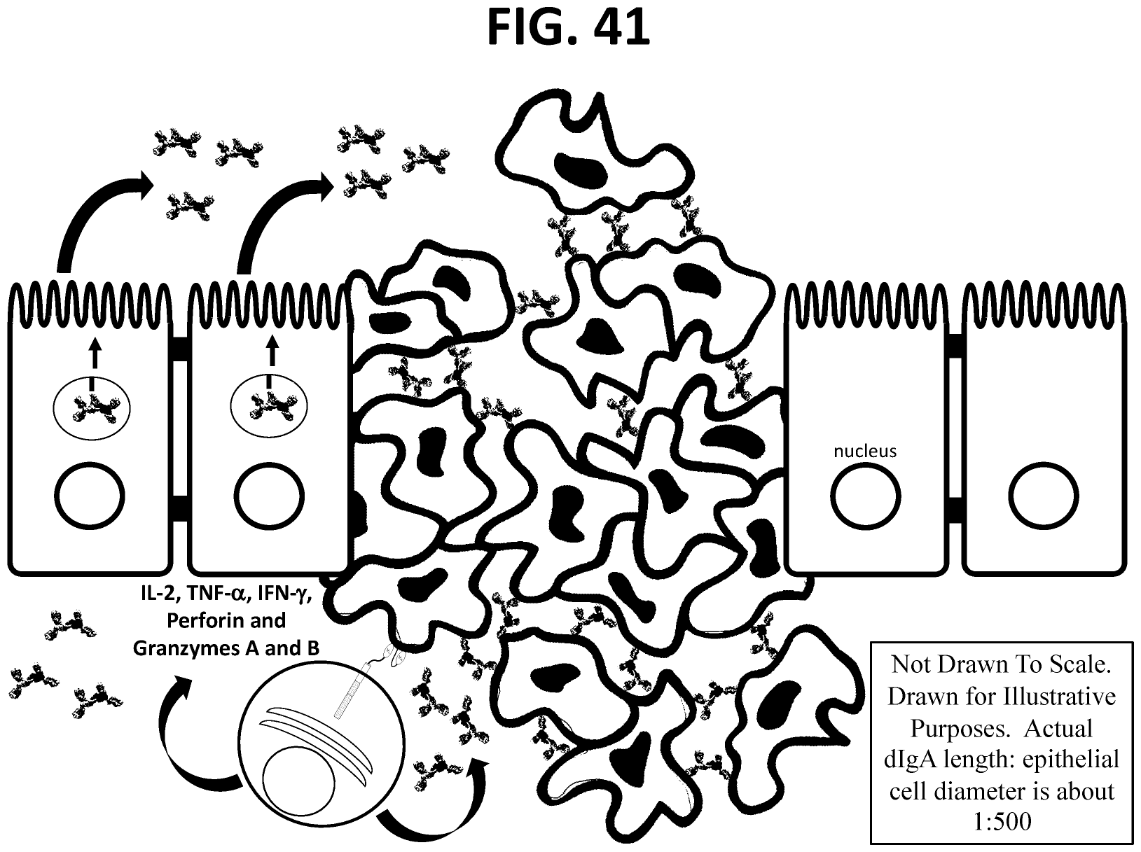

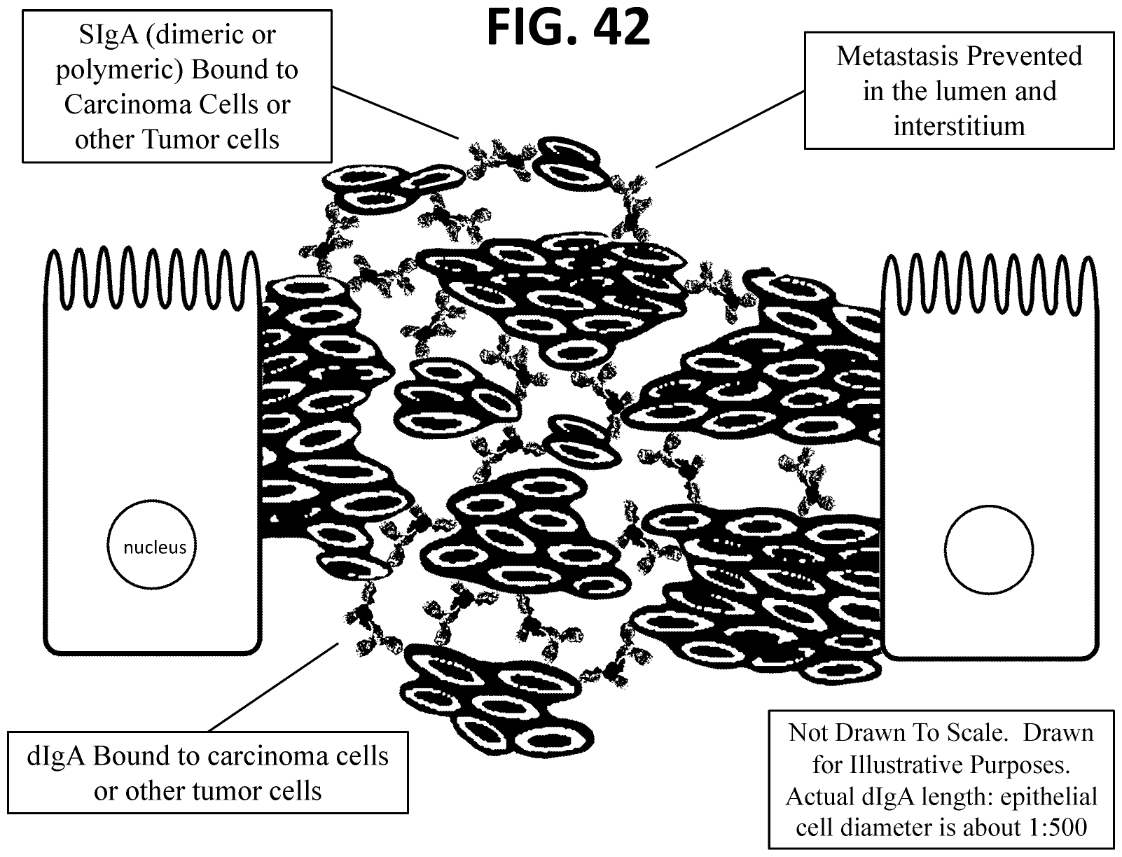

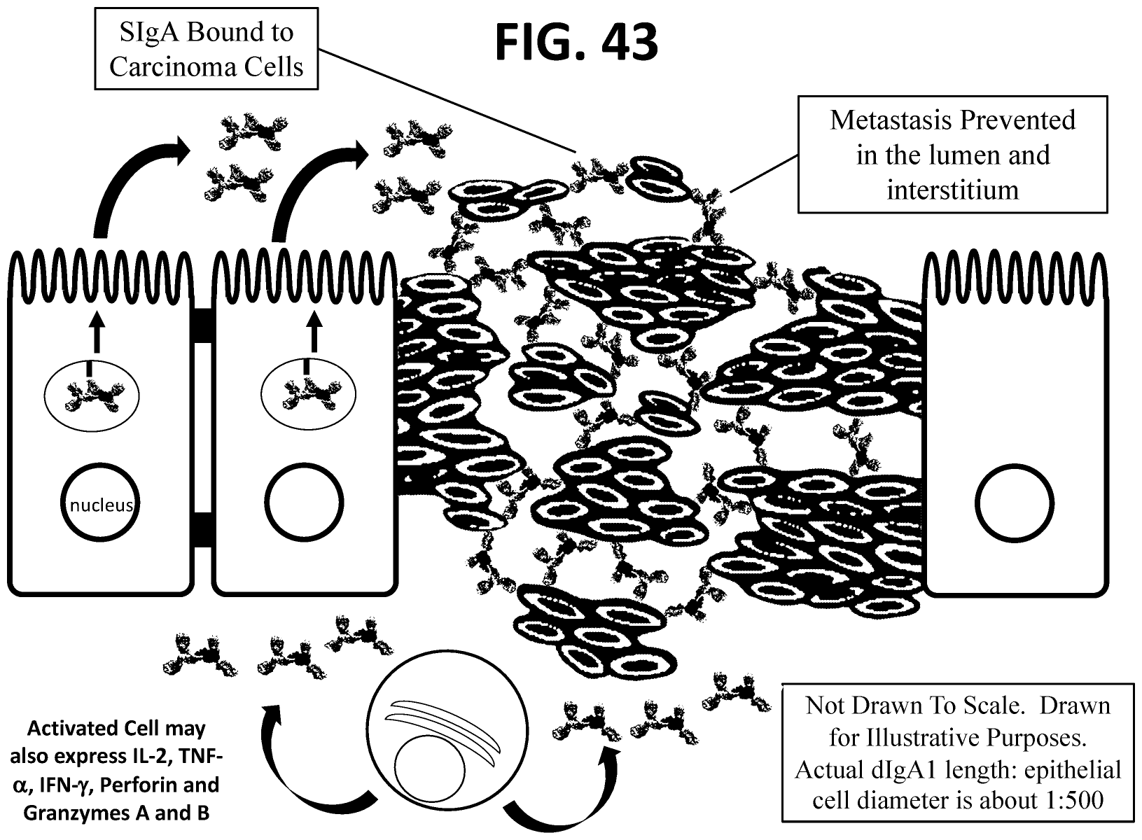

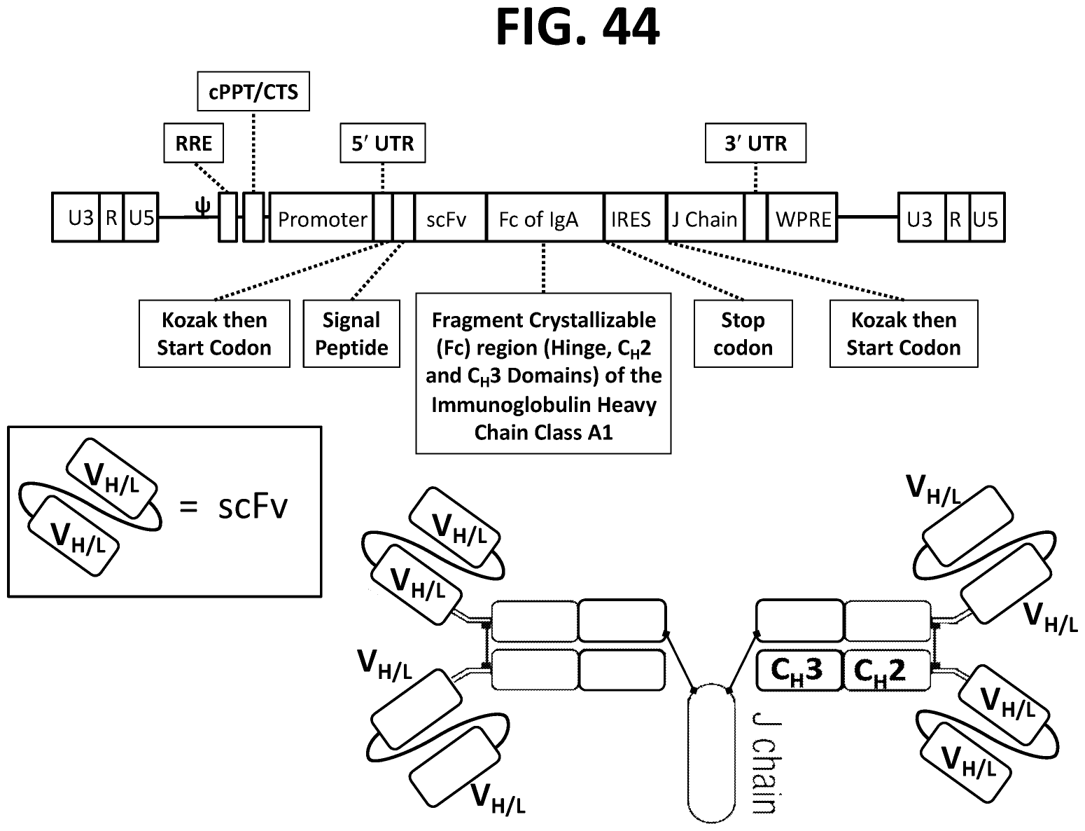

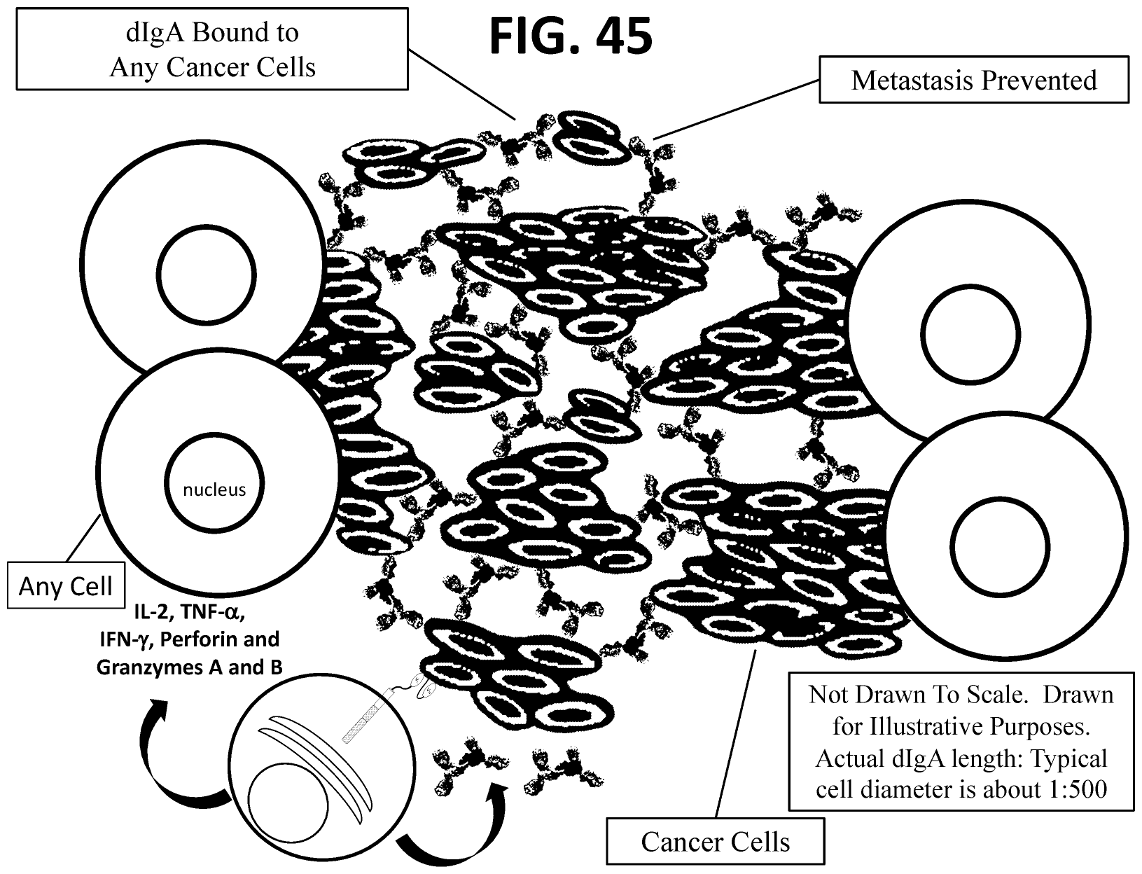

Cancer This patent contemplates the use of dIgA1 (inclusive of polymeric immunoglobulin A1), dIgA2 (inclusive of polymeric immunoglobulin A2) and engineered variants and even bispecific dIgA to target cancer and further dscFV-FcIgA (FIG. 44) to target cancer. Cancer is a complex pathology that is made up of many different forms. Key characteristics of cancer are abnormal and nonspecific cell growth taking the place of healthy cells while not supporting and even impairing organ function, immune system function or tissue function. The most common types of cancer in males include lung cancer, colorectal cancer, prostate cancer and stomach cancer. In females the most common types of cancer include breast cancer (80% invasive ductal carcinomas), lung cancer, colorectal cancer and cervical cancer. In other words most of the more common types of cancer involve mucosal or exocrine tissue and is often referred to as a carcinoma which makes up 80%-90% of all cancer cases. Thus, dIgA and polymeric immunoglobulin A has privileged access to the malignancy in the mucosal lumen or exocrine channel to a greater degree than other immunoglobulins as a result of its active transport by polymeric immunoglobulin secreting receptor (pIgR) to the apical face of the epithelial cells lining the mucosal or exocrine tissue (see FIGS. 23, 41, 42 and 43). A further benefit of dIgA1 (inclusive of polymeric immunoglobulin A1), dIgA2 (inclusive of polymeric immunoglobulin A2) and engineered variants as well as SIgA1 and SIgA2 over other immunoglobulins is that they can prevent “metathesis” or the spread of cancers cells to other areas of the body because of its two or more binding faces. When a cancer cell departs from the malignant tumor polymeric immunoglobulin A, dIgA or SIgA in the mucosa or exocrine duct (channel) can capture the escaping tumor cell(s) by binding the cell on the immunoglobulin face that is opposite the immunoglobulin face bound to the malignancy (see FIGS. 11, 23, 42, 42, 42 and 45). If there are interstitial spaces in the tumor Immunoglobulins including dimeric immunoglobulin A and polymeric immunoglobulin A and even dscFV-FcIgA can penetrate through the interstitial spaces of the tumor that can accommodate their size. Polymeric immunoglobulin A and further Dimeric immunoglobulin A specific to a receptor on the tumor cell such as but not limited to Epidermal Growth Factor Receptor (EGFR), Human Epidermal Growth Factor Receptor 2 (HER-2/mu or HER-2/μ) or Programmed Death-Ligand-1 (PD-L1) can prevent metastasis. There are a number of factors that will influence the degree to which polymeric immunoglobulin A and dIgA and engineered variants will have multifaced or multivalency binding to neighboring tumor cells or even a tumor cell and a neighboring healthy cell. Those factors include but are not limited to overall antibody concentration in the body, the antigen concentration on the tumor cells, the binding affinity of the antibodies and the rate of endocytosis and lysosomal degradation of the antibodies. Other factors include the diameters of the interstitial spaces between the cancer cells, the rate of antibody flow or diffusion through the tumor. (See e.g., Thurber, G. M., Schmidt, M. M., & Wittrup, K. D., 2008, Advanced drug delivery reviews, 60:1421-1434; Also see, Xenaki, K. T., Oliveira, S., & van Bergen En Henegouwen, P., 2017, Frontiers in immunology, 8:1287).

Overall, a dIgA1 (inclusive of polymeric immunoglobulin A1), dIgA2 (inclusive of polymeric immunoglobulin A2) and engineered variants gene therapy for cancer specific to a cell surface receptor that is either 1) upregulated on the tumor, 2) that has an opportunistic mutation or 3) both at the ideal binding affinity can enhance the clinical outcome over the equivalent IgG or IgA recombinant variant as a stand-alone treatment. The dIgA and polymeric immunoglobulin A being actively transported across the epithelium provides a higher level of concentration of SIgA in the mucosal and exocrine channel. Thus, the dIgA encoding gene therapy will have a more effective outcome on lower doses. In addition, SIgA will have privileged access to the carcinoma on the apical face of the epithelium. Where dIgA is actively transported across epithelial cells expression pIgR to become SIgA. Non-dimeric and non-polymeric antibodies such as IgG and IgA must rely on passive diffusion across the epithelium if the particular epithelium where the carcinoma is found can even be passively diffused across. IgG and IgA ability to access to the apical face of the epithelium and in turn the face of the tumor facing into the mucosal or exocrine duct is at far lower levels than SIgA. IgG and IgA would have to then rely more on diffusion through the interstitial spaces of the tumor facing in the direction of the basolateral face of the epithelium to reach the face of the tumor facing into the mucosa or exocrine duct to reach therapeutically relevant concentrations on that face of the tumor. Carcinomas make up 80-90% of all cancer diagnosis and cancer metathesis can be reduced with dIgA and polymeric immunoglobulin A by the agglutination of cancer cells in the malignancy (see FIGS. 11, 23, 42, 42, 42 and 45) and also in the early stages of cancer cells attempting to depart or metastasize from the tumor thereby mitigating metastatic cancer the leading cause of cancer death.

Many proteins on the cell surface can move within the plasma membrane through a process called membrane diffusion. In some cases cell surface proteins have restricted flow (diffusion or lateral movement) through the lipid bilayer of the cell due to their interactions with the cytoskeleton. In other cases there is a degree of organization of the membrane that may also restrict lateral movement of cell surface proteins. Lateral movement of cell surface proteins enhances the rate of simultaneous binding of a single dIgA or single polymeric immunoglobulin A, to two neighboring cancer cells. Similarly, cells have viscoelastic behavior, that is the cell surface has both viscous and elastic characteristic that results in changes in cell shape that further enhance the rate of simultaneous binding of a many dIgAs and many polymeric immunoglobulin As to two neighboring cancer cells. That is these two properties of lateral movement of cell surface receptors and the viscoelastic behavior of the cell enhances the degree to which dIgA and polymeric immunoglobulin A can prevent metastasis because it creates increased likelihoods that these multivalent immunoglobulins bound to one cell on one face will bind to a neighboring cell on the other distinct face of the dIgA. The cell is in constant motion both from both the perspective of lateral flow of cell surface proteins and in terms of minor changes in shape as a result of the viscoelastic behavior. (See e.g., Nicolson, G. L., 2014, Biochim Biophys Acta.; 1838:1451-66.)

Further, pIgR is often upregulated and less often down regulated on epithelial cells proximal to the tumorous growth. One example of a defining feature of most cancers is an upregulation of secreted proteins and membrane bound proteins. The upregulation is significant enough that therapeutic antibodies are often used to mitigate the cancer despite healthy cells in the body also producing the same cell surface markers. Thus, other immunoglobulins may only have access to part of the parts of the cancerous growth that are not facing into the lumen or exocrine channel or bordered by epithelial cells lining the mucosa and exocrine gland. Many organs with a mucosa and exocrine glands have their own lymphatic support system. A common therapeutic strategy to battle cancer includes use immunoglobulins to target cancer.

The epidermal growth factor receptor (EGFR) is a transmembrane protein that is implicated in cancer. Mutations that cause EGFR overexpression have been associated with a number of cancers including Adenocarcinoma of the lung is the most common type of lung cancer, colon cancer, epithelium tumors of the head and neck at about 80-100% of cases. Somatic mutations involving EGFR lead to its constant activation, which produces uncontrolled cell division. In a wide variety of tissues EGFR expression is at a low level. Excessive expression or activation of EGFR can induce malignancies or malignant transformations. EGFR is observed in 45-75% of non-small cell lung cancer (LSCLC) and is associated with aggressive clinical behavior including increased metastatic rate, high tumor proliferation and advanced stages of cancer. (See, e.g. Zhang, X., & Chang, A., 2007, Journal of medical genetics, vol. 44, pp. 166-172.) Non-small cell lung cancer remains the leading cause of cancer death in the U.S. and accounts for 85% of lung cancer cases in the U.S.

Another important receptor class involved in mucosal cancers is the PD-1/PD-L1 pathway which controls the maintenance and induction of immune tolerance within the tumor microenvironment. Programmed cell death protein 1 (PD-1) is a T-cell surface protein that regulates the immune response by down regulating the immune system and promoting self-tolerance. This pathway can thus attenuate the immune response of T cells to cancer cells. PD-1 has been targeted in immunotherapies to block the activity of this receptor in T-cells. Effectively, PD-1 antagonists can enhance the T-cell response against cancer cells and also blocks through competitive inhibition particular cancer cell receptors referred to as Programmed Death Ligand 1 (PD-L1) and PD-L2 from binding PD-1 on T-cells. When PD-L1 and PD-L2 bind T-cells they reduce T-cell survival and proliferation, in addition to reducing their cytokine secretion while making it more difficult for such PD-1 bound T-cells from being activated. It has been shown for example, that mice which lack PD-1 can steadily develop autoimmunity. Anti-PD-1/PD-L1 inhibitors have seen success in reducing cancerous tumor growths a number of mechanisms that including enhancing the T-cell response, activating a series of immune system responses that combat tumor cells in addition to inducing infiltration into tumors. Additionally, CTLA-4 (CD152) agonist also controls the proliferation of T-cells and thus binding CTLA-4 with an antagonist enhances T-cell proliferation in the presence of cancer. It has also been shown that CTLA-4 activated T-cells can inhibit the proliferation of other T-cells.

### T-Cell Developmental Stages and the Development of Other Lymphocytes

Lymphocytes are derived from pluripotent hematopoietic stem cells (HSC) in the bone marrow. HSCs give rise to common lymphoid progenitors. Common lymphoid progenitors are the precursors to all B-cells, all T-cells, all NK cells and some dendritic cells. Common myeloid progenitors on the other hand are also derived from HSC but are the precursors to all other innate immune cells including dendritic cells, neutrophils, eosinophils, basophils mast cells, monocytes and macrophages. The common lymphoid progenitor that differentiates into T-cells migrate from the bone marrow to the thymus or other tissue.

T-cell development somewhat mirrors that of B-cell development. The T-cell receptor similar to the B-cell receptor (BCR) or immunoglobulin has 2 chains: an alpha and beta chain each consisting of gene segments that are intended for recombination to give rise to a large diversity of T-cell receptors (TCRs). Similar to the B-cell receptors (BCRs), the alpha and beta chain locus of the T-cell undergo recombination events first in the beta chain and then in the alpha chain. During the early development stage the T-cell is referred to as double negative for CD4− and CD8−. In this stage the beta chain undergoes D-J rearrangement followed by V-DJ rearrangement. If the rearrangement is productive, that is if the rearrangement results in the amino acids necessary for a functional beta chain then the pre T-cell receptor formation between the beta chain and surrogate alpha chain forms. Although, if the beta chain rearrangement is not productive it may be rescued with a second rearrangement event at a second downstream cluster of D and J segments at the beta chain locus. Following productive beta chain rearrangement and formation of the TCR between the beta chain and surrogate alpha chain which triggers the expression of CD4+ and CD8+ and the T-cell is now double positive for CD4+ CD8+. The alpha chain then undergoes recombination events if the rearrangement is not productive then successive rearrangements of the alpha chain locus will occur until the T-cell is rescued or the T-cell will undergo apoptosis after so many unsuccessful iterations. The resulting double positive T-cell will then sample the Thymus by engaging MHC-I or MHC-II presenting cells on stromal on bone marrow derived cells, mainly dendritic cells and macrophages, that present self-antigens. If the T-cell does not interact strongly enough with the MHC or if it is reactive to self MHC peptides it undergoes apoptosis by negative selection. Whether a double positive T-cell becomes a CD4 or CD8 T-cell depends on whether it interacts with an MHC-I or MHC-II. Those double positive T-cells that interact well with MHC-I are destined to become CD8+ cytotoxic T-cells whereas those that interact well will MHC-II are destined to become CD4+ helper T-cells (T_{H}-cells). This overall process of T-cell development results in the death of 95-99% of developing thymocytes and the preservation of a population of cells bearing carefully selected TCRs that are classified as naïve T-cells.

These naïve T-cells are destined to further differentiate into T-cell classes and subclasses. For example, CD4+ T-Helper Cells (T_{H}-cells) assist other lymphocytes with activation including B-cell, CD8+ T-cells and macrophages. T_{H}-cells become activated through their TCR by interacting with MHC-II presenting cells. T_{H}-cells have several subsets including T_{H1}-cells that activate macrophages, T_{H2} cells and T_{H17}-cells that recruit and activate other innate immune cells, T_{FH}-cells (T follicular helper cells) that activate B-cells in the lymphnodes and regulatory T-cells (T_{reg}-cells) are involved in preventing autoimmunity during an immune response. T_{H1}-cells also are involved in the regulation of formation of CD8+ T-cell memory. This process includes but is not limited to a professional antigen presenting cell such as a dendritic cell or macrophage that must be activated by a T_{H}-cell via an MHC-II interaction so that a CD8+ T-cell can be activated to have immunological memory. Memory T-cells can subsequently undergo receptor revision in a RAG mediated process that is similar to how diversity is generated in immunoglobulins. Although, regulatory T-cells are excluded from receptor revision.

During an immune response naïve T-cells proliferate in response to foreign peptide presentation in MHC-I or MHC-II. During the priming phase T-cells become activated and expand to substantially increase their number—presenting the same T-cell receptor as the parent cell—over the course of a week giving rise to an extensive number of daughter cells that carry our effector functions. In the contraction phase T-cell numbers drop by 90-95%, in the memory maintenance phase a stable population of long-lived memory T-cells reside in lymphoid, non-lymphoid tissue, circulating the blood and some will become tissue resident memory T-cells that will stay confined to that tissue. Activation of memory T-cells is similar to that naïve T-cells. Upon activation of memory T-cells there is an expansion phase where the memory T-cells clones itself producing many daughter cells capable of a secondary effector T-cell response. The effector phase is followed by a contraction phase where the reactivated T-cell population drops to 90-95% and the resulting surviving cells become memory T-cells. The sensitivity of CD8+ memory T-cells to reactivation depends in part on whether the T-cell activation occurred from professional antigen presenting DCs that were also activated by CD4+T_{H} cells that also secrete various cytokines to aid in CD8+ T-cell memory.

Tissue Residency and Migratory Capacity of T-cells and other cell Types.

This instant patent contemplates the engineering of T-cells to express dIgA1 (inclusive of polymeric immunoglobulin A1), dIgA2 (inclusive of polymeric immunoglobulin A2) and engineered variants in addition to dscFV-FcIgA with an upregulation of dIgA and polymeric immunoglobulin A expression upon activation; and optionally to be engineered to express a chimeric antigen receptor (CAR); with optional engineering to disrupt TCR expression to eliminate GVHD and optionally generate an off-the shelf product. In one embodiment the dIgA1 (inclusive of polymeric immunoglobulin A1), dIgA2 (inclusive of polymeric immunoglobulin A2) or engineered variant expressing T-cell is a CD8+ T-cell. In another embodiment a subpopulation T-cells such as memory T-cells with a tissue-resident markers (T_{RM}) are isolated or positively selected for following expansion to T-cells. T_{RM} that have properties to favor taking up residency in the lungs such as the resident memory marker CD69+ and optionally also favoring taking up mucosal resident memory such as CD103+ can be positively selected from a blood sample or from an expanded T-cell sample. Further in some embodiments enhancement to CD103+ expression on T-cells can be made by cross priming T-cells with CD103+ DCs: Such a process occurs in the lung enhance the expression or differentiation of CD103+ on T-cells. Further in some embodiments the cell may be further engineered to reduce or eliminate the expression of PD-1 in the CAR T-cells and further to optionally disrupt TCR expression to generate an off the shelf CAR T-cell product.

T-cells take up residency is many tissues especially those that have exposure to invading pathogens including the lungs, intestinal tract and skin. Taking up residence in tissues ensures a more rapid response to pathogens that would observe a delay if immunity was relied solely upon circulating T-cells. There are three known subsets memory T-cells that are characterized by their locale. Effector memory (T_{EM}) T-cells circulate in the blood through non-lymphoid tissues, Central memory T-cells (T_{EM}) generally reside in secondary lymphoid organs finally tissue-resident memory T-cells (T_{RM}) are found in the peripheral tissues. T_{RM} has distinctive properties related to TEM and T_{EM} that are related to migration and adhesion that allow for directed entry and maintenance in specific tissues. There is a learning process associated with T_{RM} formation. Generally, upon infection at a mucosal surface antigen is transported to the draining lymphnode by dendritic cells or drained directly via lymphatics. Dendritic cells present antigen to naïve T-cells to induce their proliferation and differentiation. Activated T-cells circulating through the blood enter inflamed tissue though signaling that activates integrins that enable strong adhesion and entry into the tissue. While T-cell migration is more promiscuous during the effector phase of an immune response as the T-cell homes in on the infected tissue, the migration of memory T-cells at steady state is more restricted. T_{EM} expressing the CCR7+ marker are more restricted while T_{EM} or CCR7− memory T-cells express other integrins and chemokine receptors that allow for recruitment into some peripheral tissues at steady state. For example, resting memory CD8+ T-cells can equilibrate into secondary lymphoid organs, lung alveoli, and liver. Although, the entry of resting memory CD8+ T-cells into the brain, intestinal mucosa, skin, most of the lung mucosa and genital tract is restricted. This is not surprising given that upon the T-cell priming stage phase the T-cells in the distal lymphnodes of the infected tissue undergo an upregulation of receptors necessary for adhesion and entry into associated tissues. Although, these tissue localization cell surface proteins on T-cells are rapidly downregulated during the effector stage. If a memory T-cell becomes a resident memory T-cell (T_{RM}) transfer experiments have demonstrated that such T-cells retain their ability to migrate back to the tissue of origin when placed into the circulating blood. This instant patent contemplates positively selecting T-cells with T_{RM} markers to be engineered to express a Chimeric Antigen Receptor (CAR) and also dIgA1 (inclusive of polymeric immunoglobulin A1), dIgA2 (inclusive of polymeric immunoglobulin A2), engineered variants or dscFV-FcIgA antibodies as a means to mitigate cancers. Upon inflammation of tissue memory T-cells experience an upregulation of cell surface proteins that allow for their entry into the inflamed tissue. At the site of inflammation E-selectin and P-selectin is upregulated on the endothelial cells that enables the memory T-cell to be directed to the site of inflammation through interactions with these selectins. Inflammation can cause an upregulation of CXCR3 on T_{H1} helper cells and CD8+ T-cells. CXCR3 regulates recruitment of effector T-cells into tissues that are otherwise excluded from T-cell entry such as the CNS. In the lung memory CD8+ T-cells are recruited into the airways via CCR5 that is ligated by multiple inflammatory cytokines including CCL5, macrophage inflammatory protein 1α (MIP-1α), CCL2 and MIP-1β. Although, after the effector phase effector permissive tissues become inaccessible to circulating memory CD8+ T-cells. (See, Shin, H., & Iwasaki, A., 2013, Immunological reviews, 255:165-181.)

Multiple peripheral tissues contain tissue resident memory CD8+ T-cells including the CNS, skin and lungs. Additionally, while CD8+ and T-cells appear to take residence in the specific tissue CD4+ T-cells do so to a lesser degree. Tissue resident memory T-cells are often identified according to two specific receptors CD103 and CD69. Tissue-resident memory T-cells (T_{RM}) can be distinguished from circulating T-cells based on the expression of the T-cell activation and retention marker CD69 and integrin αE (CD103). CD103 is expressed on Tim cells in many peripheral tissues including the skin, gut, brain and lung airway while the expression is low on circulating T-cells. CD103 is said to define mucosal resident memory CD8+ T-cells whereas CD69+ is said to define peripheral tissue memory T-cells (T_{RM}). CD103 is the αE subunit of the integrin that binds E-cadherin expressed on epithelial cells. Thus, CD103 is by its nature a defining feature of CD8+ T_{RM} cells in mucosal tissues. CD103 is thought to allow T_{RM} to anchor to specific locations within the mucosal epithelium. CD4+ and CD8+T_{RM} exhibit elevated levels of transcripts encoding inflammatory cytokines and cytotoxicity associated gene in comparison to their circulating memory T-cell counterparts. This suggests that T_{RM} can mount a rapid response upon activation. At the same time, they also are upregulated with multiple inhibitory cells surface markers including programmed death-1 (PD-1) thought to prevent unnecessary excess inflammation. This may be a contributing factor that CAR T-cells have been ineffective in treating carcinomas that often have an increased expression of PD-L1. That is their sensitivity to PD-1 is increased and carcinomas that express PD-1 will thus enhance the rate of CAR T-cell exhaustion. There is some evidence to support that optimal formation of CD8+T_{RM} in the lungs requires cross priming with DCs within the local lymphnodes. CD103+ DCs in the lung facilitate CD103 upregulation and maintenance of CD8+ T_{RM} through the production of TGF-β. (See, e.g. Szabo, P. A., Miron, M., & Farber, D. L., 2019, Science immunology, 4: eaas9673. Also see, Shin, H., & Iwasaki, A., 2013, Immunological reviews, 255:165-181.) This instant patent further contemplates the generation of CD103+CD69+ T-cells that optionally express a CAR and also one of dIgA1 (inclusive of polymeric immunoglobulin A1), dIgA2 (inclusive of polymeric immunoglobulin A2), engineered variants or dscFV-FcIgA with optional downregulation of PD-1 and optional engineering disruption of TCR expression.

### Natural Killer (NK) Cells Developmental Stages, Licensing and Memory

This patent contemplates the use of integration competent and integration deficient retroviruses with retroviral vectors encoding for one of dIgA1 (inclusive of polymeric immunoglobulin A1), dIgA2 (inclusive of polymeric immunoglobulin A2), engineered variants or dscFV-FcIgA to transduce NK-cells both ex vivo and in vivo as a means of treating solid tumors, lymphoma and carcinomas. Both CAR engineered NK cells and non-CAR engineered NK cells expressing one of dIgA1 (inclusive of polymeric immunoglobulin A1), dIgA2 (inclusive of polymeric immunoglobulin A2), engineered variants or dscFV-FcIgA are contemplated in this patent with an optional emphasis on NK cell memory that may be for specific forms of cancer. In one embodiment this may be accomplished ex vivo by isolating NK cells with the CD56+ marker and co-culturing them with typical activating protocols such as IL-12, IL-15 and IL-18 or K562 chronic myelogenous leukemia derived cell line that are transduced with both membrane bound IL-15 and membrane bound 4-1BBL (K562-mbIL-15-4-1BBL) and even combining IL-12, IL-15 and IL-18 or with a tumor like environment or K562-mbIL-15-4-1BBL with a tumor like environment while potentially adding other cytokines to result in the selective expansion of NK Cells based on pattern recognition-based memory or the development of pattern recognition-based memory. Alternatively, NK cell stimulation with Toll Like Receptor 9 (TLR-9) activated plasmacytoid dendritic cells (pDCs) that results in TNF-related apoptosis inducing ligand (TRAIL) and CD69 upregulation on NK cells and IFNγ production may be used as a means to enhance NK cells response to cancer. In another embodiment an anti CD56+ pseudotyped integration competent retrovirus encoding for the dIgA and polymeric immunglobulin A can be added to expanding NK cells—where gammaretroviruses or lentiviruses may be used—or quiescent NK cells, where lentiviruses may be used, where such NK cells are later expanded. In another embodiment a pseudotyped retrovirus containing a vector encoding for dIgA and polymeric immunglobulin A is added to any population of blood derived cells that contain NK cells. Vectors used for practicing the invention in NK cells include vectors that encode for dIgA and polymeric immunglobulin A but not a Chimeric Antigen Receptor (CAR) as well as vectors that encode for both dIgA and polymeric immunglobulin A and a CAR.

NK cells mainly develop in the bone marrow although there may be a subset that develops in the thymus. In the bone marrow and other peripheral tissue NK cells can develop from a series of complex signaling events, from interaction with the microenvironment through various multistep adhesion cascades that include chemotactic signaling, selectin-mediated tethering and integrin-mediated firm arrest and diapedesis. NK cell diversity is generated by the combinatorial assortment of germline-encoded activating and inhibitory receptors expressed at the cell surface. (See, e.g. Lee, B. J., & Mace, E. M., 2020, Molecular biology of the cell, 31: 981-991.) That is NK cells rely of combinatorial signaling from surface receptors. Healthy subjects can have 150 to 800 different NK cell types from the combinatorial diversity of signaling receptors. There some key distinctions among NK cells including circulating vs. resident NK cells was well as memory vs. effector NK cells. For example, markers associated with circulating NK cells include CD56^{dim}CD16⁺ in addition to CD57high NKG2A^{low}KIRs⁺. Whereas resident NK cells in the lung include CD56^{bright}CD16⁺ in addition to CD57^{low}, NKG2A^{high}KIRs^{low} which is an immature stage of differentiation. The degree of CD56 expression is ubiquitously used to define human NK cell maturation, functional, and tissue-specific subsets, yet a unifying implication for the degree of CD56 expression in NK cells remains elusive. (See, e.g., Poznanski, S. M., Ashkar, A. A., 2018, Cell Mol Immunol 15:1071-1073). CD56^{bright} NK cells are generally thought to be more proliferative, to have a higher capacity for cytokine production after stimulation with IL-12 and IL-18, and to have poor cytotoxic effector activity at rest. CD16 is also known as FcγRIII which is used to detect IgG binding to its target antigen by binding to the immunoglobulin Fc receptor region and triggers ADCC. CD57 is another maturation marker of NK cells and its expression level is correlated with the level of cytotoxicity. (Nielsen, C. M., White, M. J., Goodier, M. R., & Riley, E. M., 2013, Frontiers in immunology, 4:422.) NKG2A dimerizes with CD94 to make an inhibitory receptor that upon binding to MHC-I of self inhibits the immune cells effector functions. Disrupting the NKG2A gene or blocking NKG2A with an antibody has been explored as a means to enhance NK cell response to tumor cells. For example, it has been shown that blocking expression of inhibitory receptor NKG2A overcomes tumor resistance to NK cells.

Memory cell markers of NK cells can be related to the specific pathogen and antigen. For example, murine cytolomegavirus (CMV)-induced memory NK cells are predominantly Ly6C^{hi}, DNAM1^{lo}, CD27-CD11b^{hi}, KLRG1^{hi}. These cell surface markers are specific to prior NK expansion and contraction in response to a CMV virus infection. Additionally, aside for NK cell detection of a CMV virus infection via an innate Ly49H+ receptor that recognizes the CMV virus encoded m157 protein that is presented on the surface of infected cells, the cognate recognition of recall antigens related to that used by B-cells and T-cells, that is the antigen specific receptor of memory NK cells has yet to be established. (Sun, J. C., Beilke, J. N., & Lanier, L. L., 2009, Nature, 457: 557-561) Functional differences between NK cells are also a consequence of the NK cell learning process where for example, NK cells use Killer-cell immunoglobulin-like receptors (KIRs), to interact with self (MHC)-I complexes and learn to become self-tolerant. Inhibitory receptors on NK cells recognize specific surface molecules that are constitutively expressed at high levels by most cells, and the loss of these molecules is referred to as “missing self.” As an example, reduction in MHC class I expression increases the chance that an NK cell will kill the target cell. It is thought that the balance of signals from “stress-induced self” detected by activating receptors and “missing self” detected by inhibitory receptors determines whether an individual NK cell will be triggered to kill a particular target cell. (See, e.g. Abel, A. M., Yang, C., Thakar, M. S., & Malarkannan, S., 2018, Frontiers in immunology, 9:1869.) The repertoire of receptors on an NK cell are tuned to detect changes in expression of various surface proteins on a target cell. The activating receptors for example can recognize cell-surface proteins that are induced on target cells by metabolic stress, such as malignant transformation or microbial infection any of which is referred to as ‘stress-induced self’ Specific cellular events, associated with “stress-induced self” include but are not limited to DNA damage, signals related to proliferation, heat-shock related stress, and signaling by innate sensors including TLRs can lead to expression by host cells of surface proteins that bind to the activating receptors on NK cells. Stimulation of activating receptors increases the chance that the NK cell will release cytokines such as IFNγ and TNFα and activate the killing of the stimulating cell through the release of granules containing perforin and granzyme. (See, e.g. Brillantes, M., & Beaulieu, A. M., 2020, Frontiers in cellular and infection microbiology, 10:102). The immunological memory feature of NK cells mirrors that of the adaptive immune system. Upon activation NK cells undergo the first phase of expansion in the spleen and liver. For NK cells expressing the virus specific Ly49H receptor proliferate 100-fold in the spleen and 1000-fold in the liver from infection. The NK cells are capable of effector functions and during the 2-3 weeks following expansion there is a dramatic decrease (80-85% decrease) in the number of NK cells where memory NK cells remain among the surviving NK cells. In the recall phase upon viral challenge expansion is greater than 100-fold in the spleen over the period of about 7 days.

Acute lymphoblastic leukemia (ALL) a leading cause of cancer death in children with a cure rate of 50% has been shown to be resistant to NK cells which play a major role in graft vs. leukemia (GvL) effect after hematopoietic stem cell transplantation (HSCT). But that resistance can be overcome by NK cell stimulation with Toll Like Receptor 9 (TLR-9) activated plasmacytoid dendritic cells (pDCs) that results in TNF-related apoptosis inducing ligand (TRAIL) and CD69 upregulation on NK cells and IFNγ production. NK cell activation by TRL-via pDCs also enhances degranulation of lytic granules containing perforin and granzyme. NK cell cytotoxicity was enhanced by the inhibition of engagement with killer immunoglobulin-like receptors (KIR). This instant patent contemplates engineering NK cells to express one of dIgA1 (inclusive of polymeric immunoglobulin A1), dIgA2 (inclusive of polymeric immunoglobulin A2), engineered variants or dscFV-FcIgA for treatment of Acute lymphoblastic leukemia (ALL). NK cells engineered to express dimeric immunoglobulin A and polymeric immunoglobulin A or engineered variants will be stimulation with Toll Like Receptor 9 (TLR-9) activated plasmacytoid dendritic cells (pDCs) that results in TNF-related apoptosis inducing ligand (TRAIL) and CD69 upregulation on NK cells and IFNγ production. Additionally, the vectors constructs encoding for the dIgA and polymeric immunoglobulin A can use promoters and even enhancers based IL-2, IFNγ, TNFα, granzyme, or perforin so that the immunoglobulin expression is upregulated upon activation of the NK cell. As part of pattern recognition associated memory NK cells express several activating receptors including C-type lectin NKG2D, the DNAX accessory molecule-1 (DNAM-1) and cytotoxicity receptors such as NKp30, NPp44, and NKp46. Cytotoxic activity of NK cells can be further enhanced by cytokine stimulation e.g. type I interferon (IFN) or via interactions with dendritic cells. The activating signals are counterbalanced with inhibiting signals with NK cell inhibitory receptors including KIR and heterodimer NKG2A/CD94 via interactions with MHC class I. To induce NK cell mediated lysis inhibitory signals must be repressed by MHC-I downregulated or detected via a KIR/MHC-I mismatch. Cytolytic pathways engaged by NK cells to kill target cells include polarized release of cytotoxic granules and induction of apoptosis via ligands of the TNF family including FAS-Ligand (FAS-L) and TRAIL. NK cells use FAS-L and TRAIL to induce apoptosis through cross linking of their respective death receptors, DR4 (TRAIL-R1) DR5 (TRAIL-R5) and FAS on target cells. Target cells that present high levels of HLA class I expression and low levels of NKG2A specific ligands and low levels of adhesion molecules are poorly targeted by NK cells in the absence of NK cell memory. TLR-9 activation by plasmacytoid DCs (pDCs) is thought to help NK cells overcome this requirement and induce cytotoxicity, expansion and memory and also upregulated CD69 and TRAIL on NK cells. Further IFNγ levels produced by the NK cells were significantly increased and NK cells were dependent on IFNγ production by pDCs. (See e.g. Lelaidier, M., Diaz-Rodriguez, Y., Cordeau, M., Cordeiro, P., Haddad, E., Herblot, S., & Duval, M., 2015, Oncotarget, 6:29440-29455.)

NK cells possess the ability to detect cancer cells in the absence of suitable surface antigen targets mitigating off target toxic effects associated with CAR T-cell technology. NK cells recognize tumor cells with a set of stimulatory and inhibitory receptors. Collectively, these receptors can determine whether a neighboring cell or the immediate microenvironment corresponds with cancer. Where a tumor associated profile triggers NK cell mediated cytotoxicity. NK cells lack TCRs and do not cause graft-versus-host disease (GVHD) and thus, may be considered as an off-the shelf cell therapy product. Additionally, mounting evidence supports a rapid memory function of NK for a specific recall response. Activation of NK cells involves a collection of activating co-stimulatory and inhibitory receptors such as KIR. Chimeric antigen receptors (CARs) can be important to NK cells especially to overcome KIR inhibition. Cancer cell populations often have a profile of upregulating ligands for the NKG2D receptor on NK cells that results in NK cell activation and cytotoxicity. Some signals can be important for the NK activation and survival including IL-2 and IL-15. For example, IL-2 enhances signals from activating receptors and IL-15 further promotes NK cell survival and proliferation. Additionally, IL-12 and IL-18 can stimulate NK cells and induce IFNγ production when used in combination. Adoptive transfer of NK cells exposed to IL-12, IL-15 and IL-18 produced increased antitumor activity and NK cell persistence in mice. NK cells use two mechanisms of killing target cells. Generally, initially upon the formation of an immunological synapse between the effector cell and target cell followed by activation NK cells release granules containing perforin and granzyme. Although, if the rate of replenishment of granules is slower that the rate of their release the NK cell may switch over to a death receptor mediated killing CD95L-induced apoptosis via a caspase 8 mediated apoptotic cascade of target cells that is inherently a slower process of cell killing. Some of the challenges associated with tumor resistance to NK cells include a reduction in the surface expression of ligands that are activation markers for NK cells. Similarly, prolonged activation by activating ligands expressed by tumor cells can induce resistance in NK cells. Additionally, tumor absorption of IFNγ increases MHC-I expression and results in KIR inhibition. NK cells are typically activated ex vivo in one of two primary methods: They can be exposed to a cocktail containing IL-12, IL-15 and IL18 can result in an NK memory cell like state that includes improved functioning upon restimulation and hyper-responsiveness to IL-2, greater IFNγ production and improved cytotoxicity. Alternatively, NK cells may be co-cultured with the K562 chronic myelogenous leukemia derived cell line that are transduced with both membrane bound IL-15 and membrane bound 4-1BBL (K562-mbIL-15-4-1BBL) results in vigorous expansion of NK cells. CARs known to be most effective for signaling in NK cells use a an scFv bound to a CD8α hinge and transmembrane domain, a 4-1BB co-stimulatory domain and a CD3ζ signaling domain. For example, using an anti CD-19 scFv in this CAR construct allows NK cells to overcome HLA-mediated inhibitory signals and efficiently kill autologous ALL cells. (See, e.g. Shimasaki N., Jain A., Campana D., 2020, Nat. Rev. Drug Discov. 19:200-218; Also see, Lelaidier M., Diaz-Rodriguez Y., Cordeau M., Cordeiro P., Haddad E., Herblot S., Duval M., 2015, Oncotarget. 6:29440-29455.)

### Chimeric Antigen Receptors T Cell (CAR T Cells) and Non-T Cells Expressing Dimeric Immunoglobulin A

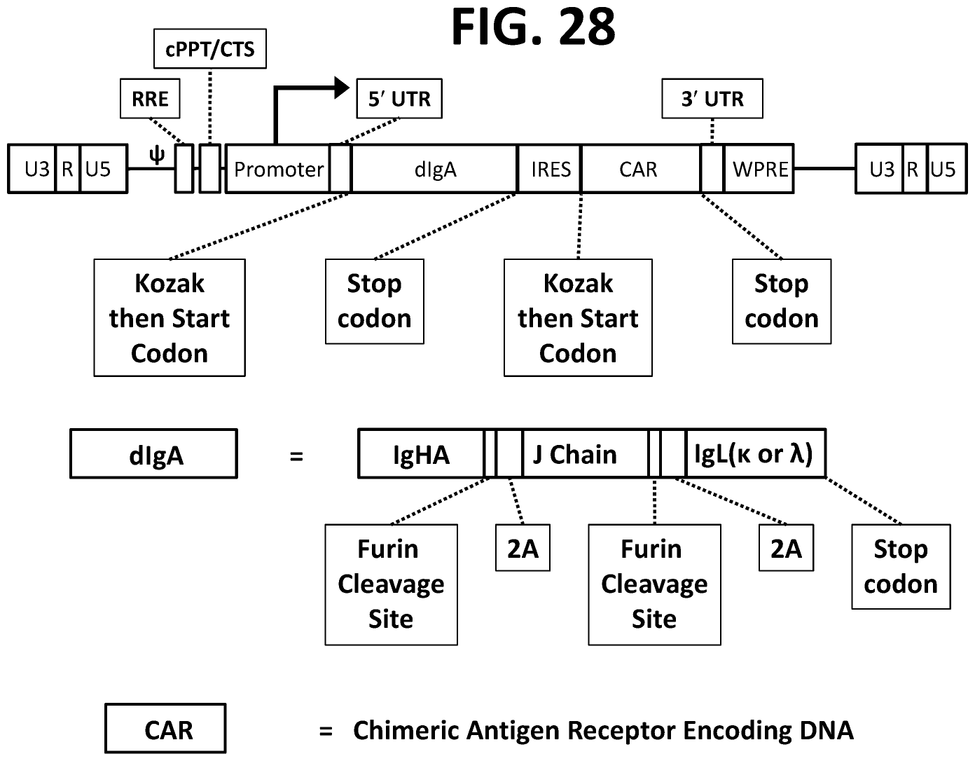

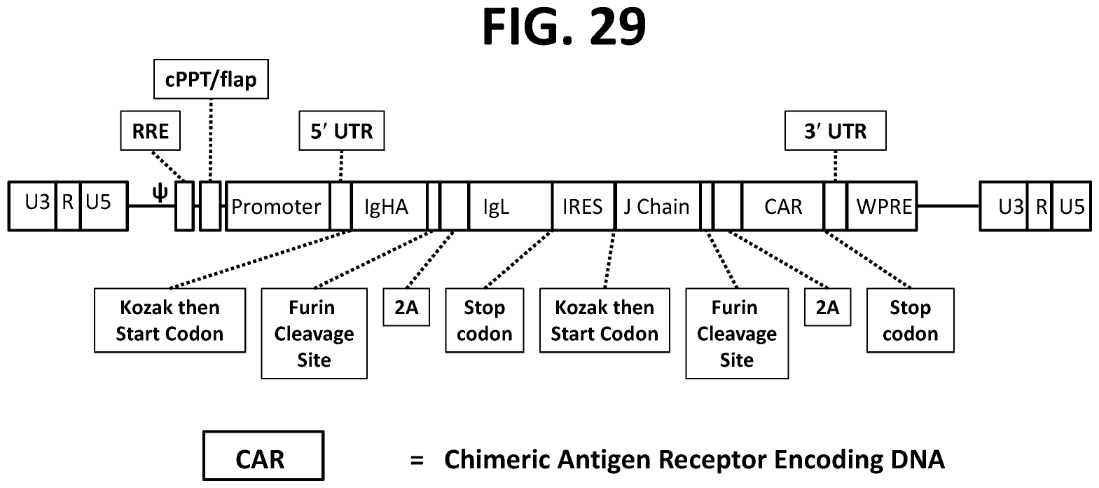

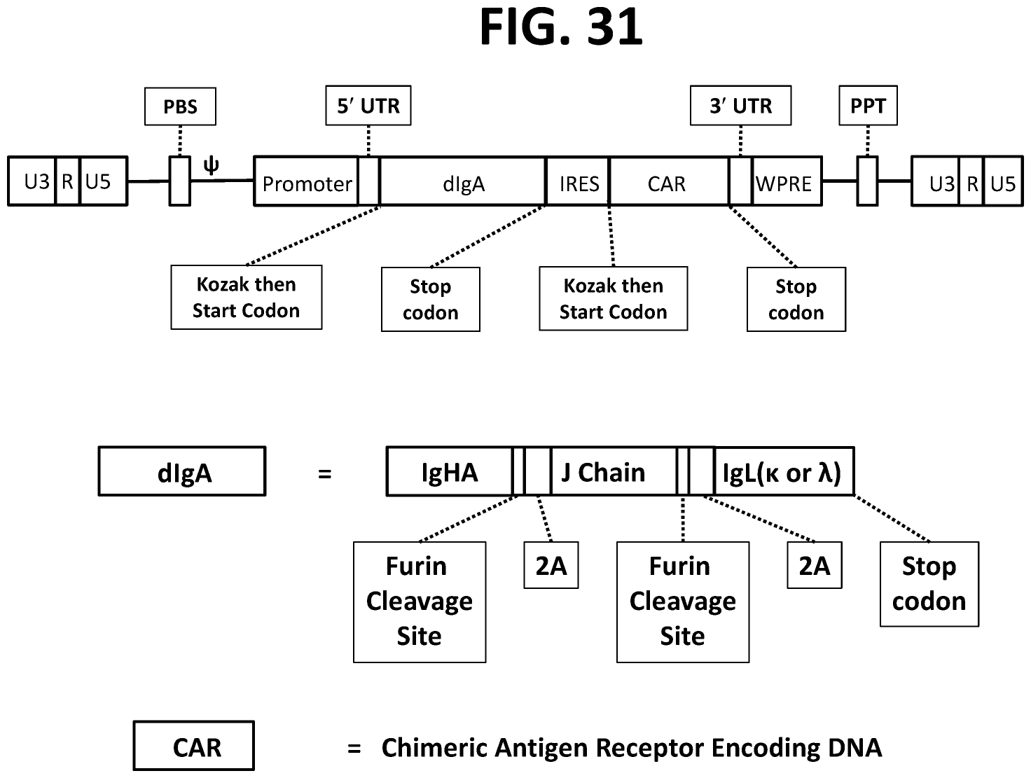

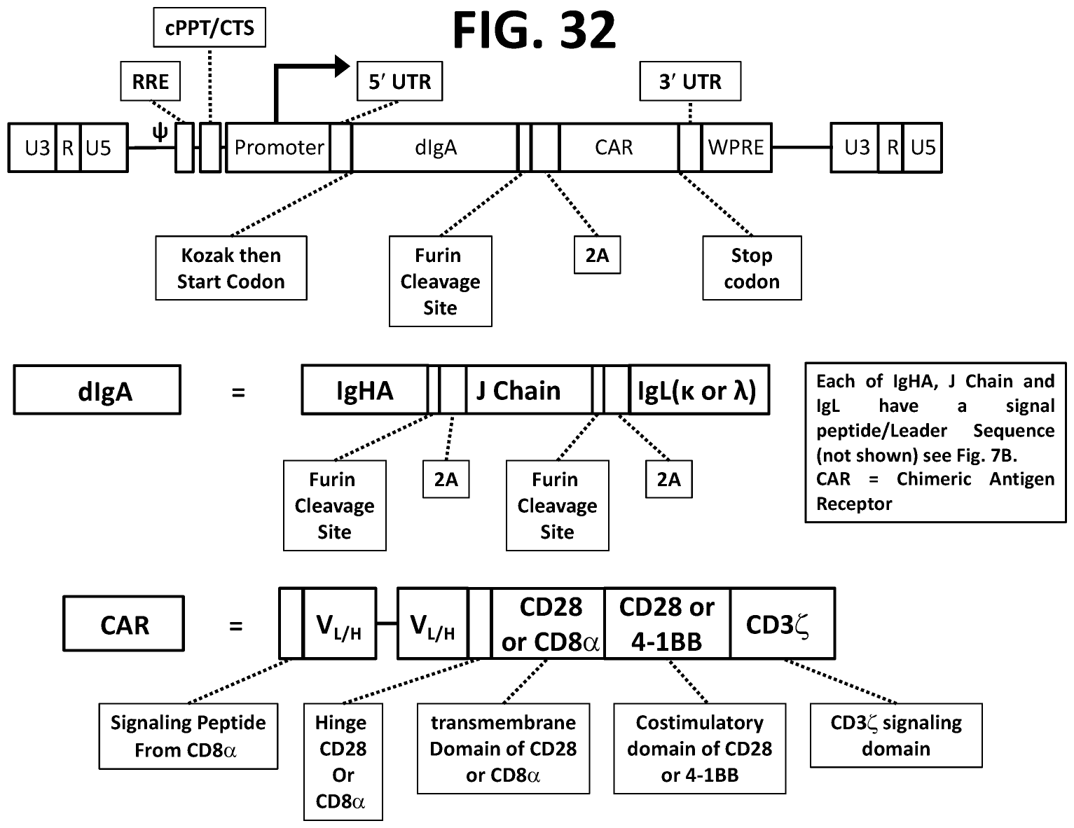

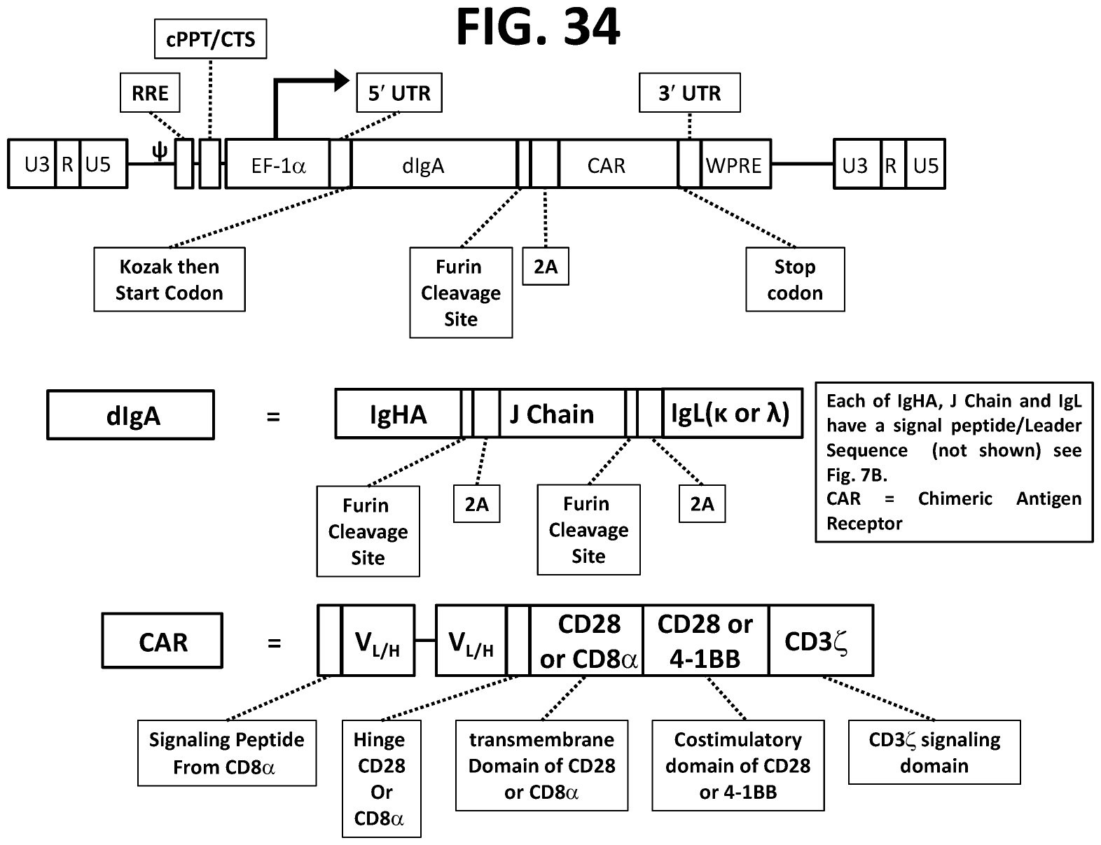

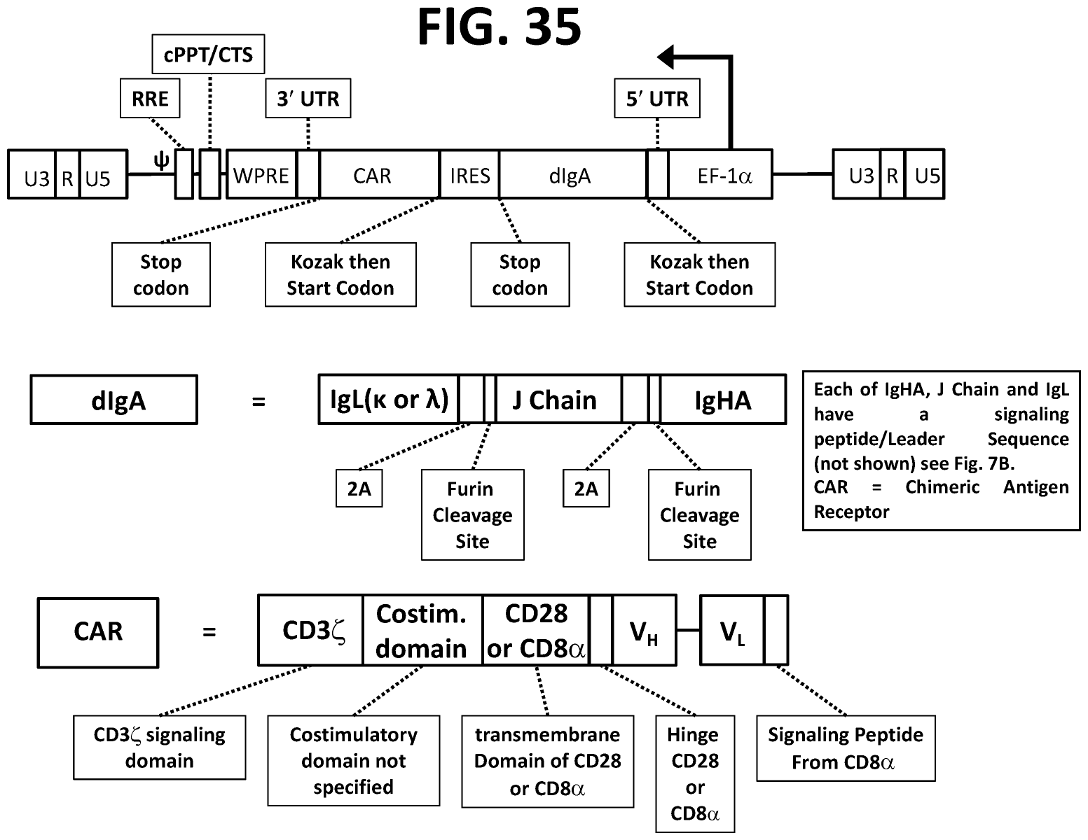

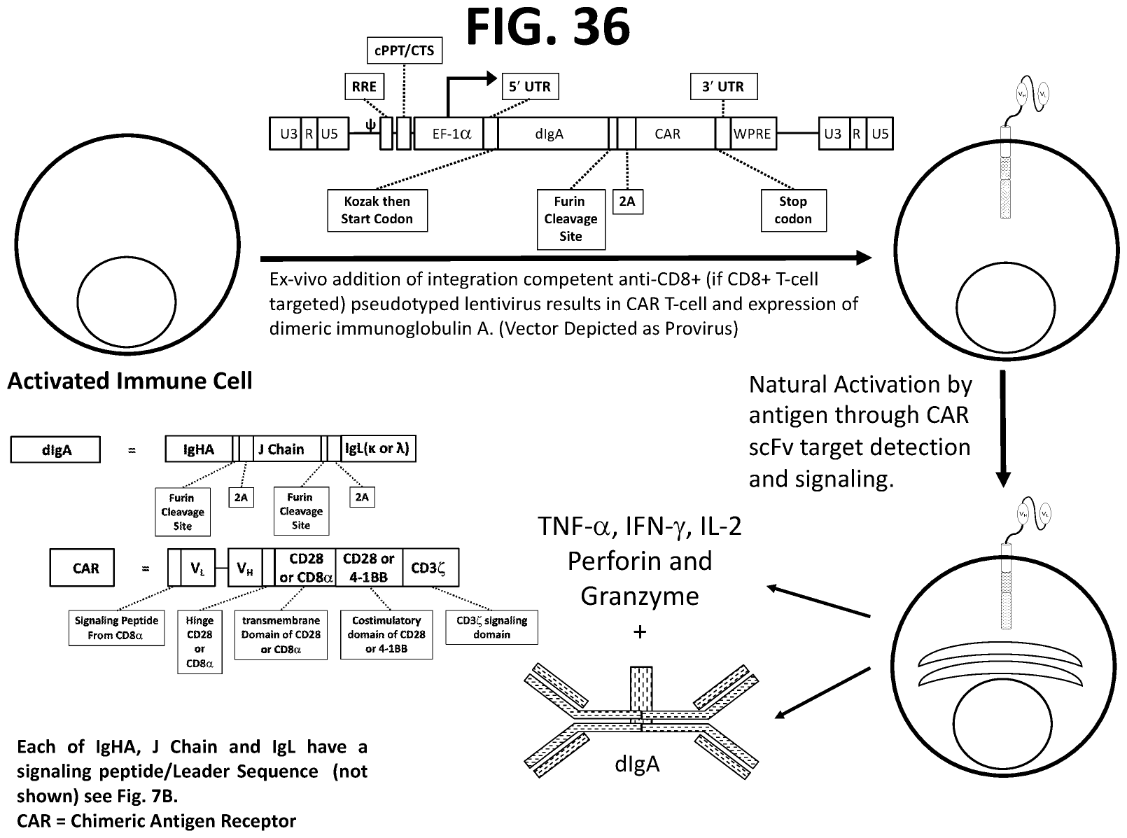

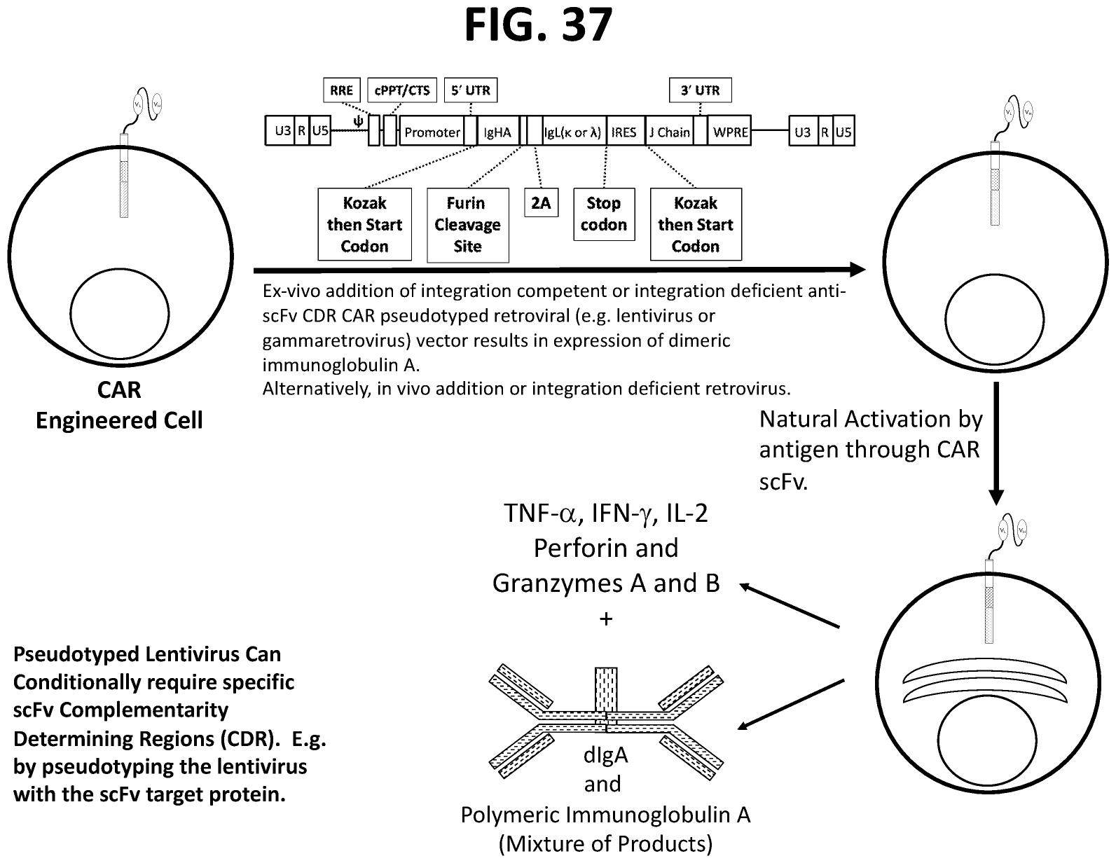

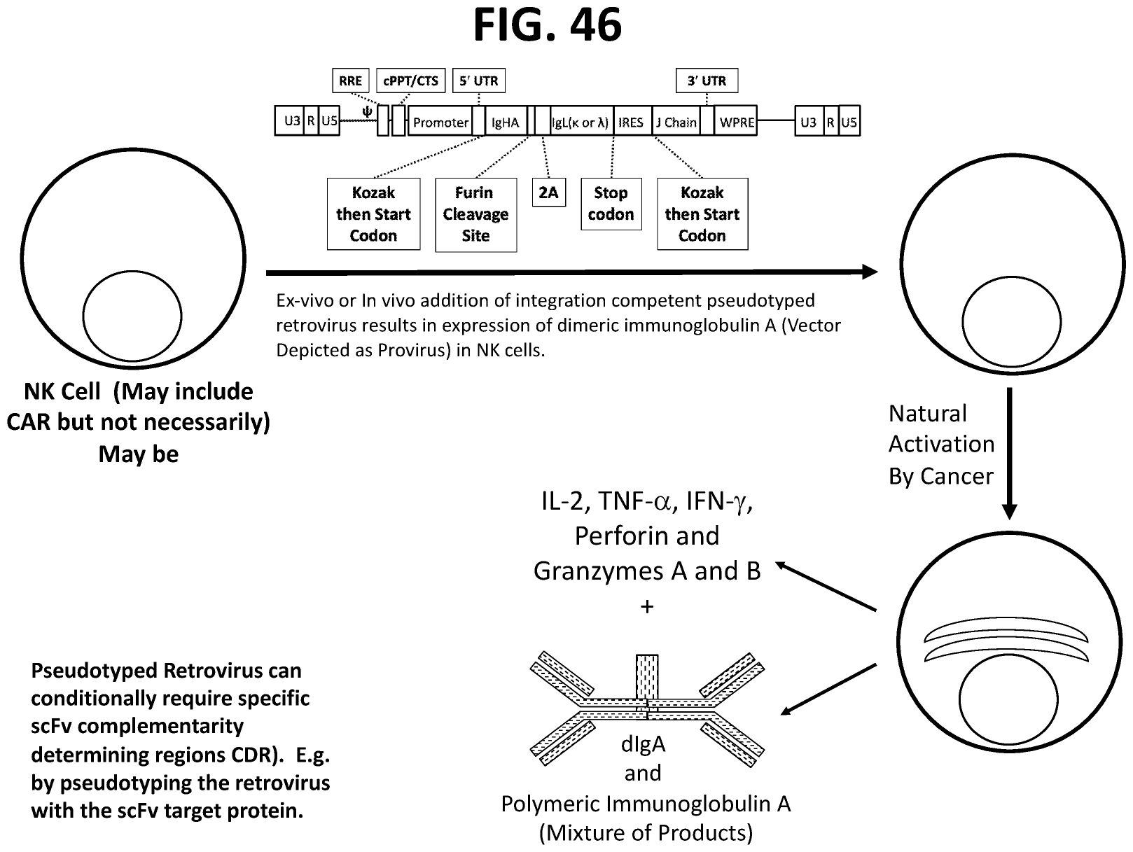

This instant patent contemplates the use of CAR T-cells as well as other CAR engineered immune cells to co-express one of dIgA1 (inclusive of polymeric immunoglobulin A1), dIgA2 (inclusive of polymeric immunoglobulin A2), engineered variants or dscFV-FcIgA (See FIGS. 28, 29, 30, 31, 32, 34, 35, 36, 37, 41, 43 and 45) as a means of delivering dIgA and polymeric immunglobulin A at the site of the tumor or malignancy thereby reducing the total required effective dose of dIgA and polymeric immunglobulin A necessary to achieve therapeutic benefit while also minimizing side effects associated with a systems level administration of dIgA and polymeric immunglobulin A or even a gene therapy that results in the systems level expression of dIgA and polymeric immunglobulin A. This may be accomplished with a single vector encoding for both the CAR and dIgA and polymeric immunglobulin A (see FIGS. 28, 29, 30, 31, 32, 34, 35, 36, 37 and 46) or alternative both the CAR and dscFV-FcIgA (FIG. 44) or alternatively through the sequential addition of the retrovirus (that may be integration competent or integration deficient) encoding for the CAR followed by the anti scFv CAR pseudotyped retrovirus (that may be integration competent or integration deficient) encoding for dIgA and polymeric immunglobulin A (see FIG. 37) or even dscFV-FcIgA where the transduction of the retrovirus encoding for dIgA and polymeric immunglobulin A or dscFV-FcIgA may be conditional in that the cell must express the CAR in order for the retrovirus to be transduced thereby avoiding the production of unintended T-cells or other unintended immune cells that express dIgA and polymeric immunglobulin A without expressing the CAR. Although, the lentivirus or gammaretrovirus is not required to be pseudotyped in this way. In other embodiments the CAR engineered T-cell co-expressing dIgA1 (inclusive of polymeric immunoglobulin A1), dIgA2 (inclusive of polymeric immunoglobulin A2) or engineered variants may be further engineered to disrupt the expression of the TCR thereby avoiding graft vs. host disease (GVHD) and also enabling an off the shelf universal product. Further, this instant patent contemplates the addition or retroviruses encoding for one of dIgA1 (inclusive of polymeric immunoglobulin A1), dIgA2 (inclusive of polymeric immunoglobulin A2), engineered variants or dscFV-FcIgA to transduce an off the shelf CAR engineered cell lines and other off the shelf cell lines resulting in an off the shelf multivalent immunoglobulin expressing cell line.

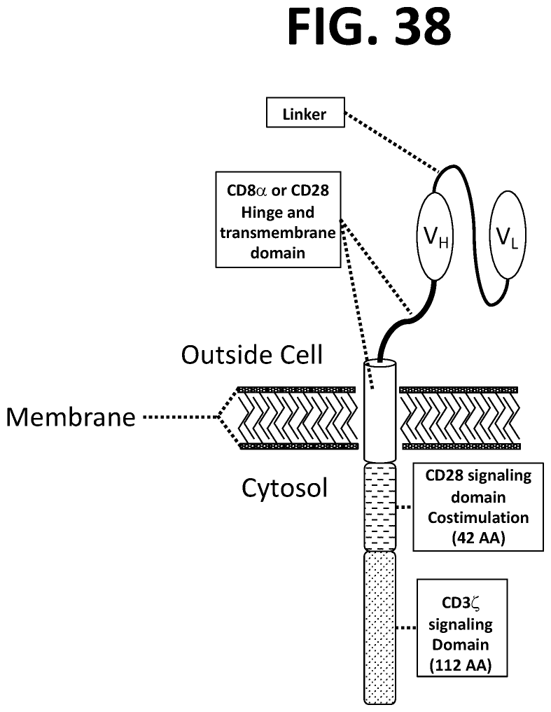

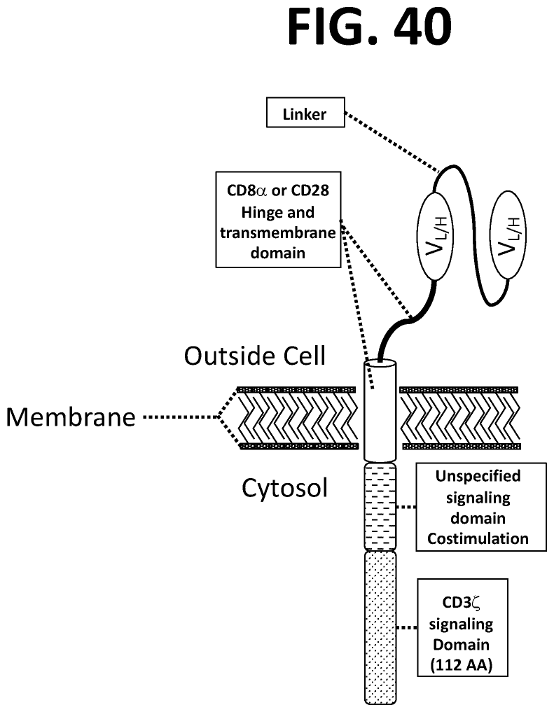

Chimeric Antigen Receptor T cells are modified T-cell receptors that contain a single chain variable fragment (scFv) that may optionally be a V_{H} V_{H} pair, V_{L} V_{L} pair, or V_{H} V_{L} pair derived from the variable region of an antibody that is selected for its high affinity against a tumor associated antigen expressed as a cell surface protein on the tumor (See FIGS. 38, 39 and 40). CAR T-cells represents a growing field in cancer therapy with a number of FDA approved therapies that are essentially a ex vivo gene therapy approach where the CAR containing scFv antibody is encoded for through transduction of an integration competent lentiviral vector or gammaretroviral vector. There are multiple generations of CAR T cells. In the first-generation CAR T-cell the scFv CAR is linked to the intracellular signaling motif of the CD3ζ protein of the endogenous T cell receptor (TCR) that facilitates T cell activation following binding of antigen fragments presented in the Major Histocompatibility Complex class I (MHCI) of the antigen presenting cell. In the first-generation CAR T-cell design the endogenous CD28 receptor continued to rely on the co-stimulatory signal from binding the Cluster of Differentiation 80 (CD80) ligand of an antigen presenting cell. Although, the first-generation CAR T cells lacked efficacy in clinical trials. In second-generation CAR T-cells the Cluster of Differentiation 28 (CD28) receptor or Cluster of Differentiation 137 (CD137) referred to as the 4-1BB ligand (4-1BB) costimulatory endodomain (cytoplasmic domain) was placed between the transmembrane domain of the TCR and the Cluster of Differentiation 3 zeta (CD3C) signaling domain (See FIGS. 38, 39 and 40). The result was the CAR scFv detection of the target antigen resulted in CD3ζ signaling and co-stimulation of either the CD28 or 4-1BB signaling providing therapeutically effective T cell activation during CAR scFv recognition of the antigen. The second-generation CAR T cell technology resulted in a significantly improved response over the first-generation design and led to FDA approved therapies. All current FDA approved CAR T cell therapies are part of the second-generation design that uses either a CD28 or 4-1BB co-stimulatory domain. (See e.g. Larson, R. C., Maus, M. V., 2021, Nat Rev Cancer. 21:145-161.)

Single chain variable fragments (scFv) derived from immunoglobulins used in the CAR on T cells and other immune cells are typically linked together with repeated glycine or serine residues or a Whitlow linker. The hinge or polypeptide region between the scFv and the transmembrane domain is derived or developed from other human proteins such as from CD8α and CD28. In some reports the levels of cytokines released by CAR-expressing T cells significantly depended on the hinge and transmembrane domains included in the CAR. For example, it was demonstrated that CARs with CD8α hinge and transmembrane domains caused weaker T-cell activation and lower levels of cytokine release than did CARs incorporating CD28 hinge and transmembrane domains. (See, e.g. Brudno, J. N., Lam, N., Vanasse, D. et al., 2020, Nat Med, 26:270-280. 2020).

Upon activation CD8α or the CD8α co-stimulatory domain cytotoxic T-cells and CAR T-cells respectively results in the proliferation of the T cells and release of cytokines such as perforin and granzyme in addition to cytokine signaling via interferon gamma (IFNγ), Tumor Necrosis Factor alpha (TNFα) and interleukin 2 (IL-2). Perforin is a pore forming cytolytic protein found in the granules of cytotoxic T lymphocytes and natural killer (NK) cells. The formation of pores allows for granzymes, serine proteases released by cytoplasmic granules within cytotoxic T cells and natural killer (NK) cells to diffuse into the cell where the granzymes induce programmed cell death (apoptosis) in the target cell. To be able to secrete large amounts of cytokines the CAR T-cell and NK cells must engage in the expansion of the Endoplasmic Reticulum (ER) and Golgi—relative to the cell size of other cells that do not secrete large amounts of proteins or granule—to support the secretion of perforin, granzyme, IFNγ, TNFα and IL-2. This property has enabled T-cells and CAR T-cells to secrete scFv-Fc-IgG1 and scFv-Fc-IgG4 antibodies upon activation via the CAR. (See e.g., Chang, d., Sun, J., Signoretti, S., Zhu, Q., & Marasco, W. A., et al., 2016, Oncotarget, 7: 34341-34355. Also See, Powell, A. B., Ren, Y., Korom, M., Saunders, D., Hanley, P. J., Goldstein, H., Nixon, D. F., Bollard, C. M., Lynch, R. M., Jones, R. B., & Cruz, C., 2020, Methods & clinical development, 19:78-88. Further See, Qin, V. M., D'Souza, C., Neeson, P. J., & Zhu, J. J., 2021, Cancers, 13: 404.) Additionally, the CAR technology has extended beyond T-cells and has including immune cells such as Natural Killer cells, Natural Killer T-cells, Macrophages, Dendritic Cells, B cells.

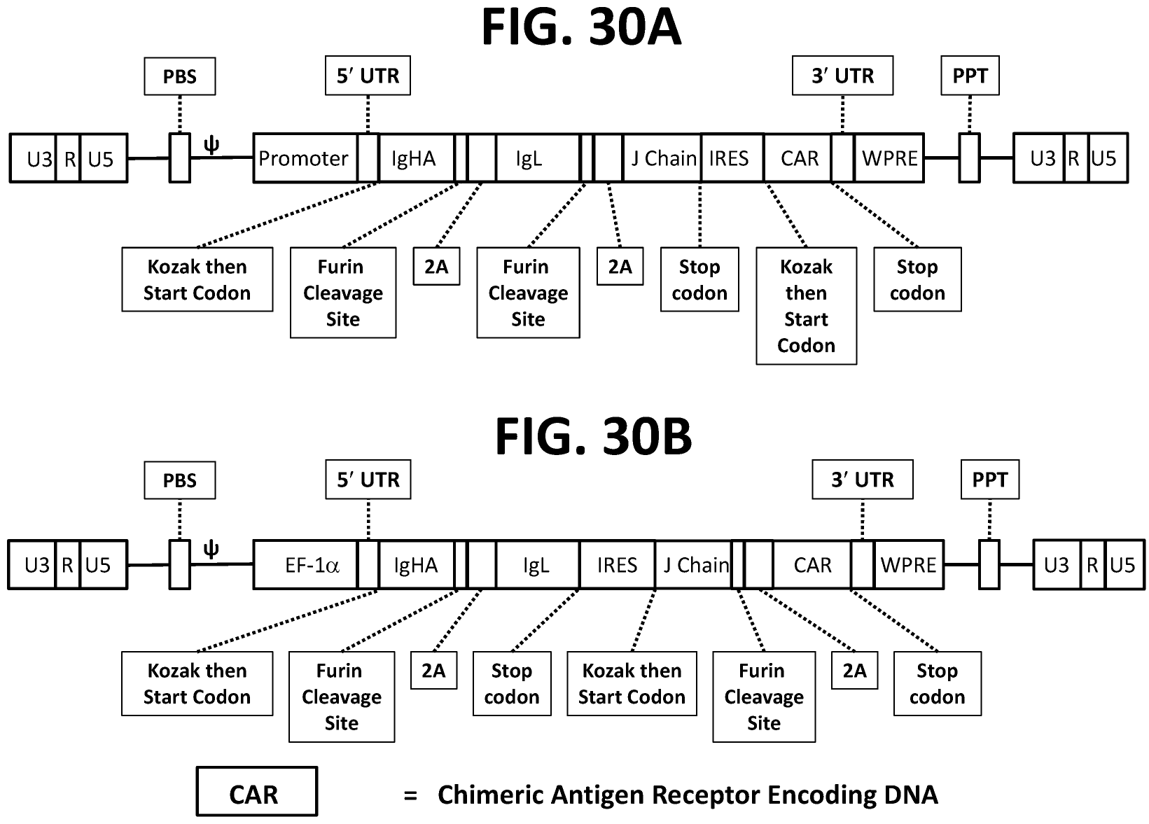

This patent contemplates the use of pseudotyped lentivirus and pseudotyped gammaretorviruses (see FIGS. 30A and 30B) delivering vectors encoding for one of dIgA1 (inclusive of polymeric immunoglobulin A1), dIgA2 (inclusive of polymeric immunoglobulin A2), engineered variants or dscFV-FcIgA to be transfected into CAR T-cells, CAR NK cells, CAR NK T-cells, as well as other CAR immune cells that secrete granules or large amounts of cytokines upon activation (See FIGS. 28, 29, 30, 31, 32, 34, 35, 36, 37, 41, 43 and 45). In one embodiment the pseudotyped lentivirus or pseudotyped gammaretorvirus contains a vector only encoding for dIgA1 (inclusive of polymeric immunoglobulin A1), dIgA2 (inclusive of polymeric immunoglobulin A2) or engineered variants, both of a specific subclass or both of a specific engineered variant, specific to the same target protein that the CAR is specific to or even an alternate protein is contemplated (See FIG. 37). The target protein dIgA and polymeric immunglobulin A is specific to and the CAR target protein may be specific to a similar epitope, nearly the same or different epitope on the target protein. Similarly the specific dIgA and polymeric immunglobulin A may use the same, a similar or a different paratope as the CAR's scFv to detect for the target protein. In another embodiment a single lentiviral vector or gammaretroviral vector is used to encode for both dIgA1 (inclusive of polymeric immunoglobulin A1), dIgA2 (inclusive of polymeric immunoglobulin A2) or engineered variants and also the CAR (see FIGS. 28, 29, 31, 32, 34, 35, 36 and 41). A common promoter used in the CAR encoded vector is the Eukaryotic translation elongation factor 1 alpha (EF-1α) Promoter (see SEQ ID NO: 104). In some embodiments promoters of specific chemokines and cytokines that are upregulated or secreted by cytotoxic T-lymphocytes or NK cells upon activation are used as the promoters in the vector constructs. Such promoters may include truncated promoters and their upstream or downstream regions and optionally their associated enhancers such as that for perforin (SEQ ID NO: 110), granzyme B (SEQ ID NO: 113), IFNγ (SEQ ID NO: 111 and SEQ ID NO:112), TNFα (SEQ ID NO: 106, SEQ ID NO: 107, SEQ ID NO:108 and SEQ ID NO: 109) and IL-2 (SEQ ID NO: 115, SEQ ID NO:116 and SEQ ID NO: 117)

CAR T cells may be generated ex vivo by an unstimulated leukapheresis of the patient's blood that separates white blood cells from the patient's blood and returns the remaining blood to circulation. T-cells are then activated by providing signals that results in T-cell division after which the T-cells are transduced with an integration deficient or integration competent pseudotyped lentivirus or pseudotyped gammaretrovirus specific to a receptor on the T-cells. Those signals necessary for T-cell activation may include a cytokine cocktail that stimulates the TCR and CD28. Although, all three of the TCR, CD28 and CD137 (4-1BB) may be stimulated. In some cases T-cells may be isolated or particular lymphocytes may be removed from the white blood cell sample. For example, the use magnetic bead cell sorting kits has been used to deplete one or more of monocytes, Natural Killer (NK) cells and B-cells from the white blood cells isolated via leukophersis. In addition, the complete isolation of untouched T-cells from the white bloods cells isolated via leukophersis is also possible with this approach. (See, e.g. Noaks, E., Peticone, C., Kotsopoulou, E., & Bracewell, D. G., 2021, Molecular therapy. Methods & clinical development, 20:675-687. Also see Mhaidly, R., & Verhoeyen, E., 2019, Molecular therapy: the journal of the American Society of Gene Therapy, 27: 707-709.)

Signals used for T-cell activation in cytokine cocktails may include interleukins that can influence the T-cell population. Additionally, interleukins may be used to rescue T-cells during the contraction phase where apoptosis occurs thereby increasing the number of resulting memory CD8+ T-cells. For example, interleukin 7 (IL-7) and IL-15 can play roles to maintain CD8+ T-cells following antigen stimulation during the contraction phase of the immune response. In the absence of IL-7 T-cell survival may be impaired. Additionally, in one report IL-7 played an important role in facilitating the formation of memory T-cells from T-cells that express both CD8+ and CD127+. (Rubinstein, M. P., Lind, N. A., Purton, J. F., Filippou, P., Best, J. A., McGhee, P. A., Surh, C. D., & Goldrath, A. W., 2008, Blood, 112:3704-3712)

This instant patent contemplates the generation of CAR expressing immune cell lines that co express one of dIgA1 (inclusive of polymeric immunoglobulin A1), dIgA2 (inclusive of polymeric immunoglobulin A2), engineered variants or dscFV-FcIgA with enhanced fitness via favoring fatty acid oxidation as a source of energy consumption. In some embodiments, as part of CAR T-cell development for CAR T-cells that co express dIgA1 (inclusive of polymeric immunoglobulin A1), dIgA2 (inclusive of polymeric immunoglobulin A2) or engineered variants, CD62L+ naïve T-cells will be expanded with CD3/CD28 followed by exposure to IL-followed by transducing the T-cell with the lentiviral vector encoding for the CAR and one of dIgA1 (inclusive of polymeric immunoglobulin A1), dIgA2 (inclusive of polymeric immunoglobulin A2) or an engineered variant. Enhancing the fitness of the CAR T-cells is possible as a function of the cytokine cocktail. When CD62L+naïve T-cells are expanded with CD3/CD28 then exposed to different cytokine cocktails focusing on IL-2 and IL-15 they yield significantly improved properties with IL-15 vs. IL-2 or IL-2/IL-15. Those enhanced properties included reduced apoptosis, increased proliferation, enhanced tumor killing capacity, enhanced overall response to tumor challenge and tumor rechallenge and overall improved metabolic properties. Overall, such enhanced properties make these CAR T-cell subsets potentially appropriate for solid tumors especially when additional genetic engineering of the CAR T-cell takes place such as encoding dIgA and polymeric immunglobulin A to be expressed upon CAR activation. CAR T-cells are normally generated from a cytokine cocktail that includes IL-2 resulting in a heterogenous mix of T-cells that include antigen experienced T-cells and differentiated T-cells that include effector T-cells and effector memory T-cells. In one study it was shown that starting with a less differentiated subset of T-cells for CAR T-cell therapy such as naïve T-cells Tₛₑₘ and central memory T-cells results in a more potent antitumor immune response that effector-memory T-cells and effector T-cells (T_{eff}). T_{eff} cells have high glycolytic activity while less differentiated cells rely of fatty acid oxidation for energy production. Favoring one metabolic state over the other can promote increased populations of T-cells relying on that metabolic pathway. T-cells that rely on fatty acid oxidation conserve respiratory capacity providing a metabolic advantage for survival. It is thought that T-cells with reduced mTOR signaling activity have less glycolytic activity than cells with increased mTOR signaling. IL-15 not only favors reduced mTOR signaling but favors increased expression of CPT1a that both favors fatty acid oxidation and a less differentiated state and favoring increased numbers of CD8+ memory T-cells that is expected to improve CAR engineered T-cell fitness resulting in a more lasting effect of a single administration of a CAR T-cell therapy. (See, e.g. Alizadeh, D., Wong, R. A., Yang, X., Wang, D., Pecoraro, J. R., Kuo, C. F., Aguilar, B., Qi, Y., Ann, D. K., Starr, R., Urak, R., Wang, X., Forman, S. J., & Brown, C. E., 2019, Cancer immunology research, 7:759-772.) There is broad agreement in the field of CAR T-cell technology that what makes one CAR T-cell favorable to another depends on the type of cancer. Although, overall among the most important factors including expansion of CAR T-cells, persistence and a memory phenotype. Such enhanced T-cell properties when coupled with the capacity for the T-cells to express dIgA and polymeric immunglobulin A upon activation can confer superior solid tumor cell killing capacity.

Lentiviral vectors are the system of choice to transduce activated T-cells with the CAR technology. Although gammaretroviral vectors have also been used in clinical trials. While both lentiviral vectors and gammaretroviral vectors randomly integrate into the host genome. One advantage of lentiviral vectors over gammaretroviral vectors is the lentiviral vectors are less likely to integrate at locations on the genome that can upregulate other genes potentially causing lymphoma. For example, the gammaretrovirus of murine lukemia virus exhibits preferential insertion near transcriptional start sites, while lentiviral vectors preferentially insert within transcriptional units. Gammaretroviral vector integration is prone to insertional mutagenesis by enhancer interactions when integration occurs nearby cellular promoters and provide strong enhancer elements that are bound by ubiquitous transcription factors. This in turn overrides cellular transcriptional control mechanisms. When the gammaretroviral vector integrates into the host genome antisense and upstream of the promoter of the endogenous gene the viral enhancer of the 5′ Long Terminal Repeat (5′ LTR) activates the cellular promoter of the endogenous gene and the resulting gene expression of the endogenous gene with be amplified. Similarly, when the gammaretroviral vector integrates between the endogenous promoter and the first coding exon the endogenous promoter becomes uncoupled from the gene and expression is facilitated by the viral promoter encoded in the U3 5′ LTR. When such integrations occurs close to proto-oncogenes this can lead to malignant transformation. (See, e.g., Maetzig, T., Galla, M., Baum, C., & Schambach, A., 2011, Viruses, 3: 677-713., Also See, e.g. Stein, S.; Ott, M. G.; Schultze-Strasser, S.; Jauch, A., Glimm, H.; et al., 2010, Nat. Med., 16:198-204) Gammaretroviral vectors on the other hand are more amenable to manipulation and less cumbersome to work with that Lentiviral vectors and may be more well suited than lentiviral vectors to transduce dividing cells.

Universal CAR T-cell technology (UCART) is an off-the shelf allogeneic variant of CAR T-cell technology that is derived from T-cells typically from healthy donors and that is gene editing technologies including ZFN (zinc finger nuclease), TALEN (transcription activator-like effector nuclease), and CRISPR-Cas9 are being used to generate the UCART technology. To avoid graft-versus-host disease (GVHD) the endogenous T-cell receptors are disrupted to prevent endogenous TCR T-cell activation by MHC-II activation from non-cancerous host tissue presenting what the TCR would otherwise recognize as foreign material. Additionally, when multiple myeloma is the target cancer the CS1 antigen is also disrupted so that the UCART technology does not attack self with the CS1 specific CAR used to home in on multiple myeloma cells. Such a study is currently in phase I clinical trials under the identifier NCT04142619 where the TCR and CS1 genes were disrupted with the TALEN gene editing technology. TALENs are nucleases that include a separate DNA binding domain comprised of amino acids that are easily engineered to target virtually any DNA sequence. The TALEN technology utilizes engineered nucleases. The DNA binding domain of TALENS are made up on highly conserved repeats that are derived from transcription activator-like effectors (TALEs). TALES are proteins that are secreted by Xanthomonas spp. Bacteria that are used to alter gene transcription in plants. In TALENs DNA binding is mediated by conserved arrays of 33-35 amino acid repeats that are flanked by additional TALE-derived domains at the N-terminal and C-terminal ends of the array. Individual TALE repeats in an array specifically bind to a single base of DNA that is determined by two hypervariable residues commonly at positions 12 and 13 of the 33 to 35 amino acid array.

### CAR Technology in a Variety of Immune Cell Lines to Fight Cancer.

This instant patent contemplates the use of retroviruses to transduce and generate a variety of immune cell lines to express a CAR and one of dIgA1 (inclusive of polymeric immunoglobulin A1), dIgA2 (inclusive of polymeric immunoglobulin A2), engineered variants or dscFV-FcIgA. The CAR technology has been successfully engineered into other immune cell lines including Natural Killer (NK) cells, Natural Killer T-cells (NKT), γδ T-cells and mucosal associated invariant T-cell (MAIT). While T-cells have achieve remarkable success in treating hematological malignancies there is less support for their efficacy in solid tumors. γδ T-cells are T-cells that express heterodimeric T-cell receptors (TCRs) comprised of γ and δ chains. A subset of γδ T-cells circulates through the blood while a smaller population of γδ T-cells of the Vδ1 subsets are residents in the mucosal epithelium. Upon ligation with the TCR of NK receptor γδ T-cells target tumors through Th1-based cytokines, antibody dependent cellular toxicity (ADCC), antigen presentation and cytotoxic activity via perforin and granzyme. Additionally, γδ T-cells do not rely of MHC molecules and thus do not need additional engineering to prevent GVHD. NK cells have a critical role in cancer immunosurveillance. NK cells require joint signals from different activating and inhibitory receptors to target tumors. Additionally, NK cells can induce cytotoxicity via perforin and granzyme. Additionally, NK cells can use apoptosis pathways via FasL or TNF-related apoptosis-inducing ligand. CAR NK cells have seen success in clinical trials (see NCT03056339 and NCT03415100) with a consistently favorable safety profile while sustaining potent reactivity. A phase I/II trial or umbilical cord blood derived HLA-mismatched CD19-CAR-NK cells with an escalating does was conducted in relapsed or refractory chronic lymphocytic leukemia (CLL) or non-Hodgkin's lymphoma patients. Neither Cytokine Release Syndrome (CRS), nor neurotoxicity nor GVHD was observed where 7 of 11 patients had complete remission. Additionally, 6 patients tested negative for minimal residual disease. Additionally, NK-92 derived CD33-CAR-NK cells were tolerated in a phase I trials of 3 patients with acute myeloid leukemia (AML). Repeated infusion with a high dose of 3rd generation CAR-NK cells only caused mild elevation of CRS cytokines. CAR NK cells both have great potential as an off the shelf product and they are safer than CAR T-cells. A number of methods have been used to improve the persistence of CAR NK cells including deleting cytokine-inducible SH2-containing protein (CIS) or approaches to mimic Toll like receptor signaling also resulted in enhanced persistence. One drawback of CAR NK cells is the short lifespan of mature CAR NK cells. (See, e.g. Qin, V. M., D'Souza, C., Neeson, P. J., & Zhu, J. J., 2021, “Chimeric Antigen Receptor beyond CAR-T Cells.” Cancers, 13: 404)

### CAR Signaling and CAR Induced Exhaustion

The success of CAR engineered cell therapies significantly hinges on the success of CARs targeting CD19 in hematological malignancies. Although, success has been seen in other target antigens in hematological malignancies including CD22 where FDA approved therapies have resulted. Solid tumors and the tumor microenvironment, on the other hand is considered more inhibitory on CAR T-cells promoting exhaustion. An additional complicating factor is that there is evidence that the development of scFv to be incorporated into CARs for specific tumor antigens often yields scFv that while potent result in clustering via interactions in the variable region framework domains of the scFv of the CAR on CAR T-cells. This clustering promotes tonic signaling including CD3ζ phosphorylation triggered by the antigen independent clustering of the CARs. This persistent tonic signaling in turn promotes T-cell exhaustion increasing the CAR T-cell's sensitivity to the inhibitory tumor microenvironment resulting in limited tumor efficacy. T-cell exhaustion is characterized by low proliferative and cytokine producing capacity, high rates of apoptosis, and the expression of high levels of inhibitory receptors including PD-1, T-cell immunoglobulin and mucin-domain containing-3 (TIM-3) and Lymphocyte-activation gene 3 (LAG-3). The 4-1BB costimulatory domain has emerged as an important driver of T-cell fitness even in CD19 specific CAR T-cells. It has been shown that the CD28 costimulatory domain exacerbates exhaustion while the 4-1BB endodomain (cytoplasmic domain) reduces exhaustion. Antitumor efficacy correlates with CAR T-cell expansion and persistence in vivo. Generally, the degree with which CAR T-cells show increased activation during ex vivo expansion serves as a predictor to exhaustion in vivo. CAR T-cells that are prone to CAR clustering show that the use of a 4-1BB costimulatory domain expresses lower levels of exhaustion markers and higher levels of cytokines resulting in improved anti-tumor effects and higher levels of transcription factors associated with memory including KLF6, JUN and JUNB in comparison to CD28 costimulatory domain. Additionally, the use of a 4-1BB co stimulatory domain results in reduced expression of PD-1 and genes involved in regulation of cellular metabolism in addition to having an upregulation in pathways that result in an improved response to hypoxia and apoptosis. Although, the 4-1BB of the 2^{nd} generation CAR does not ablate spontaneous phosphorylation of CD3ζ. Of further note, optimization studies have shown that CD8α transmembrane domain induced less apoptosis of CAR T-cells relative to a CD28 derived transmembrane domain. This instant patent contemplates a CAR engineered cell that coexpresses a CAR and one of dIgA1 (inclusive of polymeric immunoglobulin A1), dIgA2 (inclusive of polymeric immunoglobulin A2), engineered variants or dscFV-FcIgA where the CAR uses a CD8α transmembrane domain and 4-1BB costimulatory domain. These CAR T-cells can be stimulated to favor fatty acid oxidation to resist T-cell exhaustion by isolating CD62L+naïve T-cells expanding them with CD3/CD28 followed by exposure to IL-15 followed by transducing the T-cell with the lentiviral vector encoding for the CAR and one of dIgA1 (inclusive of polymeric immunoglobulin A1), dIgA2 (inclusive of polymeric immunoglobulin A2), engineered variants or dscFV-FcIgA. (See e.g., Long, A. H., Haso, W. M., Shern, J. F., Wanhainen, K. M., Murgai, M., Ingaramo, M., Smith, J. P., Walker, A. J., Kohler, M. E., Venkateshwara, V. R., Kaplan, R. N., Patterson, G. H., Fry, T. J., Orentas, R. J., & Mackall, C. L., 2015, Nature medicine, 21:581-590. Also, see, e.g. Benmebarek, M. R., Karches, C. H., Cadilha, B. L., Lesch, S., Endres, S., Kobold, S., 2019, Int J Mol Sci. 2019, 20:1283.)

CAR signaling cascades are complex in that a multitude of pathways are upregulated. The 2^{nd} generation CAR has been characterized to have signaling through the CD3ζ signaling domain consistent with activation of endogenous TCRs irrespective of the costimulatory domain. The third generation CAR that has two costimulatory domains such as CD28 and 4-1BB does not yield an enhancement in T-cell response nor T-cell fitness. That is the 1^{st} and 3^{rd} generation CARs show reduced phosphorylation of CD3ζ and reduced levels of downstream secondary messengers relative to that of the 2^{nd} generation CAR. In CAR T-cells many signaling pathways are significantly upregulated including calcium signaling, Tₕ-cell differentiation signaling, Tₕ₂ pathway signaling, diapedesis signaling that attracts immune cells passing through the nearby vasculature to the pathology site and agranulocyte adhesion signaling that is used to attract agranulocytes to the tumor including monocytes, T-cells and B-cells. Central for T-cell receptor and CAR signaling is ZAP70, a critical tyrosine kinase that initiates a signal pathway downstream of an activated T cell receptor and activated CAR. (See e.g., Long, A. H., Haso, W. M., Orentas, R. J., & Mackall, C. L., et al., 2015, Nature medicine, 21:581-590. Also, see, e.g. Benmebarek, M. R., Karches, C. H., Cadilha, B. L., Lesch, S., Endres, S., Kobold, S., 2019, Int J Mol Sci. 2019, 20:1283.)

### Gammaretroviral Vectors and Other Retroviruses for Practicing the Invention

Retroviruses are evolutionary optimized gene carriers that have common properties. They are enveloped particles about 100 nm in diameter with an outer lipid envelope consisting of glycoprotein derived from the host cell plasma membrane as a result of the budding process of the capsid surrounded by matrix proteins. Incorporated into the host derived plasma membrane is the env encoded glycoprotein encoded for by the viral genome that is anchored to matrix proteins that facilitates binding to the host cell. They contain two complementary based-paired ssRNAs that are 7-11 kilobases in length that are reverse-transcribed into DNA and incorporated into the host genome during the viral infection process. Additionally, they all contain long terminal repeats with common sections where the DNA provirus includes the U3 promoter enhancer, the R repeat region and the U5 polyadenylation signal. Other common features include the polypurine tract (PPT) that increases gene transfer efficiency, the psi (Ψ) packaging signal and the viral genome also includes the gag (group-specific antigen) gene that encodes for the viral capsid and facilitates interactions with the surrounding matrix proteins and pol genes responsible for synthesis and integration of viral DNA.

Gammaretroviral Vectors are currently in use in to engineer cells with Chimeric Antigen Receptors (CAR) used in clinical trials (see e.g. NCT01454596, NCT01087294 and NCT01822652). This instant patent contemplates the use of a gammaretorvirus to integrate gammaretroviral vectors encoding for one of dIgA1 (inclusive of polymeric immunoglobulin A1), dIgA2 (inclusive of polymeric immunoglobulin A2), engineered variants or dscFV-FcIgA into the genomic DNA of CAR engineered cells or to add a vector encoding for both the CAR and dIgA. Gammaretroviral Vectors function similar to lentiviral vectors and MLV functions similar to lentiviruses. Gammaretroviral vectors for gene therapy and delivery of a therapeutic gene such as those derived from MLV use similar packaging systems to that of the 2^{nd} generation lentiviral packaging system. One important difference between Gammaretroviruses and lentiviruses is that lentiviruses upon fusing with the host cell the human protein transportin-1 binds to incoming capsids, triggers their uncoating and promotes viral nuclear import. In addition the lentivirus capsid is has resistance against cellular restriction factors and an instability that via its interaction with transportin-1 and transportin-3 supports the transduction into cells regardless of the cell's lifecycle status. Gammaretroviruses on the other hand lack active nuclear import elements such as those that allow for interactions with transportin-1 and transportin-3 and in the case of MLV is encapsulated by a highly stable capsid core that depends on nuclear envelope breakdown and thus can only retrovirally transduce dividing cells. Since, T-cells and NK cells for example divide following activation gammaretroviral vectors can be used to produce CAR engineered T-cells and NK cells as well as virtually all other immune cells such as but not limited to dendritic cells a variety of T-cell classes and B-cells. (See, e.g. Maetzig, T., Galla, M., Baum, C., & Schambach, A., 2011, Viruses, 3: 677-713.)

As part of minimizing biosafety risks gammaretroviral leader sequences of the gammaretroviral vector have been made devoid of ATG initiating potential open reading frames minimizing the risk to produce immunogenic peptides derived from pre-canonical translation initiation. Further, similar to the 2^{nd} generation lentiviral packaging technology gammaretroviral packaging systems have split packaging as part of biosafety protocols. Gammaretroviral vectors used in gene therapy include the 5′ and 3′ LTRs, the psi packaging signal and primer binding site (PBS), the polypurine tract (PPT) and the splice donor (SD). In the leader region, the natural splice donor together with the env splice acceptor placed after the psi packaging signal improves transgene expression. Similar to lentiviruses, gammaretroviruses can also be pseudotyped to target particular cell receptors thereby selectively transducing target cells. (See, e.g., Maetzig, T., Galla, M., Baum, C., & Schambach, A., 2011, Viruses, 3: 677-713.)

A major limitation of gammaretroviral vectors is the risk insertional mutagenesis that resulted in cancer in a gene therapy trial for human severe immunodeficiency. Additionally, there are some constraints over lentiviruses used in gene therapy. Such as the expression cassette should be without introns limiting expression to a single gene variant reducing suitability for conditional gene expression based on cell differentiation status. Additionally, repetitive sequences should be avoided due to a possible interference with reverse transcription. Gammaretroviral vector integration is prone to epigenetic modification and gene silencing via methylation by the host cell and this may be exacerbated by the gammaretroviral vectors propensity to integrate near endogenous cellular promoters. Incorporating a ubiquitously acting chromatin opening element (UCOE) that consists of CpG islands into the gammaretroviral vectors within or proximal to the transgene promoter and enhancer is one approach to make chromatin architecture more accessible at the site of the gammaretroviral vector insertion. UCOEs consist of a methylation-free CpG island extending over closely spaced, dual divergently transcribed promoters derived from humans. UCOEs are thought to possess a dominant chromatin remodeling or opening function and is therefore able to resist transcriptional silencing effects.

The propensity of gammaretrovirusis to integrate close to proto-oncogenes can lead to malignant transformation. In one gammaretrovirus gene therapy clinical trial for the treatment of Severe Combined Immunodeficiency (SCID-X1) in Paris and London while initially improving upon the condition of 17 of 20 participants by transplanting the genetically engineered hematopoietic stem cells, later 5 patients were diagnosed with T cell acute lymphoblastic leukemia (T-all) that were attributed to the gammaretroviral vector integration sites proximal to the promoter of proto-oncogenes. SCID-X1 is due to a gene defect of the X-chromosome encoded interleukin-2 common gamma chain that results in the complete absence of T-cells and NK cells and dysfunction B-cells. Similar adverse events were observed in two additional gene therapy trials for X-chromosome linked chronic granulomatous disease (X-CGD) and Wiskott Aldrich syndrome (WAS). In X-CGD patients the therapeutic benefit was short-lived due to progressive silencing of the CAR. Additionally, the gammaretrovial vector insertion occurred proximal to the promoter of the proto-oncogene for MDS/EVI1 resulting in its upregulation and later resulting in myelodyspastic syndrome. It should be pointed out that the number of vector integration events per target cell while originally thought to be important in minimizing the risk of insertional mutagenesis proved to be incorrect. Rather insertional mutagenesis by a single vector integration sufficiently close to a proto-oncogene promoter site—whether acting as an enhancer for the endogenous promoter or substituting in the transgene promoter for the endogenous promoter—can trigger the transformation of cells from a clonal selective advantage resulting in accelerated growth and division rates. Thus, a relatively few number of cells with the insertional mutagenesis can be sufficient to trigger cancer with severe adverse effects. While gammaretroviruses integrate in close proximity to transcriptional start sites, lentiviruses integrate within the transcriptional unit and alpha retroviruses locate relatively uniformly within the whole genome. In general lentiviral vectors are the safest retroviral delivery system least likely to result in a cancer. Integration deficient gammaretroviral vectors can be an effective mode of delivery for episomes. Integration deficient gammaretroviruses may be generated by mutating the retroviral integrase. Integrase may be inactivated with point mutations in the part of the gene encoding for the catylitic core such as the DDE motif that is located at D125, D184 and E220. (See, Maetzig, T., Schambach, A., et al., 2011, Viruses, 3: 677-713.)

dIgA and Polymeric Immunglobulin A coExpression by CAR Engineered Cell Lines

This patent contemplates the expression of dIgA1 (inclusive of polymeric immunoglobulin A1), dIgA2 (inclusive of polymeric immunoglobulin A2), engineered variants or dscFV-FcIgA from CAR engineered cells. CAR engineered cells may include T-cells, NK-cells, dendritic cells, Natural Killer T-cells (NKT), and mucosal associated invariant T-cell (MAIT). CAR T-cells may be exposed to a mixture of interleukins and chemokines that includes one or more of CCL5, MIP-1α, CCL2, MIP-1β, TGF-β to influence the CAR T-cell to differentiate with properties representative of resident memory T-cells (T_{RM}) thereby influencing the dIgA and polymeric immunglobulin A expressing CAR T-cell to more readily migrate to mucosal regions where there is tumor activity.

Previously, it was demonstrated that a single chain fragment variable-fragment crystallizable fusion IgG (scFv-FcIgG) antibody specific to PD-L1 was secreted by CAR T-cells at the tumor site (See, e.g. Suarez, E. R., Chang, d., Sun, J., Signoretti, S., Zhu, Q., Marasco, W., et al., A., 2016, Oncotarget, 7:34341-34355. https://doi.org/10.18632/oncotarget.9114. Also, see U.S. Pat. Publication No. US 2017/0362297 A1) This strategy was used in part to address the immunosuppressive environment of solid tumors that can attenuate the CAR T-cell response by using PD-L1 to bind to PD-1 on T-cells. The authors engineered a bicistronic lentiviral vector to express the anti-CAIX scFv linked to CD28 and CD3ζ signaling domains in the first cassette and anti PD-L1 IgG1 or IgG4 in the second cassette following an IRES site. The lentiviruses containing the vectors were transfected into CD8+ T-cells in the presence of IL-21 or IL-2. In this report IgG levels secreted by the transduced CD8+ T-cells were 300-650 ng/ml after 4 days. The CAR T-cells secreting the anti PD-L1 antibodies saw a greater reduction in tumor weight than CAR T-cells secreting an equivalent scFv-FcIgG antibody specific to SARS that was not specific to the tumor. In addition, the CAR T-cells secreting the anti PD-L1 scFv-FcIgG1 and scFv-FcIgG4 antibodies saw a reduction in the expression of exhaustion markers associated with PD-L1 binding to PD-1 on T-cells supporting greater longevity. While, it is likely that a similar result would be possible with direct administration of the anti PD-L1 scFv-FcIgG1 or scFv-FcIgG4 immunogloubulin the site-specific delivery of the anti PD-L1 immunoglobulins likely confers benefits associated with reduced side effects in comparison to a direct administration of anti PD-L1 scFv-FcIgG antibodies.

This advantages of having CAR engineered immune cells secrete dIgA1 (inclusive of polymeric immunoglobulin A1), dIgA2 (inclusive of polymeric immunoglobulin A2), engineered variants or dscFV-FcIgA antibodies at the tumor site of carcinomas is five-fold. The secretion of dIgA and polymeric immunglobulin A at the tumor site will reduce side effects associated with dIgA and polymeric immunglobulin A binding the related protein of heathy tissue while also allowing for lower dosing of dIgA and polymeric immunglobulin A to be effective while maintaining a high concentration at the tumor site. dIgA and polymeric immunglobulin A will also enable the reduction in metastasis of the tumor as a result of the multivalency and distinct binding faces of dIgA and polymeric immunglobulin A and their rigid backbone. That is the dIgA and polymeric immunglobulin A can diffuse through the tumor and because of their multivalent nature can bind one tumor cell on one distinct face of dIgA and polymeric immunglobulin A and another tumor cell or adjacent neighboring healthy cell on a different distinct binding face of dIgA and polymeric immunglobulin A (see FIGS. 23, 41, 42 and 43). Multiple dIgA's and polymeric immunoglobulin A binding a tumor cell to its neighboring cell ensures that even if some of the dIgA's and polymeric immunoglobulin As are temporarily unbound to the tumor cell on one of the distinct binding faces the effect of multiple dIgA's and polymeric immunoglobulin A's binding the same two cells with their multivalent faces ensures that the cells stay anchored together and also has the effect of serving as more potent binding that the equivalent binding of IgG. That is there is an apparent decrease in the time unbound to the target protein as a result of dIgA and polymeric immunglobulin A being kept in close proximity to its target protein when it dissociates. This results in an increase in apparent binding affinity and is significantly enhanced with increased cell surface expression of the target protein on the tumor cell. Another major benefit of dIgA and polymeric immunglobulin A is that it can allow for reduced specificity to the target protein on the tumor in such a way that healthy tissue is even less compromised in instances where there is an upregulation of the target protein on cancer cells which is typically the deciding factor as to whether such an antibody administration is warranted in the treatment of cancer. Developing antibodies effective against tumor cells and cancer cells requires that the antibodies are not so potent as to cause too great a degree of pathology at healthy tissue but not so low in potency that the provide insufficient benefit in mitigating the effects of the tumor. The multivalency property of dIgA and polymeric immunglobulin A enables additional benefits. First, the dIgA and polymeric immunglobulin A can exhibit an on off ligation type coordination to one tumor cell without diffusing away from the cell surface protein because another distinct face of the dIgA and polymeric immunglobulin A will bind the neighboring tumor cell such that the neighboring tumor cell effectively makes the dIgA and polymeric immunglobulin A a multivalent ligand. This is a direct result of both the tumor cell size and that there will be multiple dIgAs and polymeric immunoglobulin As binding the upregulated protein target on neighboring cancer cells thereby allowing for a reduction in the binding affinity of dIgA and polymeric immunglobulin A without loss of therapeutic effect while reducing the toxicity over IgG based antibodies. dIgA and polymeric immunglobulin A being actively transported across the epithelium to become SIgA also ensures that dIgA and polymeric immunglobulin A can reach both faces of the carcinoma effectively without solely relying on passive diffusion through epithelial cell or diffusion through the tumor at the face facing in the direction of the basolateral face of the epithelium to reach the face of the tumor facing into the lumen or exocrine channel.

A lentiviral vector or gammaretroviral vector encoding for both the CAR and also one of dIgA1 (inclusive of polymeric immunoglobulin A1), dIgA2 (inclusive of polymeric immunoglobulin A2), engineered variants or dscFV-FcIgA is possible with the use of an internal ribosome entry site (IRES). Additionally, separating the CAR vector element from the dIgA and polymeric immunglobulin A vector element with the use of a 2A self-processing peptide (2A) followed by a Furin cleavage site is also possible to encode the vector. Lentiviral vectors and gammaretroviral vectors have a little more than 10 Kilobases of capacity or about 8-8.5 Kilobases of capacity to incorporate all the genetic information beginning with the promoter for the beneficial gene and ending with the WPRE for the beneficial genes. This is more than sufficient to accommodate the vector capacity needs to express dIgA and polymeric immunglobulin A and also the CAR. The promoter/enhancer may be up to 1200 bases, the entire vector element to express dIgA and polymeric immunglobulin A is about 2800 bases if separate each chain with a 2A self-processing peptide and furin cleavage site, the WPRE requires about 600 bases of vector capacity, the Chimeric Antigen Receptor ScFv second generation (CAR) requires about 1600 bases of vector capacity and the IRES requires about 600 bases of vector capacity. One may conservatively allocate an additional 500 bases for the 5′ and 3′ untranslated regions as more than sufficient. Even here the sum of all these vector elements is 7300 bases leaving more than 1,000 bases of additional vector capacity if necessary.

It is further contemplated in the invention to use a CAR engineered cell and add integration competent retroviral vector such as a lentiviral vector or gammaretroviral vector. A pseudotyped lentivirus or pseudotyped gammaretrovirus may be pseudotyped with a protein that is the target of the CAR scFv thereby causing the lentivirus or gammaretrovirus to fuse with the cellular membrane of only those cells that successfully achieve CAR expression (see FIG. 37).

This is effectively, a CAR scFv conditional integration competent or integration deficient retroviral delivery system. This CAR scFv conditional retrovirus ensures that if a cell targeted for a CAR does not successful become engineered with the CAR it will not absorb the anti scFv CAR pseudotyped retrovirus. This may result in overall higher expression levels of both the CAR and also dIgA1 (inclusive of polymeric immunoglobulin A1), dIgA2 (inclusive of polymeric immunoglobulin A2), engineered variants or dscFV-FcIgA than in comparison to incorporating all the genetic information to encode for dIgA and polymeric immunglobulin A and the CAR into a single lentiviral vector or single gammaretroviral vector.

### 2^{nd} Generation Chimeric Antigen Receptor (CAR) Elements

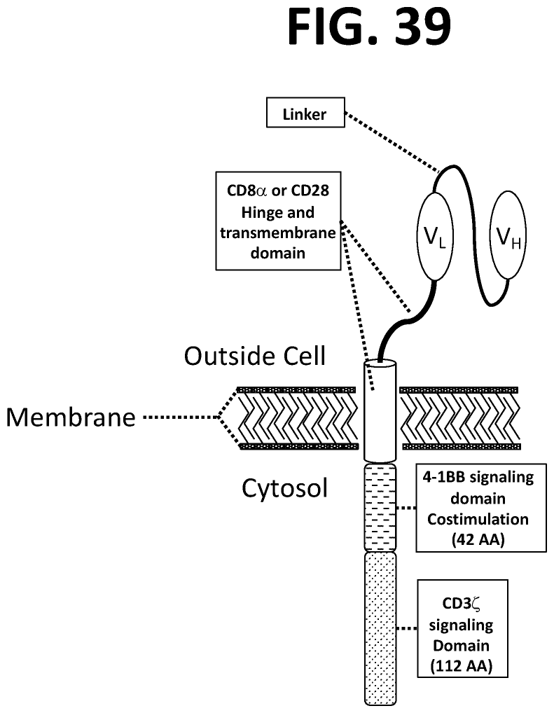

Vectors to use in conjunction with the invention may employ a wide variety of CARs that are not limited to the CARs described in this section. The CAR framework for 2^{nd} generation CARs from the N-terminal to C-terminal direction of the polypeptide includes a signal peptide or leader sequence for human Cluster of Differentiation 8 alpha (CD8α), followed by the binding region which is typically the single chain variable fragment (scFv), followed by a hinge, followed by the transmembrane domain, followed by the first signaling domain referred to as the costimulatory signaling domain followed by the CD3ζ signaling domain (see FIGS. 38, 39 and where the CD8α signal peptide is not included in these figures since it is removed as part of processing of the CAR by the endogenous cellular machinery). (For seminal reports of the 2nd Generation design see e.g. Krause, A., Guo, H. F., Latouche, J. B., Tan, C., Cheung, N. K., & Sadelain, M., 1998, The Journal of experimental medicine, 188:619-626. Also see Vandenberghe, P., Freeman, G. J., Nadler, L. M., Fletcher, M. C., Kamoun, M., Turka, L. A., Ledbetter, J. A., Thompson, C. B., & June, C. H., 1992, The Journal of experimental medicine, 175:951-960.) A number of co-stimulatory domains have been employed in 2^{nd} generation CARs including CD28 and 4-1BB as the most common. (For examples of patents using the 2^{nd} generation CAR design framework see e.g. U.S. Pat. No. 9,605,049 March, 2017, U.S. Pat. No. 10,442,867 October 2019, U.S. Pat. No. 10,538,588 January 2020, U.S. Pat. No. 10,603,380 March 2020.)

The hinge in the 2^{nd} generation CAR framework is derived from either CD28 or CD8α. The hinge is important in that it both gives the nearest scFv variable region flexibility to sample the extracellular space while also enabling effective transmission of the signal from scFv binding through the transmembrane domain to the costimulatory domain and the CD3ζ signaling domain. Using a flexible polypeptide linker between the two V regions (typically V_{H} then V_{L} or alternatively, V_{L} then V_{H} and even in some cases V_{H}1 then V_{H}2 or VIA then V_{L}2 where such V region pairs may be referred to as V_{H}/L and V_{H}/L pairs meaning that the variable light region or variable heavy region may be used at either position where the selection at one position does not restrict the selection at the other position in the scFv) of the scFv allow for positional flexibility between V_{H} and V_{L} so they may situate their Complementarity Determining Regions (CDRs) with minimal restriction if any imposed by the linker from the positioning of the V_{H} and V_{L} for optimal binding to the target. Linkers used in the present patent include repeating units of GGGGS of at least 4 repeats (GGGGSGGGGSGGGGSGGGGS) (SEQ ID NO: 115) and up to 7 repeats (SEQ ID NO: 121) where a sequence with at least 80%, 85%, 90%, 95% or 99% identity to (SEQ ID NOs: 118, 119, 120 or 121) may be used in some embodiments. Additionally, a Whitlow linker (GSTSGSGKPGSGEGSTKG) (SEQ ID NO: 122) may be used as the linker between the V_{H}/L and V_{H}/L with at least 80%, 85%, 90%, 95% or 99% identity to the Whitlow linker (See, e.g. Whitlow, M., Bell, B. A., Feng, S. L., Filpula, D., Hardman, K. D., Hubert, S. L., Rollence, M. L., Wood, J. F., Schott, M. E., Milenic, D. E., et al., 1993, Protein Eng. 6:989-95.) Although, the linkers need not be restricted in this way as the linkers serve the function of flexibility of the Variable region pairs in the scFv and there is a wide variety of ways to generate them.

The V_{H} and V_{L} used in the scFv may be derived from immunoglobulins with ideal binding affinity to the target protein. Ideal binding affinity is that binding affinity that allows sufficiently potent binding affinity while not being so potent that the antibody binds the healthy tissue with such great affinity that pathology of the cancer treatment on healthy tissue outweighs benefit. Thus, depending on other factors such as upregulated antigen concentration on the cancer cell surface or taking advantage of specific common mutations of the cancer cell target protein that allow for enhanced affinity are central towards identifying V_{H} and V_{L} scFv elements ideal to be incorporated into a CAR framework. The V_{H} and V_{L} regions like that used in common immunoglobulins includes three CDRs and four framework regions. Where the V_{H} region is typically about 120 amino acids and the V_{L} region is typically about 106-110 amino acids. The sequence of V_{H} and V_{L} are dependent on the target protein and thus are not provided.

This signal peptide of the CAR is commonly the natural signal peptide used for endogenous CD8α processing. The full sequence of CD8α including the signal peptide is (SEQ ID NO: 124) where a representative human coding DNA is (SEQ ID NO: 125). This sequence is also identified on Ensemble as transcript (Transcript ID ENST00000283635.8). This sequence provided includes the signal peptide of CD8α as amino acids 1-21 of (SEQ ID NO: 124) which is the sequence (MALPVTALLLPLALLLHAARP) that is identified as (SEQ ID NO: 123) where a sequence that is at least 80%, 85%, 90%, 95% or 99% identity to (SEQ ID NO: 123). The signal peptide, extracellular, transmembrane and cytoplasmic regions of the fully human CD8α protein (SEQ ID NO: 124) are identified on Uniprot as P01732 https://www.uniprot.org/uniprot/P01732-1.

The hinge region that follows the V_{H} or the V_{L} region of the scFv in the CAR is derived from either CD28 or CD8α. The full sequence of CD28 that includes the signal peptide is (SEQ ID NO: 127) where a representative human coding DNA is (SEQ ID NO: 128). This sequence is also identified on Ensemble as transcript CD28-201 (Transcript ID ENST00000324106.9). This sequence provided includes the signal peptide of CD28 as amino acids 1-18 of (SEQ ID NO: 127) which is the sequence (MLRLLLALNLFPSIQVTG) that is identified as (SEQ ID NO: 126). The extracellular, transmembrane and cytoplasmic regions of the fully human CD28 protein (SEQ ID NO: 127) are identified on Uniprot as P10747 https://www.uniprot.org/uniprot/P10747. The CAR hinge derived from CD28 is identified as amino acids 111-152 of (SEQ ID NO: 127) which is the following sequence (IEVMYPPPYLDNEKSNGTIIHVKGKHLCPSPLFPGPSKP) that is identified as (SEQ ID NO: 129) where a sequence with at least 95%, 96%, 97%, 98% or 99% identity to (SEQ ID NO: 129) may be used in some embodiments as the CAR hinge. (See e.g., Alabanza, L., Pegues, M., Geldres, C., Shi, V., Wiltzius, J., Sievers, S. A., Yang, S., & Kochenderfer, J. N., 2017, Molecular Therapy: The journal of the American Society of Gene Therapy, 25:2452-2465) The transmembrane domain of CD28 is identified as amino acids 153-179 of (SEQ ID NO: 127) which is the following sequence (FWVLVVVGGVLACYSLLVTVAFIIFWV) that is identified as (SEQ ID NO: 130) where a sequence with at least 92% or 96% identity to (SEQ ID NO: 130) may be used in some embodiments as the CAR transmembrane domain. Although, the transmembrane domain is not necessarily limited in this way and can include portions of the hinge from CD28 as well as portions of the endo domain of CD28. The signaling domain—that in some embodiments is used as the costimulatory sequence—of CD28 is identified as amino acids 180-220 of (SEQ ID NO: 127) which is the following sequence (RSKRSRLLHSDYIVINMTPRRPGPTRKHYQPYAPPRDFAAYRS) that is identified as (SEQ ID NO: 131) where a sequence with at least 92%, 95% or 97% identity to (SEQ ID NO: 131) may be used in some embodiments as the CAR costimulatory signaling domain. Although, the CD28 costimulatory sequence can optionally include a few peptides of the CD28 transmembrane domain even if a full CD8α transmembrane domain is used in the CAR design.

In some embodiments all of the hinge derived from CD28, CD28 transmembrane domain and CD28 costimulatory signaling domain may be used in the CAR construct and is identified as amino acids 111-220 from (SEQ ID NO: 127) of the full CD28 protein before the signal peptide is removed which is the following sequence (IEVMYPPPYLDNEKSNGTIIHVKGKHLCPSPLFPGPSKPFWVLVVVGGVLACYSLLVTV AFIIFWVRSKRSRLLHSDYIVINMTPRRPGPTRKHYQPYAPPRDFAAYRS) that is identified as (SEQ ID NO: 132).

The full sequence of CD8α including the signal peptide can be identified from (SEQ ID NO: 124) where the human coding DNA is (SEQ ID NO: 125). This sequence is also identified on Ensemble as transcript (Transcript ID ENST00000283635.8). This sequence provided includes the signal peptide of CD8α as amino acids 1-21 of (SEQ ID NO: 124) which is the sequence (MALPVTALLLPLALLLHAARP) that is identified as (SEQ ID NO: 123). The extracellular, transmembrane and cytoplasmic regions of the fully human CD8α protein (SEQ ID NO: 124) are identified on Uniprot as P01732 https://www.uniprot.org/uniprot/P01732-1. The hinge of CD8α is identified as amino acids 130-182 of (SEQ ID NO: 124). Although, the 2^{nd} generation CAR design uses a CD8α is identified as amino acids 136-182 of (SEQ ID NO: 124) which is the following sequence TTTPAPRPPTPAPTIASQPLSLRPEACRPAAGGAVHTRGLDFACDIY) that is identified as (SEQ ID NO: 135) where a sequence with at least 95%, 96%, 97%, 98% or 99% identity to (SEQ ID NO: 135) may be used in some embodiments as the CAR hinge. The transmembrane domain of CD8α is identified as amino acids 183-203 of (SEQ ID NO: 124) which is the following sequence (IWAPLAGTCGVLLLSLVITLYC) that is identified as (SEQ ID NO: 134) where a sequence with at least 90% or 95% identity to (SEQ ID NO: 134) may be used in some embodiments as the CAR transmembrane domain. In some embodiments the demarcation of the transmembrane domain is extended into the hinge region or endodomain of CD8α. In some embodiments all of the CD8α hinge and CD8α transmembrane domain may be used in the CAR construct and is identified as amino acids 136-203 from (SEQ ID NO: 124) of the full CD8α protein before the signal peptide is removed which is the following sequence

(TTTPAPRPPTPAPTIASQPLSLRPEACRPAAGGAVHTRGLDFACDIY
IWAPLAGTCGVLLLSLVITLYC).

In some embodiments the 4-1BB signaling domain is used as the co-stimulatory signaling domain of the CAR. The full sequence of 4-1BB including the signal peptide can be identified from (SEQ ID NO: 137) where the human coding DNA is (SEQ ID NO: 138). This sequence is also identified on Ensemble as transcript (Transcript ID ENST00000377507.8). This sequence provided includes the signal peptide of 4-1BB as amino acids 1-23 of (SEQ ID NO: 137) which is the sequence (MGNSCYNIVATLLLVLNFERTRS) that is identified as (SEQ ID NO: 136). The extracellular, transmembrane and cytoplasmic regions of the fully human 4-1BB protein (SEQ ID NO: 138) are identified on Uniprot as Q07011 https://www.uniprot.org/uniprot/Q07011. The signaling domain—that in some embodiments is used as the costimulatory sequence—of 4-1BB is identified as amino acids 214-255 of (SEQ ID NO: 137) which is the following sequence (KRGRKKLLYIFKQPFMRPVQTTQEEDGCSCRFPEEEEGGCEL) that is identified as (SEQ ID NO: 139) where a sequence with at least 92%, 93%, 94%, 95%, 96%, 97%, 98% or 99% identity to (SEQ ID NO: 139) may be used in some embodiments for CAR costimulatory signaling.

The CD3ζ signaling domain is used as the major stimulatory domain in the CAR framework to signal scFv binding to its target. The full sequence of CD3ζ including the signal peptide can be identified from (SEQ ID NO: 141) where the human coding DNA is (SEQ ID NO: 142). This sequence is also identified on Ensemble as transcript (Transcript ID ENST00000362089.10). This sequence provided includes the signal peptide of CD3ζ as amino acids 1-21 of (SEQ ID NO: 141) which is the sequence (MKWKALFTAAILQAQLPITEA) that is identified as (SEQ ID NO: 140). The extracellular, transmembrane and cytoplasmic regions of the fully human CD3ζ protein (SEQ ID NO: 141) are identified on Uniprot as P20963 https://www.uniprot.org/uniprot/P20963-1

The signaling domain of CD3ζ is identified as amino acids 52-164 of (SEQ ID NO: 141) which is the following sequence

(RVKFSRSADAPAYQQGQNQLYNELNLGRREEYDVLDKRRGRDPEMGG
KPRRKNPQEGLYNELQKDKMAEAYSEIGMKGERRRGKGHDGLYQGLST
ATKDTYDALHMQALPPR)

that is identified as (SEQ ID NO: 143) where a sequence with at least 88%, 91%, 94%, 97% or 99% identity to (SEQ ID NO: 143) may be used in some embodiments as the CAR CD3ζ signaling domain.
Dimeric Single Chain Variable Fragment-Fragment-Crystallizable Immunoglobulin A Fusion (dscFV-FcIgA) Design

This instant patent contemplates a dimeric single chain variable fragment-fragment-crystallizable immunoglobulin A fusions (dscFV-FcIgA) to be encoded for by the vector. Including a lentiviral vector, gammaretroviral vector, AAV vector or mRNA. In the dscFV-FcIgA configuration the protein coding part of the vector related to the single chain variable fragment-fragment-crystallizable immunoglobulin A fusion (scFv-FcIgA) encodes in the 5′ to 3′ direction the signal peptide, the single chain variable fragment (scFv) followed by the Immunoglobulin Class A fragment crystallizable (Fc) region which includes the hinge the C_{H}2 domain and C_{H}3 domain. The Immunoglobulin J Chain is incorporated into the vector construct in conjunction with the scFv-FcIgA. The immunoglobulin J Chain sequence may be encoded for following an internal ribosome entry site (IRES) that is directly 3′ following the stop codon of the scFv-FcIgA. Alternatively, in some embodiments the immunoglobulin J Chain is encoded for in the vector preceding or following the scFv-FcIgA where a 2A self-processing peptide or concomitant furin cleavage site followed by a 2A self-processing peptide is placed between and concomitant to the genes encoding for J Chain and the scFv-FcIgA where the first of the two gene elements in the 5′ to 3′ direction does not have a stop codon.

The Fc region of immunoglobulin A1 may be identified from Immunoglobulin Heavy Constant A1 (IgHCA1) (SEQ ID NO: 23) where the Fc region comprising amino acids 104-353 of (SEQ ID NO: 23) which is the following sequence (PSTPPTPSPSTPPTPSPS CCHPRLSLHRPALEDLLLGSEANLTCTLTGLRDASGVTFTWTPSSGKSAVQGPPERDLC GCYSVSSVLPGCAEPWNHGKTFTCTAAYPESKTPLTATLSKSGNTFRPEVHLLPPPSEEL ALNELVTLTCLARGFSPKDVLVRWLQGSQELPREKYLTWASRQEPSQGTTTFAVTSILR VAAEDWKKGDTFSCMVGHEALPLAFTQKTIDRLAGKPTHVNVSVVMAEVDGTCY). Additionally, the hinge of IgHCA1 is identified as amino acids 104-121 of (SEQ ID NO: 23) that is the following sequence (PSTPPTPSPSTPPTPSPS). Although, the hinge can be understood to extend into the C_{H}1 domain for 2 additional amino acids (PV) as the delineation of the C_{H}1 domain terminates 2 amino acids following the final amino acid participating as a beta strand amino acid as part of in Beta sheet formation in the C_{H}1 domain. Thus, one may consider the “extended hinge” to be defined as (PVPSTPPTPSPSTPPTPSPS) that is amino acids 102-121 of (SEQ ID NO: 23). Where a sequence with at least 80%, 85%, 90%, 95% or 99% identity to the hinge or “extended hinge” may be used in some embodiments. In other embodiments the hinge or extended hinge may be shortened by removing up to 14 consecutive amino acids from the hinge where a sequence with at least 80%, 85%, 90% or 95% identity to the remaining amino acids may be used in some embodiments. In other embodiments the hinge may be extended.

The Fc region of immunoglobulin A2 may be identified from Immunoglobulin Heavy Constant A2 (IgHCA2) (SEQ ID NO: 24) where the Fc region comprising amino acids 103-340 of (SEQ ID NO: 24) which is the following sequence (VPPPPPCCHPRLSLHRPALEDLLLGSEANLTCTLTGLRDASGATFTWTPSSGKSAVQGPP ERDLCGCYSVSSVLPGCAQPWNHGETFTCTAAHPELKTPLTANITKSGNTFRPEVHLLPP PSEELALNELVTLTCLARGFSPKDVLVRWLQGSQELPREKYLTWASRQEPSQGTTTYAV TSILRVAAEDWKKGETFSCMVGHEALPLAFTQKTIDRMAGKPTHINVSVVMAEADGTC Y). Additionally, the hinge of IgHCA1 is identified as amino acids 103-108 of (SEQ ID NO: 24) that is the following sequence (VPPPPP). Although, the hinge can be understood to extend into the C_{H}1 domain for 2 additional amino acids (RV) as the delineation of the C_{H}1 domain terminates 2 amino acids following the final amino acid participating as a beta strand amino acid as part of in Beta sheet formation in the C_{H}1 domain. Thus, one may consider the “extended hinge” to be defined as (RVVPPPPP) that is amino acids 101-108 of (SEQ ID NO: 24). Where a sequence with at least 80%, 85%, 90%, 95% or 99% identity to the hinge or “extended hinge” may be used in some embodiments. In other embodiments the hinge or extended hinge may be shortened by removing up to 3 amino acids from the hinge where a sequence with at least 80%, 85%, 90% or 95% identity to the remaining amino acids may be used in some embodiments. In other embodiments the hinge may be extended.

The V_{H} and V_{L} used in the scFv—whose sequences are dependent on the target antigen or allergen—used in the dscFV-FcIgA may be derived from immunoglobulins with ideal binding affinity to the target protein. Ideal binding affinity is that binding affinity that allows therapeutic binding affinity that depends on target. Where the V_{H} region is typically about 119-120 amino acids in length—whose sequence is dependent on the target antigen or allergen—and the V_{L} region is typically about 106-110 amino acids in length—whose sequence is dependent on the target antigen or allergen. The linker used in the present patent between V_{H} and V_{L} used in the scFv include repeating units of GGGGS of at least 4 repeats (GGGGSGGGGSGGGGSGGGGS) (SEQ ID NO: 115) and up to 7 repeats (SEQ ID NO: 121) where a sequence with at least 80%, 85%, 90%, 95% or 99% identity to (SEQ ID NOs: 118, 119, 120 or 121) may be used in some embodiments. Additionally, a Whitlow linker (GSTSGSGKPGSGEGSTKG) (SEQ ID NO: 122) may be used as the linker between the V_{H} and V_{L} with at least 80%, 85%, 90%, 95% or 99% identity to the Whitlow linker (See, e.g. Whitlow, M., Bell, B. A., Feng, S. L., Filpula, D., Hardman, K. D., Hubert, S. L., Rollence, M. L., Wood, J. F., Schott, M. E., Milenic, D. E., et al., 1993, Protein Eng. 6:989-95.) Although, the linkers need not be restricted in this way as the linkers serve the function of flexibility of the Variable region pairs in the scFv and there is a wide variety of ways to generate them

The region directly following the scFv used in the dscFV-FcIgA is Immunoglobulin Heavy Constant A where that includes the hinge. (For identification of the IgA1 and IgA2 hinge see e.g., Senior B W, Woof J M., 2005, J Immunol. 174:7792-9.) Following the immunoglobulin heavy chain constant region a variety of configurations are contemplated to encode for the immunoglobulin J Chain as a separate polypeptide in the final product. The full sequence of immunoglobulin J Chain including the signal peptide is (SEQ ID NO: 11). This sequence is also identified on Ensemble as transcript (Transcript ID ENST00000254801.9). This sequence provided includes the signal peptide of J Chain as amino acids 1-22 of (SEQ ID NO: 11) which is the sequence (MKNHLLFWGVLAVFIKAVHVKA) that is identified as (SEQ ID NO: 27). The signal peptide, and immunoglobulin J Chain of the fully human J Chain protein (SEQ ID NO: 11) are identified on Uniprot as P01591 at web address: https://www.uniprot.org/uniprot/P01591. The Immunoglobulin J Chain may be incorporated into the vector construct in conjunction with the scFv-FvIgA. The immunoglobulin J Chain sequence may be encoded for following an internal ribosome entry site (IRES) (SEQ ID NO: 2) that is directly 3′ following the stop codon of the scFv-FvIgA. Alternatively, the immunoglobulin J Chain may be encoded for in the vector preceding or following the scIgA where either of a A) a 2A self-processing peptide or B) a concomitant furin cleavage site followed by a 2A self-processing peptide is placed between J Chain and scFv-FvIgA.

dIgA and Polymeric Immunglobulin A Encoding Vector Design Framework

Either subclass (A1 or A2 and even engineered variants) of dIgA and polymeric immunglobulin A is made up of three immunoglobulin chains including the immunoglobulin J Chain, the immunoglobulin light chain and the immunoglobulin heavy chain (subclass A1, A2 or an engineered variant). The immunoglobulin J Chain is 159 amino acids in length (SEQ ID NO: 11) before cleavage of the 22 amino acid signal peptide (SEQ ID NO: 27). The immunoglobulin light chain is a single polypeptide that is made up of the immunoglobulin variable region that is about 106-110 amino acids in length and the immunoglobulin light chain constant region that differs based on which light chain encoding locus the immunoglobulin is encoded for on. That immunoglobulin light chain may be derived from the kappa (κ) chain that is encoded for on the immunoglobulin Kappa locus on chromosome 22 or the immunoglobulin lambda (λ) chain that is encoded for on the immunoglobulin lambda locus on chromosome 2. The amino acid sequence of the immunoglobulin Kappa Light Chain Constant Region Allotype Km(1) is (SEQ ID NO: 26). The amino acid sequence of the immunoglobulin Lambda Light Chain Constant Region Allotype is (SEQ ID NO: 25). Additionally, there are three allotypes—or common variants found in the human population—of the constant region of the immunoglobulin Kappa chain Km(1), Km(2) and Km(3) that differ by only one or two amino acids between each allotype. The immunoglobulin lambda chain does not have reported allotypes. There are several variants of the signal peptide found in nature for the immunoglobulin Kappa and lambda chains. For examples of signal peptides used for the immunoglobulin Kappa chain see (SEQ ID NOs: 30-34). For representative examples of signal peptides used for the immunoglobulin lambda chain see (SEQ ID NOs: 35-41). Although, there is a wider variety of signal peptides for the immunoglobulin lambda chain found in the population making no single signal peptide highly representative.

The immunoglobulin heavy chain is made up of a single polypeptide that includes the signal peptide that is cleaved during the processing of the protein followed by the immunoglobulin Variable Heavy (V_{H}) region that is about 119-120 amino acids in length and for IgA1 the constant region made up of Constant Heavy 1 (C_{H}1) domain that is approximately amino acids 1-103 of (SEQ ID NO: 23), the hinge, Constant heavy 2 (C_{H}2) domain that is approximately amino acids 122-222 of (SEQ ID NO: 23) and constant heavy 3 (C_{H}3) domain that is approximately amino acids 223-353 of (SEQ ID NO: 23) for immunoglobulin of A1 (IgA1) (SEQ ID NO: 23). For IgA2 the constant region made up of Constant Heavy 1 (C_{H}1) domain that is approximately amino acids 1-102 of (SEQ ID NO: 24), the hinge, Constant heavy 2 (C_{H}2) domain that is approximately amino acids 109-209 of (SEQ ID NO: 24) and constant heavy 3 (C_{H}3) domain that is approximately amino acids 210-340 of (SEQ ID NO: 24) for immunoglobulin of A1 (IgA1) (SEQ ID NO: 24). Although the C_{H}2 domain could be understood to also include amino acids 210-211 of (SEQ ID NO: 24) although, not necessarily.

In some embodiments one or more of the C_{H}1 domain, C_{H}2 domain or hinge domain of the dimeric immunoglobulin of class A1 or A2 encoded in the vector may be respectively replaced with a one or more of the C_{H}1 domain, C_{H}2 domain or hinge of any other immunoglobulin class and subclass and even more than one class and subclass such as using the hinge of immunoglobulin class G1 and C_{H}2 of immunoglobulin G2. One may determine C_{H}1, Hinge, C_{H}2 and C_{H}3 domains of any immunoglobulin class by observing the crystal structure of the immunoglobulin, the Fab or the Fc region of that immunoglobulin and noting what amino acid is the final amino acid participating in a beta strand or beta sheet. Alternatively, one may also determine domain demarcations (domain and hinge demarcations by looking at exons on the human immunoglobulin heavy chain constant region genes for a specific subclass. Although, in some cases the hinge sequence does not have a separate exon as is the case for the constant domains of IgA1 and IgA2 where the hinge sequence is found in the exon that includes the C_{H}2 domain. Discussed in earlier embodiments any of the C_{H}1, C_{H}2 and C_{H}3 domain may be engineered to modify effector functions.

Polymeric immunoglobulin A1, Dimeric Immunoglobulin class A1 (dIgA1) and immunoglobulin class A1 (IgA1) has a constant region encoded for by the C_{α1} constant region gene found in the cluster of constant region genes for the immunoglobulin heavy chain isotypes. Similarly, polymeric immunoglobulin A2, dimeric Immunoglobulin class A2 (dIgA2) and immunoglobulin class A2 (IgA2) has a constant region encoded for by the C_{α2} constant region found in the cluster of constant region genes for the immunoglobulin heavy chain isotypes. There are two allotypes found in nature for the C_{α2} constant region gene and the C_{α1} constant region gene has a single form found in nature. There are variants of the signal peptide found in nature for the immunoglobulin heavy chains. For representative examples of signal peptides used for the immunoglobulin heavy chain see (SEQ ID NOs: 28-29).

Due to space constraints of the vector and a need for relatively high levels of expression of the genes to reach a therapeutic level of expression the vector encodes in the 5′ to 3′ direction a furin cleavage site followed by a 2A self-processing peptide between the immunoglobulin heavy chain and the immunoglobulin light chain where the relative positions of the immunoglobulin heavy chain and the immunoglobulin light chain may be reversed. This use of a 2A self-processing peptide also ensures that a relatively similar amount of the immunoglobulin heavy and light chains are produced. Additionally, a 2A self-processing peptide may be used in the absence of a furin cleavage site between the immunoglobulin heavy chain and the immunoglobulin light chain where their relative positions may be reversed. Vectors for use in the invention employ the use of signal peptides to lead each immunoglobulin chain. Additionally, the immunoglobulin J Chain sequence (SEQ ID NO:11) may be encoded for following an internal ribosome entry site (IRES) (SEQ ID NO: 2) that is directly 3′ following the stop codon of the immunoglobulin heavy chain or immunoglobulin Kappa light chain. Alternatively, the immunoglobulin J Chain may be encoded for in the vector preceding, none, one or all of the immunoglobulin heavy chain and the immunoglobulin light chain were a 2A self-processing peptide or concomitant furin cleavage site followed by a 2A self-processing/self-cleaving peptide is placed between each of two consecutive transgenes that do not have a stop codon. In some embodiments MZB1 (SEQ ID NO: 8) is also encoded for in the vector following an IRES or MZB1 may be encoded for in the vector preceding, none, one, two or all of the immunoglobulin heavy chain, the immunoglobulin light chain and the immunoglobulin J Chain were a 2A self-processing peptide or concomitant furin cleavage site followed by a 2A self-processing/self-cleaving peptide is placed between each of two consecutive transgenes that do not have a stop codon.

### Lipid Nanoparticles

This instant patent contemplates Lipid Nanoparticles as delivery vehicles for mRNA encoding for any of 1) dIgA1 and polymeric immunoglobulin A1, 2) dIgA2 and polymeric immunoglobulin A2, 3) an engineered variant of 1) or 2) Lipid Nanoparticles have seen unprecedented advances in medicine. LNPs have advantages over capsid delivered vectors in that they are not immunogenic, are biodegradable, have an impressive safety profile and can be delivered with adjuvants. In addition, LNPs have control over drug release, facilitate endosomal escape and can be effective scaled up for manufacturing. LNPs surface features such as the addition of antibodies are typically added after the formation of the LNP with its payload. LNPs range in size from 1 nm to 1000 nm. Although ideally LNPs are less then 150 nm to facilitate their ability to reach their target destination. Lipid nanoparticles can efficiently target liver cells. (See e.g. Truong, B., et. al., 2019, PNAS, 116:21150-21159; Kim, M., et. al., 2021, 7:1-12, 2021.) LNPs can effectively be made to target any cell type through adding monoclonal antibodies or other binding proteins on their surface that are specific to the target cell surface receptor or other CD receptor. This can be done in a variety of ways such as modular manner where a (See, e.g. Veiga, N., Goldsmith, M., Granot, Y. et al., 2018, Nat Commun, 9:4493) LNPs are very effective at delivering the payload to the cytosol of the cell but not the nucleus of the cell. In one embodiment when mRNA is intended for translation in the rough ER or other destination that information can effectively be encoded for on the 3′ UTR. In another embodiment using the evolutionary conserved 3′ UTR for the immunoglobulin as an example or other 3′ UTR for example the 3′ UTR of a protein that is highly expressed and secreted in the target-cell can be incorporated into the mRNA.

LNPs are generally made up of an aqueous core surrounded by a lipid bilayer shell that can include a number of lipids that serve different functions. LNPs generally rely on cationic lipids to efficiently complex negatively charged RNA. Although, anionic and neutral formulations have proven to be effective. Cationic lipids bearing a permanent positive charge are more toxic. Although, amine groups can be incorporated into LNPs allowing them to maintain a cation surface charge at physiological pH which both reduces nonspecific lipid-protein interactions and also allows for RNA release in the cytosol. The cationic charge on the LNP surface allows for efficient interaction with the negatively charged membranes on cell surfaces. This in turn also facilitates engulfment by the cell where the resulting negatively charged endosome is disrupted by the positively charged LNP that facilitates escape of the mRNA. Phospholipids also contribute to the disruption of the endosome. (See e.g., Reichmuth, A. M., Oberli, M. A., Jaklenec, A., Langer, R., Blankschtein, D., 2016, Therapeutic delivery, 7:319-334) There are an extensive number of methods involved in incorporating mRNA in LNPs. In one of many approaches mRNA molecules self-assemble in ionizable molecules in acidic conditions to form uniform LNPs by combining the ingredients of LNPs with the drug. In another approach preassembled LNPs are combined with the drug through a directed microfluidic integration process. The method used in LNP synthesis influences the LNP size and encapsulation efficiency. One method to form LNPs involves condensing lipids from an ethanol solution in water. Aqueous phase mRNA may be encapsulated during condensation. Lipid rafts may be converted into LNPs in a controlled microfluidic process that can accelerate or decelerated the rate of LNP closure influencing the size of the LNP and in turn the total mRNA packaged within it. (See e.g., Reichmuth, A. M., Oberli, M. A., Jaklenec, A., Langer, R., & Blankschtein, D., 2016, Therapeutic delivery, 7:319-334)

### Chimeric Antibodies

This instant patent contemplates the use of chimeric antibodies in the dIgA1 (inclusive of polymeric immunoglobulin A1), dIgA2 (inclusive of polymeric immunoglobulin A2), engineered variants or dscFV-FcIgA gene therapy constructs as an effective means to develop potent affinity for the target of interest. Using the immune system of Mice or other non-human vertebrates can be an effective means to develop potent binding affinity against the target of interest. However, attempting to take an antibody discovered in mouse for use in human often results in a human anti-murine antibody response. Although, there are a few examples of a fully murine FDA approved monoclonal antibody for cancer and kidney transplant rejection. This response can be to the constant region of the mouse antibody or even part of the V-region of the mouse antibody. As a result, potent antibodies developed in mice have had all or part of the genetic information coding for the binding region incorporated in a human antibody construct that preferably utilizes all of the constant region from one human antibody heavy chain (IgG1, IgG2, IgG3 and IgG4) and part of the V-region that the binding element of the mouse antibody can tolerate without losing binding affinity to its target. This patent contemplates the same hybridization techniques to be used in IgA1 and IgA2 constructs intendent for chimeric dIgA and polymeric immunoglobulin A gene therapy encoded vectors. Further, such hybridization techniques may use any mouse IgG or IgA antibody V region and incorporate the necessary components of the V region into an IgA1 or IgA2 antibody. All antibody heavy chains are based on V-regions derived from the same genetic loci and subjected to the same conditions related to affinity maturation making dIgA1 (inclusive of polymeric immunoglobulin A1), dIgA2 (inclusive of polymeric immunoglobulin A2), engineered variants and dscFV-FcIgA highly suitable as a chimeric antibody derived from a mouse or other non-human vertebrate. Additionally, there is no significant different in the principal constructs and function behind IgA1 or IgA2 and that of IgG1, IgG2, IgG3 and IgG4 in their engagement with the V-region. That is a V-region that is effective in one construct is generally effective in all constructs. In antibodies the residues in the variable domains (V region) in each of the heavy and light chains of the region are partitioned into three hypervariable complementarity-determining regions (CDRs) and four framework regions (FRs). The FRs, which make up about 85% of the V region, are a more stable amino acid sequence and function as the scaffolds for the CDRs that directly contact the antigen. The FRs can influence the orientation, relative conformation, phi an psi angles of some amino acids in the CDRs and thus can ultimately improve or disrupt the binding affinity of the CDR derived from a mouse monoclonal antibody. Thus, some of the mouse FRs may be used in the chimeric antibody with potentially some amino acid substitution in the human FRs or non-human vertebrate derived CDRs may be made to ensure the binding affinity remains potent. In one embodiment the CDRs from a mouse IgG1, IgG2A, IgG2B, IgG2C or IgA antibody are incorporated into an otherwise fully human IgA1 or IgA2 antibody intended to be expressed as dIgA1 or dIgA2 respectively. In another embodiment all the CDRs and one or more of FR1, FR2, FR3 and FR4 from a mouse IgG1, IgG2A, IgG2B, IgG2C or IgA antibody are incorporated into an otherwise fully human IgA1 or IgA2 antibody intended to be expressed as dIgA1 or dIgA2 respectively. In a further embodiment amino acid substitutions, additions or deletions are made into the CDRs or FRs from mice or human in the chimeric dIgA1 or dIgA2 antibodies. In a further embodiment another non-human vertebrate such as a rat or rabbit is used in place of a mouse.

TABLE 2

| | | | |
|---|---|---|---|
| SEQ | | | |
| ID NO: | 5′ UTR | Length | Protein |

Table of 5′ Human UTRs

| | | | |
|---|---|---|---|
| 42 | agaagaagtg aagtcaag | 18 | J Chain |
| 43 | tgagcgcaga aggcaggact cgggacaatc | 36 | Immunoglobulin Lamba Variable |
| | ttcatc | | Variant 1 |
| 44 | gctgcgggta gagaagacag gactcaggac | 40 | Immunoglobulin Lamba Variable |
| | aatctccagc | | Variant 2 |
| 45 | gcaggaatca gtcccactca ggacacagc | 29 | Immunoglobulin Kappa Variable |
| | | | Variant 1 |
| 46 | aggctggaca cacttcatgc aggagtcagac | 53 | Immunoglobulin Kappa Variable |
| | cctgtcaggac acagcatagac | | Variant 2 |
| 47 | gagagcatca cccagcaacc acatctgtcc | 63 | Immunoglobulin Heavy Variable |
| | tctagagatc ccctgagagc tccgttcctc | | Variant 1 |
| | acc | | |
| 48 | agtgactcct gtgccccacc | 20 | Immunoglobulin Heavy Variable |
| | | | Variant 2 |
| 49 | actcttctgg tccccacaga cttagagaga | 37 | hemoglobin subunit alpha 1 |
| | acccacc | | |
| 50 | actcttctgg tccccacaga cttagagaga | 37 | hemoglobin subunit alpha 2 |
| | acccacc | | |
| 51 | gacagtgctg acactacaag gctcggagct | 47 | fibrinogen-gamma chain |
| | ccgggcactc agacatc | | |
| 52 | aagtctac | 8 | fibrinogen-beta chain |
| 53 | aatcctttct ttcagctgga gtgctcctca | 55 | fibrinogen-alpha chain |
| | ggagccagcc ccacccttag aaaag | | |
| 54 | ctagcttttc tcttctgtca accccacacg | 41 | albumin |
| | cctttggcac a | | |
| 42 | agaagaagtg aagtcaag | 18 | J Chain |
| 43 | tgagcgcaga aggcaggact cgggacaatc | 36 | Immunoglobulin Lamba Variable |
| | ttcatc | | Variant 1 |
| 44 | gctgcgggta gagaagacag gactcaggac | 40 | Immunoglobulin Lamba Variable |
| | aatctccagc | | Variant 2 |
| 45 | gcaggaatca gtcccactca ggacacagc | 29 | Immunoglobulin Kappa Variable |
| | | | Variant 1 |
| 46 | aggctggaca cacttcatgc aggagtcagac | 53 | Immunoglobulin Kappa Variable |
| | cctgtcaggac acagcatagac | | Variant 2 |
| 47 | gagagcatca cccagcaacc acatctgtcc | 63 | Immunoglobulin Heavy Variable |
| | tctagagatc ccctgagagc tccgttcctc | | Variant 1 |
| | acc | | |
| 48 | agtgactcct gtgccccacc | 20 | Immunoglobulin Heavy Variable |
| | | | Variant 2 |
| 49 | actcttctgg tccccacaga ctcagagaga | 37 | hemoglobin subunit alpha 1 |
| | acccacc | | |
| 50 | actcttctgg tccccacaga ctcagagaga | 37 | hemoglobin subunit alpha 2 |
| | acccacc | | |
| 51 | gacagtgctg acactacaag gctcggagct | 47 | fibrinogen-gamma chain |
| | ccgggcactc agacatc | | |
| 52 | aagtctac | 8 | fibrinogen-beta chain |
| 53 | aatcctttct ttcagctgga gtgctcctca | 55 | fibrinogen-alpha chain |
| | ggagccagcc ccacccttag aaaag | | |
| 54 | ctagcttttc tcttctgtca accccacacg | 41 | albumin |
| | cctttggcac a | | |

b (Table 2 as mRNA)
Table of 5′ Human UTRs as mRNA

| | | | |
|---|---|---|---|
| 74 | agaagaagug aagucaag | 18 | J Chain |
| 75 | ugagcgcaga aggcaggacu cgggacaauc | 36 | Immunoglobulin Lamba Variable |
| | uucauc | | Variant 1 |
| 76 | gcugcgggua gagaagacag gacucaggac | 40 | Immunoglobulin Lamba Variable |
| | aaucuccagc | | Variant 2 |
| 77 | gcaggaauca gucccacuca ggacacagc | 29 | Immunoglobulin Kappa Variable |
| | | | Variant 1 |
| 78 | aggcuggaca cacuucaugc aggagucagac | 53 | Immunoglobulin Kappa Variable |
| | ccugucaggac acagcauagac | | Variant 2 |
| 79 | gagagcauca cccagcaacc acaucugucc | 63 | Immunoglobulin Heavy Variable |
| | ucuagagauc cccugagagc uccguuccuc | | Variant 1 |
| | acc | | |
| 80 | agugacuccu gugccccacc | 20 | Immunoglobulin Heavy Variable |
| | | | Variant 2 |
| 81 | acucuucugg uccccacaga cucagagaga | 37 | hemoglobin subunit alpha 1 |
| | acccacc | | |
| 82 | acucuucugg uccccacaga cucagagaga | 37 | hemoglobin subunit alpha 2 |
| | acccacc | | |
| 83 | gacagugcug acacuacaag gcucggagcu | 47 | fibrinogen-gamma chain |
| | ccgggcacuc agacauc | | |
| 84 | aagucuac | 8 | fibrinogen-beta chain |
| 85 | aauccuuucu uucagcugga gugcuccuca | 55 | fibrinogen-alpha chain |
| | ggagccagcc ccacccuuag aaaag | | |
| 86 | cuagcuuuuc ucuucuguca accccacacg | 41 | albumin |
| | ccuuuggcac a | | |

TABLE 3
Table of 3′ Human UTRs

| SEQ | | | |
|---|---|---|---|
| ID | | | |
| NO: | 3′ UTR | Length | Protein |
| 55 | tttaagtcat tgctgactgc atagctcttt ttcttgagag gctctccatt ttgattcaga | 788 | J Chain |
| | aagttagcat atttattacc aatgaatttg aaaccagggc tttttttttt ttttgggtga | | |
| | tgtaaaacca actccctgcc accaaaataa ttaaaatagt cacattgtta tctttattag | | |
| | gtaatcactt cttaattata tgttcatact ctaagtatcaa aatcttccaa ttatcatgct | | |
| | cacctgaaag aggtatgctc tcttaggaat acagtttcta gcattaaaca aataaacaag | | |
| | gggagaaaat aaaactcaag gactgaaaat caggaggtgt aataaaatgt tcctcgcatt | | |
| | cccccccgct tttttttttt tttttgactt tgccttggag agccagagct tccgcatttt | | |
| | ctttactatt ctttttaaaa aaagtttcac tgtgtagaga acatatatgc ataaacatag | | |
| | gtcaattata tgtctccatt agaaaaataa taattggaaa acatgttcta gaactagtta | | |
| | caaaaataat ttaaggtgaa atctctaata tttataaaag tagcaaaata aatgcataat | | |
| | taaaatatat ttggacataa cagacttgga agcagatgat acagacttct ttttttcata | | |
| | atcaggttag tgtaagaaat tgccatttga aacaatccat tttgtaactg aaccttatga | | |
| | aatatatgta tttcatggta cgtattctct agcacagtct gagcaattaa atagattcat | | |
| | aagcata | | |
| 56 | gtgccacggc cggcaagccc ccgctcccca ggctctcggg gtcgcgcgag gatgcttggc | 154 | IgHG1 Immunoglobulin |
| | acgtaccccg tgtacatact tcccaggcac ccagcatgga aataaagcac ccagcgcttc | | Heavy Constant Chain |
| | cctgggcccc tgcg | | |
| 57 | gtgccacggc cggcaagccc ccgctcccca ggctctcggg gtcgcgcgag gatgcttggc | 135 | IgHG2 Immunoglobulin |
| | acgtaccccg tctacatact tcccgggcac ccagcatgga aataaagcac ccagcgctgc | | Heavy Constant Chain |
| | cctgggcccc tgcga | | |
| 58 | gtgccacggc cggcaagccc ccgctcccca ggctctcggg gtcgcgcgag gatgcttggc | 135 | IgHG3 Immunoglobulin |
| | acgtaccccg tctacatact tcccgggcac ccagcatgga aataaagcac ccagcgctgc | | Heavy Constant Chain |
| | cctgggcccc tgcga | | |
| 59 | gtgccacggc cggcaagccc ccgctcccca ggctctcggg gtcgcgcgag gatgcttggc | 135 | IgHG4 Immunoglobulin |
| | acgtaccccg tctacatact tcccgggcac ccagcatgga aataaagcac ccagcgctgc | | Heavy Constant Chain |
| | cctgggcccc tgcga | | |
| 60 | gcaggagccg gcaaggcaca gggaggaagt gtggaggaac ctcttggaga agccagctat | 113 | IgHAl Immunoglobulin |
| | gcttgccaga actcagccct ttcagacatc accgacccgc ccttactcac atg | | Heavy Constant Heavy |
| | | | A1 for memory B-cell |
| | | | Receptor |
| 61 | gccgcccgcc tgtccccacc cctgaataaa ctccatgctc ccccaagcag | 50 | IgHAl Immunoglobulin |
| | | | Heavy Constant Heavy |
| | | | A1 |
| 62 | gcgggagccg gcaaggcaca gggaggaagt gtggaggaac ctcttggaga agccagctat | 144 | IgHA2 Immunoglobulin |
| | gcttgccaga actcagccct ttcagacatc accgacccgc ccttactcac gtggcttcca | | Heavy Constant Heavy |
| | ggtgcaataa agtggcccca agga | | A2 for memory B-cell |
| | | | Receptor |
| 63 | gccgcccgcc tgtccccacc cctgaataaa ctccatgctc ccccaagc | 49 | IgHA2 Immunoglobulin |
| | | | Heavy Constant Heavy |
| | | | A2 |
| 64 | gttcccaact ctaaccccac ccacgggagc ctggagctgc aggatcccag gggaggggtc | 140 | IgKL Immunoglovulin |
| | tctctcccca tcccaagtca tccagccctt ctccctgcac tcatgaaacc ccaataaata | | A2 Light Constant |
| | tcctcattga caaccagaaa | | Chain |
| 65 | gttcccaact ctaaccccac ccacgggagc ctggagctgc aggatcccag gggaggggtc | 140 | IgAL Immunoglovulin |
| | tctctcccca tcccaagtca tccagccctt ctccctgcac tcatgaaacc ccaataaata | | Lambda Light Constant |
| | tcctcattga caaccagaaa | | Chain |
| 66 | gctggagcct cggtggccat gcttcttgcc ccttgggcct ccccccagcc cctcctcccc | 111 | HBA1 hemoglobin |
| | ttcctgcacc cgtacccccg tggtctttga ataaagtctg agtgggcggc a | | subunit alpha 1 |
| 67 | gctggagcct cggtagccgt tcctcctgcc cgctgggcct cccaacgggc cctcctcccc | 110 | HBA2 hemoglobin |
| | tccttgcacc ggcccttcct ggtctttgaa taaagtctga gtgggcagca | | subunit alpha 2 |
| 68 | aaaattatgt ctttttaata tggtttttgt tttgttatat attcacaggc tggagacgtt | 232 | fibrinogen-gamma |
| | taaaagaccg tttcaaaaga gatttacttt tttaaaggac tttatctgaa cagagagata | | chain |
| | taatattttt cctattggac aatggacttg caaagcttca cttcatttta agagcaaaag | | |
| | accccatgtt gaaaactcca taacagtttt atgctgatga taatttatct ac | | |
| 69 | actaagttaa atatttctgc acagtgttcc catggcccct tgcatttcct tcttaactct | 219 | fibrinogen-alpha |
| | ctgttacacg tcattgaaac tacacttttt tggtctgttt ttgtgctaga ctgtaagttc | | chain |
| | cttgggggca gggcctttgt ctgtctcatc tctgtattcc caaatgccta acagtacaga | | |
| | gccatgactc aataaataca tgttaaatgg atgaatgaa | | |
| 70 | catcacatt taaaagcatc tcagcctacc atgagaataa gagaaagaaa atgaagatca | 414 | Albumin |
| | aaagcttatt catctgtttt tctttttcgt tggtgtaaag ccaacaccct gtctaaaaaa | | |
| | cataaatttc tttaatcatt ttgcctcttt tctctgtgct tcaattaata aaaaatggaa | | |
| | agaatctaat agagtggtac agcactgtta tttttcaaag atgtgttgct atcctgaaaa | | |
| | ttctgtaggt tctgtggaag ttccagtgtt ctctcttatt ccacttcggt agaggatttc | | |
| | tagtttcttg tgggctaatt aaataaatca ttaatactct tctaagttat ggattataaa | | |
| | cattcaaaat aatattttga cattatgata attctgaata aaagaacaaa aacca | | |

TABLE 3b
(Table 3 as mRNA)
Table of 3′ Human UTRs as mRNA

| SEQ ID | | | |
|---|---|---|---|
| NO: | 3′ UTR | Length | Protein |
| 87 | uuuaagucau ugcugacugc auagcucuuu uucuugagag gcucuccauu uugauucaga aaguuagcau | 788 | J Chain |
| | auuuauuacc aaugaauuug aaaccagggc uuuuuuuuuu uuuuggguga uguaaaacca acucccugcc | | |
| | accaaaauaa uuaaaauagu cacauuguua ucuuuauuag guaaucacuu cuuaauuaua uguucauacu | | |
| | cuaaguaucaa aaucuuccaa uuaucaugcu caccugaaag agguaugcuc ucuuaggaau acaguuucua | | |
| | gcauuaaaca aauaaacaag gggagaaaau aaaacucaag gacugaaaau caggaggugu aauaaaaugu | | |
| | uccucgcauu cccccccgcu uuuuuuuuuu uuuuugacuu ugccuuggag agccagagcu uccgcauuuu | | |
| | cuuuacuauu cuuuuuaaaa aaaguuucac uguguagaga acauauaugc auaaacauag gucaauuaua | | |
| | ugucuccauu agaaaaauaa uaauuggaaa acauguucua gaacuaguua caaaaauaau uuaaggugaa | | |
| | aucucuaaua uuuauaaaag uagcaaaaua aaugcauaau uaaaauauau uuggacauaa cagacuugga | | |
| | agcagaugau acagacuucu uuuuuucaua aucagguuag uguaagaaau ugccauuuga aacaauccau | | |
| | uuuguaacug aaccuuauga aauauaugua uuucauggua cguauucucu agcacagucu gagcaauuaa | | |
| | auagauucau aagcaua | | |
| 88 | gugccacggc cggcaagccc ccgcucccca ggcucucggg gucgcgcgag gaugcuuggc acguaccccg uguacauacu | 154 | IgHG1 Immunoglobulin Heavy Constant Chain |
| | ucccaggcac ccagcaugga aauaaagcac ccagcgcuuc ccugggcccc ugcg | | |
| 89 | gugccacggc cggcaagccc ccgcucccca ggcucucggg gucgcgcgag gaugcuuggc acguaccccg ucuacauacu | 135 | IgHG2 Immunoglobulin Heavy Constant Chain |
| | ucccgggcac ccagcaugga aauaaagcac ccagcgcugc ccugggcccc ugcga | | |
| 90 | gugccacggc cggcaagccc ccgcucccca ggcucucggg gucgcgcgag gaugcuuggc acguaccccg ucuacauacu | 135 | IgHG3 Immunoglobulin Heavy Constant Chain |
| | ucccgggcac ccagcaugga aauaaagcac ccagcgcugc ccugggcccc ugcga | | |
| 91 | gugccacggc cggcaagccc ccgcucccca ggcucucggg gucgcgcgag gaugcuuggc acguaccccg ucuacauacu | 135 | IgHG4 Immunoglobulin Heavy Constant Chain |
| | ucccgggcac ccagcaugga aauaaagcac ccagcgcugc ccugggcccc ugcga | | |
| 92 | gcaggagccg gcaaggcaca gggaggaagu guggaggaac cucuuggaga agccagcuau gcuugccaga | 113 | IgHA1 Immunoglobulin Heavy Constant Heavy |
| | acucagcccu uucagacauc accgacccgc ccuuacucac aug | | A1 for memory B-cell Receptor |
| 93 | gccgcccgcc uguccccacc ccugaauaaa cuccaugcuc ccccaagcag | 50 | IgHA1 Immunoglobulin Heavy Constant Heavy |
| | | | A1 |
| 94 | gcgggagccg gcaaggcaca gggaggaagu guggaggaac cucuuggaga agccagcuau gcuugccaga | 144 | IgHA2 Immunoglobulin Heavy Constant Heavy |
| | acucagcccu uucagacauc accgacccgc ccuuacucac guggcuucca ggugcaauaa aguggcccca agga | | A2 for memory B-cell Receptor |
| 95 | gccgcccgcc uguccccacc ccugaauaaa cuccaugcuc ccccaagc | 49 | IgHA2 Immunoglobulin Heavy Constant Heavy |
| | | | A2 |
| 96 | guucccaacu cuaaccccac ccacgggagc cuggagcugc aggaucccag gggagggguc ucucucccca ucccaaguca | 140 | IgKL Immunoglovulin Kappa Light Constant |
| | uccagcccuu cucccugcac ucaugaaacc ccaauaaaua uccucauuga caaccagaaa | | Chain |
| 97 | guucccaacu cuaaccccac ccacgggagc cuggagcugc aggaucccag gggagggguc ucucucccca ucccaaguca | 140 | IgλL Immunoglovulin Lambda Light Constant |
| | uccagcccuu cucccugcac ucaugaaacc ccaauaaaua uccucauuga caaccagaaa | | Chain |
| 98 | gcuggagccu cgguggccau gcuucuugcc ccuugggccu ccccccagcc ccuccucccc uuccugcacc cguacccccg | 111 | HBA1 hemoglobin subunit alpha 1 |
| | uggucuuuga auaaagucug agugggcggc a | | |
| 99 | gcuggagccu cgguagccgu uccuccugcc cgcugggccu cccaacgggc ccuccucccc uccuugcacc ggcccuuccu | 110 | HBA2 hemoglobin subunit alpha 2 |
| | ggucuuugaa uaaagucuga gugggcagca | | |
| 100 | aaaauuaugu cuuuuuaaua ugguuuuugu uuuguuauau auucacaggc uggagacguu uaaaagaccg | 232 | fibrinogen-gamma chain |
| | uuucaaaaga gauuuacuuu uuuaaaggac uuuaucugaa cagagagaua uaauauuuuu ccuauuggac | | |
| | aauggacuug caaagcuuca cuucauuuua agagcaaaag accccauguu gaaaacucca uaacaguuuu | | |
| | augcugauga uaauuuaucu ac | | |
| 101 | acuaaguuaa auauuucugc acaguguucc cauggccccu ugcauuuccu ucuuaacucu cuguuacacg | 219 | fibrinogen-alpha chain |
| | ucauugaaac uacacuuuuu uggucuguuu uugugcuaga cuguaaguuc cuugggggca gggccuuugu | | |
| | cugucucauc ucuguauucc caaaugccua acaguacaga gccaugacuc aauaaauaca uguuaaaugg | | |
| | augaaugaa | | |
| 102 | caucacauu uaaaagcauc ucagccuacc augagaauaa gagaaagaaa augaagauca aaagcuuauu | 414 | Albumin |
| | caucuguuuu ucuuuuucgu ugguguaaag ccaacacccu gucuaaaaaa cauaaauuuc uuuaaucauu | | |
| | uugccucuuu ucucugugcu ucaauuaaua aaaaauggaa agaaucuaau agagugguac agcacuguua | | |
| | uuuuucaaag auguguugcu auccugaaaa uucuguaggu ucuguggaag uuccaguguu cucucuuauu | | |
| | ccacuucggu agaggauuuc uaguuucuug ugggcuaauu aaauaaauca uuaauacucu ucuaaguuau | | |
| | ggauuauaaa cauucaaaau aauauuuuga cauuaugaua auucugaaua aaagaacaaa aacca | | |

TABLE 4
Limited Table of Human Promoters and Enhancers (DNA)

| Seq | | Sequence | |
|---|---|---|---|
| ID NO | Sequence | Length | Sequence Name |
| 104 | ggctcggtgcccgtcagtgggcagagcgcacatcgccca | 1179 | Eukaryotic |
| | cagtccccgagaagttggggggaggggtcggcaattgaa | | Translation |
| | ccggtgcctagagaaggtggcgcggggtaaactgggaaa | | Elongation |
| | gtgatgtcgtgtactggctccgcctttttcccgagggtg | | Factor 1 alpha |
| | ggggagaaccgtatataagtgcagtagtcgccgtgaacg | | (EF-1alpha) |
| | ttctttttcgcaacgggtttgccgccagaacacaggtaa | | Promoter |
| | gtgccgtgtgtggttcccgcgggcctggcctctttacgg | | |
| | gttatggcccttgcgtgccttgaattacttccacctggc | | |
| | tgcagtacgtgattcttgatcccgagcttcgggttggaa | | |
| | gtgggtgggagagttcgaggccttgcgcttaaggagccc | | |
| | cttcgcctcgtgcttgagttgaggcctggcctgggcgct | | |
| | ggggccgccgcgtgcgaatctggtggcaccttcgcgcct | | |
| | gtctcgctgctttcgataagtctctagccatttaaaatt | | |
| | tttgatgacctgctgcgacgctttttttctggcaagata | | |
| | gtcttgtaaatgcgggccaagatctgcacactggtattt | | |
| | cggtttttggggccgcgggcggcgacggggcccgtgcgt | | |
| | cccagcgcacatgttcggcgaggcggggcctgcgagcgc | | |
| | ggccaccgagaatcggacgggggtagtctcaagctggcc | | |
| | ggcctgctctggtgcctggcctcgcgccgccgtgtatcg | | |
| | ccccgccctgggcggcaaggctggcccggtcggcaccag | | |
| | ttgcgtgagcggaaagatggccgcttcccggccctgctg | | |
| | cagggagctcaaaatggaggacgcggcgctcgggagagc | | |
| | gggcgggtgagtcacccacacaaaggaaaagggcctttc | | |
| | cgtcctcagccgtcgcttcatgtgactccacggagtacc | | |
| | gggcgccgtccaggcacctcgattagttctcgagctttt | | |
| | ggagtacgtcgtctttaggttggggggaggggttttatg | | |
| | cgatggagtttccccacactgagtgggtggagactgaag | | |
| | ttaggccagcttggcacttgatgtaattctccttggaat | | |
| | ttgccctttttgagtttggatcttggttcattctcaagc | | |
| | ctcagacagtggttcaaagtttttttcttccatttcagg | | |
| | tgtcgtga | | |
| 105 | taaaccggtgagtttcatggttacttgcctgagaagatt | 904 | Feek Promoter |
| | aaaaaaagtaatgctaccttatgagggagagtcccaggg | | |
| | accaagatagcaactgtcatagcaaccgtcacactgctt | | |
| | tggtcaaggagaagaccctttggggaactgaaaacagaa | | |
| | ccttgagcacatctgttgctttcgctcccatcctcctcc | | |
| | aacagggctgggtggagcactccacaccctttcaccggt | | |
| | cgtacggctcagccagagtaaaaatcacacccatgacct | | |
| | tdgccactgagggcttgatcaattcactttgaatttggc | | |
| | attaaataccattaaggtatattaactgattttaaaata | | |
| | agatatattcgtgaccatgtttttaactttcaaaaatgt | | |
| | agctgccagtgtdtgattttatttcagttgtacaaaata | | |
| | tctaaacctatagcaatgtgattaataaaaacttaaaca | | |
| | tattttccagtaccttaattctgtdataggaaaatttta | | |
| | atctgagtattttaatttcataatctctaaaatagttta | | |
| | atgatttgtcttgtgttgctgtcgtttaccccagctgat | | |
| | ctcaaaagtgatatttaaggagattattttggtctgcaa | | |
| | caacttgatagggctcagcctctcccacccaacgggtgg | | |
| | aatcccccagagggggatttccaagaggccacctggcag | | |
| | ttgctgagggtcagaagtgaagctagccacttcctctta | | |
| | ggcaggtggccaagattacagttgacccgtacgtgcagc | | |
| | tgtgcccagcctgccccatcccctgctcatttgcatgtt | | |
| | cccagagcacaacctcctgccctgaagccttattaatag | | |
| | gctggtcacactttgtgcaggagtcagactcagtcagga | | |
| | cacagct | | |
| 106 | ttgtgtgtccccaactttccaaatccccgcccccgcgat | 201 | Tumor Necrosis |
| | ggagaagaaaccgagacagaaggtgcagggcccactacc | | Factor Alpha |
| | gcttcctccagatgagctcatgggtttctccaccaagga | | (TNF alpha) |
| | agttttccgctggttgaatgattctttccccgccctcct | | Promoter (−200 |
| | ctcgccccagggacatataaaggcagttgttggcacacc | | to |
| | cagcca | | Transcription |
| | | | Start Site |
| | | | (TSS)) |
| 107 | ttgtgtgtccccaactttccaaatccccgcccccgcgat | 377 | Tumor Necrosis |
| | ggagaagaaaccgagacagaaggtgcagggcccactacc | | Factor Alpha |
| | gcttcctccagatgagctcatgggtttctccaccaagga | | (TNF alpha) |
| | agttttccgctggttgaatgattctttccccgccctcct | | Promoter (−200 |
| | ctcgccccagggacatataaaggcagttgttggcacacc | | to TSS) |
| | cagccagcagacgctccctcagcaaggacagcagaggac | | followed by |
| | cagctaagagggagagaagcaactacagaccccccctga | | 5′ UTR |
| | aaacaaccctcagacgccacatcccctgacaagctgcca | | |
| | ggcaggttctcttcctctcacatactgacccacggctcc | | |
| | accctctctcccctggaaaggacacc | | |
| 108 | tttcctgcatcctgtctggaagttagaaggaaacagacc | 397 | Tumor Necrosis |
| | acagacctggtccccaaaagaaatggaggcaataggttt | | Factor Alpha |
| | tgaggggcatggggacggggttcagcctccagggtccta | | (TNF alpha) |
| | cacacaaatcagtcagtggcccagaagacccccctcgga | | Promoter (−400 |
| | atcggagcagggaggatggggagtgtgaggggtatcctt | | to TSS) |
| | gatgcttgtgtgtccccaactttccaaatccccgccccc | | |
| | gcgatggagaagaaaccgagacagaaggtgcagggccca | | |
| | ctaccgcttcctccagatgagctcatgggtttctccacc | | |
| | aaggaagttttccgctggttgaatgattctttccccgcc | | |
| | ctcctctcgccccagggacatataaaggcagttgttggc | | |
| | acaccca | | |
| 109 | tagttattaatagtaatcaattacggggtcattagttca | 565 | Tumor Necrosis |
| | tagcccatatatggagttccgcgttacataacttacggt | | Factor Alpha |
| | aaatggcccgcctggctgaccgcccaacgacccccgccc | | Promoter (−200 |
| | attgacgtcaataatgacgtatgttcccatagtaacgcc | | to TSS) with |
| | aatagggactttccattgacgtcaatgggtggagtattt | | upstream CMV |
| | acggtaaactgcccacttggcagtacatcaagtgtatca | | Enhancer |
| | tatgccaagtacgccccctattgacgtcaatgacggtaa | | |
| | atggcccgcctggcattatgcccagtacatgaccttatg | | |
| | ggactttcctacttggcagtacatctacgtattagtcat | | |
| | cgctattaccatgatgcttgtgtgtccccaactttccaa | | |
| | atccccgcccccgcgatggagaagaaaccgagacagaag | | |
| | gtgcagggcccactaccgcttcctccagatgagctcatg | | |
| | ggtttctccaccaaggaagttttccgctggttgaatgat | | |
| | tctttccccgccctcctctcgccccagggacatataaag | | |
| | gcagttgttggcacaccca | | |
| 110 | ggaggctgcagtttctagaagagggtggggacactgcgg | 545 | Perforin |
| | agagaagatggggccagattccgagaagacagcataagc | | Promoter |
| | ccctgttcctgtaagagcagggacggaagcagggacata | | Trunacted |
| | aacgcaagggatgagccccaaagtgtgacccatgagaca | | includes the |
| | tgatgtcacatgtggtctggtgttcatcaacaccagggc | | 5′ UTR |
| | cgagtctcaaagtcctcagcgccccgccctcctccgcct | | |
| | gtgtgccctgagtccccgagccccagcagctctactcgg | | |
| | cagatgagcctctggccctgctgctcgcttcctgagggc | | |
| | tgtcagtggggagccggatgagggctgaggacagggtgg | | |
| | gtgcttgtgggaggggagagcacaaaggacctgtgacca | | |
| | cagctgggggcggggcaggaagtagaagtgatgtgagtg | | |
| | gtggctggtgcaaggagccacagtgggctgcctgggggg | | |
| | ctgatgccaccattccaggagcctcggtgaagagaggat | | |
| | atccatctgtgtagccgcttctctatacgggattccag | | |
| 111 | tgatctagagcaatttgaaacttgtggtagatattttac | 611 | Interferon |
| | taaccaactctgatgaaggacttcctcaccaaattgttc | | Gamma (IFN- |
| | ttttaaccgcattctttccttgctttctggtcatttgca | | gamma) |
| | agaaaaattttaaaaggctgcccctttgtaaaggtttga | | Promoter (−546 |
| | gaggccctagaatttcgtttttcacttgttcccaaccac | | to +64 |
| | aagcaaatgatcaatgtgctttgtgaatgaagagtcaac | | Relative to |
| | attttaccagggcgaagtggggaggtacaaaaaaatttc | | TSS) partially |
| | cagtccttgaatggtgtgaagtaaaagtgccttcaaaga | | inclusive of |
| | atcccaccagaatggcacaggtgggcataatgggtctgt | | 5′ UTR |
| | ctcatcgtcaaaggacccaaggagtctaaaggaaactct | | |
| | aactacaacacccaaatgccacaaaaccttagttattaa | | |
| | tacaaactatcatccctgcctatctgtcaccatctcatc | | |
| | ttaaaaaacttgtgaaaatacgtaatcctcaggagactt | | |
| | caattaggtataaataccagcagccagaggaggtgcagc | | |
| | acattgttctgatcatctgaagatcagctattagaagag | | |
| | aaagatcagttaagtcctttggacct | | |
| 112 | tgatctagagcaatttgaaacttgtggtagatattttac | 671 | Interferon |
| | taaccaactctgatgaaggacttcctcaccaaattgttc | | Gamma (IFN- |
| | ttttaaccgcattctttccttgctttctggtcatttgca | | gamma) |
| | agaaaaatttaaaaggctgcccctttgtaaaggtttgag | | Promoter (−546 |
| | aggccctagaatttcgtttttcacttgttcccaaccaca | | to +125 |
| | agcaaatgatcaatgtgctttgtgaatgaagagtcaaca | | Relative to |
| | ttttaccagggcgaagtggggaggtacaaaaaaatttcc | | TSS) fully |
| | agtccttgaatggtgtgaagtaaaagtgccttcaaagaa | | inclusive of |
| | tcccaccagaatggcacaggtgggcataatgggtctgtc | | 5′ UTR |
| | tcatcgtcaaaggacccaaggagtctaaaggaaactcta | | |
| | actacaacacccaaatgccacaaaaccttagttattaat | | |
| | acaaactatcatccctgcctatctgtcaccatctcatct | | |
| | taaaaaacttgtgaaaatacgtaatcctcaggagacttc | | |
| | aattaggtataaataccagcagccagaggaggtgcagca | | |
| | cattgttctgatcatctgaagatcagctattagaagaga | | |
| | aagatcagttaagtcctttggacctgatcagcttgatac | | |
| | aagaactactgatttcaacttctttggcttaattctctc | | |
| | ggaaacg | | |
| 113 | tgggtgatgatgtggtcactgcatgattctcacacaagc | 230 | Granzyme B |
| | acccagaggacgtcatcaggcagaggcagtgggggtggg | | Promoter fully |
| | cagcatttacagaaaatctgtgatgagacaccacaaaac | | inclusive of |
| | cagaggggaacatgaagtcactgagcctgctccacctct | | the 5′ UTR |
| | ttcctctcccaagagctaaaagagagcaaggaggaaaca | | |
| | acagcagctccaaccagggcagccttcctgagaag | | |
| 114 | agctccaaccagggcagccttcctgagaag | 30 | Granzyme B |
| | | | 5′ UTR |
| 115 | agtgtagttttatgacaaagaaaattttctgagttactt | 346 | Interleukin-2 |
| | ttgtatccccaccccttaaagaaaggaggaaaaactgtt | | (IL-2) |
| | tcatacagaaggcgttaattgcatgaattagagctatca | | Promoter |
| | cctaagtgtgggctaatgtaacaaagagggatttcacct | | Truncated |
| | acatccattcagtcagtctttgggggtttaaagaaattc | | (−346 to TSS) |
| | caaagagtcatcagaagaggaaaaatgaaggtaatgttt | | |
| | tttcagacaggtaaagtctttgaaaatatgtgtaatatg | | |
| | taaaacattttgacacccccataatatttttccagaatt | | |
| | aacagtataaattgcatctcttgttcaagagtt | | |
| 116 | agtatttctctagctgacatgtaagaagcaatctatctt | 540 | Interleukin-2 |
| | attgtatgcaattagctcattgtgtggataaaaaggtaa | | (IL-2) Full |
| | aaccattctgaaacaggaaaccaatacacttcctgtttt | | Promoter (−540 |
| | atcaacaaatctaaacatttattcttttcatctgtttac | | to TSS) |
| | tcttgctcttgtccaccacaatatgctattcacatgttc | | |
| | agtgtagttttatgacaaagaaaattttctgagttactt | | |
| | ttgtatccccacccccttaaagaaaggaggaaaaactgt | | |
| | ttcatacagaaggcgttaattgcatgaattagagctatc | | |
| | acctaagtgtgggctaatgtaacaaagagggatttcacc | | |
| | tacatccattcagtcagtctttgggggtttaaagaaatt | | |
| | ccaaagagtcatcagaagaggaaaaatgaaggtaatgtt | | |
| | ttttcagacaggtaaagtctttgaaaatatgtgtaatat | | |
| | gtaaaacattttgacacccccataatatttttccagaat | | |
| | taacagtataaattgcatctcttgttcaagagtt | | |
| 117 | gacattgattattgactagttattaatagtaatcaatta | 921 | Interleukin-2 |
| | cggggtcattagttcatagcccatatatggagttccgag | | (IL-2) Full |
| | ttacataacttacggtaaatggcccgcctggctgaccgc | | Promoter (−540 |
| | ccaacgacccccgcccattgacgtcaataatgacgtatg | | to TSS) with |
| | ttcccatagtaacgccaatagggactttccattgacgtc | | upstream CMV |
| | aatgggtggagtatttacggtaaactgcccacttggcag | | Immediate |
| | tacatcaagtgtatcatatgccaagtagccccctattga | | Early Enhancer |
| | cgtcaatgacggtaaatggcccgcctggcattatgccca | | |
| | gtacatgaccttatgggactttcctacttggcagtacat | | |
| | ctacgtattagtcatcgctattaccatgagtatttctct | | |
| | agctgacatgtaagaagcaatctatcttattgtatgcaa | | |
| | ttagctcattgtgtggataaaaaggtaaaaccattctga | | |
| | aacaggaaaccaatacacttcctgttttatcaacaaatc | | |
| | taaacatttattcttttcatctgtttactcttgctcttg | | |
| | tccaccacaatatgctattcacatgttcagtgtagtttt | | |
| | atgacaaagaaaattttctgagttacttttgtatcccca | | |
| | cccccttaaagaaaggaggaaaaactgtttcatacagaa | | |
| | ggcgttaattgcatgaattagagctatcacctaagtgtg | | |
| | ggctaatgtaacaaagagggatttcacctacatccattc | | |
| | agtcagtctttgggggtttaaagaaattccaaagagtca | | |
| | tcagaagaggaaaaatgaaggtaatgttttttcagacag | | |
| | gtaaagtctttgaaaatatgtgtaatatgtaaaacattt | | |
| | tgacacccccataatatttttccagaattaacagtataa | | |
| | attgcatctcttgttcaagagtt | | |

TABLE 5
Table of Chimeric Antigen Receptor (CAR) Related Sequences

| Seq | | Sequence | | DNA, RNA or | |
|---|---|---|---|---|---|
| ID NO | Sequence | Length | Sequence Name | Protein | Organism |
| 118 | GGGGSGGGGSGGGGSGGGGS | 20 | Artificail Linker 4 | Protein | Artificial |
| | | | Repeat of SGGGG | | |
| 119 | GGGGSGGGGSGGGGSGGGGSGGGGS | 25 | Artificial Linker 5 | Protein | Artificial |
| | | | Repeat of SGGGG | | |
| 120 | GGGGSGGGGSGGGGSGGGGSGGGGSGGGGS | 30 | Artificial Linker 6 | Protein | Artificial |
| | | | Repeat of SGGGG | | |
| 121 | GGGGSGGGGSGGGGSGGGGSGGGGSGGGGSGGGGS | 35 | Artificial Linker 7 | Protein | Artificial |
| | | | Repeat of SGGGG | | |
| 122 | GSTSGSGKPGSGEGSTKG | 16 | Artificial Whitlow | Protein | Artificial |
| | | | Linker | | |
| 123 | MALPVTALLLPLALLLHAARP | 21 | Human Cluster of | Protein | Human |
| | | | Differentiation 8 | | |
| | | | alpha (CD8 alpha) | | |
| | | | Signal Peptide | | |
| 124 | MALPVTALLLPLALLLHAARPSQFRVSPLDRTWNLGETVEL | 235 | Human Cluster of | Protein | Human |
| | KCQVLLSNPTSGCSWLFQPRGAAASPTFLLYLSQNKPKAAE | | Differentiation 8 | | |
| | GLDTQRFSGKRLGDTFVLTLSDFRRENEGYYFCSALSNSIM | | alpha (CD8 alpha) | | |
| | YFSHFVFVFLPAKPTTTPAPRPPTPAPTIASQPLSLRPEAC | | Protein with Signal | | |
| | RPAAGGAVHTRGLDFACDIYIWAPLAGTCGVLLLSLVITLY | | Peptide | | |
| | CNHRNRRRVCKCPRPVVKSGDKPSLSARYV | | | | |
| 125 | atggccttaccagtgaccgccttgctcctgccgctggcctt | 708 | Human Derived Coding | DNA | Human |
| | gctgctccacgccgccaggccgagccagttccgggtgtcgc | | DNA of Cluster of | | |
| | cgctggatcggacctggaacctgggcgagacagtggagctg | | Differentiation 8 | | |
| | aagtgccaggtgctgctgtccaacccgacgtcgggctgctc | | alpha (CD8 alpha) | | |
| | gtggctcttccagccgcgcggcgccgccgccagtcccacct | | encoding for Protein | | |
| | tcctcctatacctctcccaaaacaagcccaaggcggccgag | | with Signal Peptide | | |
| | gggctggacacccagcggttctcgggcaagaggttggggga | | | | |
| | caccttcgtcctcaccctgagcgacttccgccgagagaacg | | | | |
| | agggctactatttctgctcggccctgagcaactccatcatg | | | | |
| | tacttcagccacttcgtgccggtcttcctgccagcgaagcc | | | | |
| | caccacgacgccagcgccgcgaccaccaacaccggcgccca | | | | |
| | ccatcgcgtcgcagcccctgtccctgcgcccagaggcgtgc | | | | |
| | cggccagcggcggggggcgcagtgcacacgagggggctgga | | | | |
| | cttcgcctgtgatatctacatctgggcgcccttggccggga | | | | |
| | cttgtggggtccttctcctgtcactggttatcaccctttac | | | | |
| | tgcaaccacaggaaccgaagacgtgtttgcaaatgtccccg | | | | |
| | gcctgtggtcaaatcgggagacaagcccagcctttcggcga | | | | |
| | gatacgtctaa | | | | |
| 126 | MLRLLLALNLFPSIQVTG | 18 | Human T-cell-specific | Protein | Human |
| | | | surface glycoprotein | | |
| | | | CD28 (CD28) Signal | | |
| | | | Peptide | | |
| 127 | MLRLLLALNLFPSIQVTGNKILVKQSPMLVAYDNAVNLSCK | 220 | Human T-cell-specific | Protein | Human |
| | YSYNLFSREFRASLHKGLDSAVEVCVVYGNYSQQLQVYSKT | | surface glycoprotein | | |
| | GFNCDGKLGNESVTFYLQNLYVNQTDIYFCKIEVMYPPPYL | | CD28 (CD28) Protein | | |
| | DNEKSNGTIIHVKGKHLCPSPLFPGPSKPFWVLVVVGGVLA | | with Signal Peptide | | |
| | CYSLLVTVAFIIFWVRSKRSRLLHSDYMNMTPRRPGPTRKH | | | | |
| | YQPYAPPRDFAAYRS | | | | |
| 128 | atgctcaggctgctcttggctctcaacttattcccttcaat | 663 | Human Derived Coding | DNA | Human |
| | tcaagtaacaggaaacaagattttggtgaagcagtcgccca | | DNA of T-cell-specific | | |
| | tgcttgtagcgtacgacaatgcggtcaaccttagctgcaag | | surface glycoprotein | | |
| | tattcctacaatctcttctcaagggagttccgggcatccct | | CD28 (CD28) encoding | | |
| | tcacaaaggactggatagtgctgtggaagtctgtgttgtat | | for Protein with | | |
| | atgggaattactcccagcagcttcaggtttactcaaaaacg | | Signal Peptide | | |
| | gggttcaactgtgatgggaaattgggcaatgaatcagtgac | | | | |
| | attctacctccagaatttgtatgttaaccaaacagatattt | | | | |
| | acttctgcaaaattgaagttatgtatcctcctccttaccta | | | | |
| | gacaatgagaagagcaatggaaccattatccatgtgaaagg | | | | |
| | gaaacacctttgtccaagtcccctatttcccggaccttcta | | | | |
| | agcccttttgggtgctggtggtggttggtggagtcctggct | | | | |
| | tgctatagcttgctagtaacagtggcctttattattttctg | | | | |
| | ggtgaggagtaagaggagcaggctcctgcacagtgactaca | | | | |
| | tgaacatgactccccgccgccccgggcccacccgcaagcat | | | | |
| | taccagccctatgccccaccacgcgacttcgcagcctatcg | | | | |
| | ctcctga | | | | |
| 129 | IEVMYPPPYLDNEKSNGTIIHVKGKHLCPSPLFPGPSKP | 39 | Human Derived T-cell- | Protein | Human |
| | | | specific surface | | |
| | | | glycoprotein CD28 | | |
| | | | (CD28) Hinge | | |
| 130 | FWVLVVVGGVLACYSLLVTVAFIIFWV | 27 | Human Derived T-cell- | Protein | Human |
| | | | specific surface | | |
| | | | glycoprotein CD28 | | |
| | | | (CD28) transmembrane | | |
| | | | Domain | | |
| 131 | RSKRSRLLHSDYMNMTPRRPGPTRKHYQPYAPPRDFAAYRS | 41 | Human Derived T-cell- | Protein | Human |
| | | | specific surface | | |
| | | | glycoprotein CD28 | | |
| | | | (CD28) co-stimulatory | | |
| | | | signaling Sequence | | |
| 132 | IEVMYPPPYLDNEKSNGTIIHVKGKHLCPSPLFPGPSKPFW | 107 | Human Derived T-cell- | Protein | Human |
| | VLVVVGGVLACYSLLVTVAFIIFWVRSKRSRLLHSDYMNMT | | specific surface | | |
| | PRRPGPTRKHYQPYAPPRDFAAYRS | | glycoprotein CD28 | | |
| | | | (CD28) Hinge | | |
| | | | Transmembrane domain | | |
| | | | and co-stimulatory | | |
| | | | signaling sequence | | |
| 133 | IWAPLAGTCGVLLLSLVITLYC | 22 | Human Derived Cluster | Protein | Human |
| | | | of Differentiation 8 | | |
| | | | alpha (CD8 alpha) | | |
| | | | transmembrane Domain | | |
| 134 | TTTPAPRPPTPAPTIASQPLSLRPEACRPAAGGAVHTRGLD | 47 | Human Derived Cluster | Protein | Human |
| | FACDIY | | of Differentiation 8 | | |
| | | | alpha (CD8 alpha) | | |
| | | | hinge | | |
| 135 | MGNSCYNIVATLLLVLNFERTRS | 23 | Human Cluster of | Protein | Human |
| | | | Differentiation 137 | | |
| | | | (4-1BB) Signal Peptide | | |
| 136 | | | Skip | Protein | Human |
| 137 | MGNSCYNIVATLLLVLNFERTRSLQDPCSNCPAGTFCDNNR | 255 | Human Cluster of | Protein | Human |
| | NQICSPCPPNSFSSAGGQRTCDICRQCKGVFRTRKECSSTS | | Differentiation 137 | | |
| | NAECDCTPGFHCLGAGCSMCEQDCKQGQELTKKGCKDCCFG | | (CD137) referred to as | | |
| | TFNDQKRGICRPWTNCSLDGKSVLVNGTKERDVVCGPSPAD | | 4-1BB Ligand (4-1BB) | | |
| | LSPGASSVTPPAPAREPGHSPQIISFFLALTSTALLFLLFF | | Protein with Signal | | |
| | LTLRFSVVKRGRKKLLYIFKQPFMRPVQTTQEEDGCSCRFP | | Peptide | | |
| | EEEEGGCEL | | | | |
| 138 | atgggaaacagctgttacaacatagtagccactctgttgct | 768 | Human Derived Coding | DNA | Human |
| | ggtcctcaactttgagaggacaagatcattgcaggatcctt | | DNA for Cluster of | | |
| | gtagtaactgcccagctggtacattctgtgataataacagg | | Differentiation 137 | | |
| | aatcagatttgcagtccctgtcctccaaatagtttctccag | | (CD137) referred to as | | |
| | cgcaggtggacaaaggacctgtgacatatgcaggcagtgta | | 4-1BB Ligand (4-1BB) | | |
| | aaggtgttttcaggaccaggaaggagtgttcctccaccagc | | encoding for Protein | | |
| | aatgcagagtgtgactgcactccagggtttcactgcctggg | | with Signal Peptide | | |
| | ggcaggatgcagcatgtgtgaacaggattgtaaacaaggtc | | | | |
| | aagaactgacaaaaaaaggttgtaaagactgttgctttggg | | | | |
| | acatttaacgatcagaaacgtggcatctgtcgaccctggac | | | | |
| | aaactgttctttggatggaaagtctgtgcttgtgaatggga | | | | |
| | cgaaggagagggacgtggtctgtggaccatctccagccgac | | | | |
| | ctctctccgggagcatcctctgtgaccccgcctgcccctgc | | | | |
| | gagagagccaggacactctccgcagatcatctccttctttc | | | | |
| | ttgcgctgacgtcgactgcgttgctcttcctgctgttcttc | | | | |
| | ctcacgctccgtttctctgttgttaaacggggcagaaagaa | | | | |
| | actcctgtatatattcaaacaaccatttatgagaccagtac | | | | |
| | aaactactcaagaggaagatggctgtagctgccgatttcca | | | | |
| | gaagaagaagaaggaggatgtgaactgtga | | | | |
| 139 | KRGRKKLLYIFKQPFMRPVQTTQEEDGCSCRFPEEEEGGCE | 42 | Human Derived Cluster | Protein | Human |
| | L | | of Differentiation 137 | | |
| | | | (CD137) referred to as | | |
| | | | 4-1BB Ligand (4-1BB) | | |
| | | | co-stimulatory | | |
| | | | Sequence | | |
| 140 | MKWKALFTAAILQAQLPITEA | 21 | Human Cluster of | Protein | Human |
| | | | Differentiation 3 Zeta | | |
| | | | Chain (CD3 Zeta) | | |
| | | | Signal Peptide | | |
| 141 | MKWKALFTAAILQAQLPITEAQSFGLLDPKLCYLLDGILFI | 164 | Human Cluster of | Protein | Human |
| | YGVILTALFLRVKFSRSADAPAYQQGQNQLYNELNLGRREE | | Differentiation 3 Zeta | | |
| | YDVLDKRRGRDPEMGGKPQRRKNPQEGLYNELQKDKMAEAY | | Chain (CD3 Zeta) | | |
| | SEIGMKGERRRGKGHDGLYQGLSTATKDTYDALHMQALPPR | | Protein with Signal | | |
| | | | Peptide | | |
| 142 | atgaagtggaaggcgcttttcaccgcggccatcctgcaggc | 495 | Human Derived Coding | DNA | Human |
| | acagttgccgattacagaggcacagagctttggcctgctgg | | DNA of Cluster of | | |
| | atcccaaactctgctacctgctggatggaatcctcttcatc | | Differentiation 3 Zeta | | |
| | tatggtgtcattctcactgccttgttcctgagagtgaagtt | | (CD3 Zeta) encoding | | |
| | cagcaggagcgcagacgcccccgcgtaccagcagggccaga | | for Protein with | | |
| | accagctctataacgagctcaatctaggacgaagagaggag | | Signal Peptide | | |
| | tacgatgttttggacaagagacgtggccgggaccctgagat | | | | |
| | ggggggaaagccgcagagaaggaagaaccctcaggaaggcc | | | | |
| | tgtacaatgaactgcagaaagataagatggcggaggcctac | | | | |
| | agtgagattgggatgaaaggcgagcgccggaggggcaaggg | | | | |
| | gcacgatggcctttaccagggtctcagtacagccaccaagg | | | | |
| | acacctacgacgcccttcacatgcaggccctgccccctcgc | | | | |
| | taa | | | | |
| 143 | RVKFSRSADAPAYQQGQNQLYNELNLGRREEYDVLDKRRGR | 112 | Human Derived Cluster | Protein | Human |
| | DPEMGGKPRRKNPQEGLYNELQKDKMAEAYSEIGMKGERRR | | of Differentiation 3 | | |
| | GKGHDGLYQGLSTATKDTYDALHMQALPPR | | Zeta Chain (CD3 Zeta) | | |
| | | | Signaling Domain | | |

Mice or Other Non-Human Vertebrate with Engineered Immune Systems (Transgenic Mice):

This instant patent contemplates the use of mice with engineered immune systems or other non-human vertebrate with engineered immune systems so that it produces fully human antibodies in place of the murine or non-human vertebrate antibodies is an important for the discovery of antibodies with therapeutic potential. Engineered animals such as mice for this purpose are capable of producing a fully antibody repertoire capable of affinity maturation towards the antigen target. Engineered animals are capable of highly evolved in-vivo mechanisms such as hypermutation in germinal centers resulting in fully human (comprising human variable and constant regions) high affinity antibodies with optimal biophysical properties that because they are developed in vivo in the host non-human vertebrate are less likely to cause immunogenicity in humans. Use of non-human vertebrates with engineered immune systems provides a straight-forward approach to rapidly obtain antigen specific antibodies that have undergone recombination, junctional diversification, affinity maturation and isotype switching in vivo in a non-human vertebrate system. Fully human antibodies avoid the problem of attenuating antibody characteristics when humanizing the constant region of chimeric antibodies and thus can lead to more exquisite specificity for the target with reduced overall effort and cost. The earliest in vivo non-human vertebrate used for this purpose in vivo was Xenomouse™ that used completely human transgenic heavy chain loci which comprise human variable regions (human V_{H}, D and JH gene segments) upstream of human constant regions. Subsequently, it has been discovered that the use of 60 totally human transgenic loci in such in vivo systems is detrimental and B-cell development is hampered, leading to relatively small B-cell compartments and restricted utility for generating antibodies. Later-generation transgenic animals (e.g., the Velocimouse™) have been created which have 65 chimeric heavy chain loci in which a human variable region is upstream of endogenous (e.g., mouse or rat) constant expression. US2008/0196112A1 (Innate Pharma) discloses transgenic animals comprising a single, predetermined human rearranged VDJ from a lead antibody, together with one or more human constant region genes in a locus. (See e.g. For example, see U.S. Pat. Nos. 10,251,377 B2 and 10,667,501 B2 expressly incorporated by reference herein in their entirety; Also see Lee, E. C., Liang, Q., Ali, H. et al., 2014, Nat. Biotechnol., 32:356-363). These mice with engineered immune systems serve as an efficient means to discover immunoglobulins and immunoglobulin V regions that are potent for the target of interest. Potent antibodies may be discovered through FACS cell sorting or magnetic beads biotinylated to the antigen of interest. In one embodiment the incorporation of such V regions is made into dIgA1 and polymeric immunoglobulin A1 or engineered variants DNA and mRNA vector constructs. In another embodiment the incorporation of such V regions is made into dIgA2 and polymeric immunoglobulin A2 or engineered variants DNA and mRNA vector constructs.

### Tissue Targeting

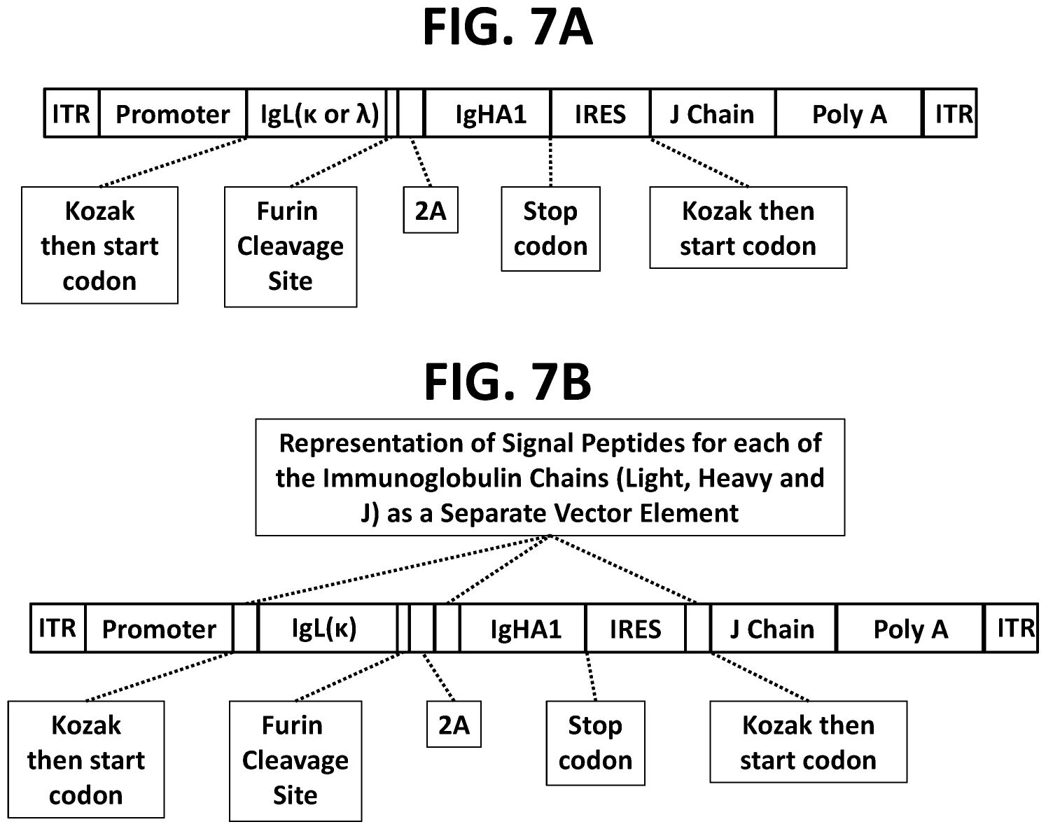

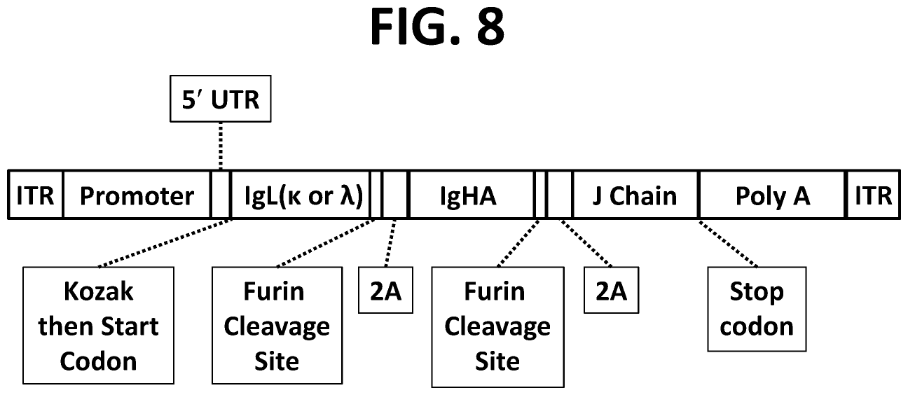

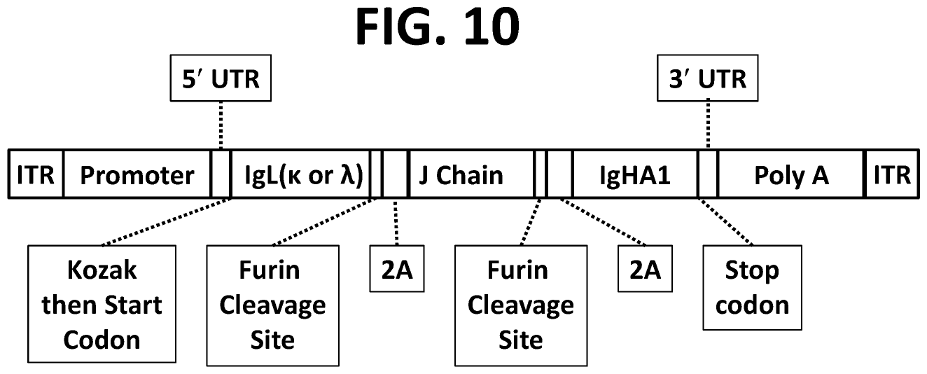

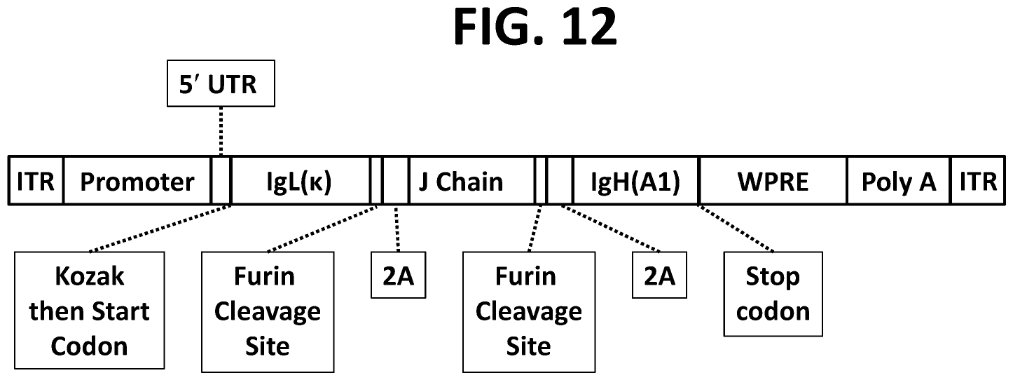

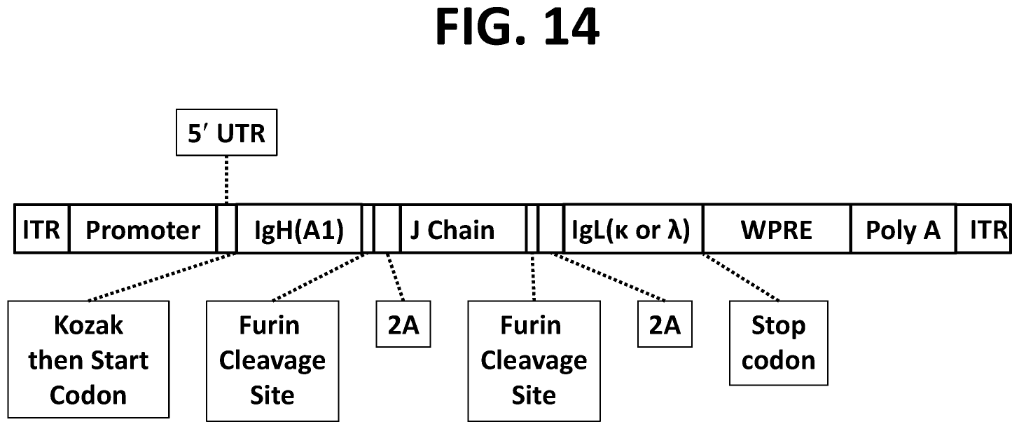

The instant patent invention contemplates the delivery of an episomally-maintained gene therapy based vaccination/immunization as an intramuscular injection, intravenous injection or injection proximal to lymph nodes that can be an intramuscular injection and administration to the lamina propria via endoscopic injection as well as both ex vivo administration of the gene therapy to immune cells. AAV vectors (See FIGS. 6, 7, 8, 9, 10, 11, 12, 13, 14, 15 and 16 as examples) may be delivered intramuscularly where that AAV capsid may transduce the skeletal muscle cell (myocyte) and deliver the vector to muscle nuclei where the vectors may form concatemers or reside as monomeric circular gene elements. Skeletal muscles are particularly beneficial because they are a non-dividing cell population. Thus, episomes not capable of self-replication may persist for years in the skeletal muscle nuclei. Also, targeted in the liver are hepatocytes that have half-lives of 200-400 days and thus are ideal for longer term expression of dIgA1 and polymeric immunoglobulin A1 and dIgA2 and polymeric immunoglobulin A2. AAV capsids are also effective at targeting hepatocytes. In one embodiment naked vector DNA is delivered to muscle cells with the use of electroporation. It has been shown that electroporation increases gene transfection efficiency to the muscle nuclei by 100-fold. Electroporation may be defined in this instance as low voltage pulses to the muscle, which allows macromolecules such as DNA vectors to enter the cells more efficiently. (See e.g., Tjelle, T. E., et. al., 2004, Mol. Ther., 9:328-336; Andrews, C. D., et al., 2017, Methods and Clinical Development, 7:74-82) Additionally, pseudotyped lentiviral vectors, and pseudotyped gammaretroviral vectors and or lipid nanoparticles with site specific targeting ligands may target liver cells (hepatocytes) bearing specific CD receptors. In another embodiment hepatocytes are targeted such as with AAV8 another privileged cell type capable of expressing Immunoglobulins (See e.g. Lei, Y., Huang, T., Su, M. et al., 2014, Lab Invest 94:1283-1295) and have a half-lives of 200-400 days.) In a further embodiment the lamina propria of the lungs e.g. trachea or the stomach and duodenum are be targeted to deliver lentiviral vectors or gammaretroviral vectors with the aim of delivering such vectors to the germinal center memory B-cells in the supporting lymph nodes and memory B-cells in the interstitium for the particular tissue.

### Promoters, Enhancers, Entry Sites and Cis Acting Signals Used in Vectors

The vectors of the invention include heterologous control sequences, which include, but are not limited to, constitutive promoters, such as the human cytomegalovirus (CMV) immediate early enhancer/promoter (SEQ ID NO: 1), Rous sarcoma virus (RSV), simian virus 40 (SV40) and mammalian elongation factor 1 alpha (EF-1α), are non-specific promoters and are commonly used in gene therapy vectors. Other promoters that are commonly used in gene therapy include cytomegalovirus enhancer/chicken beta-actin (CAG). Other promoters include mouse phosphoglycerate kinase (mPGK) and human synapsin (hSYN) promoter. Preferred promoters include the EF1-alpha promoter (Kim et al., Gene 91(2): 217-23 (1990)) and Guo et al., Gene Ther. 3(9):802-10 (1996)). Highly preferred promoters include the human cytomegalovirus (CMV) immediate early gene enhancer/promoter, elongation factor 1-alpha (EF1a) promoter, a cytomegalovirus enhancer/chicken beta-actin (CAG) promoter and a simian virus 40 (SV40) promoter. Other promoters include a tetracycline responsive promoter (TRE), a transthyretin promoter (TTR), and a CK6 promoter. The sequences of these and numerous additional promoters are known in the art. The relevant sequences may be readily obtained from public databases and incorporated into vectors for use in practicing the present invention. In some cases the relevant sequences are published in a Journal Article. In some instances human promoters and abbreviated forms of those promoters for cytokines and chemokines are considered.

Other promoters that may be used in practicing the invention include promoters for cytokines and chemokines used as part of the immune response. Considered are promoters and their upstream or downstream regions and even truncated promoters with the necessary signals for gene expression collectively specific to upregulated cytokines and chemokines that CAR engineered cell lines secrete or upregulate upon activation including perforin (SEQ ID NO: 110), granzyme B (SEQ ID NO: 113), IFNγ (SEQ ID NO: 111 and SEQ ID NO:112), TNFα (SEQ ID NO: 106, SEQ ID NO: 107, SEQ ID NO:108 and SEQ ID NO: 109) and IL-2 (SEQ ID NO: 115, SEQ ID NO:116 and SEQ ID NO: 117). (For granzyme B promoter identification see e.g., Wargnier, A., Legros-Maida, S., Bosselut, R., Bourge, J. F., Lafaurie, C., Ghysdael, C. J., Sasportes, M., & Paul, P., 1995, Proceedings of the National Academy of Sciences of the United States of America, 92: 6930-6934. For IFNγ promoter identification see e.g. Penix, L., Weaver, W. M., Pang, Y., Young, H. A., & Wilson, C. B., 1993, The Journal of experimental medicine, 178: 1483-1496. For IL-2 promoter identification see e.g. Attema J L, Reeves R, Murray V, Levichkin I, Temple M D, Tremethick D J, Shannon M F., 2002, J Immunol. 169:2466-76. For TNFα promoter identification see e.g., Tsai, E. Y., Falvo, J. V., Tsytsykova, A. V., Barczak, A. K., Reimold, A. M., Glimcher, L. H., Dunn, I. F., & Goldfeld, A. E. et al., 2000, Molecular and cellular biology, 20: 6084-6094. For perforin promoter identification see e.g., Yu C R, Ortaldo J R, Curiel R E, Young H A, Anderson S K, Gosselin P., 1999, J Immunol. 162:2785-90.)

Typically, in order to express 2 or more proteins in a vector two or more promoters are required. As an example, an internal ribosome entry site (IRES) sequence is often used to drive expression of individual genes when those genes are located 5′ to at least one gene. Alternatively, an elongation factor 1-alpha promoter (EF1α) may be used where a polyadenylation site may be used to process the mRNA of the preceding gene. When two genes are linked with an IRES sequence, the expression level of the second gene is often significantly weaker than the first gene (Furler et al., Gene Therapy 8:864-873, 2001). However, one may use as will be understood by those of skill in the art, expression vectors typically include a promoter operably linked to the coding sequence or sequences to be expressed, as well as ribosome binding sites, RNA splice sites, a polyadenylation site, and transcriptional terminator sequences, as appropriate to the coding sequence(s) being expressed. Other sequences such as a Kozak sequence (SEQ ID NO: 20) such as “GCCACC” are introduced directly before the transcription start codon to allow for the expression of a transgene. The Kozak sequence is also important for the second transcription start codon that is directly preceded by an IRES or directly preceded by a second promoter. In general the Kozak sequence is necessary for translation initiation at any place on mRNA.

Increased levels of gene expression generally occurs when a Woodchuck hepatitis-virus posttranscriptional regulatory element (WPRE) is incorporated as DNA (SEQ ID NO: 3) and as RNA (SEQ ID NO: 72). WPRE is most effective when placed downstream of the transgene, following the 3′ UTR. Although, in some embodiments the WPRE is placed upstream of the 3′ UTR. WPRE are incorporated into vectors to ensure high levels of gene expression. In some embodiments WPRE is incorporated into the vectors. In other embodiments two consecutive WPRE regulatory elements are used. Generally, WPRE and a polyadenylation signal can substantially increase gene expression over the vector that only includes the polyadenylation signal. When a retroviral vector is used such as a lentiviral vector or gammaretroviral vector are used to express the beneficial gene the polyadenylation signal is identified as the U5 of the LTR.

### Prolonged Gene Expression

Episomally maintained vectors with self-replicating elements are capable of long-term episomal persistence in human cells and may be referred to as episomally-maintained self-replicating vectors. Typically, these vectors are based on few designs. One common design contains components derived from either EBV or bovine papilloma virus, where there is a greater emphasis for gene therapy related applications with EBV. EBV is a human herpesvirus that can maintain its genome extra-chromosomally as an episome in dividing mammalian cells. Maintenance is also achieved with the EBV viral latent origin of replication oriP and EBV nuclear antigen 1 (EBNA1) that act together to replicate and retain the viral genome in the nucleus. Thus, inclusion of these sequences allows them to be maintained in dividing cells. (See e.g., Conese, M., et. al., 2004, Gene Ther., 11:1735-1741) Although, there are substantial safety and immunotolerance challenges associated with the EBNA1 protein making it unsuitable for present application.

### Vectors for Use in Practicing the Invention

The present invention contemplates the use of non-viral vectors, viral vectors and retroviral vectors as an efficient and effective means to express immunoglobulins whose polypeptide sequence is derived from humans especially human memory B-cells or plasmablasts, a transgenic animal, a mouse with humanized immune system, or a mouse any of which that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment 8) an allergen or cause of allergies, (9) a target protein or variant (10) an immune system protein such as Immunoglobulin class E (IgE) or a cytokine. Additionally, scF_{V} or Fab identified from phage display technology may be used to identify such potent immunoglobulin V or Fab regions. In another context only the V-regions (V_{L} and V_{H}), CDR regions or Fab regions may be used from humans B-cells, B-cells from transgenic mice, B-cells from mice or other non-human vertebrate with humanized immune systems and in some cases portions of the constant regions such as one or more of C_{H}1 and CL polypeptide sequence or either of Fab or F(ab′)2 polypeptide are derived from the immunoglobulin polypeptide sequence of memory B-cells or plasmablasts. This strategy provides flexibility to use entire immunoglobulin polypeptide sequences or only V-region sequences in conjunction with other constant region sequences or including isotype switched constant region sequences, engineered constant region sequences that cannot be detected by Fc receptors and even a combination of constant region sequences from two sources. E.g. using one or more of C_{H}1, the hinge and C_{H}2 polypeptide sequences from another immunoglobulin isotype. Although, C_{H}3 must be that from IgA1 or IgA2 due to its importance in forming a cysteine bond with J Chain (see FIG. 1) and contributing to the formation of a central Beta sheet complex (Beta sandwich) with J Chain that takes place between each immunoglobulin heavy chain and J Chain. This requirement does not preclude the C_{H}3 domain of IgA1 or IgA2 from being engineered such as to enhance formation of high valency polymeric immunoglobulin A1 or A2 respectively. Additionally, CDRs may be used in conjunction with Framework regions from another human or even mouse source. When mice are used to identify potent immunoglobulins for (1) a virus (s) (2) a systemic ailment such as allergies (3) fungi (4) bacterial infection (4) cancerous tumor (5) biowarfare agent (6) microbial infection, (7) any ailment 8) an allergen or cause of allergies, (9) a target protein or variant (10) an immune system protein such as Immunoglobulin class E (IgE) or a cytokine chimeric immunoglobulins will be developed.

In the invention the vector constructs non-viral, viral and retroviral delivery systems then effectively serve as the mechanism to realize that immunoglobulin-based binding affinity that was discovered in humans, transgenic mice or other non-human vertebrate with an engineered immune system, mice with a resulting chimeric antibody, or mice with humanized immune systems when considering the expression of dIgA1 and polymeric immunoglobulin A1, dIgA2 and polymeric immunoglobulin A2, or engineered variants. However, the present invention further considers the use of vector constructs to express dIgA and polymeric immunoglobulin A that encode for the immunoglobulin heavy chain, the immunoglobulin light chain (kappa or lambda), J Chain and optional use of MZB1 in some embodiments on a single vector construct in order to also engender recipients with mucosal immunity, epithelial immunity or cancer immunity against the pathogen of interest. In these contexts the present invention considers the use of various vectors to introduce DNA constructs that comprise these coding sequences. The expression of dIgA1 that would include the expression of polymeric immunoglobulin A1 or dIgA2 that would include polymeric immunoglobulin A2 in a single vector construct is important since one can be assured that if the cell receives a single vector the vector contains all the information necessary to encode for the desired immunoglobulin without requiring another delivery system to deliver the gene therapy to the same cell.

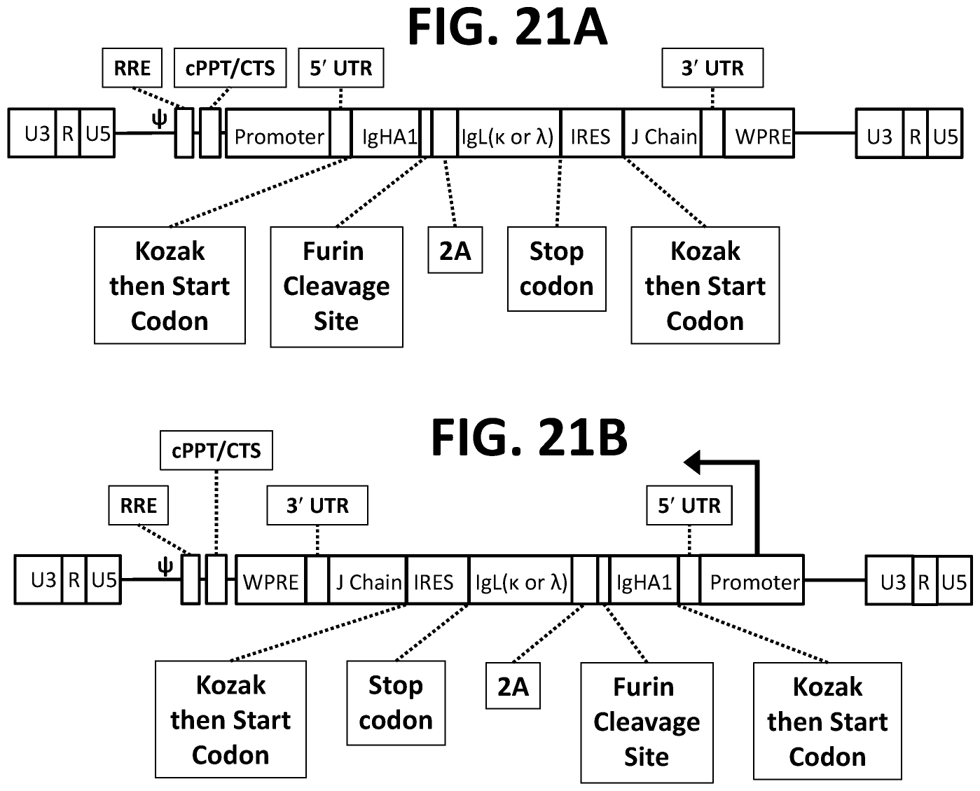

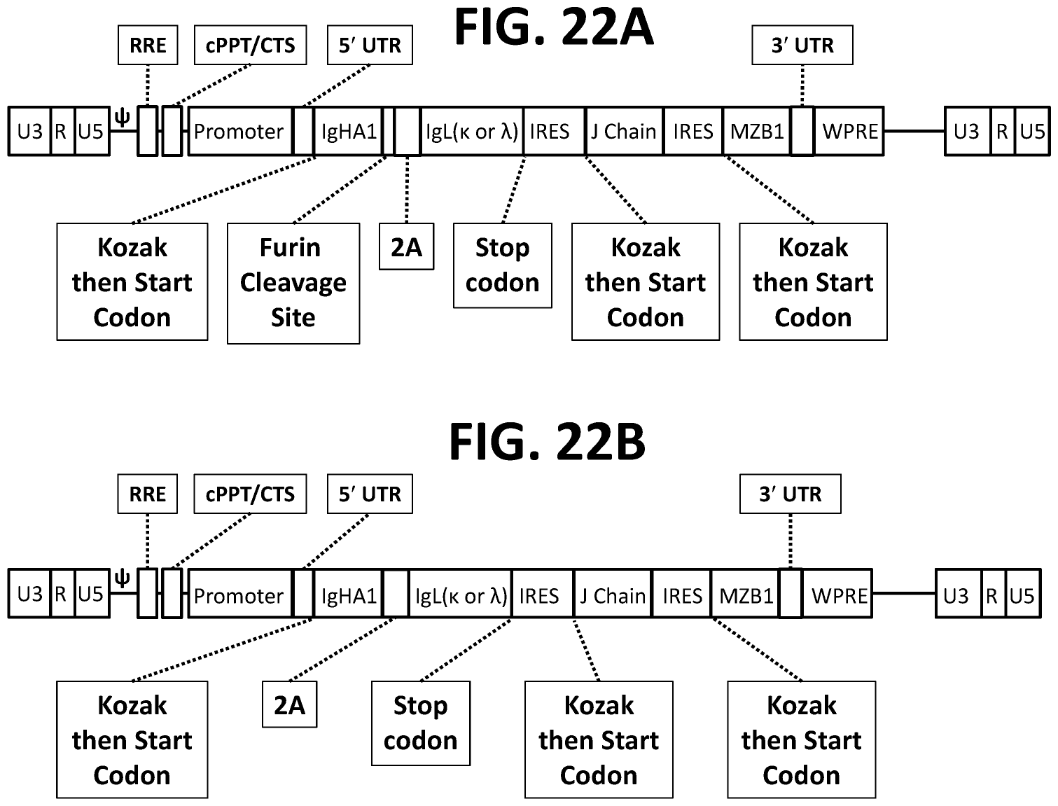

Any of a variety of vectors for introduction of constructs (See FIGS. 6 through 24 as examples) comprising the coding sequence for immunoglobulins that in some embodiments includes the use of 2A self-processing peptide sequences and furin cleavage sites. There are an extensive number of examples of gene expression vectors that are known in the art and such vectors may be viral and non-viral as well as self-replicating or replication deficient. Although, the use of self-replicating vectors to express immunoglobulins with integration competent lentiviral based gene delivery methods excludes the use of rAAV vectors that have a 4.9 Kb capacity. Non-viral, viral and retroviral gene delivery methods that may be employed in the practice of the invention include but are not limited to plasmids, lipid nanoparticles, liposomes, nucleic acid/liposome complexes and cationic lipids.

Reference to a vector as “recombinant” refers to the linkage of DNA sequences which are not known in nature and which come from two or more organisms in nature. Reference to the “transgene” refers to the gene encoding for a polypeptide in the vector that is intended for some function in the organism that is unrelated to the maintenance or support of the vector function. Expression of the transgene is regulated by sequences that are operatively linked to a polypeptide coding sequence when the expression and/or control sequences regulate the transcription and/or translation of the nucleic acid sequence. Thus expression and/or control sequences can include promoters, enhancers, transcription terminators, polyadenylation sites, furin cleavage sites, 2A self-processing peptide, replicating elements, a start codon (e.g., ATG) 5′ to the coding sequence, Kozak sequences, splicing signals for introns, a 5′ UTR, a 3′ UTR and stop codons. (For example, see U.S. Pat. Nos. 7,498,024 B2 and 7,709,224 B2 expressly incorporated by reference herein in their entirety)

Recombinant Adeno Associated Virus (rAAV) vectors are established to exhibit strong transient expression with excellent titer and the ability to enter dividing and non-dividing human cells without eliciting an immune response for most—upon first time exposure—to the AAV capsid protein structure. However, multiple exposures to an AAV capsid may elicit an immune response. Although, advances in science may likely overcome the immunogenicity of AAV that are also able to attain the same safety profile as currently clinically approved rAAVs. rAAV vectors delivered via AAV capsids have the ability to enter cells that depends in part on AAV serotype. Further, AAVs are favored for in vivo episomal gene transfer of therapeutic genes because of their low immunogenicity, strong safety record, and high efficiency of transduction of a number of cell types in animal models. (Wu, Z., et. al., 2006, Mol Ther. 14:316-327.) Replication-defective AAV virions encapsulating the recombinant AAV vectors of the instant invention are made by standard techniques known in the art using AAV packaging cells and packaging technology. The coding sequence for two or more polypeptides or proteins of interest is commonly inserted into the adenovirus in the deleted E3 region of the virus genome. The recombinant vectors of this invention comprise the ability to express dIgA1 (inclusive of polymeric immunoglobulin A1), dIgA2 (inclusive of polymeric immunoglobulin A2), engineered variants or dscFV-FcIgA. Further the recombinant vectors of this invention encode for immunoglobulins that are naturally developed in humans especially human memory B-cells or plasmablasts, discovered from a transgenic animal, discovered from a mouse or other non-human vertebrate with ahumanized immune system, discovered from a mouse, discovered from a mouse and intended for a chimeric antibody any of which that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment 8) an allergen or cause of allergies, (9) a target protein or variant (10) an immune system protein such as Immunoglobulin class E (IgE) or a cytokine. In some embodiments only a portion—that must include one or more of the CDRs and V-region segments of both the immunoglobulin light and heavy chains—of the polypeptide sequence encoding for the potent immunoglobulins will be used in the vector construct. The elements of the recombinant AAV vectors may comprise (A) a packaging site that enables the vector to be placed in replication incompetent AAV virions; (B) the coding sequence for immunoglobulin gene elements e.g. heavy chain (IgH), light chain (IgL), J Chain and optionally an associated proteins e.g. MZB1; (C) the optional use of the sequence encoding for a 2A self-processing peptide site located C-terminal to a furin cleavage site. (D) a modified 2A self-processing peptide may be substituted for a full length 2A self-processing peptide to minimally contain the consensus sequence (C) in some cases with potentially some loss in efficiency (E) the optional use of a multi-promoter rAAV vector for immunoglobulin expression. (F) the optional use of both (C) and (D) in a single vector. (See FIGS. 6A, 7A, 7B, 8, 9A, 10, 12, 13, 14, 15 and 16 as examples). Other elements necessary for or which improve the formation and/or function of the vectors or vector packaging including ITRs. All these methods are well within the practice of those skilled in the art. (For example, see U.S. Pat. Nos. 7,498,024 B2 and 7,709,224 B2)

Adeno-associated virus (AAV) is a helper-dependent human parvovirus, which is able to infect cells latently by chromosomal integration or delivery of an episomally maintain gene element to the nucleus. AAV has significant potential as a human gene therapy vector. For use in practicing the present invention rAAV virions may be produced using standard methodology, known to those of skill in the art and are constructed such that they include, as operatively linked components in the direction of transcription, control sequences including transcriptional initiation and termination sequences, and the coding sequence(s) of interest. More specifically, in the invention in one embodiment the recombinant AAV vectors of the instant invention comprise: (1) a packaging site enabling the vector to be incorporated into replication-defective AAV virions; (2) the coding sequence for two or more polypeptides or proteins of interest, e.g., heavy and light chains of an immunoglobulin of interest; (3) the optional use of a sequence encoding a 2A self-processing cleavage site alone or in combination with an additional furin cleavage site. (4) optional use of separate promoters and regulatory elements for each coding sequence (5) Optional use of a combination of (3) and (4). AAV vectors for use in practicing the invention are constructed such that they also include, as operatively linked components in the direction of transcription, control sequences including transcriptional initiation and termination sequences. In another embodiment these components are flanked on the 5′ and 3′ end by functional AAV ITR sequences. By “functional AAV ITR sequences” is meant that the ITR sequences function as intended for the rescue, replication and packaging of the AAV vectors. Further, AAV vectors for use in practicing the invention in one embodiment will code for (A) immunoglobulins that are derived from immunoglobulins identified from the blood of leukocytes from humans especially human B-cells, memory B-cells, plasmablasts, discovered from a transgenic animal, a mouse with humanized immune system, or a mouse any of which that was infected with, exposed to, has immune specificity to, or lives with any of (1) a virus (s) (2) a systemic ailment such as allergies (3) fungi (4) bacterial infection (4) cancerous tumor (5) biowarfare agent (6) microbial infection, (7) any ailment 8) an allergen or cause of allergies, (9) a target protein or variant (10) an immune system protein such as Immunoglobulin class E (IgE) or a cytokine. Or such scFv identified from Phage Display technology. (B) immunoglobulins whose CDR or V-regions (V_{L} and V_{H}) are derived from immunoglobulins identified in identified from any of the same sources discussed in part (A) of this section. (C) immunoglobulins whose antibody binding fragments (Fab): V-regions (V_{L} and V_{H}) and constant heavy domain 1 C_{H}1 and constant light (CL) chain domain are derived from immunoglobulins identified from any of the same sources discussed in part (A) of this section. (D) immunoglobulins whose antibody binding fragments that results from pepsin cleavage (Fab)₂: V-regions (V_{L} and V_{H}) and constant heavy region 1 C_{H}1, hinge and constant light (C_{L}) chain domain are derived from immunoglobulins identified from any of the same sources discussed in part (A) of this section. (E) Dimeric Immunoglobulin A (dIgA) that requires cDNA coding for or the polypeptide sequence of the immunoglobulin class A immunoglobulin heavy and light chains, J Chain with optional use of the signal peptide (see SEQ IDs 11 and 7) and optional encoding of MZB1. (For similar examples, see U.S. Pat. Nos. 7,498,024 B2 and 7,709,224 B2; Also see e.g., Fang, J., et al., 2005, Nat. Biotechnol., 23: 584-590; Krysan, D. J., et. al., 1999, J Biol Chem. 274:23229-23234)

Recombinant AAV (rAAV) vectors are capable of directing the expression and production of selected recombinant polypeptide or protein products in target cells. Thus, the recombinant vectors comprise at least all of the sequences of AAV essential for encapsidation of the vector and the physical structures or inverted terminal repeats (ITRs) for infection of the recombinant AAV virions. Thus, AAV ITRs for use in the vectors of the invention need not have a wild-type nucleotide sequence (e.g., as described in Kotin, H., 1994, Gene Ther., 5:793-801), and may be altered by the insertion, deletion or substitution of nucleotides or the AAV ITRs may be derived from any of several AAV serotypes. Generally, an AAV vector is a vector derived from an adeno-associated virus serotype, including without limitation, AAV-1, AAV-2, AAV-3, AAV-4, AAV-5, AAV-6, AAV-7, AAV-8, etc. Preferred rAAV vectors have the wild type REP and CAP genes deleted in whole or part, but retain functional flanking ITR sequences that are necessary for the vector to be efficiently encapsidated by the AAV capsid to form the rAAV virion. (For example, see U.S. Pat. Nos. 7,498,024 B2 and 7,709,224 B2 expressly incorporated by reference herein in their entirety) Among the most widely used platforms for producing rAAV involves transfecting Human Embryonic Kidney 293 (HEK293) cells with either two or three plasmids; one encoding the gene of interest, one carrying the AAV rep/cap genes, and another containing helper genes provided by either adeno or herpes viruses which contains the E4, E2a and VA helper genes that mediate AAV replication (See e.g., Janik, J. E., et. al., 1981, “Proc. Natl. Acad. Sci., 78:1925-1929; Buller, R. M. L., et al., 1981, J, Virol. vol. 40:241-24; Matsushita, T., et. al., 1998, Gene Ther., 5:938-945) The helper construct includes AAV coding regions that are capable of being expressed in the producer cell and which complement the AAV helper function that is absent in the AAV vector. Without such helper genes AAV production is generally reduced by at least a factor of 100. The helper construct may be designed to down regulate the expression of the large Rep proteins (Rep78 and Rep68), typically by mutating the start codon following p5 from ATG to ACG. (See e.g., as described in U.S. Pat. No. 6,548,286 expressly incorporated by reference herein in their entirety.) This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by Standard techniques known in the art using AAV packaging cells and packaging technology. Examples of these methods may be found, for example, in U.S. Pat. Nos. 5,436,146, 6,040,183, 6,093,570 and 6,548,286, expressly incorporated by reference herein in their entirety. Further compositions and methods for packaging are described in Wang et al. (US 2002/0168342), expressly incorporated by reference herein in its entirety. In practicing the invention, host cells for producing rAAV virions include mammalian cells, insect cells, microorganisms and yeast. HEK293 is a commonly used host cell that has seen significant success and has become among the most preferred host cells of choice for AAV production. Host cells can also be packaging cells in which the AAV rep and cap genes are stably maintained in the host cell or producer cells in which the AAV vector genome is stably maintained and packaged. AAV vectors are purified and formulated using techniques known to those familiar in the art.

Lentiviral Vectors and Lentiviral Design for B-Cell Targeting and Potentially Other Cell Type Targeting

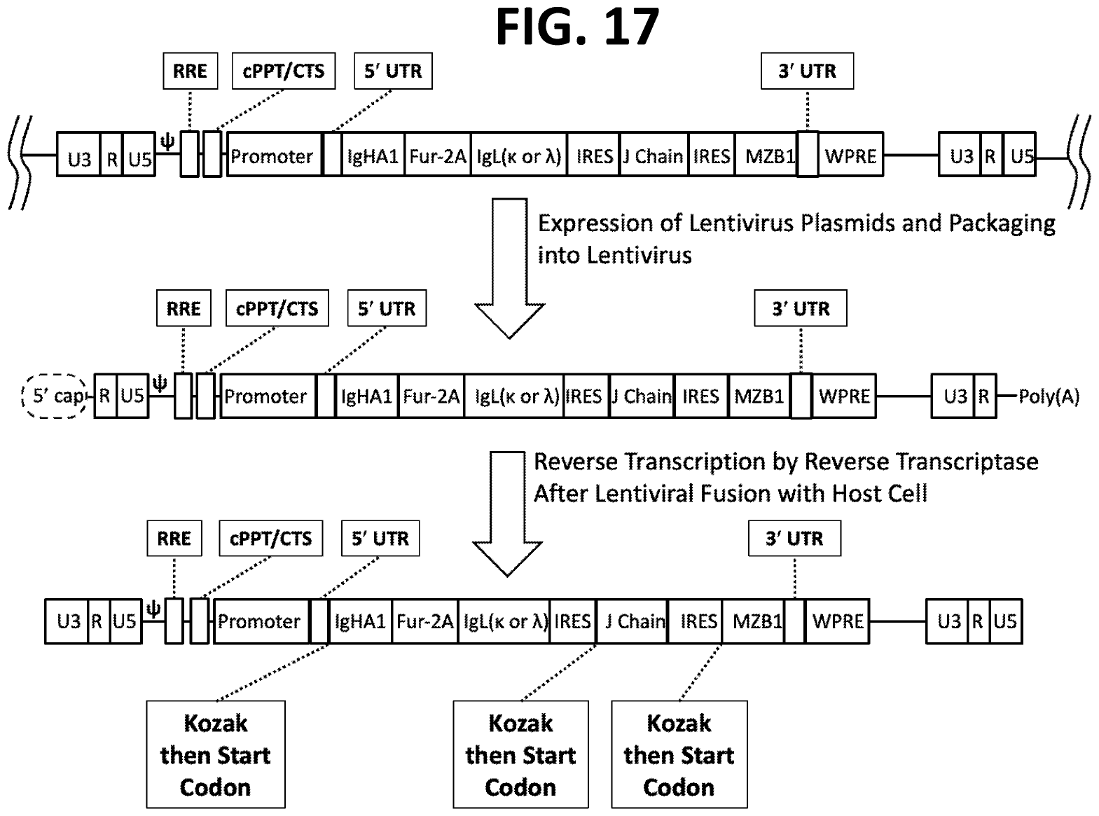

Lentiviral vectors have been extensively investigated and optimized over the past 20 years (See e.g., Milone, M. C., et al., 2017, Leukemia, 32:1529-1541). Lentiviral vectors may be used in practicing the present invention (See FIGS. 17, 18, 19, 20, 21, 24) and 26 (vector) as examples). It should be noted that while FIGS. 17, 18, 19, 20, 21, 24) and 26 (vector) depict the dIgA genes encoded for using the coding strand for the LTRs then genes could also be incorporated into the vector using the template strand for the LTRs meaning that transcription of the dIgA1 mRNA would begin from the end closest to the 3′ LTR in the direction of the 5′ LTR. In some cell types and even dependent on promoter selection this may result in a higher yield of the dIgA1 immunoglobulin and in other cells types it may result in a lower yield of the dIgA1 immunoglobulin. The relative yields can also differ based on the differentiation state of the cell. This likely has to do with the accessibility of the promoter or accessibility of the matrix associations regions (MAR) if used. Retroviral vectors have been tested and found to be suitable delivery vehicles of genes of interest into the genome of a broad range of target cells. Modified forms of lentiviral vectors may deliver episomes or extrachromosomal elements known as integration-deficient lentiviral vectors that are viral vectors may be used in the invention in one embodiment (See e.g., Wanisch, K., et al., 2009, The journal of the American Society of Gene Therapy, 17:1316-1332). One major advantage of integration-deficient and integration competent lentiviral vectors over AAV vectors is their packaging capacity. The total packaging capacity of an AAV vector is 4.9 kilobases or about 4.6 kilobases for the transgenes and cis-acting signals after subtracting the ITRs. However, in the instant invention there are vector constructs such as related to the expression of dIgA that involve 3 or 4 transgenes including the immunoglobulin light and heavy chain as well as the J Chain protein and optionally MZB1. With 4 transgenes one may not consider the use of a separate promoter for each gene if an AAV capsid is considered as the delivery vehicle and will be limited in what regulatory elements such as posttranscriptional regulatory elements may be used due to the AAV packaging capacity. This instant invention contemplates the expression of dIgA with use of a single open reading frame that can be achieved in the tight space of the AAV viral vector. Alternatively, this instant invention contemplates the use of an integration-deficient lentivirus in one embodiment and in another embodiment to deliver the retroviral vector or the use of an integration-competent lentivirus to integrate the vector into the host genomic DNA with use of a single open reading frame and potentially a separate promoter for each transgene in some embodiments. In one embodiment two separate promoters are used to express two or three genes in a lentiviral vector such that the maximum capacity of the lentiviral vector is utilized to maximally separate the first encoded transgene and the second encoded transgene. The packaging capacity of the integration-deficient lentiviral vector dedicated to the transgenes and transgene specific cis-acting signals is roughly about 8 kilobases.

This patent further contemplates the use of pseudotyped lentivirus to efficiently transduce targeted cells. Lentiviruses have been shown to transduce B-cells of a murine model with persistent expression of the transduced gene. This was accomplished by generating a lentivirus pseudotyped with an anti-CD19 antibody. The lentivirus anti-CD19 antibody targeted the B-cell surface receptor CD19 that mediated viral fusion upon binding the lentivirus to the B-cell, which permitted persistent expression of the transduced gene. (See e.g., Cascalho, M., et al., 2018, Sci Rep, 8:11143). Additionally, B-cells have been effectively targeted with a lentivirus pseudotyped with an envelope glycoprotein derived from the Baboon endogenous virus. (See, e.g., Fusil, F., Cosset, F. L. et. al., 2015, Molecular therapy: the journal of the American Society of Gene Therapy, 23:1734-1747) Lentivirus have seen use in clinical trials and has received FDA approval for immune based gene therapies. (See e.g., Milone, M. C., et al., 2017, Leukemia, 32:1529-1541; Campochiaro, P. A., et al., 2017, Hum Gene Ther. 28: 99-111; Milani, M., et al. 2017, EMBO Mol Med. 9:1558-73). The ability to direct the delivery of lentivirus vectors encoding one or more target protein coding sequences to specific target cells is desirable in practice of the present invention. Pesudotyping Lentivirus is well known to those familiar with the art. In all lentivirus packaging system the envelope protein or alternatively the pseudotyped protein is encoded for in a separate plasmid under the control of a CMV promoter. If the pseudotyped protein involves two separate proteins such as an immunoglobulin two separate promoters are typically used in the plasmid. This instant patent contemplates the use of pseudotyped integration deficient and also integration competent lentivirus to integrate the vector encoding for an immunoglobulin including dIgA1 and dIgA2 into genomic DNA. In one embodiment (See FIG. 24) the memory B-cell (CD 27+ receptor), naïve B-cell (CD20+ receptor or CD19+ receptor) or (CD38+ receptor or CD138+ receptor) plasma memory B-cells will have present on its surface both the endogenous heavy and light chain immunoglobulins as a B-cell receptor as well as the endogenous heavy chain in a heterodimer with the genome integrated lentiviral vector encoded light chain immunoglobulin as a B-cell receptor. Thus, having the gene therapy encoded immunoglobulin light chain presented on the naïve B-cells or memory B-cell will increase the probability that it will be activated from the target of interest. Where upon activation of memory B-cell they will give rise to either a long-lived plasma secreting cell or germinal center B-cell. The long lived plasma secreting B-cell (memory plasma B-cell) would produce the vector encoded dIgA, an engineered variants or dscFV-FcIgA specific to the target of interest at a much higher level than the naturally encoded immunoglobulin due to a strong promoter such as a Feek Promoter (SEQ ID NO: 105) in the lentivirus integrated vector vs. endogenous or naturally encoded Ig. The production of the long-lived plasma cell has the added benefit of producing B-cells that can persist for decades. (For the Feek Promoter Sequence see International Patent Application Number WO 2017/005923 Fusil et al. and the U.S. patent filing by the authors US 2018/0371064 A1 expressly incorporated by reference herein in its entirety; Also see Fusil, F., Cosset, F. L. et. al., 2015, Molecular therapy: the journal of the American Society of Gene Therapy, 23:1734-1747) Additionally, in other embodiments the use of pseudotyped lentivirus for delivering the vector targets other cell populations.

The light chain is ideally suited for antigen recognition as it tends to play a dominant role in target binding. Thus, the light chain binding the target will be sufficient in most cases to induce the memory B-cell to be activated and differentiate into a memory plasma B-cell (long lived memory B-cell) or in some cases into a Germinal center memory B-cell. (See, e.g. Sun, M., Li, L., Sheng Gao, Q., Pad S., 1994, The Journal of Biological Chemistry, 269:734-738; Also see, Hadzidimitriou, A., Darzentas, N., Belessi, C., et. al., 2009, Blood, 113:403-411). Other B-cells that express CD27+ markers would also be targeted including memory plasma B-cells that can survive for decades. If creating a gene therapy for an HIV immunization incorporating the HGN194 recombinant isotype of dIgA1 into a lentiviral vector construct in one embodiment one may consider to first administer the anti-CD20+ pseudotyped lentivirus carrying the lentiviral vector gene therapy to B-cells with a CD20+ marker. Alternatively, one may encode the vector to produce an HGN194 recombinant isotype of dIgA2.

This can be followed by a future exposure to the HIV envelope glycoprotein such as through an mRNA-based vaccine some period later that will activate the CD20+ B-cells containing the dIgA1 vector that would include immature B-cells and memory B-cells that received the integration-competent or optionally integration-deficient lentiviral vector. Alternatively, an ex vivo approach may be considered. Although, an ex vivo approach is generally not preferred for the administration of a broad scale vaccine for HIV. This will result in long-term persistence of memory plasma B-cells and Germinal Center Cells that can differentiate into memory plasma B-cells that will encode for an HGN194 recombinant isotype of dIgA1 or dIgA2 in addition to other B-cells specific to other regions of the HIV glycoprotein that resulted from the mRNA administration of the HIV glycoprotein. Vaccine boosters of the envelope glycoprotein can increase the count of memory plasma B-cells and T follicular helper T-cells would be appropriately developed to aid in the formation of memory B-cell from Germinal Center B-cells. A similar strategy could be used in an H. pylori vaccination as well as for other lethal viruses and pathogens. Because memory plasma B-cells both migrate to the bone marrow and to the lamina propria of some of the GI tract they can provide an extra layer of protection potentially for decades if not a lifetime. (See, e.g. Akkaya, M., Kwak, K. & Pierce, S. K., 2020, Nat Rev Immunol., 20:229-238; Also see, Landsverk, O. J., Jahnsen, F. L, et al., 2017, J. Exp. Med. 214:309-317; Khodadadi, L., Cheng, Q., Radbruch, A., & Hiepe, F., 2019, Frontiers in immunology, 10:721.) In another embodiment an anti CD27+ pseudotyped lentivirus (see FIG. 24) is used to target memory B-cells that would express both the naturally encoded heavy chain and vector encoded light chain as a B-cell receptor. Upon administration of the antigen the light chain binding the antigen will most cases be sufficient to activate the B-cell to differentiate into a memory plasma B-cell. This can also be an effective strategy to develop decades long immunity against HIV and other dangerous viruses.

The present invention provides integration-deficient replication-incompetent self-inactivating system, integration-competent replication-incompetent self-inactivating system and integration-competent replication incompetent systems where lentiviral vectors comprising one or more transgene sequences and lentiviral packaging vectors comprising one or more packaging elements. Additionally, the present invention provides pseudotyped lentiviral vectors encoding a heterologous or functionally modified envelope protein for producing pseudotyped lentivirus.

The term “vector” can refer to a ribonucleic acid (RNA) or deoxyribonucleic acid (DNA) molecule capable transferring or transporting another nucleic acid molecule. The transferred nucleic acid is generally linked to, e.g., inserted into, the vector nucleic acid molecule. A vector may include sequences that direct autonomous replication in a cell. An RNA vector is packaged into the lentivirus packaging system and is reverse transcribed into DNA following viral fusion with the transduced cell.

As is evident to one of skill in the art, the term “retroviral vector” is widely used to refer either to a nucleic acid molecule (e.g., a transfer plasmid) that includes virus-derived nucleic acid elements that typically facilitate transfer of the nucleic acid molecule by integration into the genome of a cell or to a viral particle that mediates nucleic acid retroviral transfer. Retroviral particles will typically include various viral components and sometimes also host cell components in addition to nucleic acid(s).

Once the virus is integrated into the host genome, it is referred to as a “provirus.”, The provirus serves as a template for RNA polymerase II and directs the expression of RNA molecules, which encode the structural proteins and enzymes needed to produce new viral particles. At each end of the provirus are structures called “long terminal repeats” or “LTRs.”, The term “long terminal repeat (LTR)” refers to domains of base pairs located at the ends of lentiviral vector which, in their natural sequence context, are direct repeats and contain U3, R and U5 regions. LTRs generally provide functions fundamental to the expression of lentiviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer-binding site and the R region and contains the polyadenylation sequence. The R (repeat) region is flanked by the U3 and U5 regions. Typically, the LTR composed of U3, R and U5 regions and appears at both the 5′ and 3′ ends of the viral genome where in the third generation system the 5′ LTR of the RNA vector has the U3 missing.

The term “packaging signal” or “packaging sequence” refers to sequences located within the lentiviral genome which are required for insertion of the viral RNA into the viral capsid or particle, (See e.g., Clever et al., 1995. J. of Virology, Vol. 69, No. 4; pp. 2101-2109.) Many lentiviral vectors use the minimal packaging signal (referred to as psi (′P) or (Ψ+) sequence) needed for encapsidation of the viral genome. Thus, as used in this instant patent, the terms “psi” and the symbol “Ψ,” are used in reference to the non-coding sequence required for encapsidation of retroviral RNA strands during viral particle formation. Although, in some embodiments additional nucleotides are used at both the 5′ and 3′ ends of the psi signal to maximize efficiency of the lentivirus. “Self-inactivating” vectors refers to replication-defective vectors, e.g., lentiviral vectors, in which the 3′ LTR enhancer-promoter region, known as the U3 region, has been modified (e.g., by deletion and/or substitution) to prevent viral transcription beyond the first round of viral replication this is also a characteristic of the third-generation lentiviral packaging system. This is because the 3′ LTR U3 region is used as a template for the left 5′ LTR U3 region during viral replication and, thus, the viral transcript cannot be made without the U3 enhancer-promoter. Thus, the U3 of the 5′ LTR is inactivated. To render a lentiviral vector replication deficient a large part of the U3 region of the 3′ LTR is deleted or modified which eliminates the viral promoter activity and also allowing for the transgene expression to be controlled by the incorporation of an internal promoter. Additionally, this large deletion of the U3 region has the added benefit of increasing transgene expression. (For example, see e.g., U.S. Pat. No. 2013/0004471 paragraph 82). The U3 region of the 5′ LTR may be replaced with a heterologous promoter to drive transcription of the viral genome during production of viral particles. Examples of heterologous promoters, which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system.

In some embodiments for maximum biosafety the 3^{rd} Generation lentiviral packaging systems are preferred. The replication-incompetent 3rd generation lentiviral packaging system splits the viral genome into 4 plasmids: (A) a plasmid containing only the genes necessary for packaging the lentiviral vector which are gag and pol which are operatively linked to a promoter, a rev gene and a polyA site, (B) A plasmid that contains only the regulatory gene rev that is operatively linked to a promoter and a polyA site, (C) A plasmid that only contains the envelope gene known as env operatively linked to a promoter and a polyA site and (D) The transgene plasmid that contains the genes whose expression is desired as part of gene therapy. The transgene plasmid also contains modified LTRs at the 5′ and 3′ ends. The LTR includes a deleted or inactivated U3. Also in the LTR are the R and U5 cis-acting signals. The deleted or inactivated U3 enhancer/promoter (known as ΔU3) of the 5′ LTR is replaced with a human cytomegalovirus (CMV) immediate early enhancer and promoter (SEQ ID NO: 1) to eliminate the need for the transcriptional activator tat that has been deleted or inactivated. The viral RNA is stored in the lentivirus delivery system with a 5′ LTR as a 5′-cap-R-U5 and the 3′ LTR contains the mutated or deleted U3 (ΔU3) and R followed by a poly A tail of about 200 adenosines. That is the 5′ U3 is missing and the 3′ U5 is missing. The provirus that results following reverse transcription in the third generation lentiviral packaging systems contains the ΔU3-R-U5 sequences at the 5′ end and U5-R-ΔU3 sequences at the 3′ end. The first and second generation lentivirus packaging system also store the viral RNA with a 5′ LTR as 5′-cap-R-U5 and the 3′ LTR as U3-R followed by about 200 adenosines. The provirus that results following reverse transcription in the first and second generation lentiviral packaging systems contains the U3-R-U5 sequences at the 5′ end and U5-R-U3 sequences at the 3′ end. Additional details that describe the transgene plasmid and LTRs are described in later embodiments. The third generation lentiviral packaging system typically employs a heterologous or functionally modified envelope protein for safety. The third generation lentiviral packaging system builds on the first generation packaging system by additionally deleting the accessory genes vif, vpr, vpu and nef. Integration-deficient lentiviral vectors refer to lentiviral vectors that cannot act as retroviruses. They are viral vectors that are episomally-maintained. Both the U5 and U3 regions may be mutated at integrase (IN) attachment (att) sites to eliminate their retroviral function. This occurs through mutating specific recognition sequences necessary for integrase (IN) to attach to the vector and also cut by IN. A common strategy used to generate integration deficient lentiviral vectors is to mutate specific amino acid residues on the integrase, which is found on the pol gene, that are necessary for retroviral function. Such mutations often include either D64 or D116 of the catalytic triad. Mutating RRK (262-264) motif at the C-terminal domain causes IN to fail to bind target genomic DNA. Mutating Lysines at integrase protein positions 264, 266 and 273 impairs target DNA binding and stand transfer. These lysines residues may be acetylated in the target cell where the acetylation is required for strand transfer. Other mutations might occur at N120 or W235 that is necessary for DNA binding and thus either of these mutations block integration. If the gene encoding for the integrase protein is not mutated then the lentivirus would be integration competent and would be expected to integrate the vector into the host genomic DNA, that is also contemplated in some embodiments in this instant patent. (See e.g. Yáñez-Muñoz, R. J., et al., 2006, Nat Med. 12:348-353; Conese, M., et. al., 2004, Gene Ther., 11:1735-1741; Karwacz, K., et al., 2009, J. Virol., 83:3094-3103).

U.S. AND INTERNATIONAL PATENT DOCUMENTS

| | |
|---|---|
| 5,436,146 A | July 1995 Shenk et al. |
| 6,040,183 A | March 2000 Ferrari et al. |
| 6,093,570 A | July 2000 Ferrarietal et al. |
| 6,548,286 B1 | April 2003 Samulski et al. |
| 7,498,024 B2 | March 2009 Fang et al. |
| 7,709,224 B2 | May 2010 Fang et al. |
| 8,133,727 | March 2012 Baltimore et al. |
| 9,605,049 | March 2017 Campana et al. |
| 10,251,377 B2 | April 2019 Clube et al. |
| 10,442,867 | October 2019 Orentas et al. |
| 10,538,588 | January 2020 Adusumilli et al. |
| 10,603,380 | March 2020 Witzius, J. et al. |
| US 2002/0168342 | November 2002 Wang et al. |
| US 2017/0362297 A1 | December 2017 Marasco et al. |
| US 2018/0371064 A1 | December 2018 Fusil et. al. |
| WO 2011/0925W A2 | August 2011 Corti et. al. |
| WO 2017/005923 | January 2017 Fusil et. al. |

### REFERENCED PUBLICATIONS

Lu, R M., Hwang, Y C., Liu, I J. et al. Development of therapeutic antibodies for the treatment of diseases. J Biomed Sci 27, 1 (2020). https://doi.org/10.1186/s12929-019-0592-z
Iwasaki, A. & Yang, Y. “The potential danger of suboptimal antibody responses in COVID-19.”, Nat. Rev. Immunol., vol. 20, pp. 339-341, 2020. https://doi.org/10.1038/s41577-020-0321-
Suarez, E. R., Chang de, K., Sun, J., Sui, J., Freeman, G. J., Signoretti, S., Zhu, Q., Marasco, W. A. “Chimeric antigen receptor T cells secreting anti-PD-L1 antibodies more effectively regress renal cell carcinoma in a humanized mouse model.” Oncotarget. 2016 vol. 7, pp. 34341-55, 2016. doi: 10.18632/oncotarget.9114.
Powell, A. B., Ren, Y., Korom, M., Saunders, D., Hanley, P. J., Goldstein, H., Nixon, D. F., Bollard, C. M., Lynch, R. M., Jones, R. B., & Cruz, C., “Engineered Antigen-Specific T Cells Secreting Broadly Neutralizing Antibodies: Combining Innate and Adaptive Immune Response against HIV.” Molecular therapy. Methods & clinical development, vol. 19, pp. 78-88, 2020. https://doi.org/10.1016/j.omtm.2020.08.015
Lohse, S., Derer, S., Beyer, T., Klausz, K., Peipp, M., Leusen, J. H., van de Winkel, J. G., Dechant, M., Valerius, T., “Recombinant dimeric IgA antibodies against the epidermal growth factor receptor mediate effective tumor cell killing.” J Immunol. Vol. 186, pp. 3770-8, 2011.
Pandey, S., & Vyas, G. N. “Adverse effects of plasma transfusion.”, Transfusion, vol. 52, supp. 1 (Suppl 1), pp. 65S-79S, 2012. https://doi.org/10.1111/j.1537-2995.2012.03663.x
Boe, D. M., Boule, L. A., & Kovacs, E. J. “Innate immune responses in the ageing lung.”, Clinical and experimental immunology, vol. 187, No. 1, pp. 16-25, 2017. https://doi.org/10.1111/cei.12881
Vale, A. M., & Schroeder, H. W., Jr. “Clinical consequences of defects in B-cell development.”, The Journal of allergy and clinical immunology, vol. 125, No. 4, 778-787, 2010. https://doi.org/10.1016/j.jaci.2010.02.018
Pilette, C., Ouadrhiri, Y., Sibille, Y., et al. “Lung mucosal immunity: immunoglobulin-A revisited.”, European Respiratory Journal, vol. 18, No. 3, pp. 571-588, 2001. https://erj.ersjournals.com/content/18/3/571
Terauchi, Y., Sano, K., Hasegawa, H., et al. “IgA polymerization contributes to efficient virus neutralization on human upper respiratory mucosa after intranasal inactivated influenza vaccine administration.”, Human vaccines & immunotherapeutics, vol. 14, No. 6, pp. 1351-1361, 2018. https://doi.org/10.1080/21645515.2018.1438791
Bakema, J., van Egmond, M. “The human immunoglobulin A Fc receptor FcαRI: a multifaceted regulator of mucosal immunity.” Mucosal Immunol vol 4, pp. 612-624, 2011. https://doi.org/10.1038/mi.2011.36
Melamed, D., Benschop, R. J., Cambier, J. C., & Nemazee, D., “Developmental regulation of B lymphocyte immune tolerance compartmentalizes clonal selection from receptor selection.”, Cell., vol. 92, No. 2, pp. 173-182, 1998. https://doi.org/10.1016/s0092-8674(00)80912-5
Akkaya, M., Kwak, K. & Pierce, S. K. “B cell memory: building two walls of protection against pathogens.” Nat Rev Immunol., vol. 20, pp. 229-238, 2020.
Ziegner, M., Steinhauser, G., & Berek, C., “Development of antibody diversity in single germinal centers: selective expansion of high-affinity variants.”, Eur. J. Immunol. Vol. 24, pp. 2393-2400, 1994.
Shlomchik, M. J., Litwin, S., & Weigert, M., “The Influence of Somatic Mutation on Clonal Expansion.”, Prog. in Immunol. Proc. 7^{th} Int. Cong. Immunol. Vol. 7, pp. 415-423, 1990. https://doi.org/10.1007/978-3-642-83755-5_55
Berek, C., & Milstein, C., “Mutation drift and repertoire shift in the maturation of the immune response.”, Immunol Rev. vol. 96, pp. 23-41, 1987. doi:10.1111/j.1600-065x.1987.tb00507.x
Tiller, T., Tsuiji, M., Wardemann, H., et al. “Autoreactivity in human IgG+ memory B cells.”, Immunity, vol. 26, No. 2, pp. 205-213, 2007. https://doi.org/10.1016/j.immuni.2007.01.009
Fsuiji, M., Yurasov, S., Wardemann, H., et al. “A checkpoint for autoreactivity in human IgM+ memory B cell development.”, The Journal of Experimental Medicine vol. 203, No. 2, pp. 393-400, 2006. doi:10.1084/jem.20052033
Wardemann, H., Yurasov, S., Nussenzweig, M. C., et al. “Predominant autoantibody production by early human B cell precursors.”, Science vol. 30, pp. 1374-1377, 2003. doi:10.1126/science.1086907
Cao, Y., Su, B., Geng, C., et al. “Potent neutralizing antibodies against SARS-CoV-2 identified by high-throughput single-cell sequencing of convalescent patients' B cells.”, Cell, vol. 182, pp. 73-84, 2020. https://doi.org/10.1016/j.ce11.2020.05.025.
Weiss, G. E., Ndungu, F. M., & Pierce, S. K. “High efficiency human memory B cell assay and its application to studying Plasmodium falciparum-specific memory B cells in natural infections.”, Journal of immunological methods, vol. 375, No. 1-2, pp. 68-74. 2012. https://doi.org/10.1016/j.jim.2011.09.006
Hunter, S. A., & Cochran, J. R., “Cell-Binding Assays for Determining the Affinity of Protein-Protein Interactions: Technologies and Considerations.”, Methods Enzymol. vol. 580, pp. 21-44, 2016. doi:10.1016/bs.mie.2016.05.002
Li, P., Selvaraj, P., & Zhu, C. “Analysis of competition binding between soluble and membrane-bound ligands for cell surface receptors.”, Biophysical journal, vol. 77, No. 6, pp. 3394-3406, 1999. https://doi.org/10.1016/S0006-3495(99)77171-7
Amanna, I. J., & Slifka, M. K., “Quantitation of rare memory B cell populations by two independent and complementary approaches.”, Journal of immunological methods, vol. 317, No. 1-2, pp. 175-185, 2006. https://doi.org/10.1016/j.jim.2006.09.005
Shen S, et al. (2007) Expression, glycosylation, and modification of the spike (S) glycoprotein of SARS CoV. Methods Mol Biol. 379: 127-35.
Du L, et al. (2009) The spike protein of SARS-CoV—a target for vaccine and therapeutic development. Nat Rev Microbiol. 7 (3): 226-36.
Xiao X, et al. (2004) The SARS-CoV S glycoprotein. Cell Mol Life Sci. 61 (19-20): 2428-3
Setliff, I., Shiakolas, A. R Ronsard, L., et al., “High-Throughput Mapping of B Cell Receptor Sequences to Antigen Specificity.” Cell, vol. 179, pp. 1636-1646, 2019.
Wu, X., Yang, Z. Y., Kwong, P. D., et al. “Rational design of envelope identifies broadly neutralizing human monoclonal antibodies to HIV-1.” Science, vol. 3295, pp. 856-861, 2010. https://doi.org/10.1126/science.1187659
Niu, X., Zhao, L., Zhang, X., et al. Chen, L. (2019). Convalescent patient-derived monoclonal antibodies targeting different epitopes of E protein confer protection against Zika virus in a neonatal mouse model. Emerging microbes & infections, 8(1), 749-759. https://doi.org/10.1080/22221751.2019.1614885
Crotty, S., Aubert, R. D., Glidewell, J., & Ahmed, R. “Tracking human antigen-specific memory B cells: a sensitive and generalized ELISPOT system.”, J Immunol Methods., vol. 286, No. 1-2, pp. 111-122, 2004. doi:10.1016/j.jim.2003.12.015
Antonio, E., Muruato, C. R., Fontes-Garfias, P. R., Mariano G.-B., Vineet, D., Menachery, X. X., Pei-Yong, S. “A high-throughput neutralizing antibody assay for COVID-19 diagnosis and vaccine evaluation” bioRxiv 2020.05.21.109546; doi: https://doi.org/10.1101/2020.05.21.109546
Robbiani, D. F., Gaebler, C., Muecksch, F. et al. Convergent antibody responses to SARS-CoV-2 in convalescent individuals. Nature vol. 584, pp. 437-442 (2020). https://doi.org/10.1038/s41586-020-2456-9
Sarzotti-Kelsoe, M., Daniell, X., Montefiori, D. C., et al. “Optimization and validation of a neutralizing antibody assay for HIV-1 in A3R5 cells.”, Journal of immunological methods, vol. 409, pp. 147-160, 2014. https://doi.org/10.1016/j.jim.2014.02.013
Klein, U., Rajewsky, K., Kuppers, R., “Human immunoglobulin (Ig)M+ IgD+ peripheral blood B cells expressing the CD27 cell surface antigen carry somatically mutated variable region genes: CD27 as a general marker for somatically mutated (memory) B cells.”, The Journal of Experimental Medicine vol. 188, pp. 1679-1689, 1998.
Vale, A. M., Cavazzoni, C. B., Nobrega, A., & Schroeder, H. W. “The Global Self-Reactivity Profile of the Natural Antibody Repertoire Is Largely Independent of Germline D_{H} Sequence.”, Front. Immunol. vol. 7, No. 296., 2016. doi: 10.3389/fimmu.2016.00296
Nobrega, A., Haury, M, Coutinho, A. et al. “Global analysis of antibody repertoires. II. Evidence for specificity, self-selection and the immunological “homunculus” of antibodies in normal serum.”, Eur J Immunol., vol. 23, No. 11, pp. 2851-2859, 1993. doi:10.1002/eji.1830231119
Haury, M., Grandien, A., Sundblad, A., Coutinho, A., & Nobrega, A. “Global analysis of antibody repertoires. 1. An immunoblot method for the quantitative screening of a large number of reactivities.”, Scand. J. Immunol. vol. 39, No. 1, pp. 79-87, 1994. doi:10.1111/j.1365-3083.1994.tb03343.x
Lu, J., Chu, J., Zou, Z., Hamacher, N. B., Rixon, M. W., & Sun, P. D. (2015). Structure of FcγRI in complex with Fc reveals the importance of glycan recognition for high-affinity IgG binding. Proceedings of the National Academy of Sciences of the United States of America, 112(3), 833-838. https://doi.org/10.1073/pnas.1418812112
Pritsch, O., Hudry-Clergeon, G., Buckle, M., et al. “Can immunoglobulin C(H)1 constant region domain modulate antigen binding affinity of antibodies?.”, J Clin Invest. Vol. 98. No. 10, pp. 2235-2243, 1996. doi:10.1172/JC1119033
Li, Y., Wang, G., Li, N., Wang, Y., Zhu, Q., Chu, H., Wu, W., Tan, Y., Yu, F., Su, X D., Gao, N., Xiao, J., “Structural insights into immunoglobulin M.” Science, vol. 367, pp. 1014-1017, 2020. doi: 10.1126/science.aaz5425
Janda, A., Bowen, A., Greenspan, N. S., Casadevall, A. “Ig Constant Region Effects on Variable Region Structure and Function.”, Front Microbiol. vol. 7 No. 22, 2016. Published 2016 Feb. 4. doi: 10.3389/fmicb 0.2016.00022
Kumar Bharathkar, S., Parker, B. W., Malyutin, A., Haloi, N., Tajkhorshid, E., Stadtmueller, B. M. “The Structures of Secretory and Dimeric Immunoglobulin A” bioRxiv 2020.02.16.951780; doi: https://doi.org/10.1101/2020.02.16.951780
Suzuki, T., Kawaguchi, A., Ainai, A., Tamura, S., Ito, R., Multihartina, P., Setiawaty, V., Pangesti, K. N., Odagiri, T., Tashiro, M., & Hasegawa, H., “Relationship of the quaternary structure of human secretory IgA to neutralization of influenza virus.” Proceedings of the National Academy of Sciences of the United States of America, vol. 112, pp. 7809-7814, 2015. https://doi.org/10.1073/pnas.1503885112
Toraño, A., & Putnam, F. W., “Complete amino acid sequence of the alpha 2 heavy chain of a human IgA2 immunoglobulin of the A2m (2) allotype.” Proceedings of the National Academy of Sciences of the United States of America, vol. 75, pp. 966-969, 1978 https://doi.org/10.1073/pnas.75.2.966;
Li, Z., “The forgotten tale of immunoglobulin allotypes in cancer risk and treatment.” Exp Hematol Oncol 2, 6, 2013.
Fang, J., Qian, J., Yi, S. et al. “Stable antibody expression at therapeutic levels using the 2A peptide.”, Nat. Biotechnol., vol. 23, pp. 584-590, 2005. https://doi.org/10.1038/nbt1087
Krysan, D. J., Rockwell, N. C., Fuller, R. S. “Quantitative characterization of furin specificity. Energetics of substrate discrimination using an internally consistent set of hexapeptidyl methylcoumarinamides.”, J Biol Chem. vol. 274, No. 33, pp. 23229-23234, 1999. doi:10.1074/jbc.274.33.23229
Lewis, A. D., Chen, R., Montefiori, D. C., Johnson. P. R., Clark, K. R. “Generation of neutralizing activity against human immunodeficiency virus type 1 in serum by antibody gene transfer.”, J Virol. vol. 76, No. 17, pp. 8769-8775, 2002. doi:10.1128/jvi.76.17.8769-8775.2002
Huang, M. T., & Gorman, C. M. “The simian virus 40 small-t intron, present in many common expression vectors, leads to aberrant splicing.”, Molecular and cellular biology, vol. 10, No. 4, pp. 1805-1810, 1990. https://doi.org/10.1128/mcb.10.4.1805
Wu, C., & Alwine, J. C. “Secondary structure as a functional feature in the downstream region of mammalian polyadenylation signals.”, Molecular and cellular biology, vol. 24, No. 7, pp. 2789-2796, 2004. https://doi.org/10.1128/mcb.24.7.2789-2796.2004
Lycke, N., Erlandsson, L., Ekman, L., Scholl, K., Leanderson, T. “Lack of J Chain inhibits the transport of gut IgA and abrogates the development of intestinal antitoxic protection.”, J. Immunol., vol. 163, pp. 913-919, 1999.
Sorensen, V., Sundvold, V., Michaelsen, T. E., Sandlie, I. “Polymerization of IgA and IgM: Roles of Cys309/Cys414 and the secretory tailpiece.”, J. Immunol., vol. 162, pp. 3448-3455, 1999.
Schroeder, H., Wald, D., Greenspan, N., “Chapter 4: Immunoglobulins: Structure and Function”. In Paul, William (ed.). Fundamental Immunology (Book) (6th ed.). Philadelphia, Pa.: Lippincott Williams & Wilkins. pp. 125-151, 2008. ISBN 978-0-7817-6519-0.
Stadtmueller, B. M., Huey-Tubman, K. E., Bjorkman, P. J., et al. “The structure and dynamics of secretory component and its interactions with polymeric immunoglobulins.”, Elife. vol. 5, e10640, 2016. Published 2016 Mar. 4. doi:10.7554/eLife.10640
Castro, C. D., & Flajnik, M. F., “Putting J Chain back on the map: how might its expression define plasma cell development?” Journal of Immunology vol. 193, No. 7, pp. 3248-3255, 2014. https://doi.org/10.4049/jimmunol.1400531
Koshland, M. E., “The coming of age of the immunoglobulin J Chain.”, Annu Rev Immunol. vol. 3, pp. 425-453, 1985. doi:10.1146/annurev.iy.03.040185.002233
Bonner, A., Almogren, A., Furtado, P. et al. “Location of secretory component on the Fc edge of dimeric IgA1 reveals insight into the role of secretory IgA1 in mucosal immunity.”, Mucosal Immunol. vol. 2, pp. 74-84, 2009. https://doi.org/10.1038/mi.2008.68
Johansen, F. E., Braathen, R., Brandtzaeg, P. “Role of J Chain in Secretory Immunoglobulin Formation”. Scandinavian Journal of Immunology. vol. 52, No. 3, pp. 240-248, 2000. doi:10.1046/j.1365-3083.2000.00790.x. PMID 10972899
Wang, Y., Wang, G., Li, Y. et al. “Structural insights into secretory immunoglobulin A and its interaction with a pneumococcal adhesin.” Cell Res, vol. 30, pp. 602-609, 2020. https://doi.org/10.1038/s41422-020-0336-3
Xiong, E., Li, Y., Wang, J. Y., et al. “MZB1 promotes the secretion of J Chain-containing dimeric IgA and is critical for the suppression of gut inflammation.”, Proceedings of the National Academy of Sciences of the United States of America, vol. 116, No. 27, pp. 13480-13489, 2019. https://doi.org/10.1073/pnas.1904204116
Mak, T. W., Saunders, M. E., Jett, B. D., Primer to the Immune Response (Second Edition), Academic Cell, 2014, pp. 269-292, ISBN 9780123852458, https://doi.org/10.1016/B978-O-12-385245-8.00012-1
Ben Mkaddem, S., Benhamou, M., & Monteiro, R. C., “Understanding Fc Receptor Involvement in Inflammatory Diseases: From Mechanisms to New Therapeutic Tools.” Frontiers in immunology, vol. 10, pp. 811, 2019. https://doi.org/10.3389/fimmu.2019.00811
O'Brien, C., Flower, D. R., & Feighery, C., “Peptide length significantly influences in vitro affinity for MHC class II molecules.” Immunome research, 4, 6, 2008. https://doi.org/10.1186/1745-7580-4-6
Gehring, A. J., Sun, D., Bertoletti, A., et al. “The level of viral antigen presented by hepatocytes influences CD8 T-cell function.” J Virol., vol. 81, pp. 2940-9, 2007. https://doi:10.1128/JVI.02415-06
Arber, C., Abhyankar, H., Heslop, H. E., Brenner, M. K., Liu, H., Dotti, G., & Savoldo, B. (2013). “The immunogenicity of virus-derived 2A sequences in immunocompetent individuals.” Gene therapy, vol. 20, pp. 958-962. https://doi.org/10.1038/gt.2013.25
Nagaraju, K., Raben, N., Plotz, P., et al.“Conditional up-regulation of MHC class I in skeletal muscle leads to self-sustaining autoimmune myositis and myositis-specific autoantibodies.” Proceedings of the National Academy of Sciences of the United States of America, vol. 97, pp. 9209-9214. https://doi.org/10.1073/pnas.97.16.9209
Sips, M., Krykbaeva, M., Alter, G., et al. “Fc receptor-mediated phagocytosis in tissues as a potent mechanism for preventive and therapeutic HIV vaccine strategies.” Mucosal Immunol. vol. 9, pp. 1584-1595, 2016 doi: 10.1038/mi.2016.12.
Kruize, Z., Kootstra, N., “The Role of Macrophages in HIV Persistence and Pathogenesis” Frontiers in Microbiology, vol. 10, pp. 1-17, 2019.
Shen, R., Richter, H. E., Smith, P. D., et al. “Macrophages in vaginal but not intestinal mucosa are monocyte-like and permissive to human immunodeficiency virus type 1 infection”. Journal of virology, vol. 83, pp. 3258-3267, 2009. https://doi.org/10.1128/JVI.01796-08
Gobeil, L. A., Lodge, R., Tremblay, M. J. “Macropinocytosis-like HIV-1 internalization in macrophages is CCR5 dependent and leads to efficient but delayed degradation in endosomal compartments.” J Virol. Vol. 87, 735-45, 2013. doi: 10.1128/JVI.01802-12.
Carter, G. C., Bernstone, L., Baskaran, D., James, W. “HIV-1 infects macrophages by exploiting an endocytic route dependent on dynamin, Rac1 and Pak1.” Virology, vol. 409, pp. 234-250, 2011.
Watkins, J. D., Sholukh, A. M., Mukhtar, M. M., Siddappa, N. B., Lakhashe, S. K., Kim, M., Reinherz, E. L., Gupta, S., Forthal, D. N., Sattentau, Q. J., Villinger, F., Corti, D., Ruprecht, R. M.; CAVD Project Group. “Anti-HIV IgA isotypes: differential virion capture and inhibition of transcytosis are linked to prevention of mucosal R5 SHIV transmission.” AIDS. 2013 vol. 27, pp. F13-20, 2013.
Tomaras, G. D., Ferrari, G., Haynes, B. F. et al. “Vaccine-induced plasma IgA specific for the C1 region of the HIV-1 envelope blocks binding and effector function of IgG.” Proceedings of the National Academy of Sciences of the United States of America, vol. 110, pp. 9019-9024, 2013. https://doi.org/10.1073/pnas.1301456110
Haynes B F, et al., Immune-correlates analysis of an HIV-1 vaccine efficacy trial. N Engl J Med, vol. 366, pp. 1275-1286, 2012.
Abdullah, M., Greenfield, L. K., Bronte-Tinkew, D. et al. VacA promotes CagA accumulation in gastric epithelial cells during Helicobacter pylori infection. Sci Rep 9, 38 (2019). https://doi.org/10.1038/s41598-018-37095-4
Meng, G., X. Wei, X. Wu, M. T. Sellers, J. M. Decker, Z. Moldoveanu, J. M. Orenstein, M. F. Graham, J. C. Kappes, J. Mestecky, G. M. Shaw, and P. D. Smith., “Primary intestinal epithelial cells selectively transfer R5 HIV-1 to CCR5 cells. Nat. Med. 8:150-156, 2002.
Schweighardt, B., Roy, A. M., Meiklejohn, D. A., Grace, E. J., 2^{nd}, Moretto, W. J., Heymann, J. J., & Nixon, D. F., “R5 human immunodeficiency virus type 1 (HIV-1) replicates more efficiently in primary CD4+ T-cell cultures than X4 HIV-1.” Journal of virology, vol. 78, pp. 9164-9173, 2004. https://doi.org/10.1128/JVI.78.17.9164-9173.2004
Montefiori, D. C., Roederer, M., Morris, L., & Seaman, M. S., “Neutralization tiers of HIV-1.” Current opinion in HIV and AIDS, vol. 13, pp. 128-136, 2018. https://doi.org/10.1097/COH.0000000000000442
Atherton, J. C., Blaser, M. J., Cover, T. L., et al., “Mosaicism in vacuolating cytotoxin alleles of Helicobacter pylori. Association of specific vacA types with cytotoxin production and peptic ulceration.” J Biol Chem, Jvol. 270, pp. 17771-17777, 1995. doi: 10.1074/jbc.270.30.17771.
Kuck, D., Kolmerer, B., Iking-Konert, C., Krammer, P. H., Stremmel, W., & Rudi, J., “Vacuolating cytotoxin of Helicobacter pylori induces apoptosis in the human gastric epithelial cell line AGS.” Infection and immunity, vol. 69, pp. 5080-5087, 2001. https://doi.org/10.1128/IAI.69.8.5080-5087.2001
Palframan, S. L., Kwok, T., Gabriel, K., “Vacuolating cytotoxin A (VacA), a key toxin for Helicobacter pylori pathogenesis” Frontiers in Cellular and Infection Microbiology, v. 2, pp. 1-9, 2012. doi:10.3389/fcimb.2012.00092
Xu, C., Soyfoo, D. M., Wu, Y., & Xu, S., “Virulence of Helicobacter pylori outer membrane proteins: an updated review.” European journal of clinical microbiology & infectious diseases: official publication of the European Society of Clinical Microbiology, vol. 39, pp. 1821-1830, 2020. https://doi.org/10.1007/s10096-020-03948-y
Königer, V., Holsten, L., Harrison, U. et al. Helicobacter pylori exploits human CEACAMs via HopQ for adherence and translocation of CagA. Nat Microbiol, vol. 2, pp. 16188, 2017. https://doi.org/10.1038/nmicrobiol.2016.188
Bonsor, D. A., Zhao, Q., Sundberg, E. J., et al. The Helicobacter pylori adhesin protein HopQ exploits the dimer interface of human CEACAMs to facilitate translocation of the oncoprotein CagA. The EMBO journal, vol. 37, pp. e98664, 2018. https://doi.org/10.15252/embj.201798664
Boren, T., Falk, P., Roth, K. A., Larson, G., Normark, S., “Attachment of Helicobacter pylori to human gastric epithelium mediated by blood group antigens.” Science. Vol. 262, pp. 1892-5, 1993. doi: 10.1126/science.8018146.
Spencer, J., Sollid, L. “The human intestinal B-cell response.” Mucosal Immunol, vol. 9, pp. 1113-1124, 2016. https://doi.org/10.1038/mi.2016.59
Lycke, N., Bemark, M. “The regulation of gut mucosal IgA B-cell responses: recent developments.” Mucosal Immunol, vol. 10, pp. 1361-1374, 2017. https://doi.org/10.1038/mi.2017.62
Cifarelli, V., Eichmann, A., “The Intestinal Lymphatic System: Functions and Metabolic Implications” Cellular and Molecular Gastroenterology and Hepatology, 7, pp. 503-513, 2019
Baluk, P., Fuxe, J., McDonald, D. M., et al. “Functionally specialized junctions between endothelial cells of lymphatic vessels.” The Journal of experimental medicine, vol. 204, pp. 2349-2362, 2007. https://doi.org/10.1084/jem.20062596
Venugopal, A. M., Stewart, R. H., Laine, G. A., Dongaonkar, R. M., Quick, C. M., “Lymphangion coordination minimally affects mean flow in lymphatic vessels.” Am J Physiol Heart Circ vol. 293, pp. H1183-9, 2007. doi: 10.1152/ajpheart.01340.2006.
Fusil, F., Calattini, S., Cosset, F. L., et al. “A Lentiviral Vector Allowing Physiologically Regulated Membrane-anchored and Secreted Antibody Expression Depending on B-cell Maturation Status.” Molecular therapy: the journal of the American Society of Gene Therapy, vol. 23, pp. 1734-1747. https://doi.org/10.1038/mt.2015.148
Trepotec, Z., Aneja, M. K., Geiger, J., Hasenpusch, G., Plank, C., Rudolph, C., “Maximizing the Translational Yield of mRNA Therapeutics by Minimizing 5′-UTRs.” Tissue Eng Part A. vol 1-2, 69-79, 2019. doi: 10.1089/ten.TEA.2017.0485.
Leppek, K., Das, R. & Barna, M. “Functional 5′ UTR mRNA structures in eukaryotic translation regulation and how to find them.” Nat Rev Mol Cell Biol, vol. 19, pp. 158-174, 2018. https://doi.org/10.1038/nrm.2017.103
Calvo, S. E., Pagliarini, D. J., Mootha, V. K., “Upstream open reading frames cause widespread reduction of protein expression and are polymorphic among humans.” PNAS, v. 18, pp. 7507-7512, 2009. https://www.pnas.org/content/106/18/7507
Babendure, J. R., Babendure, J. L., Ding, J. H., & Tsien, R. Y. “Control of mammalian translation by mRNA structure near caps.” RNA, vol. 12, pp. 851-861, 2006. https://doi.org/10.1261/rna.2309906
Barrett, L. W., Fletcher, S., & Wilton, S. D., “Regulation of eukaryotic gene expression by the untranslated gene regions and other non-coding elements.” Cellular and molecular life sciences: CMLS, vol. 69, pp. 3613-3634, 2012. https://doi.org/10.1007/s00018-012-0990-9
Siepel, A., Bejerano, G., Haussler, D. et al.“Evolutionarily conserved elements in vertebrate, insect, worm, and yeast genomes.” Genome research, vol. 15, pp. 1034-1050, 2005. https://doi.org/10.1101/gr.3715005
Berkovits, B., Mayr, C. “Alternative 3UTRs act as scaffolds to regulate membrane protein localization”. Nature, vol. 522, pp. 363-367, 2015. https://doi.org/10.1038/nature14321
Yeh, H. S., & Yong, J., “Alternative Polyadenylation of mRNAs: 3′-Untranslated Region Matters in Gene Expression.” Molecules and cells, vol. 39, pp. 281-285, 2016. https://doi.org/10.14348/molcells.2016.0035
Kirk, S. J., Cliff, J. M., Thomas, J. A., Ward, T. H. “Biogenesis of secretory organelles during B cell differentiation.” J Leukoc Biol. Vol. 87, pp. 245-55, 2010. doi: 10.1189/jlb.1208774.
Lei, Y., Huang, T., Su, M. et al. “Expression and distribution of immunoglobulin Gin the normal liver, hepatocarcinoma and postpartial hepatectomy liver.” Lab Invest vol. 94, pp. 1283-1295, 2014. https://doi.org/10.1038/labinvest.2014.114
Feldmann, G., Penaud-Laurencin, J., Crassous, J., Benhamou, J. P., “Albumin Synthesis in Liver Cells: It's Morphlogical Demonstration” Gastroenterology, vol. 63, pp. 1036-1049, 1972
Woo, D. H., Kim, S. K., Kim, J. H. et al. “Direct and indirect contribution of human embryonic stem cell-derived hepatocyte-like cells to liver repair in mice.” Gastroenterology. Vol. 142, pp. 602-11, 2012. doi: 10.1053/j.gastro.2011.11.030.
Sharma, N. S., Nagrath, D., & Yarmush, M. L. “Metabolic profiling based quantitative evaluation of hepatocellular metabolism in presence of adipocyte derived extracellular matrix.” PloS one, vol. 6, e20137, 2011. https://doi.org/10.1371/journal.pone.0020137
Reichmuth, A. M., Oberli, M. A., Jaklenec, A., Langer, R., & Blankschtein, D., “mRNA vaccine delivery using lipid nanoparticles.” Therapeutic delivery, vol. 7, 319-334, 2016. https://doi.org/10.4155/tde-2016-0006
Renz, H., Allen, K., Sicherer, S. et al. “Food allergy.” Nat Rev Dis Primers, pp. 4, vol. 17098, 2018. https://doi.org/10.1038/nrdp.2017.98
He, S. H., Zhang, H. Y., Zeng, X. N., Chen, D., & Yang, P. C., “Mast cells and basophils are essential for allergies: mechanisms of allergic inflammation and a proposed procedure for diagnosis.” Acta pharmacologica Sinica, vol. 34, pp. 1270-1283, 2013. https://doi.org/10.1038/aps.2013.88
Holgate, S., Wenzel, S., Postma, D. et al. Asthma. Nat Rev Dis Primers, vol. 1, pp. 15025, 2015. https://doi.org/10.1038/nrdp.2015.25
Choy, D. F., Bradding, P., et. al. “T_{H}2 and T_{H}17 inflammatory pathways are reciprocally regulated in asthma.” Sci Transl Med., vol. 7, pp. 301, 2015. doi: 10.1126/scitranslmed.aab3142. PMID: 26290411.
Liu, J., Pang, Z., Wang, G., Guan, X., Fang, K., Wang, Z., & Wang, F. “Advanced Role of Neutrophils in Common Respiratory Diseases.” Journal of immunology research, 2017, 6710278. https://doi.org/10.1155/2017/6710278
Sun, L., Zhang, L., Yu, J. et al. “Clinical efficacy and safety of anti-PD-1/PD-L1 inhibitors for the treatment of advanced or metastatic cancer: a systematic review and meta-analysis.” Sci Rep, vol. 10, pp. 2083, 2020. https://doi.org/10.1038/s41598-020-58674-4
Thurber, G. M., Schmidt, M. M., & Wittrup, K. D., “Antibody tumor penetration: transport opposed by systemic and antigen-mediated clearance.” Advanced drug delivery reviews, vol. 60, 1421-1434, 2008. https://doi.org/10.1016/j.addr.2008.04.012
Xenaki, K. T., Oliveira, S., & van Bergen En Henegouwen, P., ‘Antibody or Antibody Fragments: Implications for Molecular Imaging and Targeted Therapy of Solid Tumors.” Frontiers in immunology, vol. 8, Article 1287, 2017. https://doi.org/10.3389/fimmu.2017.01287
Nicolson, G. L., “The Fluid-Mosaic Model of Membrane Structure: still relevant to understanding the structure, function and dynamics of biological membranes after more than 40 years.” Biochim Biophys Acta. Vol. 1838, pp. 1451-66, 2014. doi: 10.1016/j.bbamem.2013.10.019.
Zhang, X., & Chang, A., “Somatic mutations of the epidermal growth factor receptor and non-small-cell lung cancer.” Journal of medical genetics, vol. 44, pp. 166-172, 2007. https://doi.org/10.1136/jmg.2006.046102
Owens, M. B., Hill, A. D., & Hopkins, A. M., “Ductal barriers in mammary epithelium.” Tissue barriers, 1:e25933, 2013. https://doi.org/10.4161/tisb.25933
Lee, B. J., & Mace, E. M., “From stem cell to immune effector: how adhesion, migration, and polarity shape T-cell and natural killer cell lymphocyte development in vitro and in vivo.” Molecular biology of the cell, vol. 31, pp. 981-99, 2020. https://doi.org/10.1091/mbc.E19-08-0424
Poznanski, S. M., Ashkar, A. A., “Shining light on the significance of NK cell CD56 brightness.” Cell Mol Immunol vol. 15, pp. 1071-1073, 2018. https://doi.org/10.1038/s41423-018-0163-3
Nielsen, C. M., White, M. J., Goodier, M. R., & Riley, E. M., “Functional Significance of CD57 Expression on Human NK Cells and Relevance to Disease.” Frontiers in immunology, 4, 422, 2013. https://doi.org/10.3389/fimmu.2013.00422
Sun, J. C., Beilke, J. N., & Lanier, L. L. (2009). “Adaptive immune features of natural killer cells.” Nature, 457(7229), 557-561. https://doi.org/10.1038/nature07665
Abel, A. M., Yang, C., Thakar, M. S., & Malarkannan, S., “Natural Killer Cells: Development, Maturation, and Clinical Utilization.” Frontiers in immunology, 9, 1869, 2018. https://doi.org/10.3389/fimmu.2018.01869
Brillantes, M., & Beaulieu, A. M. (2020). Memory and Memory-Like NK Cell Responses to Microbial Pathogens. Frontiers in cellular and infection microbiology, 10, 102. https://doi.org/10.3389/fcimb.2020.00102
Lelaidier, M., Diaz-Rodriguez, Y., Cordeau, M., Cordeiro, P., Haddad, E., Herblot, S., & Duval, M., “TRAIL-mediated killing of acute lymphoblastic leukemia by plasmacytoid dendritic cell-activated natural killer cells.” Oncotarget, vol. 6, pp. 29440-29455, 2015. https://doi.org/I 0.18632/oncotarget.4984
Shimasaki N., Jain A., Campana D., “NK cells for cancer immunotherapy.” Nat. Rev. Drug Discov. Vol. 19, pp. 200-218, 2020. doi: 10.1038/s41573-019-0052-1
Larson, R. C., Maus, M. V. “Recent advances and discoveries in the mechanisms and functions of CART cells.” Nat Rev Cancer. vol. 21, pp. 145-161, 2021. doi: 10.1038/s41568-020-00323-z.
Brudno, J. N., Lam, N., Vanasse, D. et al., “Safety and feasibility of anti-CD19 CART cells with fully human binding domains in patients with B-cell lymphoma.” Nat Med, vol. 26, pp. 270-280, 2020. https://doi.org/10.1038/s41591-019-0737-3
Powell, A. B., Goldstein, H., Nixon, D. F., Bollard, C. M., Lynch, R. M., Jones, R. B., & Cruz, C., et al., “Engineered Antigen-Specific T Cells Secreting Broadly Neutralizing Antibodies: Combining Innate and Adaptive Immune Response against HIV. Molecular therapy.” Methods & clinical development, 19, 78-88, 2020 https://doi.org/10.1016/j.omtm.2020.08.015
Qin, V. M., D'Souza, C., Neeson, P. J., & Zhu, J. J., “Chimeric Antigen Receptor beyond CAR-T Cells.” Cancers, vol. 13, No. 404, 2021. https://doi.org/10.3390/cancers13030404
Long, A. H., Haso, W. M., Shern, J. F., Wanhainen, K. M., Murgai, M., Ingaramo, M., Smith, J. P., Walker, A. J., Kohler, M. E., Venkateshwara, V. R., Kaplan, R. N., Patterson, G. H., Fry, T. J., Orentas, R. J., & Mackall, C. L. (2015). 4-1BB costimulation ameliorates T cell exhaustion induced by tonic signaling of chimeric antigen receptors. Nature medicine, 21(6), 581-590. https://doi.org/10.1038/nm.3838
Benmebarek, M. R., Karches, C. H., Cadilha, B. L., Lesch, S., Endres, S., Kobold, S., “Killing Mechanisms of Chimeric Antigen Receptor (CAR) T Cells.” Int J Mol Sci. vol. 20, 1283, 2019. doi: 10.3390/ijms20061283.
Krause, A., Guo, H. F., Latouche, J. B., Tan, C., Cheung, N. K., & Sadelain, M., “Antigen-dependent CD28 signaling selectively enhances survival and proliferation in genetically modified activated human primary T lymphocytes.” The Journal of experimental medicine, vol. 188, pp. 619-626, 1998.
Vandenberghe, P., Freeman, G. J., Nadler, L. M., Fletcher, M. C., Kamoun, M., Turka, L. A., Ledbetter, J. A., Thompson, C. B., & June, C. H., “Antibody and B7/BB1-mediated ligation of the CD28 receptor induces tyrosine phosphorylation in human T cells.” The Journal of experimental medicine, vol. 175, pp. 951-960, 1992.
Fujiwara, K., Tsunei, A., Kusabuka, H., Ogaki, E., Tachibana, M., & Okada, N., “Hinge and Transmembrane Domains of Chimeric Antigen Receptor Regulate Receptor Expression and Signaling Threshold.” Cells, vol. 9, pp. 1182, 2020. https://doi.org/10.3390/ce11s9051182
Whitlow, M., Bell, B. A., Feng, S. L., Filpula, D., Hardman, K. D., Hubert, S. L., Rollence, M. L., Wood, J. F., Schott, M. E., Milenic, D. E., et al., An improved linker for single-chain Fv with reduced aggregation and enhanced proteolytic stability. Protein Eng., vol. 6, pp. 989-95, 1993)
Alabanza, L., Pegues, M., Geldres, C., Shi, V., Wiltzius, J., Sievers, S. A., Yang, S., & Kochenderfer, J. N., “Function of Novel Anti-CD19 Chimeric Antigen Receptors with Human Variable Regions Is Affected by Hinge and Transmembrane Domains.” Molecular therapy: the journal of the American Society of Gene Therapy, vol. 25, pp. 2452-2465, 2017. https://doi.org/10.1016/j.ymthe.2017.07.013
Noaks, E., Peticone, C., Kotsopoulou, E., & Bracewell, D. G., “Enriching leukapheresis improves T cell activation and transduction efficiency during CAR T processing.” Molecular therapy. Methods & clinical development, vol. 20, pp. 675-687, 2021. https://doi.org/10.1016/j.omtm.2021.02.002
Mhaidly, R., & Verhoeyen, E., “The Future: In Vivo CAR T Cell Gene Therapy.” Molecular therapy: the journal of the American Society of Gene Therapy, vol. 27, pp. 707-709, 2019. https://doi.org/10.1016/j.ymthe.2019.03.012
Rubinstein, M. P., Lind, N. A., Purton, J. F., Filippou, P., Best, J. A., McGhee, P. A., Surh, C. D., & Goldrath, A. W., “IL-7 and IL-15 differentially regulate CD8+ T-cell subsets during contraction of the immune response.” Blood, vol. 112, pp. 3704-3712, 2008. https://doi.org/10.1182/blood-2008-06-160945
Alizadeh, D., Wong, R. A., Yang, X., Wang, D., Pecoraro, J. R., Kuo, C. F., Aguilar, B., Qi, Y., Ann, D. K., Starr, R., Urak, R., Wang, X., Forman, S. J., & Brown, C. E., “IL15 Enhances CAR-T Cell Antitumor Activity by Reducing mTORC1 Activity and Preserving Their Stem Cell Memory Phenotype.” Cancer immunology research, vol. 7, pp. 759-772, 2019. https://doi.org/10.1158/2326-6066.CIR-18-0466
Maetzig, T., Galla, M., Baum, C., & Schambach, A., “Gammaretroviral vectors: biology, technology and application.” Viruses, vol. 3, pp. 677-713, 2011. https://doi.org/10.3390/v3060677
Stein, S.; Ott, M. G.; Schultze-Strasser, S.; Jauch, A.; Burwinkel, B.; Kinner, A.; Schmidt, M.; Kramer, A.; Schwable, J.; Glimm, H.; et al. Genomic instability and myelodysplasia with monosomy 7 consequent to EVI1 activation after gene therapy for chronic granulomatous disease. Nat. Med. 2010, 16, 198-204
Qin, V. M., D'Souza, C., Neeson, P. J., & Zhu, J. J., “Chimeric Antigen Receptor beyond CAR-T Cells.” Cancers, 13:404, 2021. https://doi.org/10.3390/cancers13030404
Suarez, E. R., Chang, d., Sun, J., Sui, J., Freeman, G. J., Signoretti, S., Zhu, Q., & Marasco, W. A., “Chimeric antigen receptor T cells secreting anti-PD-L1 antibodies more effectively regress renal cell carcinoma in a humanized mouse model.” Oncotarget, vol. 7, pp. 34341-34355, 2016. https://doi.org/10.18632/oncotarget. 9114
Szabo, P. A., Miron, M., & Farber, D. L., “Location, location, location: Tissue resident memory T cells in mice and humans.” Science immunology, 4, eaas9673, 2019.1 https://doi.org/10.1126/sciimmunol.aas9673;
Shin, H., & Iwasaki, A., “Tissue-resident memory T cells.” Immunological reviews, vol. 255, pp. 165-181, 2013. https://doi.org/10.1111/imr.12087
Truong, B., Allegri, G., Liu, X. B., Burke, K. E., Zhu, X., Cederbaum, S. D., Haberle, J. P., Martini, G. V., Lipshutz, G. S., “Lipid nanoparticle-targeted mRNA therapy as a treatment for the inherited metabolic liver disorder arginase deficiency” PNAS, vol. 116, pp. 21150-21159, 2019. DOI: 10.1073/pnas.1906182116
Kim, M., Jeong, M., Woo, H. A., Nam, K. T., Lee, K., Lee, H., “Engineered ionizable lipid nanoparticles for targeted delivery of RNA therapeutics into different types of cells in the liver” vol. 7, pp. 1-12, 2021. DOI: 10.1126/sciadv.abf4398
Senior, B. W., Woof, J. M., “The influences of hinge length and composition on the susceptibility of human IgA to cleavage by diverse bacterial IgA1 proteases.” J Immunol. Vol. 174, pp. 7792-9, 2005. doi: 10.4049/jimmunol.174.12.7792.
Veiga, N., Goldsmith, M., Granot, Y. et al. “Cell specific delivery of modified mRNA expressing therapeutic proteins to leukocytes.” Nat Commun, vol. 9, pp. 4493, 2018. https://doi.org/10.1038/s41467-018-06936-1
Yip, M. S. et al. “Antibody-dependent enhancement of SARS coronavirus infection and its role in the pathogenesis of SARS.”, Hong Kong Med. J. vol. 22, pp. 25-31, 2016
Wang, S. F. et al. “Antibody-dependent SARS coronavirus infection is mediated by antibodies against spike proteins.”, Biochem. Biophys. Res. Commun. vol. 451, pp. 208-214, 2014.
Yasui, F. et al. “Prior immunization with severe acute respiratory syndrome (SARS)-associated coronavirus (SARS-CoV) nucleocapsid protein causes severe pneumonia in mice infected with SARS-CoV.”, J. Immunol., vol. 181, pp. 6337-6348, 2008.
Janeway, C., Travers, P., Walport, M., Shlomchik, M., Janeway's Immunobiology, Ninth Edition. 2016. Garland Science: New York, N.Y. ISBN: (Paperback) 978-0815345053.
Stavnezer, J., & Schrader, C. E. “IgH chain class switch recombination: mechanism and regulation.”, Journal of immunology (Baltimore, Md.: 1950), vol. 193, No. 11, pp. 5370-5378, 2014. https://doi.org/10.4049/jimmunol.1401849
Eigenmann M. J., Karlsen, T. V., Wiig, H., et al. “Interstitial IgG antibody pharmacokinetics assessed by combined in vivo- and physiologically-based pharmacokinetic modelling approaches”. J Physiol. Vol. 595, pp. 7311-7330, 2017. doi: 10.1113/JP274819. Epub 2017 Oct. 29.
Borrok, M. J., DiGiandomenico, A., Beyaz, N., Marchetti, G. M., Barnes, A. S., Lekstrom, K. J., Phipps, S. S., McCarthy, M. P., Wu, H., Dall'Acqua, W. F., Tsui, P., & Gupta, R. “Enhancing IgG distribution to lung mucosal tissue improves protective effect of anti-Pseudomonas aeruginosa antibodies.” JCI insight, vol. 3, pp. e97844) https://doi.org/10.1172/jci.insight.97844
Kim, H. Y., Tsai, S., Izadjoo, M. J., et al. “Production and characterization of chimeric monoclonal antibodies against Burkholderia pseudomallei and B. mallei using the DHFR expression system.” PloS one, vol. 6, pp. e19867, 2011. https://doi.org/10.1371/journal.pone.0019867
Lee, E. C., Liang, Q., Ali, H. et al. “Complete humanization of the mouse immunoglobulin loci enables efficient therapeutic antibody discovery.” Nat Biotechnol vol. 32, pp. 356-363, 2014. https://doi.org/10.1038/nbt.2825
Tjelle, T. E., Corthay, A., Lunde, E., Sandlie, I., Michaelsen, T. E., Mathiesen, I., Bogen, B., “Monoclonal antibodies produced by muscle after plasmid injection and electroporation” Mol. Ther., vol. 9, pp. 328-336, 2004.
Lei, Y., Huang, T., Su, M. et al. Expression and distribution of immunoglobulin Gin the normal liver, hepatocarcinoma and postpartial hepatectomy liver. Lab Invest vol. 94, pp. 1283-1295, 2014. https://doi.org/10.1038/labinvest.2014.114
Andrews, C. D., Luo, Y., Sun, M, Ho D. D., et al. “In Vivo Production of Monoclonal Antibodies by Gene Transfer via Electroporation Protects against Lethal Influenza and Ebola Infections Molecular Therapy”—Methods and Clinical Development, vol. 7, No. 15, pp. 74-82, 2017
Kim, et al., “Use of the Human Elongation Factor 1 O. Promoter as a Versatile and Efficient Expression System’. Gene, vol. 91, pp. 217-223, 1990.
Guo, et al., “Evaluation of Promoter Strength for Hepatic Gene Expression InVivo Following Adenovirus-Mediated Gene Transfer”, Gene Ther, vol. 3, pp. 802-810, 1996.
Furler, et al., “Recombinant AAV Vectors Containing the Foot-and-Mouth Disease Virus 2A Sequence Confer Efficient Bicistronic Gene Expression in Cultured Cells and Rat Substantia Nigra Neurons’. Gene Ther, vol. 8, pp. 864-873, 2001.
Wargnier, A., Legros-Maida, S., Bosselut, R., Bourge, J. F., Lafaurie, C., Ghysdael, C. J., Sasportes, M., & Paul, P., “Identification of human granzyme B promoter regulatory elements interacting with activated T-cell-specific proteins: implication of Ikaros and CBF binding sites in promoter activation.” Proceedings of the National Academy of Sciences of the United States of America, vol. 92, pp. 6930-6934, 1995. https://doi.org/10.1073/pnas.92. 15.6930
Penix, L., Weaver, W. M., Pang, Y., Young, H. A., & Wilson, C. B., “Two essential regulatory elements in the human interferon gamma promoter confer activation specific expression in T cells.” The Journal of experimental medicine, vol. 178, pp. 1483-1496, 1995. https://doi.org/10.1084/jem.178.5.1483
Attema J L, Reeves R, Murray V, Levichkin I, Temple M D, Tremethick D J, Shannon M F. “The human IL-2 gene promoter can assemble a positioned nucleosome that becomes remodeled upon T cell activation.” J Immunol., Vol. 169, pp. 2466-76, 2002. 10.4049/jimmunol.169.5.2466.
Tsai, E. Y., Falvo, J. V., Tsytsykova, A. V., Barczak, A. K., Reimold, A. M., Glimcher, L. H., Fenton, M. J., Gordon, D. C., Dunn, I. F., & Goldfeld, A. E., “A lipopolysaccharide-specific enhancer complex involving Ets, Elk-1, Sp1, and CREB binding protein and p300 is recruited to the tumor necrosis factor alpha promoter in vivo.” Molecular and cellular biology, vol. 20, pp. 6084-6094. https://doi.org/10.1128/MCB.20.16.6084-6094.2000
Yu C R, Ortaldo J R, Curiel R E, Young H A, Anderson S K, Gosselin P., “Role of a STAT binding site in the regulation of the human perforin promoter.” J Immunol., vol. 162, pp. 2785-90, 1999.
Conese, M., Auriche, C. & Ascenzioni, F. Gene “Therapy Progress and Prospects: Episomally maintained self-replicating systems.”, Gene Ther., vol. 11, pp. 1735-1′741, 2004. https://doi.org/10.1038/sj.gt.3302362
Kotin, “Prospects for the Use of Adeno-Associated Virus as a Vector for Human Gene Therapy’. Hum. Gene Ther . . . vol. 5, pp. 793-801, 1994.
Janik, J. E., Huston, M. M., Rose, J. A., “Locations of adenovirus genes required for the replication of adenovirus-associated virus.”, Proc. Natl. Acad. Sci., vol. 78 pp. 1925-1929, 1981.
Buller, R. M. L., et al. “Herpes simplex virus types 1 and 2 completely help adenovirus-associated virus replication.”, J Virol vol. 40, pp. 241-24, 1981.
Matsushita, T., Elliger, S., Elliger, C., et al. “Adeno-associated virus vectors can be efficiently produced without helper virus.”, Gene Ther., vol. 5, No. 7, pp. 938-945, 1998) doi:10.1038/sj.gt.3300680
Milone, M. C., O'Doherty, U. “Clinical use of lentiviral vectors.”, Leukemia. vol. 32, No. 7, pp. 1529-1541, 2017. doi:10.1038/s41375-018-0106-0
Wanisch, K., & Yáñez-Muñoz, R. J., “Integration-deficient lentiviral vectors: a slow coming of age. Molecular therapy.”, The journal of the American Society of Gene Therapy, vol. 17, No. 8, pp. 1316-1332, 2009. https://doi.org/10.1038/mt.2009.122
Campochiaro P. A., Lauer, A. K., Sohn, E. H., Mir, T. A., Naylor S., Anderton, M. C., et al. “Lentiviral vector gene transfer of endostatin/angiostatin for macular degeneration (GEM) study.”, Hum Gene Ther. vol. 28, pp. 99-111, 2017.
Milani M, Annoni A, Biffi M, Di Tomaso T, et al. Genome editing for scalable production of alloantigen-free lentiviral vectors for in vivo gene therapy. EMBO Mol Med. 2017; 9:1558-73.
Cascalho, M., Huynh, D., Lefferts, A. R. et al. “Durable targeting of B-lymphocytes in living mice.”, Sci Rep, vol. 8, pp. 11143, 2018. https://doi.org/10.1038/s41598-018-29452-0
Wu, Z., Asokan, A., Samulski, R. J., “Adeno-associated virus serotypes: vector toolkit for human gene therapy.”, Mol Ther. vol. 14, pp. 316-327, 2006.
Sun, M., Li, L., Sheng Gao, Q., Pad S., “Antigen Recognition by an Antibody Light Chain” The Journal of Biological Chemistry, vol. 269, pp. 734-738, 1994.
Ramanathan, A., & Chan, S. H., “mRNA capping: biological functions and applications.” Nucleic acids research, vol. 44, pp. 7511-7526, 2016. https://doi.org/10.1093/nar/gkw551
Martin, S. A., Paoletti, E., Moss, B., “Purification of mRNA guanylyltransferase and mRNA (guanine-7-) methyltransferase from vaccinia virions.” J Biol Chem., vol. 250, pp. 9322-9, 1975.
Mockey, M., Gonçalves, C., Dupuy, F. P., Lemoine, F. M., Pichon, C., and Midoux, P., “mRNA transfection of dendritic cells: synergistic effect of ARCA mRNA capping with Poly(A) chains in cis and in trans for a high protein expression level.” Biochem. Biophys. Res. Comm. Vol. 340, pp. 1062-1068, 2006. doi: 10.1016/j.bbrc.2005.12.105
Hadzidimitriou, A., Darzentas, N., Belessi, C., et. al., “Evidence for the significant role of immunoglobulin light chains in antigen recognition and selection in chronic lymphocytic leukemia” Blood, vol. 113, pp. 403-411, 2009
Landsverk, O. J., Snir, O., Jahnsen, F. L. et al., “Antibody-secreting plasma cells persist for decades in human intestine.” The Journal of experimental medicine, vol. 214, pp. 309-317. https://doi.org/10.1084/jem.20161590
Khodadadi, L., Cheng, Q., Radbruch, A., & Hiepe, F., “The Maintenance of Memory Plasma Cells.” Frontiers in immunology, vol. 10, pp. 721. https://doi.org/10.3389/fimmu.2019.00721
Banasik, M. B., McCray, P. B. Jr. “Integrase-defective lentiviral vectors: progress and applications.” Gene Ther., vol. 17, pp. 150-7, 2010. doi: 10.1038/gt.2009.135
Apolonia, L., “Development and Application of Non-Integrating Lentiviral Vectors for Gene Therapy” Ph.D. Thesis, University College London; London, U K, 2009.
Yáñez-Muñoz, R. J., Balaggan, K. S., MacNeil, A., et al. “Effective gene therapy with nonintegrating lentiviral vectors.”, Nat Med. Vol. 12, No. 3, pp. 348-353, 2006. doi:10.1038/nm1365
Karwacz, K., Mukherjee, S., Thrasher. A. J., et al. “Nonintegrating lentivector vaccines stimulate prolonged T-cell and antibody responses and are effective in tumor therapy.”, J. Virol. Vol. 83, pp. 3094-3103, 2009.

What is claimed:

1. Episomal expression, genomic integrated lentiviral vector or gammaretroviral vector based expression or mRNA expression of monoclonal or polyclonal antibodies (immunoglobulins) of one or more of isotypes IgG1, IgG2, IgG3, dIgA1 and polymeric immunoglobulin A1, dIgA1, IgA1, dIgA2 and polymeric immunoglobulin A2, dIgA2 and IgA2 where the episome, genomic integrated lentiviral vector or mRNA encodes for the polypeptide sequence for immunoglobulins light and heavy chains as well as J Chain for dIgA1 and dIgA2 that are expressed in the same cell and are identified from one or more of (A) CD27+ IgG memory B-cells (B) CD27+ IgA memory B-cells, (C) any memory B-cell (D) memory plasma B-cell (E) plasma B-cell (F) plasmablasts (G) from any transgenic animal (H) from a mouse or rabbit with a humanized immunized system (I) from a mouse other non-human vertebrate antibody converted into a chimeric antibody. Where IgG and IgA memory B-cells are derived from the blood of persons or animals who are currently infected with, were previously infected, were previously exposed to, has immune specificity to, or are affected by one or more of (1) a virus (s) (2) a systemic ailment such as allergies (3) Allergens (4) fungi (5) bacterial infection (6) cancerous tumor (7) an unnatural virus or toxin (8) microbial infection, (9) any ailment (10) a target protein or variant including self-antigens

2. An mRNA, viral, non-viral or retroviral vector including a lentiviral vector or gammaretroviral vector coding for one or more of dimeric immunoglobulin A1 (dIgA1), and/or dIgA2, where the vector contains the transgenes in any order for (1) the immunoglobulin heavy chain of isotype A1 (IgHA1) or A2 (IgHA2), the immunoglobulin light chain that may be kappa (IgLκ) or lambda (IgLλ) as determined from gene sequencing of the B-cell of interest in claim 1 and J Chain or (2) the immunoglobulin heavy chain of isotype A1 (IgHA1) or A2 (IgHA2), the immunoglobulin light chain that may be kappa (IgLκ) or lambda (IgLλ) as determined from gene sequencing of the B-cell of interest in claim 1, J Chain and MZB1. Where the immunoglobulin light and heavy chains encoded for in any nucleic acid vector were expressed by the same B-cell. Where (A) The vector encoding for dIgA1 or dIgA2 comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site (B) The vector encoding for dIgA1 or dIgA2 comprising in the 5′ to 3′ direction the use of separate promoters and regulatory elements for each transgene (C) The vector encoding for dIgA1 or dIgA2 comprising the 5′ to 3′ direction a promoter operably linked to three or four transgenes where first transgene is separated from the second transgene in the 5′ to 3′ direction by a (1) furin cleavage site, (2) a sequence encoding 2A self-processing cleavage site where the second transgene has a stop codon and (3) in the 5′ to 3′ direction is followed by an internal ribosome entry site (IRES), J Chain and optionally followed by (IRES) and MZB1. The optional incorporation of a Woodchuck hepatitis virus post-transcriptional regulatory element (WPRE) precedes any polyadenylation element for DNA based vectors whether as part of the 5′ LTR or if used in an AAV vector where the polyadenylation tail is the terminal untemplated sequence of mRNA. (D) The mRNA vectors encoding for each of the immunoglobulin heavy chain, immunoglobulin light chain and J Chain as two or three separate vectors intended to be contained together in a single vehicle such as a vesicle or lipid nano particle.

3. An mRNA, viral vector, non-viral vector or retroviral vector coding for any one of IgG1, IgG2, IgG3, IgA1 or IgA2 where the vector contains in any order the transgenes for (1) the immunoglobulin heavy chain IgH, the immunoglobulin light chain that may be kappa (IgLκ) or lambda (IgLλ) as determined from gene sequencing of the cell of interest where (A) The vector comprising in the 5′ to 3′ direction a promoter operably linked to the two transgenes expressed as a single open reading frame where each transgene is separated from the subsequent transgene in the 5′ to 3′ direction by (1) a furin cleavage site, (2) a sequence encoding a 2A self-processing cleavage site. (B) The vector comprising in the 5′ to 3′ direction the use of separate promoters and polyadenylation elements for each transgene with the optional use of a woodchuck hepatitis virus posttranscriptional regulatory element (WPRE) to precede one or more polyadenylation elements

4. Construction of viral vectors, retroviral vectors, non-viral vectors or mRNA vectors in one or more of claims 1, 2 and 3 wherein the vector is selected from one or more of the group consisting of an adeno-associated virus (AAV) viral vector, an AAV vector, an adenovirus viral vector, a self-inactivating replication-incompetent lentivirus retroviral vector, a self-inactivating replication-incompetent gammaretroviral vector, a self-inactivating lentiviral vector, a self-inactivating gammaretroviral vector, a non-viral vector, an mRNA vector.

5. Delivery of mRNA, viral vector, non-viral vector or retroviral vector in one or more of claim 1, 2, 3, 4, 9, 10, 11, 12, 15, or 16, with an AAV capsid, a self-inactivating integration-deficient lentivirus, a self-inactivating integration-deficient gammaretrovirus, a self-inactivating integration competent lentivirus, a self-inactivating integration competent gammaretrovirus, a pseudotyped lentivirus, a pseudotyped gammaretrovirus, a vesicle based delivery system, a lipid nanoparticle, or as a naked vector via electroporation.

6. The vector according to claims 1, 2, 3, 9, 10 and 11 where the sequence encoding the furin cleavage site encodes an oligopeptide with the consensus sequence from a group consisting of RXK(R)R (SEQ ID NO: 12), RXRYKR (SEQ ID NO: 13), RXRFKR (SEQ ID NO: 14)

7. The vector according to claims 1, 2, 3, 9, 10, 11, 12, 14, 15 and 16, where the 2A self-processing cleavage site is from a group consisting of (SEQ ID NO: 15), (SEQ ID NO: 17) or (SEQ ID NO: 19).

8. In vivo Administration of the mRNA, viral, non-viral or retroviral vectors in any of claims 1, 2, 3, 4, 5, 9, 10, 11, 12, 13, 14, 15, 16, 20, 21, 22, 23 and 32 to one or more of an animal and/or a human via intramuscular administration to skeletal muscle, intramuscular administration to skeletal muscle with the use of electroporation, intravenous administration, tissue specific administration proximal to a supporting lymph node, direct injection or micro injection into the lamina propria of the stomach, direct injection into the lamina propria of the small intestine, direct injection into the lamina propria of the trachea or bronchi which may be administered with an endoscope or administration proximal to lymph nodes. Optional, one or more additions of target antigens or target proteins or mRNA encoding for them to activate B-cells that received the vectors. Ex vivo administration of the gene therapy into the target cell such as a target immune cell including B-cells, T-cells and NK cells. Ex vivo administration of the gene therapy into the target Chimeric Antigen Receptor (CAR) engineered cell such CAR T-cells and CAR NK cells.

9. Episomal, genomic integrated lentiviral vector or gammaretroviral vector, mRNA expression of monoclonal or polyclonal antibodies (immunoglobulins) whose polypeptide sequence for V_{H} and V_{L} immunoglobulin light and heavy chains are determined and identified from claims 1, 2 and 3 where IgG and IgA B-cells are derived from the blood of persons or animals who are currently infected with or were previously infected with, exposed to, has immune specificity to, or affected by one or more of (1) a virus (s) (2) a systemic ailment such as allergies (3) Allergens (4) fungi (5) bacterial infection (6) cancerous tumor (7) an unnatural virus or toxin (8) any ailment (9) a target protein or variant (10) an immune system protein such as Immunoglobulin class E (IgE) or a cytokine, are modified in the following way: V-regions (both V_{L} and V_{H}) are coded for exactly as they were identified from the source in the cell expressing the potent immunoglobulin or one or more of V_{L} and V_{H} may optionally have one or more of the Complementary Determining Regions (CDR) or Framework regions (FR) modified or replaced with another FR region. Where (A) one or more of the domains of the immunoglobulin heavy chain constant domains consisting of C_{H}1, hinge and C_{H}2 are replaced by one or more of (1) natural human derived constant regions to reduce immunogenicity and/or modulate effector functions, (2) engineered constant regions to modulate effector functions and (3) adding a furin cleavage site residue to the C-terminal end of the immunoglobulin heavy chain where in each case of (1), (2) and (3) C_{H}3 must be derived from IgA1 or IgA2 regardless of whether it is engineered or not. The immunoglobulin light chain's (IgL) constant regions (CL) is optionally added or modified by one of more of (4) changing type e.g. kappa (κ) to lambda (λ) or lambda (λ) to kappa (κ), (5) adding a furin cleavage site residue on the C-terminal end, (6) modifying the hinge length, (7) modifying the hinge amino acids or amorphous chain amino acids. Where the dIgA immunoglobulins use immunoglobulins heavy and light chains identified from a single IgA and through incorporating J Chain into the vector where J Chain may optionally be modified by adding to its C-terminal end a furin cleavage site residue.

10. Episomal, genomic integrated lentiviral vector or gammaretroviral vector or mRNA expression of monoclonal or polyclonal antibodies (immunoglobulins) whose polypeptide sequence for V_{H} and V_{L} immunoglobulin light and heavy chains in addition to the constant light chain are determined and identified from claims 1, 2 and 3 or from a previously identified potent immunoglobulin where IgG and IgA B-cells from claims 1, 2 and 3 are derived from the blood of persons or animals who are currently infected with or were previously infected with, exposed to or affected by one or more of ((1) a virus (s) (2) a systemic ailment such as but not limited to allergies (3) Allergens (4) fungi (5) bacterial infection (6) cancerous tumor (7) an unnatural virus or toxin (8) any ailment (9) a target protein or variant (10) an immune system protein such as Immunoglobulin class E (IgE) or a cytokine, are modified in the following way. An IgG1, IgG3, IgA1, IgA2 dIgA1 or dIgA2 immunoglobulin identified to be of moderate to high association constant against the protein of interest may be modified such as by generating the dIgA1 or dIgA2 recombinant isotype of that immunoglobulin that is by using the dIgA1 or dIgA2 heavy chain constant region to replace the constant region of the parent immunoglobulin. Where the resulting dIgA1 or dIgA2 antibody (A) may be modified by engineering the constant domains to modify Fc receptor binding with mutagenesis techniques or with mixes of two constant regions from two isotypes or subclasses that may be accomplished by replacing one or more of the C_{H}1, hinge or C_{H}2 regions with one or more of one or more of the C_{H}1, hinge or C_{H}2 regions respectively as a one-to-one correspondence of replacement. (B) Or by replacing the Fab or F(ab′)2—as identified from the B-cell from claims 1, 2 and 3 with the dIgA1 or dIgA2 Fab or F(ab′)2 respectively where the dIgA1 or dIgA2 may be optionally modified on the immunoglobulin light chain by adding to its C-terminal end a furin cleavage site residue as a result of a byproduct of furin cleavage. Where the dIgA immunoglobulins are expressed through incorporating, the immunoglobulin heavy and light chains of dIgA, J Chain into the vector and optionally MZB1.

11. Episomal, genomic integrated lentiviral vector or gammaretroviral vector or mRNA expression of polyclonal or monoclonal antibodies (immunoglobulins) based on one or more of dIgA1 and dIgA2 where both of the V_{H} and V_{L} regions or the antibody binding fragment (Fab) are identified or derived from single chain variable fragments (scF_{V}) or Fab from combinatorial libraries assessed by phage display technology. Where scFᵥ used to identify V_{H} and V_{L} used for the formation in one or more of dIgA1 and polymeric immunoglobulin A1 and/or dIgA2 and polymeric immunoglobulin A2 produced by random recombination and shuffling with optional mutagenesis of human V_{H} and V_{L} regions of scF_{V} from human antibody libraries derived different human B-cells including, naïve B-cells, memory B-cells and even plasma secreting B-cells where cells may be derived from the blood of humans of that recovered from the virus of interest or from another human source or from mice with humanized immune systems where the potent single chain variable fragment fragments expressed in antibody libraries are used to identify potent immunoglobulin V_{H} and V_{L} regions pairs that may be used to recombine the V_{L} with the constant regions of the immunoglobulin light chain (IgLκ) or (IgLλ) and combining the V_{H} regions with any of the constant region of IgA1 and IgA2 and their engineered variants, including modified hinge variants that may be used to reduce immunogenicity to produce engineered dimeric immunoglobulins of one or more of dIgA1 and dIgA2. Where dIgA1 and polymeric immunoglobulin A1 and dIgA2 and polymeric immunoglobulin A2 will be produced from IgA1 and IgA2 respectively from their co-expression with J-chain and optionally MZB1 in the same vector. Where up to one or more of IgL, IgH, J-chain and MZB1 may be optionally modified by adding to their C-terminal ends a furin cleavage site residue as a result of a byproduct of furin cleavage. Where MZB1 may be optionally modified by adding to its N-terminal end proline as a result of 2A self-processing peptides cleavage/ribosomal skip.

12. Modification of dIgA1 and polymeric immunoglobulin A1 or dIgA2 and polymeric immunoglobulin A2 as defined in claim 11 whose V-regions V_{H} and V_{L} are derived from single chain scFᵥ variable fragments derived phage display technology and subsequent mutagenesis to modulate effector functions or reduce antibody-dependent enhance of infection. The constant regions of such dIgA1 and dIgA2 antibodies may be modified such (A) that one or more of the C_{H}1, hinge or C_{H}2 or C_{H}3 domains of the immunoglobulin heavy chain may be modified to modulate effector functions, reduce antibody dependent enhancement of infection, increase half-life, enhance formation of the Beta sheet complex (Beta Sandwich) with J Chain to favor the formation of higher valency forms of polymeric immunoglobulin A, or modify flexibility between the Fc and the Fab afforded by the hinge amino acids and (B) optional modification of the CL domain.

13. Construction of viral vectors, retroviral vectors, episomal vectors or mRNA vectors in one or more of claims 9, 10, 11, 12, 14 and 15 wherein the vector is selected from one or more of the group consisting of an adeno-associated virus (AAV) viral vector, an AAV vector, an adenovirus viral vector, a self-inactivating replication-incompetent lentivirus retroviral vector, a self-inactivating replication-incompetent gammaretroviral vector, a self-inactivating replication-incompetent integration deficient lentivirus retroviral vector, a self-inactivating replication-incompetent integration deficient gammaretrovirus retroviral vector, a self-inactivating lentivirus vector, a self-inactivating gammaretrovirus vector, a non-viral vector, an mRNA vector.

14. The vector according to claims 1, 2, 3, 9, 10, 11, 12, 15 and 16 wherein the promoter and intermediate promoter is selected from the group consisting of an elongation factor 1-alpha promoter (EF1α) promoter, a Feek promoter, a phosphoglycerate kinase-1 promoter (PGK) promoter, a human cytomegalovirus immediate early gene promoter (CMV), an internal ribosome entry site (IRES) substitution for an intermediate promoter that has a similar function to an intermediate promoter, a chimeric liver specific promoter (LSP), a cytomegalovirus enhancer/chicken beta-actin promoter (CAG), a tetracycline responsive promoter (TRE), a transthyretin promoter (TTR), a simian virus 40 promoter (SV40), a CK6 promoter and a RNA polymerase III (Pol III) promoter, the natural promoter or truncated promoter established for any gene highly expressed by a cell in humans or highly expressed in the target cell such but not limited to a perforin promoter (SEQ ID NO: 110), a granzyme B promoter (SEQ ID NO: 113), an IFNγ promoter (SEQ ID NO: 111 and SEQ ID NO:112), a TNFα promoter (SEQ ID NO: 106, SEQ ID NO: 107, SEQ ID NO:108 and SEQ ID NO: 109) and an IL-2 promoter (SEQ ID NO: 115, SEQ ID NO:116 and SEQ ID NO: 117).

15. An mRNA, viral, non-viral or retroviral vector including a lentiviral vector or gammaretroviral vector coding for one or more of dimeric immunoglobulin A1 (dIgA1) and/or dIgA2 and polymeric immunoglobulin A2 in claims 9, 10, 11, and where the viral vector, non-viral vector or retroviral vector contains the transgenes in any order for (1) the immunoglobulin heavy chain of isotype A1 (IgHA1) or A2 (IgHA2), the immunoglobulin light chain that may be kappa (IgLκ) or lambda (IgLλ) as determined from gene sequencing of the B-cell of interest in claim 1 and J Chain or (2) the immunoglobulin heavy chain of isotype A1 (IgHA1) or A2 (IgHA2), the immunoglobulin light chain that may be kappa (IgLκ) or lambda (IgLλ) as determined from gene sequencing of the B-cell of interest in claim 1, J Chain and MZB1. Where the immunoglobulin light and heavy chains encoded for in any vector were expressed by the same B-cell. Where (A) The vector encoding for dIgA1 comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector encoding for dIgA1 comprising in the 5′ to 3′ direction the use of separate promoters and regulatory elements for each transgene with the optional the use of an internal ribosome entry site (IRES) between two transgenes in place of a promoter. (C) The vector encoding for dIgA1 comprising the 5′ to 3′ direction a promoter operably linked to three transgenes where first transgene is separated from the subsequent transgene in the 5′ to 3′ direction by a furin cleavage site, a sequence encoding 2A self-processing cleavage site where the second transgene has a stop codon and in the 5′ to 3′ direction is followed by an internal ribosome entry site (IRES), the optional incorporation of a Woodchuck hepatitis virus post-transcriptional regulatory element (WPRE) precedes any polyadenylation element for DNA based vectors whether as part of the 5′ LTR or if used in an AAV vector where the polyadenylation tail is the terminal sequence of mRNA.

16. An mRNA, viral, non-viral or retroviral vector including a lentiviral vector or gammaretroviral vector coding for any of IgG1, IgG3, IgG3 and IgA1 as it is described in claims 9 and 10 where the viral vector, non-viral vector or retroviral vector contains the transgenes for (1) immunoglobulin heavy chain (IgH) and immunoglobulin light chain that may be kappa or lambda as determined from gene sequencing of the B-cell of interest in claim 9 or 10 (IgLκ) or (IgLλ). Where (A) The vector comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector comprising in the 5′ to 3′ direction the use of separate promoters and regulatory elements for each transgene the optional incorporation of a Woodchuck hepatitis virus post-transcriptional regulatory element (WPRE) precedes any polyadenylation element for DNA based vectors whether as part of the 5′ LTR or if used in an AAV vector where the polyadenylation tail is the terminal sequence of mRNA. (C) The mRNA vectors encoding for each of the immunoglobulin heavy chain, immunoglobulin light chain and J Chain as two or three separate vectors.

17. The vector according to claims 1, 2, 3, 4, 9, 10, 11, 12, 14, 15 and 16 wherein (A) the intermediate promoter is selected from the group consisting of an elongation factor 1-alpha promoter (EF1α), or internal ribosome entry site (IRES) promoter substitute, a human cytomegalovirus immediate early gene promoter (CMV), a chimeric liver specific promoter (LSP), a cytomegalovirus enhancer/chicken beta-actin promoter (CAG) and a simian virus 40 promoter (SV40). (B) the polyadenylation site is selected from a group consisting of a simian virus 40 polyadenylation site (SV40 polyA), a Bovine Growth Hormone polyadenylation site (BGH polyA), a polyadenylation site that is located in U5 of 3′ LTR or 5′ LTR, a non-canonical polyadenylation site that is located in the 3′ UTR and (C) The optional use of one or more Woodchuck hepatitis virus posttranscriptional regulatory elements (WPRE)

18. Any combination of one of more of claims 1, 2, 3, 9, 10, 11, 12, 14, 15, 16 and 21. That is polyclonal expression of immunoglobulins may consist of a mix of naturally identified immunoglobulins, artificially modified immunoglobulins and engineered immunoglobulins.

19. The vector according to claims 9, 10, 11, 12, 14, 15 and 16 where the sequence encoding the furin cleavage site encodes an oligopeptide with the consensus sequence from a group consisting of RXKRR (SEQ ID NO: 12), RXRYKR (SEQ ID NO: 13), RXRFKR (SEQ ID NO: 14)

20. The vectors according to claims 2, 3, 9, 10, 11, 12, 14, 15, and 16 where any unnatural 5′ UTR and 3′ UTR, any natural 5′ UTR and 3′ UTR used by humans or any truncated variant of the 5′ UTR and 3′ UTR used by humans is incorporated into vector constructs (A) without one or more of the 5′ UTR and 3′ UTR (B) substituted in for vectors constructs with one or more of the 5′ UTR and 3′ UTR normally transcribed with the gene. Where the 5′ UTR is placed directly before a transgene with a start codon to start translation and directly following a promoter that is not an IRES. Where the 3′ UTR is placed directly between the final transgene stop codon and the remaining part of the vector in the direction of transgene translation.

21. The vectors and immunoglobulins according to claims 2, 9, 10, 11, 15, 16 and 20 where (A) any vector construct has optionally excluded one or more furin cleavage sequences leaving only 2A self-processing peptide sequences between two or more consecutive transgenes that are part of a single open reading frame and (B) one or more of IgH, IgL or J Chain that is modified by adding to their C-terminal ends a 2A self-processing peptide residue as a result of a byproduct 2A cleavage.

22. (canceled)

23. The vectors and immunoglobulins according to claims 2, 9, 10, 11, 15, 16 and 20 where a dimeric single chain fragment variable-fragment crystallizable fusion of immunoglobulin A (dscFV-FcIgA) is generated by replacing the antibody binding fragment (Fab) with a single chain variable fragment (scFv) and fusing at the C-terminal end of the scFv to the N-terminal end of the hinge of the fragment crystallizable region of IgA1 or IgA2 according to 2, 3, 9, 10, 11, 15, and 16 where the hinge may be a modified hinge. Where the immunoglobulin light chain encoding transgene, the immunoglobulin heavy chain encoding transgene and any furin cleavage site and 2A self-processing peptide genes or IRES gene placed between the immunoglobulin light chain and immunoglobulin heavy chain are replaced in entirety with the following vector element in the 5′ to 3′ direction the signal peptide followed by the single chain variable fragment (scFv) followed by the fragment crystallizable region (Fab) where the remaining portions of the vector including J Chain and a vector element that separates J Chain from the scIgA element such as an IRES or alternatively a furin cleavage site followed by a 2A self-processing peptide as described in 2, 3, 9, 10, 11, 15, and 16 remain intact.

24. Modification of the RNA vectors in 2, 9, 10, 11, 15, 16, 20 21, 22 and 23 where the 5′cap m⁷G(5′)ppp(5′)N^{m}- is added by a vaccina virus capping enzyme to the 5′ end of RNA. Where the poly(A) tail is between 120 and 150 adenosine nucleotides in length and may be added in vitro by a poly(A) polymerase.

25. The vectors and immunoglobulins according to claims 2, 9, 10, 11, 15, 16, 20, 22 and 23 where a gene encoding for a chimeric antigen receptor (CAR) comprising a signal peptide that is cleaved following translation followed by a single chain Variable fragment (scFv), a hinge, a transmembrane domain and an intracellular signaling domain. Where scFv that may be made up of a V_{H} V_{H} pair, a V_{L} V_{L} pair or a V_{H} V_{L} pair.

26. The CAR in claim 25 where the transmembrane domain comprises CD28 or CD8α.

27. The CAR in claim 25 comprising one or more additional costimulatory signaling domains positioned between the transmembrane domain and the intracellular signaling domain.

28. The CAR in claim 25 where the costimulatory signaling domain is 4-1BB or CD28.

29. The CAR in claim 25 where the intracellular signaling domain is CD3zeta.

30. The CAR in claim 25 where the scFv is replaced with a Fab

31. The CAR in claim 25 where the hinge comprises CD28 of CD8α.

32. The vectors and immunoglobulins according to claims 2, 9, 10, 11, 15, 16, 20, 22 and 23 where a gene encoding for a chimeric antigen receptor (CAR) in any one of the preceding claims further comprising is incorporated into the vector by (A) adding in the 5′ to 3′ direction following of the stop codon of the final transgene encoding for the dimeric antibody an IRES followed by the nucleic acid encoding for the CAR.

33. A general method to neutralize toxic bacteria in the mucosa.

34. A general method to prevent cancer metastasis, enhance immunoglobulin access to the face of a tumor or carcinoma facing in the direction of the apical face of the epithelium and increase the amount of antibody that can assess a tumor or carcinoma through dIgA active transport to the apical face of epithelium.

35. A general method to eliminate allergies due to a specific allergen by neutralizing allergens with vectors encoding for dIgA1 or dIgA2 and preventing mast cell allergen binding averting mast cell degranulation and preventing dendritic cell (DC) binding of allergens and preventing DC activation of (Tₕ₂) helper T-cells that would otherwise result in increased concentrations of IgE specific to the allergen that increases the severity of a patient's immune response to that allergen.

36. A general method to create an HIV-1 immunization.

37. A general method to treat pneumonia via an administration of an mRNA vector or mRNA vectors encapsulated in a vesicle-based delivery system such as a lipid nano particle encoding for dIgA1 specific to an epitope on a cell surface S. pneumoniae protein such as adhesin SpsA.


SEQUENCE LISTING (143 sequences)

SEQ ID NO: 1 - DNA, 589 bp, Artificial
  Vector Element
  Misc._Feature (1)..(589): Name/Key: Human Cytomegalovirus Immediate Early Enhancer/Promoter (CMV)
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 2 - DNA, 588 bp, Artificial
  Vector Element
  Misc._Feature (1)..(588): Name/Key: Internal Ribosome Entry Site (IRES)
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 3 - DNA, 598 bp, Artificial
  Vector Element
  Misc._Feature (1)..(598): Name/Key: Woodchuck Hepatitis Virus Posttranscriptional Regulatory Element (WPRE)
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 4 - DNA, 236 bp, Artificial
  Vector Element
  Misc._Feature (1)..(236): Name/Key: Simian Virus 40 Polyadenylation Signal (SV40 Poly A)
agctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgg

SEQ ID NO: 5 - DNA, 208 bp, Artificial
  Vector Element
  Misc._Feature (1)..(208): Name/Key: Bovine Growth Hormoen Polyadenylation Signal (BGH Poly A)
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 6 - skipped

SEQ ID NO: 7 - protein, 137 aa, Human
  Misc._Feature (1)..(137): Name/Key: J Chain Without Signaling Peptide
  Misc._Feature (1)..(137): Name/Key: J Chain Without Signaling Peptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 189 aa, Human
  Misc._Feature (1)..(189): Name/Key: Marginal Zone B-1 Cell Specific Protein (MZB1)
MRLSLPLLLLLLGAWAIPGGLGDRAPLTATAPQLDDEEMYSAHMPAHLRCDACRAVAYQMWQNLAKAETKLHTSNSGGRRELSELVYTDVLDRSCSRNWQDYGVREVDQVKRLTGPGLSEGPEPSISVMVTGGPWPTRLSRTCLHYLGEFGEDQIYEAHQQGRGALEALLCGGPQGACSEKVSATREEL

SEQ ID NO: 9 - DNA, 117 bp, Artificial
  Vector Element
  Misc._Feature (1)..(117): Name/Key: Intron I117 5′ Untranslated Region (5′ UTR)
gtaagtgatatctactagatttatcaaaaagagtgttgacttgtgagcgctcacaattgatacttagattcatcgagagggacacgtcgactactaaccttcttctctttcctacag

SEQ ID NO: 10 - protein, 4 aa, Artificial
  Vector Element
  Misc._Feature (1)..(4): Name/Key: Consensus Sequence of Furin Cleavage Site (Furin Cleavage Site)
  Misc._Feature (2)..(2): Xaa = any amino acid
RXKR

SEQ ID NO: 11 - protein, 159 aa, Human
  Misc._Feature (1)..(159): Name/Key: J Chain with Signaling Peptid
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 12 - protein, 5 aa, Artificial
  Vector Element
  Misc._Feature (1)..(5): Name/Key: Consensus Sequence of Furin Cleavage Site 2 (Furin Cleavage Site)
  Misc._Feature (2)..(2): Xaa = any amino acid
RXKRR

SEQ ID NO: 13 - protein, 6 aa, Artificial
  Vector Element
  Misc._Feature (1)..(6): Name/Key: Consensus Sequence of Furin Cleavage Site 3 (Furin Cleavage Site)
  Misc._Feature (2)..(2): Xaa = any amino acid
RXRYKR

SEQ ID NO: 14 - protein, 6 aa, Artificial
  Vector Element
  Misc._Feature (1)..(6): Name/Key: Consensus Sequence of Furin Cleavage Site 4 (Furin Cleavage Site)
  Misc._Feature (2)..(2): Xaa = any amino acid
RXRFKR

SEQ ID NO: 15 - protein, 25 aa, Artificial
  Vector Element
  Misc._Feature (1)..(25): Name/Key: Foot and Mouth Disease Virus 2A Self Cleaving/Self Processing Peptide (F2A) or (2A)
GSGVKQTLNFDLLKLAGDVESNPGP

SEQ ID NO: 16 - protein, 19 aa, Artificial
  Vector Element
  Misc._Feature (1)..(19): Name/Key: Porcine Teschovirus 2A Self Cleaving/Self Processing Peptide (P2A) or (2A)
ATNFSLLKQAGDVEENPGP

SEQ ID NO: 17 - protein, 18 aa, Artificial
  Vector Element
  Misc._Feature (1)..(18): Name/Key: Thosea Asigna Virus-2A Self Cleaving/Self Processing Peptide (T2A) or (2A)
EGRGSLLTCGDVEENPGP

SEQ ID NO: 18 - skipped

SEQ ID NO: 19 - protein, 20 aa, Artificial
  Vector Element
  Misc._Feature (1)..(20): Name/Key: Equine Rhinitis A Virus-2A Self Cleaving/Self Processing Peptide (E2A) or (2A)
QCTNYALLKLAGDVESNPGP

SEQ ID NO: 20 - DNA, 278 bp, Artificial
  Vector Element
  Misc._Feature (1)..(278): Name/Key: Chicken Beat Actin Promoter (CAG)
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 21 - DNA, 6 bp, Human
  Misc._Feature (1)..(6): From Page 155 of the Specification
  Misc._Feature (1)..(6): Name/Key: Kozak Sequence
gccacc

SEQ ID NO: 22 - skipped

SEQ ID NO: 23 - protein, 353 aa, Human
  Misc._Feature (1)..(353): Name/Key: Immunoglobulin Heavy Chain Constant Alpha 1 (IgHCA1 Constant Region)
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 24 - protein, 340 aa, Human
  Misc._Feature (1)..(340): Name/Key: Immunoglobulin Heavy Chain Constant Alpha 2 Allotype A2m(2) (IgHCA2 Constant Region Allotype A2m(2))
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 25 - protein, 106 aa, Human
  Misc._Feature (1)..(1): Xaa = any amino acid
  Misc._Feature (1)..(106): Name/Key: (IgLC_Lambda) Immunoglobuin Lambda Light Chain Constant Region
XQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 26 - protein, 107 aa, Human
  Misc._Feature (1)..(107): Name/Key: (IgLC_Kappa_Km(1)) Immunoglobuin Kappa Light Chain Constant Region Allotype Km(1)
  Misc._Feature (1)..(1): Xaa = any amino acid
  Misc._Feature (56)..(56): Allotype Identifier
  Misc._Feature (94)..(94): Allotype Identifier
XTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 22 aa, Human
  Misc._Feature (1)..(22): Name/Key: Signaling Peptide for J Chain
MKNHLLFWGVLAVFIKAVHVKA

SEQ ID NO: 28 - protein, 19 aa, Human
  Misc._Feature (1)..(19): Name/Key: Signaling Peptide for the Immunoglobulin Heavy Chain (Seq. ID 28)
  Misc._Feature (1)..(19): Most Common in Population
MDWTWRILFLVAAATGAHS

SEQ ID NO: 29 - protein, 19 aa, Human
  Misc._Feature (1)..(19): Name/Key: Signaling Peptide for the Immunoglobulin Heavy Chain (Seq. ID 29)
MDTLCSTLLLLTIPSWVLS

SEQ ID NO: 30 - protein, 19 aa, Human
  Misc._Feature (1)..(19): Name/Key: Signaling Peptide for the Immunoglobulin Kappa Light Chain (Seq. ID 30)
  Misc._Feature (1)..(19): Most Common in Population
MDMRVPAQLLGLLLLWLPG

SEQ ID NO: 31 - protein, 19 aa, Human
  Misc._Feature (1)..(19): Name/Key: Signaling Peptide for the Immunoglobulin Kappa Light Chain (Seq. ID 31)
MDMMVPAQLLGLLLLWFPG

SEQ ID NO: 32 - protein, 20 aa, Human
  Misc._Feature (1)..(20): Name/Key: Signaling Peptide for the Immunoglobulin Kappa Light Chain (Seq. ID 32)
MRVPAQLLGLLLLWLPGARC

SEQ ID NO: 33 - protein, 19 aa, Human
  Misc._Feature (1)..(19): Name/Key: Signaling Peptide for the Immunoglobulin Kappa Light Chain (Seq. ID 33)
MRLPAQLLGLLMLWVPGKD

SEQ ID NO: 34 - protein, 19 aa, Human
  Misc._Feature (1)..(19): Name/Key: Signaling Peptide for the Immunoglobulin Kappa Light Chain (Seq. ID 34)
MRLLAQLLGLLMLWVPGSS

SEQ ID NO: 35 - protein, 19 aa, Human
  Misc._Feature (1)..(19): Name/Key: Signaling Peptide for the Immunoglobulin Lambda Light Chain (Seq. ID 35)
MAWTPLWLTLLTLCIGSVV

SEQ ID NO: 36 - protein, 19 aa, Human
  Misc._Feature (1)..(19): Name/Key: Signaling Peptide for the Immunoglobulin Lambda Light Chain (Seq. ID 36)
MAWTPLFLFLLTCCPGSNS

SEQ ID NO: 37 - protein, 19 aa, Human
  Misc._Feature (1)..(19): Name/Key: Signaling Peptide for the Immunoglobulin Lambda Light Chain (Seq. ID 37)
MAWALLLLTLLTRDTGSWA

SEQ ID NO: 38 - protein, 19 aa, Human
  Misc._Feature (1)..(19): Name/Key: Signaling Peptide for the Immunoglobulin Lambda Light Chain (Seq. ID 38)
MPWALLLLTLLTHSAVSVV

SEQ ID NO: 39 - protein, 19 aa, Human
  Misc._Feature (1)..(19): Name/Key: Signaling Peptide for the Immunoglobulin Lambda Light Chain (Seq. ID 39)
MAWSSLLLTLLAHCTGSWA

SEQ ID NO: 40 - protein, 19 aa, Human
  Misc._Feature (1)..(19): Name/Key: Signaling Peptide for the Immunoglobulin Lambda Light Chain (Seq. ID 40)
MAWSPLFLTLITHCAGSWA

SEQ ID NO: 41 - protein, 19 aa, Human
  Misc._Feature (1)..(19): Name/Key: Signaling Peptide for the Immunoglobulin Lambda Light Chain (Seq. ID 41)
MIYEVSHRPSGVSTRFSAS

SEQ ID NO: 42 - DNA, 18 bp, Human
  Misc._Feature (1)..(18): Name/Key: 5′ Untranslated Region (5′ UTR) of J Chain
agaagaagtgaagtcaag

SEQ ID NO: 43 - DNA, 36 bp, Human
  Misc._Feature (1)..(36): Name/Key: 5′ Untranslated Region (5′ UTR) of the Immunoglobulin Lambda Light Chain (Seq. ID 43)
tgagcgcagaaggcaggactcgggacaatcttcatc

SEQ ID NO: 44 - DNA, 40 bp, Human
  Misc._Feature (1)..(40): Name/Key: 5′ Untranslated Region (5′ UTR) of the Immunoglobulin Lambda Light Chain (Seq. ID 44)
gctgcgggtagagaagacaggactcaggacaatctccagc

SEQ ID NO: 45 - DNA, 29 bp, Human
  Misc._Feature (1)..(29): Name/Key: 5′ Untranslated Region (5′ UTR) of the Immunoglobulin Kappa Light Chain (Seq. ID 45)
gcaggaatcagtcccactcaggacacagc

SEQ ID NO: 46 - DNA, 53 bp, Human
  Misc._Feature (1)..(53): Name/Key: 5′ Untranslated Region (5′ UTR) of the Immunoglobulin Kappa Light Chain (Seq. ID 46)
aggctggacacacttcatgcaggagtcagaccctgtcaggacacagcatagac

SEQ ID NO: 47 - DNA, 63 bp, Human
  Misc._Feature (1)..(63): Name/Key: 5′ Untranslated Region (5′ UTR) of the Immunoglobulin Heavy Chain (Seq. ID 47)
gagagcatcacccagcaaccacatctgtcctctagagatcccctgagagctccgttcctcacc

SEQ ID NO: 48 - DNA, 20 bp, Human
  Misc._Feature (1)..(20): Name/Key: 5′ Untranslated Region (5′ UTR) of the Immunoglobulin Heavy Chain (Seq. ID 48)
agtgactcctgtgccccacc

SEQ ID NO: 49 - DNA, 37 bp, Human
  Misc._Feature (1)..(37): Name/Key: 5′ Untranslated Region (5′ UTR) of the Hemoglobin Subunit Alpha 1
actcttctggtccccacagactcagagagaacccacc

SEQ ID NO: 50 - DNA, 37 bp, Human; identical to SEQ ID NO: 49
  Misc._Feature (1)..(37): Name/Key: 5′ Untranslated Region (5′ UTR) of the Hemoglobin Subunit Alpha 2

SEQ ID NO: 51 - DNA, 47 bp, Human
  Misc._Feature (1)..(47): Name/Key: 5′ Untranslated Region (5′ UTR) of the Fibrinogen-Gamma Chain
gacagtgctgacactacaaggctcggagctccgggcactcagacatc

SEQ ID NO: 52 - DNA, 8 bp, Human
  Misc._Feature (1)..(8): Name/Key: 5′ Untranslated Region (5′ UTR) of the Fibrinogen-Beta Chain
aagtctac

SEQ ID NO: 53 - DNA, 55 bp, Human
  Misc._Feature (1)..(55): Name/Key: 5′ Untranslated Region (5′ UTR) of the Fibrinogen-Alpha Chain
aatcctttctttcagctggagtgctcctcaggagccagccccacccttagaaaag

SEQ ID NO: 54 - DNA, 41 bp, Human
  Misc._Feature (1)..(41): Name/Key: 5′ Untranslated Region (5′ UTR) for Albumin
ctagcttttctcttctgtcaaccccacacgcctttggcaca

SEQ ID NO: 55 - DNA, 788 bp, Human
  Misc._Feature (1)..(788): Name/Key: 3′ Untranslated Region (3′ UTR) of J Chain
tttaagtcattgctgactgcatagctctttttcttgagaggctctccattttgattcagaaagttagcatatttattaccaatgaatttgaaaccagggcttttttttttttttgggtgatgtaaaaccaactccctgccaccaaaataattaaaatagtcacattgttatctttattaggtaatcacttcttaattatatgttcatactctaagtatcaaaatcttccaattatcatgctcacctgaaagaggtatgctctcttaggaatacagtttctagcattaaacaaataaacaaggggagaaaataaaactcaaggactgaaaatcaggaggtgtaataaaatgttcctcgcattcccccccgcttttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctagaactagttacaaaaataatttaaggtgaaatctctaatatttataaaagtagcaaaataaatgcataattaaaatatatttggacataacagacttggaagcagatgatacagacttctttttttcataatcaggttagtgtaagaaattgccatttgaaacaatccattttgtaactgaaccttatgaaatatatgtatttcatggtacgtattctctagcacagtctgagcaattaaatagattcataagcata

SEQ ID NO: 56 - DNA, 135 bp, Human
  Misc._Feature (1)..(135): Name/Key: 3′ Untranslated Region (3′ UTR) of the Immunoglobulin Heavy Chain Class G Subclass 1 (IgHG1)
gtgccacggccggcaagcccccgctccccaggctctcggggtcgcgcgaggatgcttggcacgtaccccgtgtacatacttcccaggcacccagcatggaaataaagcacccagcgcttccctgggcccctgcga

SEQ ID NO: 57 - DNA, 135 bp, Human; identical to SEQ ID NO: 56
  Misc._Feature (1)..(135): Name/Key: 3′ Untranslated Region (3′ UTR) of the Immunoglobulin Heavy Chain Class G Subclass 2 (IgHG2)

SEQ ID NO: 58 - DNA, 135 bp, Human; identical to SEQ ID NO: 56
  Misc._Feature (1)..(135): Name/Key: 3′ Untranslated Region (3′ UTR) of the Immunoglobulin Heavy Chain Class G Subclass 3 (IgHG3)

SEQ ID NO: 59 - DNA, 135 bp, Human; identical to SEQ ID NO: 56
  Misc._Feature (1)..(135): Name/Key: 3′ Untranslated Region (3′ UTR) of the Immunoglobulin Heavy Chain Class G Subclass 4 (IgHG4)

SEQ ID NO: 60 - DNA, 113 bp, Human
  Misc._Feature (1)..(113): Name/Key: 3′ Untranslated Region (3′ UTR) of the Immunoglobulin Heavy Chain Class A Subclass 1 (IgHA1) for B-cell Receptor
gcaggagccggcaaggcacagggaggaagtgtggaggaacctcttggagaagccagctatgcttgccagaactcagccctttcagacatcaccgacccgcccttactcacatg

SEQ ID NO: 61 - DNA, 50 bp, Human
  Misc._Feature (1)..(50): Name/Key: 3′ Untranslated Region (3′ UTR) of the Immunoglobulin Heavy Chain Class A Subclass 1 (IgHA1)
gccgcccgcctgtccccacccctgaataaactccatgctcccccaagcag

SEQ ID NO: 62 - DNA, 144 bp, Human
  Misc._Feature (1)..(144): Name/Key: 3′ Untranslated Region (3′ UTR) of the Immunoglobulin Heavy Chain Class A Subclass 2 (IgHA2) for B-cell Receptor
gcgggagccggcaaggcacagggaggaagtgtggaggaacctcttggagaagccagctatgcttgccagaactcagccctttcagacatcaccgacccgcccttactcacgtggcttccaggtgcaataaagtggccccaagga

SEQ ID NO: 63 - DNA, 48 bp, Human
  Misc._Feature (1)..(48): Name/Key: 3′ Untranslated Region (3′ UTR) of the Immunoglobulin Heavy Chain Class A Subclass 2 (IgHA2)
gccgcccgcctgtccccacccctgaataaactccatgctcccccaagc

SEQ ID NO: 64 - DNA, 140 bp, Human
  Misc._Feature (1)..(140): Name/Key: 3′ Untranslated Region (3′ UTR) of the Immunoglobulin Kappa Light Chain (IgKappaL)
gttcccaactctaaccccacccacgggagcctggagctgcaggatcccaggggaggggtctctctccccatcccaagtcatccagcccttctccctgcactcatgaaaccccaataaatatcctcattgacaaccagaaa

SEQ ID NO: 65 - DNA, 140 bp, Human; identical to SEQ ID NO: 64
  Misc._Feature (1)..(140): Name/Key: 3′ Untranslated Region (3′ UTR) of the Immunoglobulin Lambda Light Chain (IgLambdaL)

SEQ ID NO: 66 - DNA, 111 bp, Human
  Misc._Feature (1)..(111): Name/Key: 3′ Untranslated Region (3′ UTR) of the Hemoglobulin Subunit Alpha 1 (HBA1)
gctggagcctcggtggccatgcttcttgccccttgggcctccccccagcccctcctccccttcctgcacccgtacccccgtggtctttgaataaagtctgagtgggcggca

SEQ ID NO: 67 - DNA, 110 bp, Human
  Misc._Feature (1)..(110): Name/Key: 3′ Untranslated Region (3′ UTR) of the Hemoglobulin Subunit Alpha 2 (HBA2)
gctggagcctcggtagccgttcctcctgcccgctgggcctcccaacgggccctcctcccctccttgcaccggcccttcctggtctttgaataaagtctgagtgggcagca

SEQ ID NO: 68 - DNA, 232 bp, Human
  Misc._Feature (1)..(232): Name/Key: 3′ Untranslated Region (3′ UTR) of the Fibrinogen-Gamma Chain
aaaattatgtctttttaatatggtttttgttttgttatatattcacaggctggagacgtttaaaagaccgtttcaaaagagatttacttttttaaaggactttatctgaacagagagatataatatttttcctattggacaatggacttgcaaagcttcacttcattttaagagcaaaagaccccatgttgaaaactccataacagttttatgctgatgataatttatctac

SEQ ID NO: 69 - DNA, 219 bp, Human
  Misc._Feature (1)..(219): Name/Key: 3′ Untranslated Region (3′ UTR) of the Fibrinogen-Alpha Chain
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactcaataaatacatgttaaatggatgaatgaa

SEQ ID NO: 70 - DNA, 414 bp, Human
  Misc._Feature (1)..(414): Name/Key: 3′ Untranslated Region (3′ UTR) of Albumin
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaacca

SEQ ID NO: 71 - RNA, 588 nt, Artificial
  Vector Element
  Misc._Feature (1)..(588): Single Stranded RNA form of Sequence 2
  Misc._Feature (1)..(588): Name/Key: Internal Ribosome Entry Site (IRES) mRNA
gccccucucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaauauggccacaacc

SEQ ID NO: 72 - RNA, 598 nt, Artificial
  Vector Element
  Misc._Feature (1)..(598): Single Stranded RNA Form of Sequence 3.
  Misc._Feature (1)..(598): Name/Key: Woodchuck Hepatitis Virus Posttranscriptional Regulatory Element (WPRE) mRNA
cgauaaucaaccucuggauuacaaaauuugugaaagauugacugguauucuuaacuauguugcuccuuuuacgcuauguggauacgcugcuuuaaugccuuuguaucaugcuauugcuucccguauggcuuucauuuucuccuccuuguauaaauccugguugcugucucuuuaugaggaguuguggcccguugucaggcaacguggcguggugugcacuguguuugcugacgcaacccccacugguuggggcauugccaccaccugucagcuccuuuccgggacuuucgcuuucccccucccuauugccacggcggaacucaucgccgccugccuugcccgcugcuggacaggggcucggcuguugggcacugacaauuccgugguguugucggggaagcugacguccuuuccauggcugcucgccuguguugccaccuggauucugcgcgggacguccuucugcuacgucccuucggcccucaauccagcggaccuuccuucccgcggccugcugccggcucugcggccucuuccgcgucuucgccuucgcccucagacgagucggaucucccuuugggccgccuccccgcaucgg

SEQ ID NO: 73 - RNA, 6 nt, Human
  Misc._Feature (1)..(6): Single Stranded RNA form of Sequence 21
  Misc._Feature (1)..(6): Name/Key: Kozak Sequence mRNA
gccacc

SEQ ID NO: 74 - RNA, 18 nt, Human
  Misc._Feature (1)..(18): Single Stranded RNA form of Sequence 42.
  Misc._Feature (1)..(18): Name/Key: 5′ Untranslated Region (5′ UTR) of J Chain
agaagaagugaagucaag

SEQ ID NO: 75 - RNA, 36 nt, Human
  Misc._Feature (1)..(36): Single Stranded RNA form of Sequence 43
  Misc._Feature (1)..(36): Name/Key: 5′ Untranslated Region (5′ UTR) of the Immunoglobulin Lambda Light Chain (Seq. ID 75) mRNA
ugagcgcagaaggcaggacucgggacaaucuucauc

SEQ ID NO: 76 - RNA, 40 nt, Human
  Misc._Feature (1)..(40): Single Stranded RNA form of Sequence 44
  Misc._Feature (1)..(40): Name/Key: 5′ Untranslated Region (5′ UTR) of the Immunoglobulin Lambda Light Chain (Seq. ID 76) mRNA
gcugcggguagagaagacaggacucaggacaaucuccagc

SEQ ID NO: 77 - RNA, 29 nt, Human
  Misc._Feature (1)..(29): Single Stranded RNA form of Sequence 45
  Misc._Feature (1)..(29): Name/Key: 5′ Untranslated Region (5′ UTR) of the Immunoglobulin Kappa Light Chain (Seq. ID 77) mRNA
gcaggaaucagucccacucaggacacagc

SEQ ID NO: 78 - RNA, 53 nt, Human
  Misc._Feature (1)..(53): Single Stranded RNA form of Sequence 46
  Misc._Feature (1)..(53): Name/Key: 5′ Untranslated Region (5′ UTR) of the Immunoglobulin Kappa Light Chain (Seq. ID 78) mRNA
aggcuggacacacuucaugcaggagucagacccugucaggacacagcauagac

SEQ ID NO: 79 - RNA, 63 nt, Human
  Misc._Feature (1)..(63): Name/Key: 5′ Untranslated Region (5′ UTR) of the Immunoglobulin Heavy Chain (Seq. ID 79) mRNA
gagagcaucacccagcaaccacaucuguccucuagagauccccugagagcuccguuccucacc

SEQ ID NO: 80 - RNA, 20 nt, Human
  Misc._Feature (1)..(20): Single Stranded RNA form of Sequence 48
  Misc._Feature (1)..(20): Name/Key: 5′ Untranslated Region (5′ UTR) of the Immunoglobulin Heavy Chain (Seq. ID 80) mRNA
agugacuccugugccccacc

SEQ ID NO: 81 - RNA, 37 nt, Human
  Misc._Feature (1)..(37): Single Stranded RNA form of Sequence 49
  Misc._Feature (1)..(37): Name/Key: 5′ Untranslated Region (5′ UTR) of the Hemoglobin Subunit Alpha 1 mRNA
acucuucugguccccacagacucagagagaacccacc

SEQ ID NO: 82 - RNA, 37 nt, Human; identical to SEQ ID NO: 81
  Misc._Feature (1)..(37): Single Stranded RNA form of Sequence 50
  Misc._Feature (1)..(37): Name/Key: 5′ Untranslated Region (5′ UTR) of the Hemoglobin Subunit Alpha 2 mRNA

SEQ ID NO: 83 - RNA, 47 nt, Human
  Misc._Feature (1)..(47): Single Stranded RNA form of Sequence 51
  Misc._Feature (1)..(47): Name/Key: 5′ Untranslated Region (5′ UTR) of the Fibrinogen-Gamma Chain mRNA
gacagugcugacacuacaaggcucggagcuccgggcacucagacauc

SEQ ID NO: 84 - RNA, 8 nt, Human
  Misc._Feature (1)..(8): Single Stranded RNA form of Sequence 52
  Misc._Feature (1)..(8): Name/Key: 5′ Untranslated Region (5′ UTR) of the Fibrinogen-Beta Chain mRNA
aagucuac

SEQ ID NO: 85 - RNA, 55 nt, Human
  Misc._Feature (1)..(55): Single Stranded RNA form of Sequence 53
  Misc._Feature (1)..(55): Name/Key: 5′ Untranslated Region (5′ UTR) of the Fibrinogen-Alpha Chain mRNA
aauccuuucuuucagcuggagugcuccucaggagccagccccacccuuagaaaag

SEQ ID NO: 86 - RNA, 41 nt, Human
  Misc._Feature (1)..(41): Single Stranded RNA form of Sequence 54
  Misc._Feature (1)..(41): Name/Key: 5′ Untranslated Region (5′ UTR) for Albumin mRNA
cuagcuuuucucuucugucaaccccacacgccuuuggcaca

SEQ ID NO: 87 - RNA, 788 nt, Human
  Misc._Feature (1)..(788): Single Stranded RNA form of Sequence 55
  Misc._Feature (1)..(788): Name/Key: 3′ Untranslated Region (3′ UTR) of J Chain mRNA
uuuaagucauugcugacugcauagcucuuuuucuugagaggcucuccauuuugauucagaaaguuagcauauuuauuaccaaugaauuugaaaccagggcuuuuuuuuuuuuuugggugauguaaaaccaacucccugccaccaaaauaauuaaaauagucacauuguuaucuuuauuagguaaucacuucuuaauuauauguucauacucuaaguaucaaaaucuuccaauuaucaugcucaccugaaagagguaugcucucuuaggaauacaguuucuagcauuaaacaaauaaacaaggggagaaaauaaaacucaaggacugaaaaucaggagguguaauaaaauguuccucgcauucccccccgcuuuuuuuuuuuuuuuugacuuugccuuggagagccagagcuuccgcauuuucuuuacuauucuuuuuaaaaaaaguuucacuguguagagaacauauaugcauaaacauaggucaauuauaugucuccauuagaaaaauaauaauuggaaaacauguucuagaacuaguuacaaaaauaauuuaaggugaaaucucuaauauuuauaaaaguagcaaaauaaaugcauaauuaaaauauauuuggacauaacagacuuggaagcagaugauacagacuucuuuuuuucauaaucagguuaguguaagaaauugccauuugaaacaauccauuuuguaacugaaccuuaugaaauauauguauuucaugguacguauucucuagcacagucugagcaauuaaauagauucauaagcaua

SEQ ID NO: 88 - RNA, 135 nt, Human
  Misc._Feature (1)..(135): Single Stranded RNA form of Sequence 56
  Misc._Feature (1)..(135): Name/Key: 3′ Untranslated Region (3′ UTR) of the Immunoglobulin Heavy Chain Class G Subclass 1 (IgHG1) mRNA
gugccacggccggcaagcccccgcuccccaggcucucggggucgcgcgaggaugcuuggcacguaccccguguacauacuucccaggcacccagcauggaaauaaagcacccagcgcuucccugggccccugcga

SEQ ID NO: 89 - RNA, 135 nt, Human; identical to SEQ ID NO: 88
  Misc._Feature (1)..(135): Single Stranded RNA form of Sequence 57
  Misc._Feature (1)..(135): Name/Key: 3′ Untranslated Region (3′ UTR) of the Immunoglobulin Heavy Chain Class G Subclass 2 (IgHG2) mRNA

SEQ ID NO: 90 - RNA, 135 nt, Human; identical to SEQ ID NO: 88
  Misc._Feature (1)..(135): Single Stranded RNA form of Sequence 58
  Misc._Feature (1)..(135): Name/Key: 3′ Untranslated Region (3′ UTR) of the Immunoglobulin Heavy Chain Class G Subclass 3 (IgHG3) mRNA

SEQ ID NO: 91 - RNA, 135 nt, Human; identical to SEQ ID NO: 88
  Misc._Feature (1)..(135): Single Stranded RNA form of Sequence 59
  Misc._Feature (1)..(135): Name/Key: 3′ Untranslated Region (3′ UTR) of the Immunoglobulin Heavy Chain Class G Subclass 4 (IgHG4) mRNA

SEQ ID NO: 92 - RNA, 113 nt, Human
  Misc._Feature (1)..(113): Single Stranded RNA form of Sequence 60
  Misc._Feature (1)..(113): Name/Key: 3′ Untranslated Region (3′ UTR) of the Immunoglobulin Heavy Chain Class A Subclass 1 (IgHA1) for B-cell Receptor mRNA
gcaggagccggcaaggcacagggaggaaguguggaggaaccucuuggagaagccagcuaugcuugccagaacucagcccuuucagacaucaccgacccgcccuuacucacaug

SEQ ID NO: 93 - RNA, 50 nt, Human
  Misc._Feature (1)..(50): Single Stranded RNA form of Sequence 61
  Misc._Feature (1)..(50): Name/Key: 3′ Untranslated Region (3′ UTR) of the Immunoglobulin Heavy Chain Class A Subclass 1 (IgHA1) mRNA
gccgcccgccuguccccaccccugaauaaacuccaugcucccccaagcag

SEQ ID NO: 94 - RNA, 144 nt, Human
  Misc._Feature (1)..(144): Single Stranded RNA form of Sequence 62
  Misc._Feature (1)..(144): Name/Key: 3′ Untranslated Region (3′ UTR) of the Immunoglobulin Heavy Chain Class A Subclass 2 (IgHA2) for B-cell Receptor mRNA
gcgggagccggcaaggcacagggaggaaguguggaggaaccucuuggagaagccagcuaugcuugccagaacucagcccuuucagacaucaccgacccgcccuuacucacguggcuuccaggugcaauaaaguggccccaagga

SEQ ID NO: 95 - RNA, 48 nt, Human
  Misc._Feature (1)..(48): Single Stranded RNA form of Sequence 63
  Misc._Feature (1)..(48): Name/Key: 3′ Untranslated Region (3′ UTR) of the Immunoglobulin Heavy Chain Class A Subclass 2 (IgHA2) mRNA
gccgcccgccuguccccaccccugaauaaacuccaugcucccccaagc

SEQ ID NO: 96 - RNA, 140 nt, Human
  Misc._Feature (1)..(140): Single Stranded RNA form of Sequence 64
  Misc._Feature (1)..(140): Name/Key: 3′ Untranslated Region (3′ UTR) of the Immunoglobulin Kappa Light Chain (IgKappaL) mRNA
guucccaacucuaaccccacccacgggagccuggagcugcaggaucccaggggaggggucucucuccccaucccaagucauccagcccuucucccugcacucaugaaaccccaauaaauauccucauugacaaccagaaa

SEQ ID NO: 97 - RNA, 140 nt, Human; identical to SEQ ID NO: 96
  Misc._Feature (1)..(140): Single Stranded RNA form of Sequence 65
  Misc._Feature (1)..(140): Name/Key: 3′ Untranslated Region (3′ UTR) of the Immunoglobulin Lambda Light Chain (IgLambdaL) mRNA

SEQ ID NO: 98 - RNA, 111 nt, Human
  Misc._Feature (1)..(111): Single Stranded RNA form of Sequence 66
  Misc._Feature (1)..(111): Name/Key: 3′ Untranslated Region (3′ UTR) of the Hemoglobulin Subunit Alpha 1 (HBA1) mRNA
gcuggagccucgguggccaugcuucuugccccuugggccuccccccagccccuccuccccuuccugcacccguacccccguggucuuugaauaaagucugagugggcggca

SEQ ID NO: 99 - RNA, 110 nt, Human
  Misc._Feature (1)..(110): Single Stranded RNA form of Sequence 67
  Misc._Feature (1)..(110): Name/Key: 3′ Untranslated Region (3′ UTR) of the Hemoglobulin Subunit Alpha 2 (HBA2) mRNA
gcuggagccucgguagccguuccuccugcccgcugggccucccaacgggcccuccuccccuccuugcaccggcccuuccuggucuuugaauaaagucugagugggcagca

SEQ ID NO: 100 - RNA, 232 nt, Human
  Misc._Feature (1)..(232): Single Stranded RNA form of Sequence 68
  Misc._Feature (1)..(232): Name/Key: 3′ Untranslated Region (3′ UTR) of the Fibrinogen-Gamma Chain mRNA
aaaauuaugucuuuuuaauaugguuuuuguuuuguuauauauucacaggcuggagacguuuaaaagaccguuucaaaagagauuuacuuuuuuaaaggacuuuaucugaacagagagauauaauauuuuuccuauuggacaauggacuugcaaagcuucacuucauuuuaagagcaaaagaccccauguugaaaacuccauaacaguuuuaugcugaugauaauuuaucuac

SEQ ID NO: 101 - RNA, 219 nt, Human
  Misc._Feature (1)..(219): Single Stranded RNA form of Sequence 69
  Misc._Feature (1)..(219): Name/Key: 3′ Untranslated Region (3′ UTR) of the Fibrinogen-Alpha Chain mRNA
acuaaguuaaauauuucugcacaguguucccauggccccuugcauuuccuucuuaacucucuguuacacgucauugaaacuacacuuuuuuggucuguuuuugugcuagacuguaaguuccuugggggcagggccuuugucugucucaucucuguauucccaaaugccuaacaguacagagccaugacucaauaaauacauguuaaauggaugaaugaa

SEQ ID NO: 102 - RNA, 414 nt, Human
  Misc._Feature (1)..(414): Single Stranded RNA form of Sequence 70
  Misc._Feature (1)..(414): Name/Key: 3′ Untranslated Region (3′ UTR) of Albumin mRNA
caucacauuuaaaagcaucucagccuaccaugagaauaagagaaagaaaaugaagaucaaaagcuuauucaucuguuuuucuuuuucguugguguaaagccaacacccugucuaaaaaacauaaauuucuuuaaucauuuugccucuuuucucugugcuucaauuaauaaaaaauggaaagaaucuaauagagugguacagcacuguuauuuuucaaagauguguugcuauccugaaaauucuguagguucuguggaaguuccaguguucucucuuauuccacuucgguagaggauuucuaguuucuugugggcuaauuaaauaaaucauuaauacucuucuaaguuauggauuauaaacauucaaaauaauauuuugacauuaugauaauucugaauaaaagaacaaaaacca

SEQ ID NO: 103 - RNA, 117 nt, Artificial
  Vector Element
  Misc._Feature (1)..(117): Single Stranded RNA form of Sequence 9
  Misc._Feature (1)..(117): Name/Key: Intron I117 5′ Untranslated Region (5′ UTR) mRNA
guaagugauaucuacuagauuuaucaaaaagaguguugacuugugagcgcucacaauugauacuuagauucaucgagagggacacgucgacuacuaaccuucuucucuuuccuacag

SEQ ID NO: 104 - DNA, 1179 bp, Human
  Misc._Feature (1)..(1179): Name/Key: Eukaryotic Translation Elongation Factor 1 alpha (EF-1alpha) Promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 105 - DNA, 904 bp, Artificial
  Vector Element
  Misc._Feature (1)..(904): Name/Key: Feek Promoter  - Human Derived Promoter for Immunoglobulin Genes Including in the 5′ to 3′ direction a 3′ Kappa Light Chain Enhancer, a Matrix association region, an Intronic Enhancer followed by a  Kappa Light Chain Promoter.
taaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctdgccactgagggcttgatcaattcactttgaatttggcattaaataccattaaggtatattaactgattttaaaataagatatattcgtgaccatgtttttaactttcaaaaatgtagctgccagtgtdtgattttatttcagttgtacaaaatatctaaacctatagcaatgtgattaataaaaacttaaacatattttccagtaccttaattctgtdataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagct

SEQ ID NO: 106 - DNA, 201 bp, Human
  Misc._Feature (1)..(201): Name/Key: Tumor Necrosis Factor Alpha (TNF alpha) Promoter (-200 to Transcription Start Site (TSS))
ttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagcca

SEQ ID NO: 107 - DNA, 377 bp, Human
  Misc._Feature (1)..(377): Name/Key: Tumor Necrosis Factor Alpha (TNF alpha)  Promoter (-200 position of TSS and 5′ UTR)
ttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacacc

SEQ ID NO: 108 - DNA, 397 bp, Human
  Misc._Feature (1)..(397): Name/Key: Tumor Necrosis Factor Alpha (TNF alpha) Promoter (-400 to TSS)
tttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacaccca

SEQ ID NO: 109 - skipped

SEQ ID NO: 110 - DNA, 545 bp, Artificial
  Vector Element
  Misc._Feature (1)..(545): Name/Key: Perforin Promoter Trunacted includes the 5′ UTR
ggaggctgcagtttctagaagagggtggggacactgcggagagaagatggggccagattccgagaagacagcataagcccctgttcctgtaagagcagggacggaagcagggacataaacgcaagggatgagccccaaagtgtgacccatgagacatgatgtcacatgtggtctggtgttcatcaacaccagggccgagtctcaaagtcctcagcgccccgccctcctccgcctgtgtgccctgagtccccgagccccagcagctctactcggcagatgagcctctggccctgctgctcgcttcctgagggctgtcagtggggagccggatgagggctgaggacagggtgggtgcttgtgggaggggagagcacaaaggacctgtgaccacagctgggggcggggcaggaagtagaagtgatgtgagtggtggctggtgcaaggagccacagtgggctgcctggggggctgatgccaccattccaggagcctcggtgaagagaggatatccatctgtgtagccgcttctctatacgggattccag

SEQ ID NO: 111 - DNA, 611 bp, Human
  Misc._Feature (1)..(611): Name/Key: Interferon Gamma (IFN-gamma) Promoter (-546 to +64 Relative to TSS) partially inclusive of 5′ UTR
tgatctagagcaatttgaaacttgtggtagatattttactaaccaactctgatgaaggacttcctcaccaaattgttcttttaaccgcattctttccttgctttctggtcatttgcaagaaaaattttaaaaggctgcccctttgtaaaggtttgagaggccctagaatttcgtttttcacttgttcccaaccacaagcaaatgatcaatgtgctttgtgaatgaagagtcaacattttaccagggcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcagttaagtcctttggacct

SEQ ID NO: 112 - DNA, 671 bp, Human
  Misc._Feature (1)..(671): Name/Key: Interferon Gamma (IFN-gamma) Promoter (-546 to  +125 Relative to TSS)  fully inclusive of 5′ UTR
tgatctagagcaatttgaaacttgtggtagatattttactaaccaactctgatgaaggacttcctcaccaaattgttcttttaaccgcattctttccttgctttctggtcatttgcaagaaaaattttaaaaggctgcccctttgtaaaggtttgagaggccctagaatttcgtttttcacttgttcccaaccacaagcaaatgatcaatgtgctttgtgaatgaagagtcaacattttaccagggcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcagttaagtcctttggacctgatcagcttgatacaagaactactgatttcaacttctttggcttaattctctcggaaacg

SEQ ID NO: 113 - DNA, 230 bp, Human
  Misc._Feature (1)..(230): Name/Key: Granzyme B Promoter fully inclusive of the 5′ UTR
tgggtgatgatgtggtcactgcatgattctcacacaagcacccagaggacgtcatcaggcagaggcagtgggggtgggcagcatttacagaaaatctgtgatgagacaccacaaaaccagaggggaacatgaagtcactgagcctgctccacctctttcctctcccaagagctaaaagagagcaaggaggaaacaacagcagctccaaccagggcagccttcctgagaag

SEQ ID NO: 114 - DNA, 30 bp, Human
  Misc._Feature (1)..(30): Name/Key: Granzyme B  5′ UTR
agctccaaccagggcagccttcctgagaag

SEQ ID NO: 115 - DNA, 346 bp, Human
  Misc._Feature (1)..(346): Name/Key: Interleukin-2 (IL-2) Promoter Truncated (-346 to TSS)
agtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagtt

SEQ ID NO: 116 - DNA, 541 bp, Human
  Misc._Feature (1)..(541): Name/Key: Interleukin-2 (IL-2) Full Promoter (-540 to TSS)
agtatttctctagctgacatgtaagaagcaatctatcttattgtatgcaattagctcattgtgtggataaaaaggtaaaaccattctgaaacaggaaaccaatacacttcctgttttatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagtt

SEQ ID NO: 117 - DNA, 921 bp, Artificial
  Vector Element
  Misc._Feature (1)..(921): Name/Key: Interleukin-2 (IL-2) Full Promoter (-540 to TSS) with upstream CMV Immediate Early Enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgagtatttctctagctgacatgtaagaagcaatctatcttattgtatgcaattagctcattgtgtggataaaaaggtaaaaccattctgaaacaggaaaccaatacacttcctgttttatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagtt

SEQ ID NO: 118 - protein, 20 aa, Artificial
  Flexible Oligopeptide Linker
  Misc._Feature (1)..(20): Name/Key: Artificail Linker 4 Repeat of GGGGS
GGGGSGGGGSGGGGSGGGGS

SEQ ID NO: 119 - protein, 25 aa, Artificial
  Flexible Oligopeptide Linker
  Misc._Feature (1)..(25): Name/Key: Artificial Linker 5 Repeat of GGGGS
GGGGSGGGGSGGGGSGGGGSGGGGS

SEQ ID NO: 120 - protein, 30 aa, Artificial
  Flexible Polypeptide Linker
  Misc._Feature (1)..(30): Name/Key: Artificial Linker 6 Repeat of GGGGS
GGGGSGGGGSGGGGSGGGGSGGGGSGGGGS

SEQ ID NO: 121 - protein, 35 aa, Artificial
  Flexible Polypeptide Linker
  Misc._Feature (1)..(35): Name/Key: Artificial Linker 7 Repeat of GGGGS
GGGGSGGGGSGGGGSGGGGSGGGGSGGGGSGGGGS

SEQ ID NO: 122 - protein, 18 aa, Artificial
  Oligopeptide Linker
  Misc._Feature (1)..(18): Name/Key: Artificial Whitlow Linker
GSTSGSGKPGSGEGSTKG

SEQ ID NO: 123 - protein, 21 aa, Human
  Misc._Feature (1)..(21): Name/Key: Human Cluster of Differentiation 8 alpha (CD8 alpha) Signal Peptide
MALPVTALLLPLALLLHAARP

SEQ ID NO: 124 - protein, 235 aa, Human
  Misc._Feature (1)..(235): Name/Key: Human Cluster of Differentiation 8 alpha (CD8 alpha) Protein with Signal Peptide
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 125 - DNA, 708 bp, Human
  Misc._Feature (1)..(708): Name/Key: Human Derived Coding DNA of Cluster of Differentiation 8 alpha (CD8 alpha) encoding for Protein with Signal Peptide
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 126 - skipped

SEQ ID NO: 127 - protein, 220 aa, Human
  Misc._Feature (1)..(220): Name/Key: Human T-cell-specific surface glycoprotein CD28 (CD28) Protein with Signal Peptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 128 - DNA, 663 bp, Human
  Misc._Feature (1)..(663): Name/Key: Human Derived Coding DNA of T-cell-specific surface glycoprotein CD28 (CD28) encoding for Protein with Signal Peptide
atgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctga

SEQ ID NO: 129 - protein, 39 aa, Human
IEVMYPPPYLDNEKSNGTIIHVKGKHLCPSPLFPGPSKP

SEQ ID NO: 130 - protein, 27 aa, Human
  Misc._Feature (1)..(27): Name/Key: Human Derived T-cell-specific surface glycoprotein CD28 (CD28)  transmembrane Domain
FWVLVVVGGVLACYSLLVTVAFIIFWV

SEQ ID NO: 131 - protein, 41 aa, Human
  Misc._Feature (1)..(41): Name/Key: Human Derived T-cell-specific surface glycoprotein CD28 (CD28)  co-stimulatory signaling  Sequence
RSKRSRLLHSDYMNMTPRRPGPTRKHYQPYAPPRDFAAYRS

SEQ ID NO: 132 - protein, 107 aa, Human
  Misc._Feature (1)..(107): Name/Key: Human Derived T-cell-specific surface glycoprotein CD28 (CD28)  Hinge Transmembrane domain and co-stimulatory signaling sequence
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 133 - skipped

SEQ ID NO: 134 - protein, 22 aa, Human
  Misc._Feature (1)..(22): Name/Key: Human Derived Cluster of Differentiation 8 alpha (CD8 alpha)  transmembrane Domain
IWAPLAGTCGVLLLSLVITLYC

SEQ ID NO: 135 - protein, 47 aa, Human
  Misc._Feature (1)..(47): Name/Key: Human Derived Cluster of Differentiation 8 alpha (CD8 alpha) hinge - Partial Sequence
TTTPAPRPPTPAPTIASQPLSLRPEACRPAAGGAVHTRGLDFACDIY

SEQ ID NO: 136 - protein, 23 aa, Human
  Misc._Feature (1)..(23): Name/Key: Human Cluster of Differentiation 137 (4-1BB)  Signal Peptide
MGNSCYNIVATLLLVLNFERTRS

SEQ ID NO: 137 - protein, 255 aa, Human
  Misc._Feature (1)..(255): Name/Key: Human Cluster of Differentiation 137 (CD137) referred to as 4-1BB Ligand (4-1BB)  Protein with Signal Peptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 138 - DNA, 768 bp, Human
  Misc._Feature (1)..(768): Name/Key: Human Derived Coding DNA for Cluster of Differentiation 137 (CD137) referred to as 4-1BB Ligand (4-1BB) encoding for Protein with Signal Peptide
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 139 - protein, 42 aa, Human
  Misc._Feature (1)..(42): Name/Key: Human Derived Cluster of Differentiation 137 (CD137) referred to as 4-1BB Ligand (4-1BB) co-stimulatory Sequence
KRGRKKLLYIFKQPFMRPVQTTQEEDGCSCRFPEEEEGGCEL

SEQ ID NO: 140 - protein, 21 aa, Human
  Misc._Feature (1)..(21): Name/Key: Human Cluster of Differentiation 3 Zeta Chain (CD3 Zeta) Signal Peptide
MKWKALFTAAILQAQLPITEA

SEQ ID NO: 141 - protein, 164 aa, Human
  Misc._Feature (1)..(164): Name/Key: Human Cluster of Differentiation 3 Zeta Chain (CD3 Zeta) Protein with Signal Peptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 142 - DNA, 495 bp, Human
  Misc._Feature (1)..(495): Name/Key: Human Derived Coding DNA of Cluster of Differentiation 3 Zeta  (CD3 Zeta) encoding for Protein with Signal Peptide
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 143 - protein, 112 aa, Human
  Misc._Feature (1)..(112): Name/Key: Human Derived Cluster of Differentiation 3 Zeta Chain (CD3 Zeta)  Signaling Domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR